(19) United States
(12) Patent Application Publication
(10) Pub. No.: US 2023/0295110 A1
(43) Pub. Date: Sep. 21, 2023

(54) PHD INHIBITOR COMPOUNDS, COMPOSITIONS, AND METHODS OF USE

(71) Applicant: Akebia Therapeutics, Inc., Cambridge, MA (US)

(72) Inventors: Thomas P. BLAISDELL, Cambridge, MA (US); Senkara Rao ALLU, Cambridge, MA (US); Paul E. FLEMING, Cambridge, MA (US)

(73) Assignee: Akebia Therapeutics, Inc., Cambridge, MA (US)

(21) Appl. No.: 18/041,490

(22) PCT Filed: Aug. 13, 2021
(86) PCT No.: PCT/US2021/045896

§ 371 Date: Feb. 13, 2023

Related U.S. Application Data
(60) Provisional application No. 63/065,642, filed on Aug. 14, 2020.

(51) Int. Cl. C07D 401/04; C07D 213/81; C07D 413/04; C07D 401/14; C07D 417/04; C07D 405/12; C07D 405/14

(52) U.S. Cl. CPC C07D 401/04; C07D 213/81; C07D 413/04; C07D 401/14; C07D 417/04; C07D 405/12; C07D 405/14

(57) ABSTRACT

The present invention provides, in part, novel small molecule inhibitors of PHD, having a structure according to Formula (I), and sub-formulas thereof: or a pharmaceutically acceptable salt thereof. The compounds provided herein can be useful for treatment of diseases including heart (e.g. ischemic heart disease, congestive heart failure, and valvular heart disease), lung (e.g., lung inflammation, pneumonia, acute lung injury, pulmonary hypertension, pulmonary fibrosis, and chronic obstructive pulmonary disease), respiratory (e.g., respiratory infection, acute respiratory distress syndrome), liver (e.g. acute liver failure and liver fibrosis and cirrhosis), and kidney (e.g. acute kidney injury and chronic kidney disease) disease, inflammatory bowel disease (IBD), ischemic reperfusion injury (e.g., stroke), and retinopathy of prematurity (ROP).

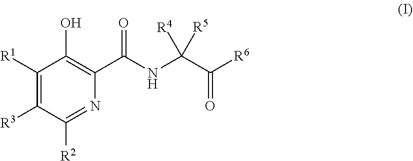

### CROSS-REFERENCE TO RELATED APPLICATIONS

The present application claims benefit of U.S. Provisional Application No. 63/065,642, filed Aug. 14, 2020, which is hereby incorporated by reference in its entirety.

### BACKGROUND

Hypoxia is a condition or state in which the supply of oxygen is insufficient for normal life function, for example, where there is low arterial oxygen supply. Hypoxia can lead to functional impairment of cells and structural tissue damage. The activation of cellular defense mechanisms during hypoxia is mediated by HIF (Hypoxia-inducible factor) protein. In response to hypoxic conditions, levels of HIFα are elevated in most cells because of a decrease in HIFα prolyl hydroxylation. Prolyl hydroxylation of HIFα is accomplished by a family of proteins variously termed the prolyl hydroxylase domain-containing proteins (PHD1, 2, and 3), also known as HIF prolyl hydroxylases (HPH-3, 2, and 1) or EGLN-2, 1, and 3. The PHD proteins are oxygen sensors and regulate the stability of HIF in an oxygen dependent manner. The three PHD isoforms function differently in their regulation of HIF and may have other non-HIF related regulatory roles.

In fact, many studies demonstrate that stabilization of HIF can dampen tissue inflammation and promote tissue repair. Accordingly, compounds that can inhibit the activity of PHD proteins may be particularly beneficial new therapies (Lee et al. (2019) Exp. Mol. Med. 51:68)

Described herein are novel small molecule PHD inhibitors that have utility for the treatment of disease including heart (e.g., ischemic heart disease, congestive heart failure, and valvular heart disease), lung (e.g., lung inflammation, pneumonia, acute lung injury, pulmonary hypertension, pulmonary fibrosis, and chronic obstructive pulmonary disease), respiratory (e.g., respiratory infection, acute respiratory distress syndrome), liver (e.g. acute liver failure and liver fibrosis and cirrhosis), and kidney (e.g. acute kidney injury and chronic kidney disease) disease, inflammatory bowel disease (IBD), ischemic reperfusion injury (e.g., stroke), and retinopathy of prematurity (ROP).

### SUMMARY

The present invention provides, among other things, novel small molecule inhibitors of PHD and have utility for the treatment of diseases, including but not limited to heart (e.g. ischemic heart disease, congestive heart failure, and valvular heart disease), lung (e.g., lung inflammation, pneumonia, acute lung injury, pulmonary hypertension, pulmonary fibrosis, and chronic obstructive pulmonary disease), respiratory (e.g., respiratory infection, acute respiratory distress syndrome), liver (e.g. acute liver failure and liver fibrosis and cirrhosis), and kidney (e.g. acute kidney injury and chronic kidney disease) disease, inflammatory bowel disease (IBD), ischemic reperfusion injury (e.g., stroke), and retinopathy of prematurity (ROP).

In an aspect, provided herein are compounds having a structure according to Formula (I)

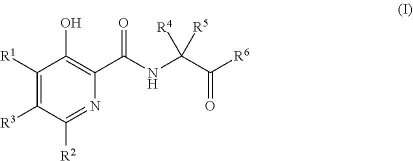

or a pharmaceutically acceptable salt thereof, wherein:

R¹ is optionally substituted C₁₋₃ alkyl, optionally substituted C₃₋₆ cycloalkyl, or optionally substituted 3- to 6-membered heterocycloalkyl;

R² is hydrogen, optionally substituted C₁₋₃ alkyl, halogen, CN, or optionally substituted cycloalkyl;

R³ is hydrogen, optionally substituted aryl, optionally substituted heteroaryl, optionally substituted cycloalkyl, optionally substituted heterocycloalkyl, carbonyl, ether, thioether, optionally substituted arylsulfonyl, optionally substituted heteroarylsulfonyl, optionally substituted arylalkyl, optionally substituted alkynyl, or optionally substituted heteroalkynyl;

R⁴ and R⁵ are independently hydrogen, optionally substituted C₁₋₃ alkyl, or R⁴ and

R⁵ together with the carbon to which they are attached form an optionally substituted cycloalkyl or heterocycloalkyl; and

R⁶ is OH or ester (e.g., OR¹⁸ as described herein).

In embodiments, a compound has a structure according to Formula (I)

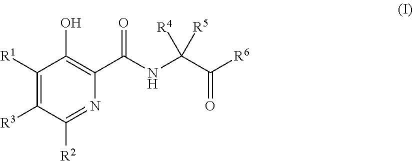

or a pharmaceutically acceptable salt thereof, wherein:

R¹ is C₁₋₃ alkyl optionally substituted with OR⁷, halogen, or aryl, which is optionally substituted with halogen, and wherein R⁷ is C₁₋₃ alkyl optionally substituted with aryl, or R¹ is C₃₋₆ cycloalkyl or 3- to 6-membered heterocycloalkyl

R² is hydrogen, halogen, CN, or C₁₋₃ alkyl optionally substituted with one or more halogens;

R³ is selected from the group consisting of:
hydrogen;

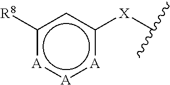

wherein X is a covalent bond, O, S, SO₂, C₁₋₄ alkylene, C₂₋₄ alkynylene, or C₂₋₄ heteroalkynylene; each A is independently N or CR⁹, R⁸ and R⁹ are independently hydrogen, halogen, OR¹⁰, or C₁₋₃ alkyl optionally substituted with one or more halogens, and R¹⁰ is C₁₋₃ alkyl or aryl;

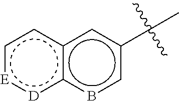

wherein B is N or CR¹¹, D is N, NH, or CR¹¹, E is N, CR¹¹, or CHR¹², and R¹¹ and R¹² are independently hydrogen or C₁₋₃ alkyl, and wherein the dashed circle represents the presence or absence of a conjugated system;

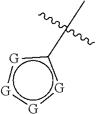

wherein each G is independently N, NH, NR¹³, or CR¹⁴; R¹³ is C₃₋₆ cycloalkyl, 3- to 6-membered heterocycloalkyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens, and R¹⁴ is hydrogen, halogen, C₃₋₆ cycloalkyl, 3- to 6-membered heterocycloalkyl, or C₁₋₃ alkyl;

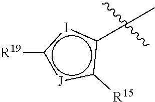

wherein I is O, S, or CH, J is N or CH, R¹⁵ is hydrogen, C₃₋₆ cycloalkyl, 3- to 6-membered heterocycloalkyl, or C₁₋₃ alkyl, and R¹⁹ is hydrogen, C₃₋₆ cycloalkyl, 3- to 6-membered heterocycloalkyl, or aryl;
OR¹⁶ wherein R¹⁶ is aryl;

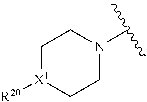

wherein X¹ is N or CH, and R²⁰ is optionally substituted aryl; and
COR¹⁷ wherein R¹⁷ is aryl;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl; and

R⁶ is OH or OR¹⁸, wherein R¹⁸ is C₁₋₆ alkyl.

In embodiments, a compound of Formula (I) has a structure according to Formula (II)

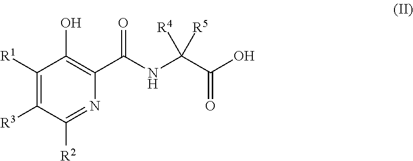

or a pharmaceutically acceptable salt thereof, wherein:

R¹ is C₁₋₃ alkyl optionally substituted with OR⁷, halogen, or aryl, which is optionally substituted with halogen, and wherein R⁷ is C₁₋₃ alkyl optionally substituted with aryl, or R¹ is C₃₋₆ cycloalkyl or 3- to 6-membered heterocycloalkyl;

R² is hydrogen, halogen, CN, or C₁₋₃ alkyl optionally substituted with one or more halogens;

R³ is selected from the group consisting of:
hydrogen;

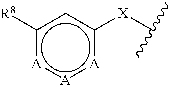

wherein X is a covalent bond, O, S, S02, C₁₋₄ alkylene, C₂₋₄ alkynylene, or C₂₋₄ heteroalkynylene; each A is independently N or CR⁹, R⁸ and R⁹ are independently hydrogen, halogen, OR¹⁰, or C₁₋₃ alkyl optionally substituted with one or more halogens, and R¹⁰ is C₁₋₃ alkyl or aryl;

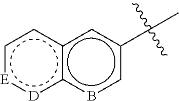

wherein B is N or CR¹¹, D is N, NH, or CR¹¹, E is N, CR¹¹, or CHR¹², and R¹¹ and R¹² are independently hydrogen or C₁₋₃ alkyl, and wherein the dashed circle represents the presence or absence of a conjugated system;

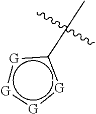

wherein each G is independently N, NR¹³, CR¹⁴, R¹³ is C₃₋₆ cycloalkyl, 3- to 6-membered heterocycloalkyl, aryl optionally substituted with one or more halogens, aryl optionally substituted with one or more optionally substituted C₁_3 alkyls, heteroaryl, heterocycloalkyl optionally substituted with t-butyloxycarbonyl, C₁₋₄ alkyl optionally substituted with aryl, which is optionally substituted with one or more halogens, and R¹⁴ is hydrogen, halogen, C₃₋₆ cycloalkyl, 3- to 6-membered heterocycloalkyl, or C₁₋₃ alkyl;

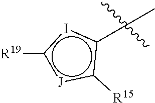

wherein I is O, S, or CH, J is N or CH, R¹⁵ is hydrogen, C₃₋₆ cycloalkyl, 3- to 6-membered heterocycloalkyl, or C₁₋₃ alkyl, and R¹⁹ is hydrogen, C₃₋₆ cycloalkyl, 3- to 6-membered heterocycloalkyl, or aryl;
OR¹⁶ wherein R¹⁶ is aryl;

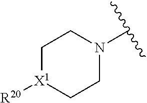

wherein X¹ is N or CH, and R²⁰ is optionally substituted aryl; and
COR¹⁷ wherein R¹⁷ is aryl;

and

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl.

In embodiments, R¹ is optionally substituted C₁₋₃ alkyl; and/or

R³ is hydrogen, optionally substituted aryl, optionally substituted heteroaryl, optionally substituted cycloalkyl, optionally substituted heterocycloalkyl, carbonyl, or ether.

In embodiments, R¹ is C₁₋₃ alkyl optionally substituted with OR⁷ or aryl, which is optionally substituted with halogen, and wherein R⁷ is C₁₋₃ alkyl optionally substituted with aryl; and/or

R³ is selected from the group consisting of: hydrogen,

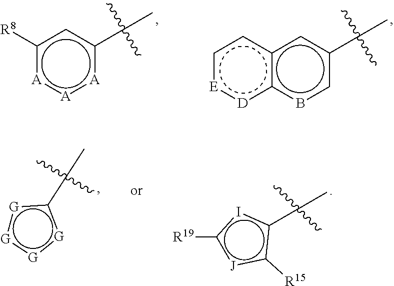

In embodiments, R³ is

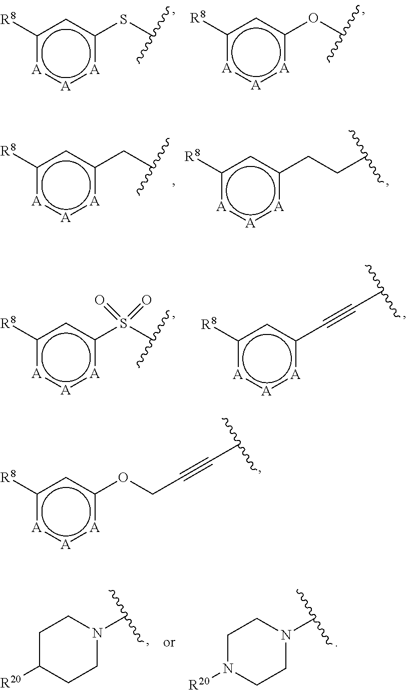

In embodiments, R¹ is unsubstituted C₁₋₃ alkyl, R² is hydrogen, R⁴ and R⁵ are each hydrogen, and R⁶ is OH.

In embodiments, each R¹ and R² is unsubstituted C₁₋₃ alkyl, R⁴ and R⁵ are each hydrogen, and R⁶ is OH.

In embodiments, R² is unsubstituted C₁₋₃ alkyl, R³ is hydrogen, R⁴ and R⁵ are each hydrogen, and R⁶ is OH.

In embodiments, a compound has a structure according to Formula (III)

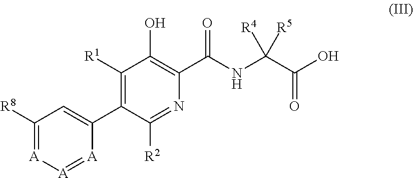

or a pharmaceutically acceptable salt thereof, wherein

each A is independently N or CR⁹;

R¹ is C₁₋₃ alkyl optionally substituted with OR⁷, halogen, or aryl, which is optionally substituted with halogen, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R⁷ is C₁₋₃ alkyl optionally substituted with aryl;

R⁸ and R⁹ are independently hydrogen, halogen, OR¹⁰, or C₁₋₃ alkyl optionally substituted with one or more halogens; and

R¹⁰ is C₁₋₃ alkyl or aryl.

In embodiments, a compound has a structure according to Formula (IV)

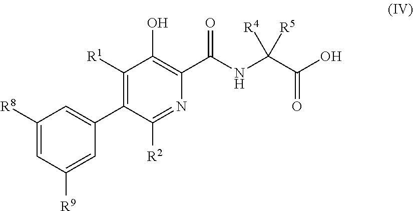

or a pharmaceutically acceptable salt thereof, wherein

R¹ is C₁₋₃ alkyl optionally substituted with OR⁷, halogen, or aryl, which is optionally substituted with halogen, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R⁷ is C₁₋₃ alkyl optionally substituted with aryl;

R⁸ and R⁹ are independently hydrogen, halogen, OR¹⁰, or C₁₋₃ alkyl optionally substituted with one or more halogens; and

R¹⁰ is C₁₋₃ alkyl or aryl.

In embodiments, a compound has a structure according to Formula (V)

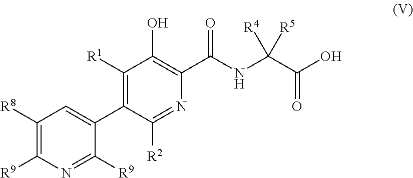

or a pharmaceutically acceptable salt thereof, wherein:

R¹ is C₁₋₃ alkyl optionally substituted with OR⁷, halogen, or aryl, which is optionally substituted with halogen, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R⁷ is C₁₋₃ alkyl optionally substituted with aryl;

R⁸ and each R⁹ are independently hydrogen, halogen, OR¹⁰, or C₁₋₃ alkyl optionally substituted with one or more halogens; and

R¹⁰ is C₁₋₃ alkyl or aryl.

In embodiments, a compound has a structure according to Formula (VI)

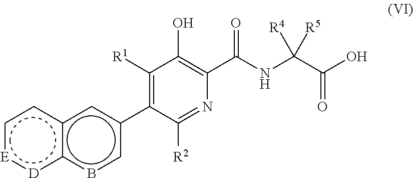

or a pharmaceutically acceptable salt thereof, wherein

B is N or CR¹¹;

D is N, NH, or CR¹¹;

E is N, CR¹¹, or CHR¹²;

R¹ is C₁₋₃ alkyl optionally substituted with OR⁷, halogen, or aryl, which is optionally substituted with halogen, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R⁷ is C₁₋₃ alkyl optionally substituted with aryl;

R¹¹ and R¹² are independently hydrogen or C₁₋₃ alkyl; and

wherein the dashed circle represents the presence or absence of a conjugated system.

In embodiments, a compound has a structure according to Formula (VII)

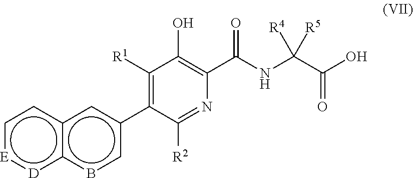

or a pharmaceutically acceptable salt thereof.

In embodiments, a compound has a structure according to Formula (VIII)

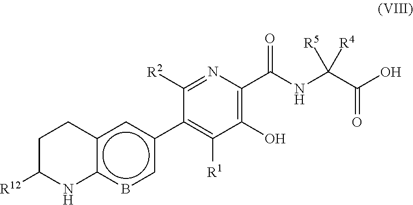

or a pharmaceutically acceptable salt thereof, wherein:

B is N or CR¹¹;

R¹ is C₁₋₃ alkyl optionally substituted with OR⁷, halogen, or aryl, which is optionally substituted with halogen, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R⁷ is C₁₋₃ alkyl optionally substituted with aryl; and

R¹² is hydrogen or C₁₋₃ alkyl.

In embodiments, a compound has a structure according to Formula (IX)

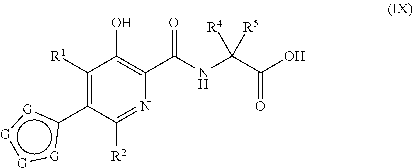

or a pharmaceutically acceptable salt thereof, wherein:

each G is independently N, NH, NR¹³ or CR¹⁴:

R¹ is C₁₋₃ alkyl optionally substituted with OR⁷, halogen, or aryl, which is optionally substituted with halogen, and wherein R⁷ is C₁₋₃ alkyl optionally substituted with aryl, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens; and

R¹⁴ is hydrogen, halogen, cyclopropyl, or C₁₋₃ alkyl.

In embodiments, a compound has a structure according to Formula (X)

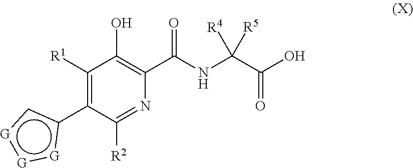

or a pharmaceutically acceptable salt thereof, wherein:

each G is independently N, NR¹³, or CR¹⁴;

R¹ is C₁₋₃ alkyl optionally substituted with OR⁷, halogen, or aryl, which is optionally substituted with halogen, and wherein R⁷ is C₁₋₃ alkyl optionally substituted with aryl, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens; and

R¹⁴ is hydrogen, halogen, cyclopropyl, or C₁₋₃ alkyl.

In embodiments, a compound has a structure according to Formula (XI)

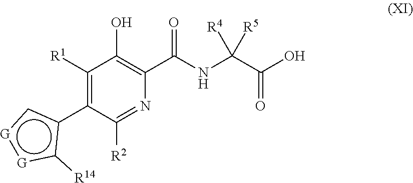

or a pharmaceutically acceptable salt thereof, wherein

each G is independently N or NR³;

R¹ is C₁₋₃ alkyl optionally substituted with OR⁷, halogen, or aryl, which is optionally substituted with halogen, and wherein R⁷ is C₁₋₃ alkyl optionally substituted with aryl, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R¹³ is cyclopropyl, heteroaryl, aryl optionally substituted with one or more halogens, aryl optionally substituted with one or more optionally substituted C₁₋₃ alkyls, heterocycloalkyl optionally substituted with t-butyloxycarbonyl, C₁₋₄ alkyl optionally substituted with aryl, which is optionally substituted with one or more halogens; and

R¹⁴ is hydrogen, halogen, cyclopropyl, or C₁₋₃ alkyl.

In embodiments, a compound has a structure according to Formula (XIIa) or Formula (XIIb)

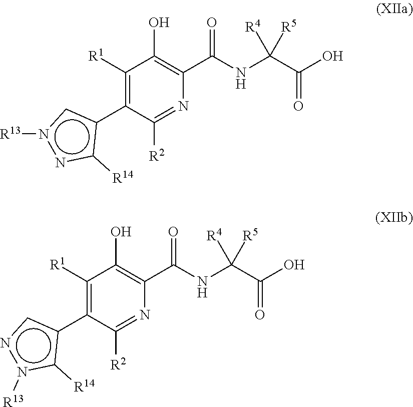

or a pharmaceutically acceptable salt thereof, wherein:

R¹ is C₁₋₃ alkyl optionally substituted with OR⁷, halogen, or aryl, which is optionally substituted with halogen, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R⁷ is C₁₋₃ alkyl optionally substituted with aryl;

R¹³ is cyclopropyl, heteroaryl, aryl optionally substituted with one or more halogens, aryl optionally substituted with one or more optionally substituted C₁₋₃ alkyls, heterocycloalkyl optionally substituted with t-butyloxycarbonyl, C₁₋₄ alkyl optionally substituted with aryl, which is optionally substituted with one or more halogens; and

R¹⁴ is hydrogen, halogen, cyclopropyl, or C₁₋₃ alkyl.

In embodiments, a compound has a structure according to Formula (XIII)

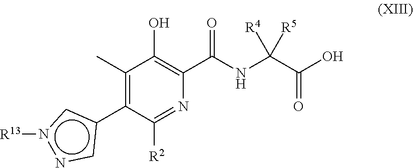

or a pharmaceutically acceptable salt thereof, wherein:

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl; and

R¹³ is aryl or heteroaryl.

In embodiments, a compound has a structure according to Formula (XIV)

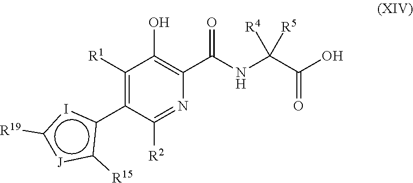

or a pharmaceutically acceptable salt thereof, wherein:

I is O, S, or CH;

J is N or CH;

R¹ is C₁₋₃ alkyl optionally substituted with OR⁷, halogen, or aryl, which is optionally substituted with halogen, and wherein R⁷ is C₁₋₃ alkyl optionally substituted with aryl, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R¹⁵ is hydrogen or C₁₋₃ alkyl; and

R¹⁹ is hydrogen or aryl.

In embodiments, a compound has a structure according to Formula (XV)

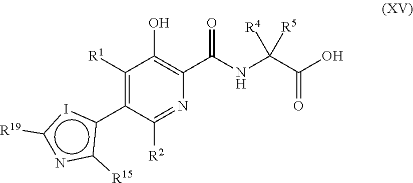

or a pharmaceutically acceptable salt thereof, wherein:

I is O, S, or CH;

R¹ is C₁₋₃ alkyl optionally substituted with OR⁷, halogen, or aryl, which is optionally substituted with halogen, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R⁷ is C₁₋₃ alkyl optionally substituted with aryl; and

R¹⁵ is hydrogen or C₁₋₃ alkyl.

R¹⁹ is hydrogen or aryl.

In embodiments, a compound has a structure according to Formula (XVI)

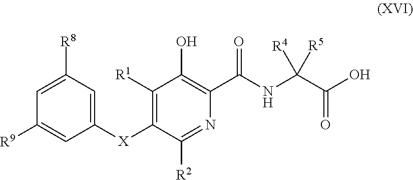

or a pharmaceutically acceptable salt thereof, wherein

X is O, S, or SO₂;

R¹ is C₁₋₃ alkyl optionally substituted with OR⁷, halogen, or aryl which is optionally substituted with halogen, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R⁷ is C₁₋₃ alkyl optionally substituted with aryl;

R⁸ and R⁹ are independently hydrogen, halogen, OR¹⁰, or C₁₋₃ alkyl optionally substituted with one or more halogens; and

R¹⁰ is C₁₋₃ alkyl or aryl.

In embodiments, a compound has a structure according to Formula (XVII)

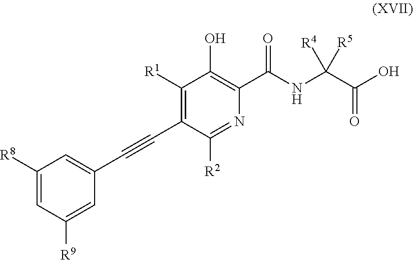

or a pharmaceutically acceptable salt thereof, wherein

R¹ is C₁₋₃ alkyl optionally substituted with OR⁷, halogen, or aryl, which is optionally substituted with halogen, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R⁷ is C₁₋₃ alkyl optionally substituted with aryl;

R⁸ and R⁹ are independently hydrogen, halogen, OR¹⁰, or C₁₋₃ alkyl optionally substituted with one or more halogens; and

R¹⁰ is C₁₋₃ alkyl or aryl.

In embodiments, a compound has a structure according to Formula (XVIII)

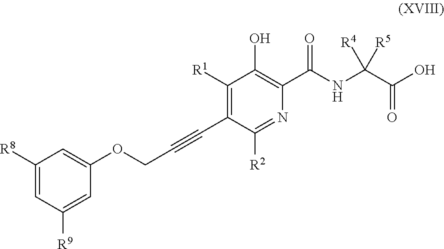

or a pharmaceutically acceptable salt thereof, wherein

R¹ is C₁₋₃ alkyl optionally substituted with OR⁷, halogen, or aryl, which is optionally substituted with halogen, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R⁷ is C₁₋₃ alkyl optionally substituted with aryl;

R⁸ and R⁹ are independently hydrogen, halogen, OR¹⁰, or C₁₋₃ alkyl optionally substituted with one or more halogens; and

R¹⁰ is C₁₋₃ alkyl or aryl.

In embodiments, a compound has a structure according to Formula (XIX)

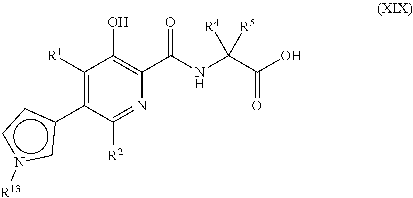

or a pharmaceutically acceptable salt thereof, wherein:

R¹ is C₁₋₃ alkyl optionally substituted with OR⁷, halogen, or aryl, which is optionally substituted with halogen, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R⁷ is C₁₋₃ alkyl optionally substituted with aryl; and

R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens.

In embodiments, a compound has a structure according to Formula (XX)

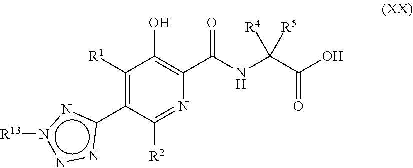

or a pharmaceutically acceptable salt thereof, wherein:

R¹ is C₁₋₃ alkyl optionally substituted with OR⁷, halogen, or aryl, which is optionally substituted with halogen, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R⁷ is C₁₋₃ alkyl optionally substituted with aryl; and

R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens.

In embodiments, a compound has a structure according to Formula (XXI)

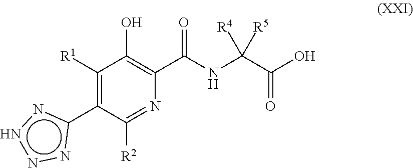

or a pharmaceutically acceptable salt thereof, wherein:

R¹ is C₁₋₃ alkyl optionally substituted with OR⁷, halogen, or aryl, which is optionally substituted with halogen, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl; and

R⁷ is C₁₋₃ alkyl optionally substituted with aryl.

In embodiments, a compound has a structure according to Formula (XXII)

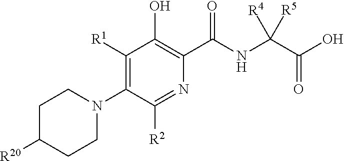

or a pharmaceutically acceptable salt thereof, wherein:

R¹ is C₁₋₃ alkyl optionally substituted with OR⁷, halogen, or aryl, which is optionally substituted with halogen, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R⁷ is C₁₋₃ alkyl optionally substituted with aryl; and

R²⁰ is optionally substituted aryl.

In embodiments, a compound has a structure according to Formula (XXIII)

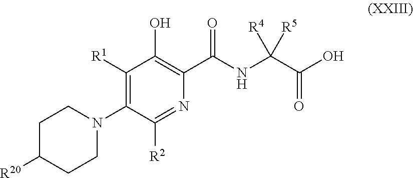

or a pharmaceutically acceptable salt thereof, wherein:

R¹ is C₁₋₃ alkyl optionally substituted with OR⁷, halogen, or aryl, which is optionally substituted with halogen, or R¹ is cyclopropyl;

R² is hydrogen, halogen, CN, or C₁₋₃ alkyl optionally substituted with one or more halogens;

R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;

R⁷ is C₁₋₃ alkyl optionally substituted with aryl; and

R²⁰ is optionally substituted aryl.

In embodiments, R³ is not hydrogen.

In embodiments, R³ is unsubstituted phenyl, fluorophenyl, chlorophenyl, difluorophenyl, dichlorophenyl, or trifluoromethylphenyl.

In embodiments, R³ is OR¹⁶, SR¹⁶, SO₂R¹⁶, CH₂R¹⁶, CH₂CH₂R¹⁶, C≡CR¹⁶, or C≡CCH₂OR¹⁶, and wherein R¹⁶ is aryl. In embodiments, R¹⁶ is phenyl.

In embodiments, R³ is pyrrolyl, tetrazolyl, triazolyl, or pyrazolyl, optionally substituted by aryl or cycloalkyl.

In embodiments, R³ is piperidinyl or piperazinyl, optionally substituted by aryl.

In embodiments, R³ is unsubstituted or substituted by cyclopropyl, unsubstituted phenyl, fluorophenyl, chlorophenyl, difluorophenyl, dichlorophenyl, or trifluoromethylphenyl.

In embodiments, R³ is COR¹⁷, and wherein R¹⁷ is aryl. In embodiments, R¹⁷ is phenyl.

In embodiments, R¹ is cyclopropyl or substituted C₁₋₃ alkyl.

In embodiments, R¹ is cyclopropyl or difluoromethyl.

In embodiments, R¹ is C₁₋₃ alkyl. In embodiments, R¹ is CH₂CH₃. In embodiments, R¹ is CH₃. In embodiments, R¹ is C₁₋₃ alkyl substituted with aryl, which is substituted with halogen. In embodiments, R¹ is

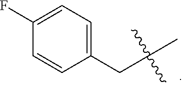 [SMILES: *Cc1ccc(F)cc1]

In embodiments, R¹ is C₁₋₃ alkyl substituted with OBn. In embodiments, R¹ is CH₂CH₂OBn.

In embodiments, R² is hydrogen. In embodiments, R² is C₁₋₃ alkyl. In embodiments, R² is CH₃.

In embodiments, R⁴ is hydrogen and R⁵ is hydrogen. In embodiments, R⁴ is hydrogen and R⁵ is C₁₋₃ alkyl. In embodiments, R⁵ is CH₃. In embodiments, R⁴ is C₁₋₃ alkyl and R⁵ is C₁₋₃ alkyl. In embodiments, R⁴ is CH₃ and R⁵ is CH₃.

In embodiments, R⁴ and R⁵ together with the carbon to which they are attached form a cycloalkyl or a heterocycloalkyl. In embodiments, the cycloalkyl is cyclopropyl. In embodiments, the cycloalkyl is cyclobutyl. In embodiments, the heterocycloalkyl is

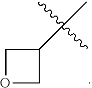 [SMILES: *C1COC1]

In embodiments, the compound is any one of Compounds 1-50:

| Cmpd | |
|---|---|
| No. | Structure |
| 1 | 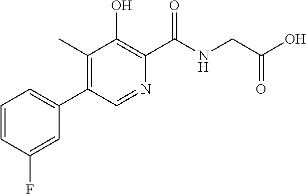 [SMILES: Cc1c(-c2cccc(F)c2)cnc(C(=O)NCC(=O)O)c1O] |
| 2 | 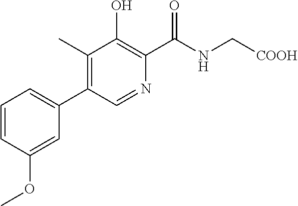 [SMILES: COc1cccc(-c2cnc(C(=O)NCC(=O)O)c(O)c2C)c1] |
| 3 | 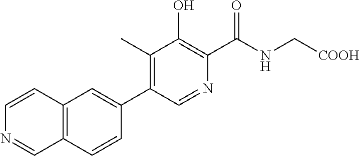 [SMILES: Cc1c(-c2ccc3cnccc3c2)cnc(C(=O)NCC(=O)O)c1O] |
| 4 | 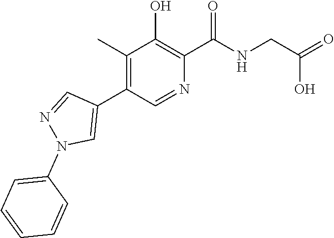 [SMILES: Cc1c(-c2cnn(-c3ccccc3)c2)cnc(C(=O)NCC(=O)O)c1O] |
| 5 | 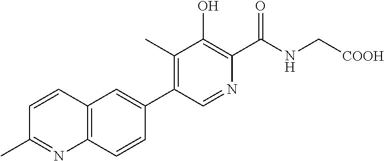 [SMILES: Cc1ccc2cc(-c3cnc(C(=O)NCC(=O)O)c(O)c3C)ccc2n1] |
| 6 | 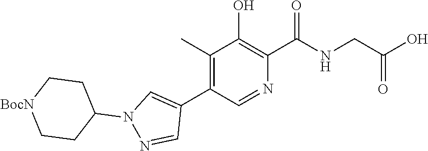 [SMILES: Cc1c(-c2cnn(C3CCN(C(=O)OC(C)(C)C)CC3)c2)cnc(C(=O)NCC(=O)O)c1O] |
| 7 | 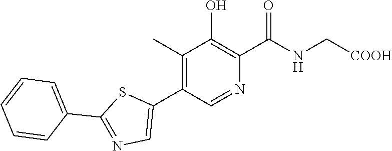 [SMILES: Cc1c(-c2cnc(-c3ccccc3)s2)cnc(C(=O)NCC(=O)O)c1O] |
| 8 | 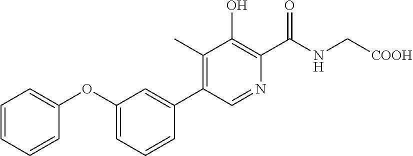 [SMILES: Cc1c(-c2cccc(Oc3ccccc3)c2)cnc(C(=O)NCC(=O)O)c1O] |
| 9 | 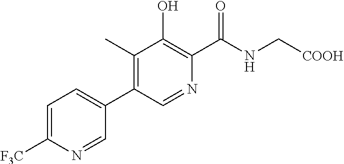 [SMILES: Cc1c(-c2ccc(C(F)(F)F)nc2)cnc(C(=O)NCC(=O)O)c1O] |
| 10 | 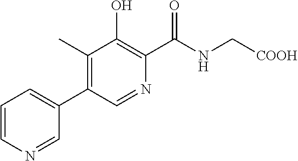 [SMILES: Cc1c(-c2cccnc2)cnc(C(=O)NCC(=O)O)c1O] |
| 11 | 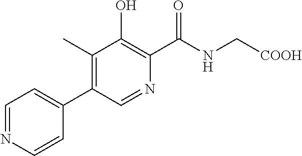 [SMILES: Cc1c(-c2ccncc2)cnc(C(=O)NCC(=O)O)c1O] |
| 12 | 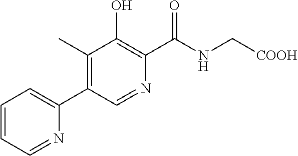 [SMILES: Cc1c(-c2ccccn2)cnc(C(=O)NCC(=O)O)c1O] |
| 13 | 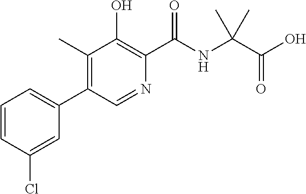 [SMILES: Cc1c(-c2cccc(Cl)c2)cnc(C(=O)NC(C)(C)C(=O)O)c1O] |
| 14 | 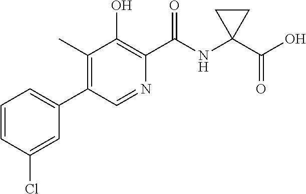 [SMILES: Cc1c(-c2cccc(Cl)c2)cnc(C(=O)NC2(C(=O)O)CC2)c1O] |
| 15 | 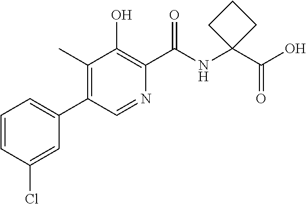 [SMILES: Cc1c(-c2cccc(Cl)c2)cnc(C(=O)NC2(C(=O)O)CCC2)c1O] |
| 16 | 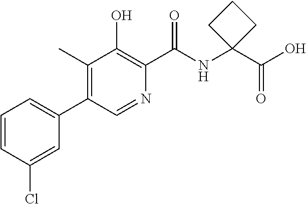 [SMILES: Cc1c(-c2cccc(Cl)c2)cnc(C(=O)NC2(C(=O)O)CCC2)c1O] |
| 17 | 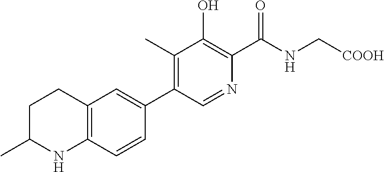 [SMILES: Cc1c(-c2ccc3c(c2)CCC(C)N3)cnc(C(=O)NCC(=O)O)c1O] |
| 18 | 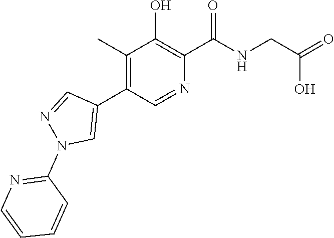 [SMILES: Cc1c(-c2cnn(-c3ccccn3)c2)cnc(C(=O)NCC(=O)O)c1O] |
| 19 | 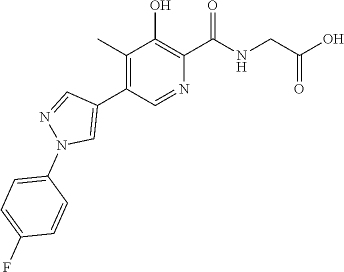 [SMILES: Cc1c(-c2cnn(-c3ccc(F)cc3)c2)cnc(C(=O)NCC(=O)O)c1O] |
| 20 | 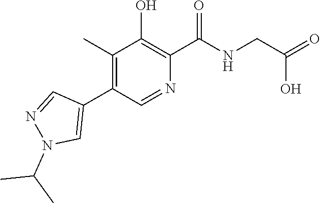 [SMILES: Cc1c(-c2cnn(C(C)C)c2)cnc(C(=O)NCC(=O)O)c1O] |
| 21 | 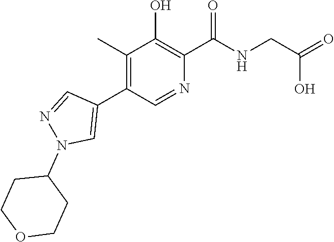 [SMILES: Cc1c(-c2cnn(C3CCOCC3)c2)cnc(C(=O)NCC(=O)O)c1O] |
| 22 | 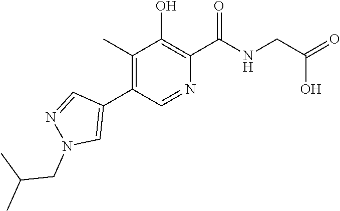 [SMILES: Cc1c(-c2cnn(CC(C)C)c2)cnc(C(=O)NCC(=O)O)c1O] |
| 23 | 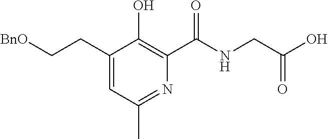 [SMILES: Cc1cc(CCOCc2ccccc2)c(O)c(C(=O)NCC(=O)O)n1] |
| 24 | 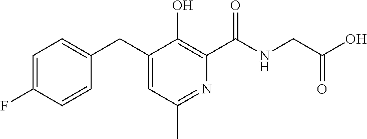 [SMILES: Cc1cc(Cc2ccc(F)cc2)c(O)c(C(=O)NCC(=O)O)n1] |
| 25 | 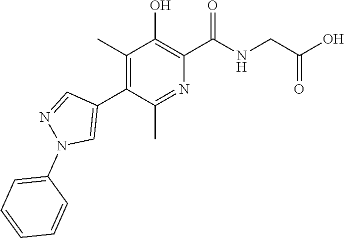 [SMILES: Cc1nc(C(=O)NCC(=O)O)c(O)c(C)c1-c1cnn(-c2ccccc2)c1] |
| 26 | 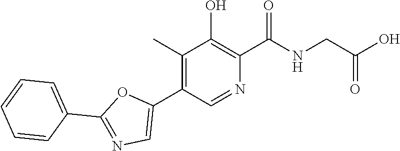 [SMILES: Cc1c(-c2cnc(-c3ccccc3)o2)cnc(C(=O)NCC(=O)O)c1O] |
| 27 | 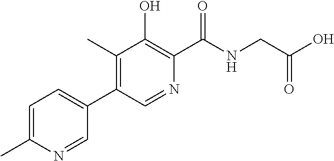 [SMILES: Cc1ccc(-c2cnc(C(=O)NCC(=O)O)c(O)c2C)cn1] |
| 28 | 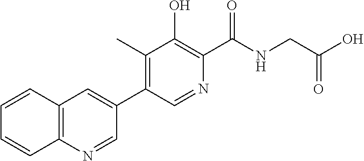 [SMILES: Cc1c(-c2cnc3ccccc3c2)cnc(C(=O)NCC(=O)O)c1O] |
| 29 | 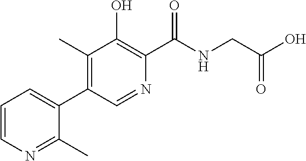 [SMILES: Cc1ncccc1-c1cnc(C(=O)NCC(=O)O)c(O)c1C] |
| 30 | 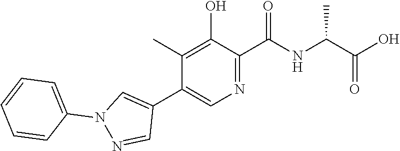 [SMILES: Cc1c(-c2cnn(-c3ccccc3)c2)cnc(C(=O)N[C@H](C)C(=O)O)c1O] |
| 31 | 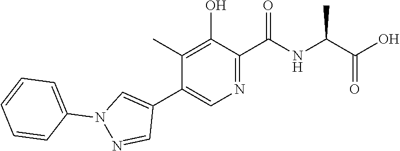 [SMILES: Cc1c(-c2cnn(-c3ccccc3)c2)cnc(C(=O)N[C@@H](C)C(=O)O)c1O] |
| 32 | 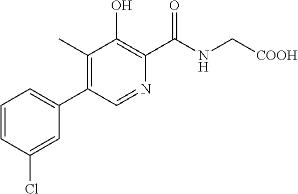 [SMILES: Cc1c(-c2cccc(Cl)c2)cnc(C(=O)NCC(=O)O)c1O] |
| 33 | 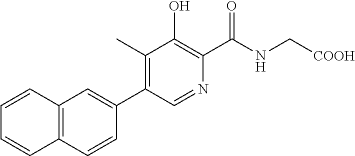 [SMILES: Cc1c(-c2ccc3ccccc3c2)cnc(C(=O)NCC(=O)O)c1O] |
| 34 | 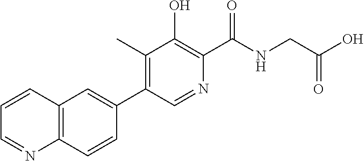 [SMILES: Cc1c(-c2ccc3ncccc3c2)cnc(C(=O)NCC(=O)O)c1O] |
| 35 | 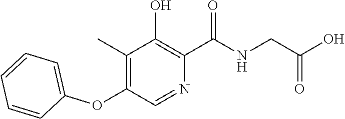 [SMILES: Cc1c(Oc2ccccc2)cnc(C(=O)NCC(=O)O)c1O] |
| 36 | 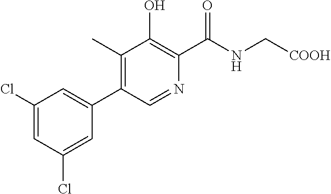 [SMILES: Cc1c(-c2cc(Cl)cc(Cl)c2)cnc(C(=O)NCC(=O)O)c1O] |
| 37 | 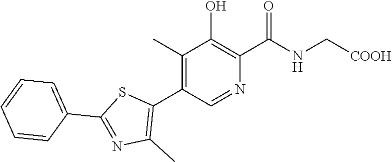 [SMILES: Cc1nc(-c2ccccc2)sc1-c1cnc(C(=O)NCC(=O)O)c(O)c1C] |
| 38 | 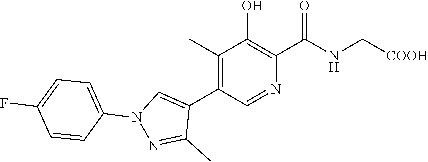 [SMILES: Cc1nn(-c2ccc(F)cc2)cc1-c1cnc(C(=O)NCC(=O)O)c(O)c1C] |
| 39 | 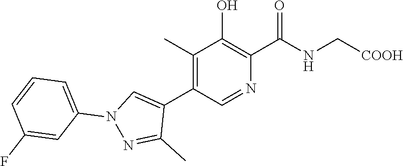 [SMILES: Cc1nn(-c2cccc(F)c2)cc1-c1cnc(C(=O)NCC(=O)O)c(O)c1C] |
| 40 | 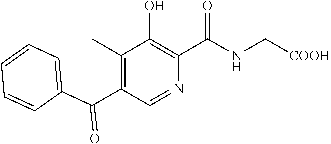 [SMILES: Cc1c(C(=O)c2ccccc2)cnc(C(=O)NCC(=O)O)c1O] |
| 41 | 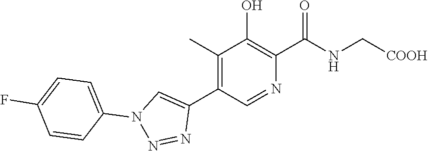 [SMILES: Cc1c(-c2cn(-c3ccc(F)cc3)nn2)cnc(C(=O)NCC(=O)O)c1O] |
| 42 | 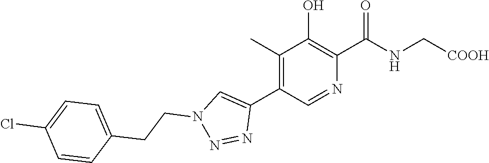 [SMILES: Cc1c(-c2cn(CCc3ccc(Cl)cc3)nn2)cnc(C(=O)NCC(=O)O)c1O] |
| 43 | 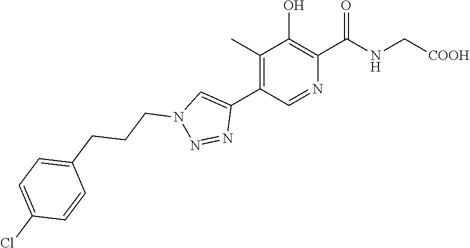 [SMILES: Cc1c(-c2cn(CCCc3ccc(Cl)cc3)nn2)cnc(C(=O)NCC(=O)O)c1O] |
| 44 | 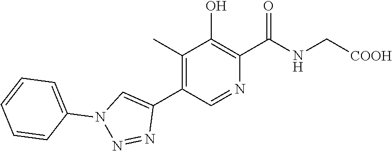 [SMILES: Cc1c(-c2cn(-c3ccccc3)nn2)cnc(C(=O)NCC(=O)O)c1O] |
| 45 | 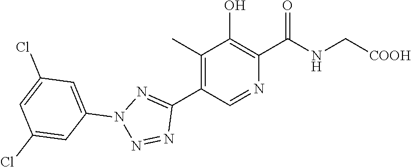 [SMILES: Cc1c(-c2nnn(-c3cc(Cl)cc(Cl)c3)n2)cnc(C(=O)NCC(=O)O)c1O] |
| 46 | 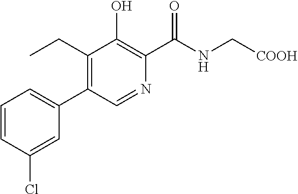 [SMILES: CCc1c(-c2cccc(Cl)c2)cnc(C(=O)NCC(=O)O)c1O] |
| 47 | 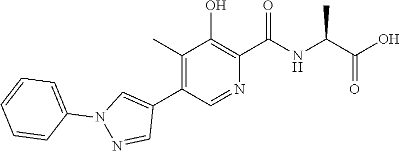 [SMILES: Cc1c(-c2cnn(-c3ccccc3)c2)cnc(C(=O)N[C@@H](C)C(=O)O)c1O] |
| 48 | 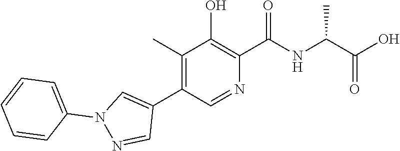 [SMILES: Cc1c(-c2cnn(-c3ccccc3)c2)cnc(C(=O)N[C@H](C)C(=O)O)c1O] |
| 49 | 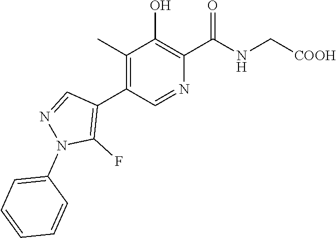 [SMILES: Cc1c(-c2cnn(-c3ccccc3)c2F)cnc(C(=O)NCC(=O)O)c1O] |
| 50 | 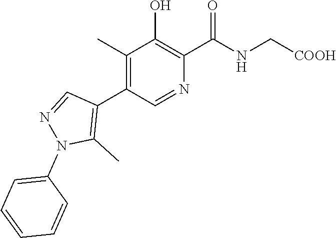 [SMILES: Cc1c(-c2cnn(-c3ccccc3)c2C)cnc(C(=O)NCC(=O)O)c1O] |

or a pharmaceutically acceptable salt thereof.

In embodiments, a compound is any one of Compounds 51-70,

| Cmpd | | Cmpd | |
|---|---|---|---|
| No. | Structure | No. | Structure |
| 51 | 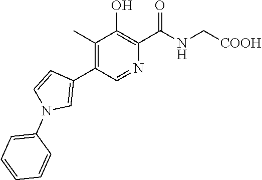 [SMILES: Cc1c(-c2ccn(-c3ccccc3)c2)cnc(C(=O)NCC(=O)O)c1O] | 52 | 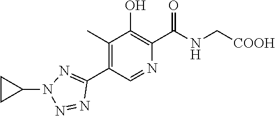 [SMILES: Cc1c(-c2nnn(C3CC3)n2)cnc(C(=O)NCC(=O)O)c1O] |
| 53 | 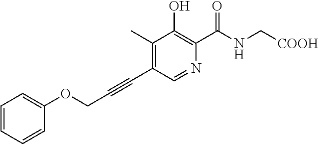 [SMILES: Cc1c(C#CCOc2ccccc2)cnc(C(=O)NCC(=O)O)c1O] | 54 | 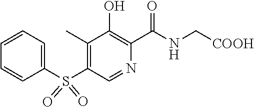 [SMILES: Cc1c(S(=O)(=O)c2ccccc2)cnc(C(=O)NCC(=O)O)c1O] |
| 55 | 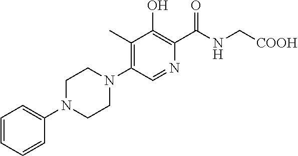 [SMILES: Cc1c(N2CCN(c3ccccc3)CC2)cnc(C(=O)NCC(=O)O)c1O] | 56 | 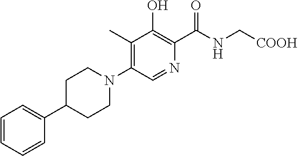 [SMILES: Cc1c(N2CCC(c3ccccc3)CC2)cnc(C(=O)NCC(=O)O)c1O] |
| 57 | 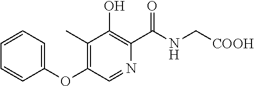 [SMILES: Cc1c(Oc2ccccc2)cnc(C(=O)NCC(=O)O)c1O] | 58 | 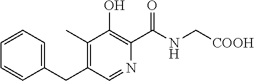 [SMILES: Cc1c(Cc2ccccc2)cnc(C(=O)NCC(=O)O)c1O] |
| 59 | 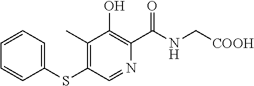 [SMILES: Cc1c(Sc2ccccc2)cnc(C(=O)NCC(=O)O)c1O] | 60 | 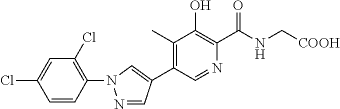 [SMILES: Cc1c(-c2cnn(-c3ccc(Cl)cc3Cl)c2)cnc(C(=O)NCC(=O)O)c1O] |
| 61 | 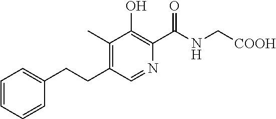 [SMILES: Cc1c(CCc2ccccc2)cnc(C(=O)NCC(=O)O)c1O] | 62 | 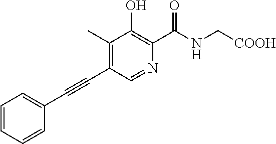 [SMILES: Cc1c(C#Cc2ccccc2)cnc(C(=O)NCC(=O)O)c1O] |
| 63 | 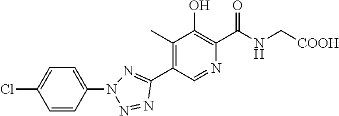 [SMILES: Cc1c(-c2nnn(-c3ccc(Cl)cc3)n2)cnc(C(=O)NCC(=O)O)c1O] | 64 | 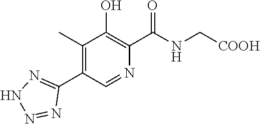 [SMILES: Cc1c(-c2nn[nH]n2)cnc(C(=O)NCC(=O)O)c1O] |
| 65 | 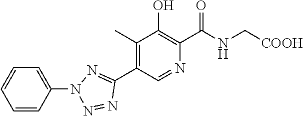 [SMILES: Cc1c(-c2nnn(-c3ccccc3)n2)cnc(C(=O)NCC(=O)O)c1O] | 66 | 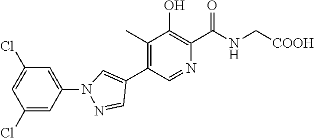 [SMILES: Cc1c(-c2cnn(-c3cc(Cl)cc(Cl)c3)c2)cnc(C(=O)NCC(=O)O)c1O] |
| 67 | 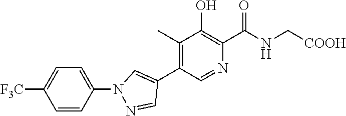 [SMILES: Cc1c(-c2cnn(-c3ccc(C(F)(F)F)cc3)c2)cnc(C(=O)NCC(=O)O)c1O] | 68 | 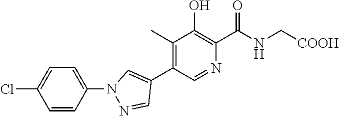 [SMILES: Cc1c(-c2cnn(-c3ccc(Cl)cc3)c2)cnc(C(=O)NCC(=O)O)c1O] |
| 69 | 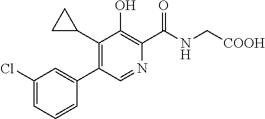 [SMILES: O=C(O)CNC(=O)c1ncc(-c2cccc(Cl)c2)c(C2CC2)c1O] | 70 | 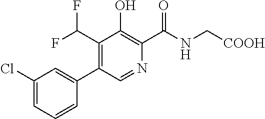 [SMILES: O=C(O)CNC(=O)c1ncc(-c2cccc(Cl)c2)c(C(F)F)c1O] |

or a pharmaceutically acceptable salt thereof.

In embodiments, in the compound of Formulas (I)-(XXIII) such as any one of Compounds 1-70 at least one hydrogen atom is replaced with a deuterium atom.

In another aspect, the invention features a pharmaceutical composition comprising any compound described herein (e.g., a compound of Formulas (I)-(XXIII) such as any one of Compounds 1-70), or a pharmaceutically acceptable salt thereof, and a pharmaceutically acceptable excipient.

In another aspect, the invention features a method for treating a disease mediated by PHD activity comprising administering to a subject any compound described herein (e.g., a compound of Formulas (I)-(XXIII) such as any one of Compounds 1-70), or a pharmaceutically acceptable salt thereof.

In embodiments, a disease mediated by PHD activity is an ischemic reperfusion injury. (e.g., stroke, myocardial infarction, or acute kidney injury).

In embodiments, a disease mediated by PHD activity is inflammatory bowel disease (e.g., ulcerative colitis or Crohn's disease).

In embodiments, a disease mediated by PHD activity is cancer (e.g., colorectal cancer).

In embodiments, a disease mediated by PHD activity is liver disease.

In embodiments, a disease mediated by PHD activity is atherosclerosis.

In embodiments, a disease mediated by PHD activity is cardiovascular disease

In embodiments, a disease mediated by PHD activity is a disease or condition of the eye (e.g., radiation retinopathy, retinopathy of prematurity, diabetic retinopathy, age-related macular degeneration, and ocular ischemia).

In embodiments, a disease mediated by PHD activity is anemia (e.g., anemia associated with chronic kidney disease).

In embodiments, a disease mediated by PHD activity is chronic kidney disease.

In embodiments, a disease mediated by PHD activity is associated with hyperoxia.

In embodiments, a disease mediated by PHD activity is retinopathy of prematurity.

In embodiments, a disease mediated by PHD activity is bronchopulmonary dysplasia (BPD).

In embodiments, a disease mediated by PHD activity is ischemic heart disease, valvular heart disease, congestive heart failure, acute lung injury, pulmonary fibrosis, pulmonary hypertension, chronic obstructive pulmonary disease (COPD), acute liver failure, liver fibrosis, or cirrhosis.

In embodiments, a disease mediated by PHD activity is a respiratory disease, a lung disease, a respiratory viral infection, or a pulmonary viral infection.

In embodiments, the respiratory disease is selected from respiratory infection, acute respiratory distress syndrome, lung inflammation, pneumonia, and acute lung injury.

In embodiments, the lung disease is acute lung injury (ALI), bronchitis, pneumonia, pulmonary fibrosis, asthma, or acute respiratory distress syndrome (ARDS).

In embodiments, a disease mediated by PHD activity is injury to and/or failure of one or more organs (e.g. acute organ injury, or organ failure).

### BRIEF DESCRIPTION OF DRAWINGS

FIG. 1 is an exemplary schematic illustration demonstrating the principle of the TR-FRET Assay for PHD enzymes (PHD1, PHD2, and PHD3). In the presence of 2-oxoglutarate and 02, PHD enzyme hydroxylates proline 564 of biotin-tagged HIF-la peptide resulting in generation of biotin-tagged HIF-1α-hydroxyproline, succinate and CO₂. The resulting proximity of the donor fluorophore complex, monoclonal antibody anti-6His-Terbium (Tb)-cryptate Gold, bound to the His-tagged VHL protein, EloB, EloC complex (His-VBC) and the acceptor fluorophore, SA-D2 complex, bound to HIF-1α-hydroxyproline results in a fluorescence resonance energy transfer signal that can be detected and quantified

### DETAILED DESCRIPTION OF THE DISCLOSURE

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, a bovine, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used in the description and the appended claims, the singular forms “a,” “an,” and “the” include plural referents unless the context clearly dictates otherwise. Thus, for example, reference to “a composition” includes mixtures of two or more such compositions.

Throughout the description and claims of this specification the word “comprise” and other forms of the word, such as “comprising” and “comprises,” means including but not limited to, and is not intended to exclude, for example, other additives, components, integers, or steps.

“Optional” or “optionally” means that the subsequently described event or circumstance may or may not occur, and that the description includes instances where the event or circumstance occurs and instances where it does not.

Improve, increase, or reduce: As used herein, the terms “improve,” “increase,” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable,” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid, or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4 alkyl)4 salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate, and aryl sulfonate. Further pharmaceutically acceptable salts include salts formed from the quarternization of an amine using an appropriate electrophile, e.g., an alkyl halide, to form a quarternized alkylated amino salt.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Aliphatic: As used herein, the term aliphatic refers to C₁-C₄₀ hydrocarbons and includes both saturated and unsaturated hydrocarbons. An aliphatic may be linear, branched, or cyclic. For example, C₁-C₂₀ aliphatics can include C₁-C₂₀ alkyls (e.g., linear or branched C₁-C₂₀ saturated alkyls), C₂-C₂₀ alkenyls (e.g., linear or branched C₄-C₂₀ dienyls, linear, or branched C₆-C₂₀ trienyls, and the like), and C₂-C₂₀ alkynyls (e.g., linear or branched C₂-C₂₀ alkynyls). C₁-C₂₀ aliphatics can include C₃-C₂₀ cyclic aliphatics (e.g., C₃-C₂₀ cycloalkyls, C₄-C₂₀ cycloalkenyls, or C₅-C₂₀ cycloalkynyls). In certain embodiments, the aliphatic may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. An aliphatic group is unsubstituted or substituted with one or more substituent groups as described herein. For example, an aliphatic may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the aliphatic is unsubstituted. In some embodiments, the aliphatic does not include any heteroatoms.

Alkyl: As used herein, the term “alkyl” means acyclic linear and branched hydrocarbon groups, e.g. “C₁-C₂₀ alkyl” refers to alkyl groups having 1-20 carbons. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentylhexyl, isohexyl, etc. The term “lower alkyl” means an alkyl group straight chain or branched alkyl having 1 to 6 carbon atoms. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkyl is substituted (e.g., with 1, 2, 3, 4, 5, or 6 substituent groups as described herein). In some embodiments, an alkyl group is substituted with a-OH group and may also be referred to herein as a “hydroxyalkyl” group, where the prefix denotes the —OH group and “alkyl” is as described herein. In some embodiments, the alkyl is substituted with a —OR′ group and may also be referred to herein as “alkoxy” group.

Affixing the suffix “-ene” to a group indicates the group is a divalent moiety, e.g., arylene is the divalent moiety of aryl, and heteroarylene is the divalent moiety of heteroaryl.

Alkylene: The term “alkylene,” as used herein, represents a saturated divalent straight or branched chain hydrocarbon group and is exemplified by methylene, ethylene, isopropylene and the like. Likewise, the term “alkenylene” as used herein represents an unsaturated divalent straight or branched chain hydrocarbon group having one or more unsaturated carbon-carbon double bonds that may occur in any stable point along the chain, and the term “alkynylene” herein represents an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In certain embodiments, an alkylene, alkenylene, or alkynylene is unsubstituted. In certain embodiments, an alkylene, alkenylene, or alkynylene does not include any heteroatoms.

Alkenyl: As used herein, “alkenyl” means any linear or branched hydrocarbon chains having one or more unsaturated carbon-carbon double bonds that may occur in any stable point along the chain, e.g. “C₂-C₂₀ alkenyl” refers to an alkenyl group having 2-20 carbons. For example, an alkenyl group includes prop-2-enyl, but-2-enyl, but-3-enyl, 2-methylprop-2-enyl, hex-2-enyl, hex-5-enyl, 2,3-dimethylbut-2-enyl, and the like. In some embodiments, the alkenyl comprises 1, 2, or 3 carbon-carbon double bond. In some embodiments, the alkenyl comprises a single carbon-carbon double bond. In some embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkenyl is unsubstituted. In some embodiments, the alkenyl is substituted (e.g., with 1, 2, 3, 4, 5, or 6 substituent groups as described herein). In some embodiments, an alkenyl group is substituted with a-OH group and may also be referred to herein as a “hydroxyalkenyl” group, where the prefix denotes the —OH group and “alkenyl” is as described herein.

Alkynyl: As used herein, “alkynyl” means any hydrocarbon chain of either linear or branched configuration, having one or more carbon-carbon triple bonds occurring in any stable point along the chain, e.g. “C₂-C₂₀ alkynyl” refers to an alkynyl group having 2-20 carbons. Examples of an alkynyl group include prop-2-ynyl, but-2-ynyl, but-3-ynyl, pent-2-ynyl, 3-methylpent-4-ynyl, hex-2-ynyl, hex-5-ynyl, etc. In some embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃alkyl). In some embodiments, R′ independently is unsubstituted C₁-C₃ alkyl. In some embodiments, the alkynyl is unsubstituted. In some embodiments, the alkynyl is substituted (e.g., with 1, 2, 3, 4, 5, or 6 substituent groups as described herein).

Aryl: The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, at least one ring in the system is aromatic and wherein each ring in the system contains 4 to 7 ring members. In some embodiments, an aryl group has 6 ring carbon atoms (“C₆ aryl,” e.g., phenyl). In some embodiments, an aryl group has 10 ring carbon atoms (“C₁₀ aryl,” e.g., naphthyl such as 1-naphthyl and 2-naphthyl). In some embodiments, an aryl group has 14 ring carbon atoms (“C₁₄ aryl,” e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryls include phenyl, naphthyl, and anthracene.

Arylene: The term “arylene” as used herein refers to an aryl group that is divalent (that is, having two points of attachment to the molecule). Exemplary arylenes include phenylene (e.g., unsubstituted phenylene or substituted phenylene).

Halogen or Halo: As used herein, the term “halogen” or “halo” means fluorine, chlorine, bromine, or iodine.

Amide: The term “amide” or “amido” refers to a chemical moiety with formula —C(O)N(R′)₂, —C(O)N(R′)—, —NR′C(O)R′, —NR′C(O)N(R′)₂—, or —NR′C(O)—, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated other-wise in the specification, each of which moiety can itself be optionally substituted as described herein, or two R′ can combine with the nitrogen atom to form a 3-, 4-, 5-, 6-, or 7-membered ring.

Amino: The term “amino” or “amine” refers to a —N(R′)₂ group, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, heterocycloalkyl (bonded through a ring carbon), sulfonyl, amide or carbonyl group, unless stated other-wise in the specification, each of which moiety can itself be optionally substituted as described herein, or two R′ can combine with the nitrogen atom to form a 3-, 4-, 5-, 6-, or 7-membered ring. In embodiments, an amino group is —NHR′, where R′ is aryl (“arylamino”), heteroaryl (“heteroarylamino”), amide or alkyl (“alkylamino”).

Ether: The term “ether” refers to a R′—O—R′ group, where each R′ is independently selected from alkyl, heteroalkyl (bonded through a chain carbon), arylalkyl, heteroarylalkyl, heterocycloalkyl (bonded through a ring carbon), cycloalkyl, aryl, heteroaryl (bonded through a ring carbon), unless stated other-wise in the specification, each of which moiety can itself be optionally substituted as described herein.

Ester: The term “ester” refers to a R′—C(═O)O—R group, where each R′ is independently selected from alkyl, heteroalkyl (bonded through a chain carbon), arylalkyl, heteroarylalkyl, heterocycloalkyl (bonded through a ring carbon), cycloalkyl, aryl, heteroaryl (bonded through a ring carbon), unless stated other-wise in the specification, each of which moiety can itself be optionally substituted as described herein.

Sulfonyl: The term “sulfonyl” refers to a —S(═O)₂R′, or —S(═O)₂— group, where R′ is selected from hydrogen, alkyl, alkenyl, alkynyl, heteroalkyl (bonded through a chain carbon), amino, cycloalkyl, aryl, arylalkyl, heteroaryl (bonded through a ring carbon), heteroarylalkyl, heterocycloalkyl (bonded through a ring carbon), unless stated other-wise in the specification, each of which moiety can itself be optionally substituted as described herein. For example, in one embodiment, the sulfonyl group is —SO₂R′, where R′ is alkyl substituted with a carbonyl group.

Sulfinyl: The term “sulfinyl” refers to a chemical moiety with formula —S(═O)R′, —S(═O)—, or —S(═O)(═NR′)—, where R′ is selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, heterocycloalkyl (bonded through a ring carbon), unless stated other-wise in the specification, each of which moiety can itself be optionally substituted as described herein.

Carbonyl: The term “carbonyl” refers to a —C(═O)R′, or —C(═O)— group, where R′ is selected from hydrogen, alkyl, alkenyl, alkynyl, heteroalkyl (bonded through a chain carbon), cycloalkyl, aryl, arylalkyl, amino, hydroxyl, heteroaryl (bonded through a ring carbon), heteroarylalkyl, heterocycloalkyl (bonded through a ring carbon), unless stated other-wise in the specification, each of which moiety can itself be optionally substituted as described herein.

Phosphoryl: The term “phosphoryl” refers to a —P(═O)(R′)₂, or —P(═O)(R′)— group, where R′ is selected from hydrogen, alkyl, alkenyl, alkynyl, heteroalkyl (bonded through a chain carbon or through the heteroatom), cycloalkyl, aryl, arylalkyl, heteroaryl (bonded through a ring carbon), heteroarylalkyl, or heterocycloalkyl (bonded through a ring carbon) group, unless stated other-wise in the specification, each of which moiety can itself be optionally substituted as described herein, or two R′ can combine with the nitrogen atom to form a 3-, 4-, 5-, 6-, or 7-membered ring.

Heteroalkyl: The term “heteroalkyl” is meant a branched or unbranched alkyl, alkenyl, or alkynyl group having from 1 to 14 carbon atoms in addition to 1, 2, 3 or 4 heteroatoms independently selected from the group consisting of N, O, S, and P. Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to six members. Examples of heteroalkyls include polyethers, such as methoxymethyl and ethoxyethyl.

Heteroalkylene: The term “heteroalkylene,” as used herein, represents a divalent form of a heteroalkyl group as described herein.

Heteroaryl: The term “heteroaryl,” as used herein, refers to a monocyclic, bicyclic, or tricyclic carbocyclic ring system having a total of six to fourteen ring members, wherein said ring system has a single point of attachment to the rest of the molecule, wherein at least one ring in the system is aromatic, wherein each ring in the system contains 4 to 7 ring members, and wherein at least one ring atom is a heteroatom such as, but not limited to, nitrogen and oxygen.

Heterocycloalkyl: The term “heterocycloalkyl,” as used herein, is a non-aromatic ring wherein at least one atom is a heteroatom such as, but not limited to, nitrogen, oxygen, sulfur, or phosphorus, and the remaining atoms are carbon. The heterocycloalkyl group can be substituted or unsubstituted.

Deuterium: The term “deuterium” (“D” or “²H”) is also called heavy hydrogen. Deuterium is isotope of hydrogen with a nucleus consisting of one proton and one neutron, which is double the mass of the nucleus of ordinary hydrogen (one proton).

Isotope: The term “isotope” refers to a variant of a particular chemical element which differs in neutron number, and consequently in nucleon number. All isotopes of a given element have the same number of protons but different numbers of neutrons in each atom.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system, e.g., the substitution results in a stable compound (e.g., a compound which does not spontaneously undergo transformation such as by rearrangement, cyclization, elimination, or other reaction). In cases where a specified moiety or group is not expressly noted as being optionally substituted or substituted with any specified substituent, it is understood that such a moiety or group is intended to be unsubstituted.

When a ring system (e.g., cycloalkyl, heterocyclyl, aryl, or heteroaryl) is substituted with a number of substituents varying within an expressly defined range, it is understood that the total number of substituents does not exceed the normal available valencies under the existing conditions. It is also understood that hydrogen atoms are presumed present to fill the remaining valence of a ring system. The substituted group encompasses only those combinations of substituents and variables that result in a stable or chemically feasible compound. A stable compound or chemically feasible compound is one that, among other factors, has stability sufficient to permit its preparation and detection.

A wide variety of substituents are well known, and methods for their formation and introduction into a variety of parent groups are also well known. Representative substituents include but are not limited to alkyl, cycloalkyl, alkenyl, cycloalkenyl, alkynyl, arylalkyl, alkylaryl, aryl, arylalkoxy, arylamino, heteroarylamino, heteroaryl, heteroarylalkoxy, heterocycloalkyl, hydroxyalkyl, aminoalkyl, haloalkyl, thioalkyl, alkylthioalkyl, carboxyalkyl, imidazolylalkyl, indolylalkyl, mono-, di- and trihaloalkyl, mono-, di- and trihaloalkoxy, amino, alkylamino, dialkylamino, amide, cyano, alkoxy, hydroxy, sulfonamide, halo (e.g., Cl and Br), nitro, oximino, COOR⁵⁰, COR⁵⁰, SO₀₋₂R⁵⁰, —SO₂NR⁵⁰R⁵¹, NR⁵²SO₂R⁵⁰, ═C(R⁵⁰R⁵¹), ═N—OR⁵⁰, ═N—CN, ═C(halo)₂, ═S, ═O, —CON(R⁵⁰R⁵¹), —OCOR⁵⁰, —OCON(R⁵⁰R⁵¹), —N(R⁵²)CO(R⁵⁰), —N(R⁵²)COOR⁵⁰ and —N(R⁵²)CON(R⁵⁰(R⁵¹), wherein R⁵⁰, R⁵¹ and R⁵² may be independently selected from the following: a hydrogen atom and a branched or straight-chain, C₁₋₆-alkyl, C₃₋₆-cycloalkyl, C₄₋₆-heterocycloalkyl, heteroaryl and aryl group, with or without substituents. When permissible, R⁵⁰ and R⁵¹ can be joined together to form a carbocyclic or heterocyclic ring system.

In preferred embodiments, the substituent is selected from halogen, —COR′, —CO₂H, —CO₂R′, —CN, —OH, —OR′, —OCOR′, —OCO₂R′, —NH₂, —NHR′, —N(R′)₂, —SR′, and —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In certain embodiments thereof, R′ independently is an unsubstituted alkyl (e.g., unsubstituted C₁-C₂₀ alkyl, C₁-C₁₅ alkyl, C₁-C₁₀ alkyl, or C₁-C₃ alkyl). Preferably, R′ independently is unsubstituted C₁-C₃ alkyl.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to embrace hydrates, solvates, and polymorphs of such compounds, and mixtures thereof.

### Compounds of the Invention

Disclosed herein are compounds that are potent inhibitors of PHD. In some embodiments, the compounds of the present invention have enzymatic half maximal inhibitory concentration (IC₅₀) values of less than 100 μM against any one of PHD1, PHD2, and PHD3. In some embodiments, the compounds of the present invention have an IC₅₀ value of less than 50 μM against any one of PHD1, PHD2, and PHD3. In some embodiments, the compounds of the present invention have an IC₅₀ value of less than 25 μM against any one of PHD1, PHD2, and PHD3. In some embodiments, the compounds of the present invention have an IC₅₀ value of less than 20 μM against any one of PHD1, PHD2, and PHD3. In some embodiments, the compounds of the present invention have an IC₅₀ value of less than 15 μM against any one of PHD1, PHD2, and PHD3. In some embodiments, the compounds of the present invention have an IC₅₀ value of less than 10 μM against any one of PHD1, PHD2, and PHD3. In some embodiments, the compounds of the present invention have an IC₅₀ value of less than 5 μM against any one of PHD1, PHD2, and PHD3. In some embodiments, the compounds of the present invention have an IC₅₀ value of less than 1 μM against any one of PHD1, PHD2, and PHD3. In some embodiments, the compounds of the present invention have an IC₅₀ value of about 3 nM to about 5 nM against any one of PHD1, PHD2, and PHD3. In some embodiments, the compounds of the present invention have an IC₅₀ value of about 5 nM to about 10 nM against any one of PHD1, PHD2, and PHD3. In some embodiments, the compounds of the present invention have an IC₅₀ value of about 10 nM to about 20 nM against any one of PHD1, PHD2, and PHD3. In some embodiments, the compounds of the present invention have an IC₅₀ value of about 20 nM to about 50 nM against any one of PHD1, PHD2, and PHD3. In some embodiments, the compounds of the present invention have an IC₅₀ value of about 50 nM to about 100 nM against any one of PHD1, PHD2, and PHD3. In some embodiments, the compounds of the present invention have an IC₅₀ value of about 100 nM to about 200 nM against any one of PHD1, PHD2, and PHD3. In some embodiments, the compounds of the present invention have an IC₅₀ value of about 200 nM to about 500 nM against any one of PHD1, PHD2, and PHD3. In some embodiments, the compounds of the present invention have an IC₅₀ value of about 500 nM to about 1000 nM against any one of PHD1, PHD2, and PHD3.

Representative examples from this class show inhibitory activity for PHD1, PHD2 and PHD3 in vitro.

Exemplary compounds are described herein.

Compounds of Formulas (I)-(XXIII)

In particular, PHD inhibitors described herein feature a 3-hydroxypicolinamide moiety,

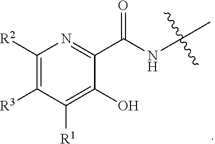

Applicant surprisingly found that substitution of the 3-hydroxypicolinamide moiety at R¹ (R¹ is not hydrogen) can significantly increase the potency of the inhibitor. Examples of such substitution include, but are not limited to, substituted or unsubstituted alkyl.

In an aspect, provided herein are compounds having a structure according to Formula (I):

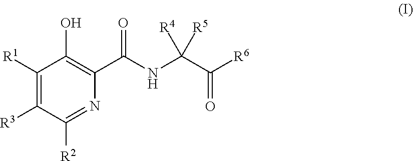

or a pharmaceutically acceptable salt thereof, wherein:

R¹ is optionally substituted C₁₋₃ alkyl, optionally substituted C₃₋₆ cycloalkyl, or optionally substituted 3- to 6-membered heterocycloalkyl;

R² is hydrogen, optionally substituted C₁₋₃ alkyl, halogen, CN, or optionally substituted cycloalkyl;

R³ is hydrogen, optionally substituted aryl, optionally substituted heteroaryl, optionally substituted cycloalkyl, optionally substituted heterocycloalkyl, carbonyl, ether, thioether, optionally substituted arylsulfonyl, optionally substituted heteroarylsulfonyl, optionally substituted arylalkyl, optionally substituted alkynyl, or optionally substituted heteroalkynyl;

R⁴ and R⁵ are independently hydrogen, optionally substituted C₁₋₃ alkyl, or R⁴ and R⁵ together with the carbon to which they are attached form an optionally substituted cycloalkyl or heterocycloalkyl; and

R⁶ is OH or ester (e.g., OR¹⁸ as described herein).

In embodiments, R¹ is unsubstituted C₁₋₃ alkyl. In embodiments, R¹ is substituted C₁₋₃ alkyl (e.g., C₁₋₃ alkyl comprising 1, 2, or 3 substituents).

In embodiments, R¹ is unsubstituted C₃₋₆ cycloalkyl (e.g., unsubstituted cyclopropyl). In embodiments, R¹ is substituted C₃₋₆ cycloalkyl (e.g., C₃₋₆ cycloalkyl comprising 1, 2, or 3 substituents).

In embodiments, R¹ is unsubstituted 3- to 6-membered heterocycloalkyl. In embodiments, R¹ is substituted 3- to 6-membered heterocycloalkyl (e.g., 3- to 6-membered heterocycloalkyl comprising 1, 2, or 3 substituents).

In embodiments, R² is hydrogen. In embodiments, R² is optionally substituted C₁₋₃ alkyl. In embodiments, R² is unsubstituted C₁₋₃ alkyl. In embodiments, R² is substituted C₁₋₃ alkyl (e.g., C₁₋₃ alkyl comprising 1, 2, or 3 substituents). In embodiments, R² is halogen. In embodiments, R² is CN. In embodiments, R² is optionally substituted cycloalkyl (e.g., a C₃₋₆ cycloalkyl). In embodiments, R² is unsubstituted cycloalkyl. In embodiments, R² is substituted cycloalkyl (e.g., cycloalkyl comprising 1, 2, or 3 substituents).

In embodiments, R³ is hydrogen. In embodiments, R³ is unsubstituted aryl (e.g., phenyl, naphthalene). In embodiments, R³ is substituted aryl (e.g., phenyl, naphthalene). In embodiments, R³ is unsubstituted heteroaryl (e.g., quinolone, isoquinoline, pyridine, pyrazole, pyrrole, triazole, tetrazole, oxazole, thiazole). In embodiments, R³ is substituted heteroaryl (e.g., quinolone, isoquinoline, pyridine, pyrazole, pyrrole, triazole, tetrazole, oxazole, thiazole). In embodiments, R³ is unsubstituted cycloalkyl. In embodiments, R³ is substituted cycloalkyl. In embodiments, R³ is unsubstituted heterocycloalkyl (e.g., N-containing heterocycloalkyl). In embodiments, R³ is substituted heterocycloalkyl (e.g., N-containing heterocycloalkyl). In embodiments, R³ is a carbonyl group (e.g., COR¹⁷, where R¹⁷ is according to any embodiment described herein). In embodiments, R³ is an ether (e.g., OR¹⁶, where R¹⁶ is according to any embodiment described herein). In embodiments, R³ is thioether (e.g., SR¹⁶, where R¹⁶ is according to any embodiment described herein). In embodiments, R³ is unsubstituted arylsulfonyl (e.g., phenylsulfonyl). In embodiments, R³ is substituted arylsulfonyl. In embodiments, R³ is unsubstituted heteroarylsulfonyl. In embodiments, R³ is substituted heteroarylsulfonyl. In embodiments, R³ is unsubstituted arylalkyl (e.g., phenylalkyl). In embodiments, R³ is substituted arylalkyl (e.g., phenylalkyl). In embodiments, R³ is unsubstituted alkynyl. In embodiments, R³ is substituted alkynyl (e.g., aryl substituted alkynyl). In embodiments, R³ is unsubstituted heteroalkynyl. In embodiments, R³ is substituted heteroalkynyl (e.g., aryl substituted heteroalkynyl). In embodiments, R³ is OR¹⁶, SR¹⁶, SO₂R¹⁶, CH₂R¹⁶, CH₂CH₂R¹⁶, C≡CR¹⁶, or C≡CCH₂OR¹⁶, and wherein R¹⁶ is aryl.

In embodiments, R⁴ and R⁵ are independently hydrogen or optionally substituted C₁₋₃ alkyl. In embodiments, R⁴ and R⁵ are independently hydrogen or unsubstituted C₁₋₃ alkyl. In embodiments, R⁴ and R⁵ are each hydrogen. In embodiments, one of R⁴ and R⁵ is hydrogen and the other is unsubstituted C₁₋₃ alkyl. In embodiments, R⁴ and R⁵ are each unsubstituted C₁₋₃ alkyl. In embodiments, R⁴ and R⁵ together with the carbon to which they are attached form an optionally substituted cycloalkyl (e.g., C₃₋₆ cycloalkyl). In embodiments, R⁴ and R⁵ together with the carbon to which they are attached form an unsubstituted cycloalkyl (e.g., unsubstituted C₃₋₆ cycloalkyl). In embodiments, R⁴ and R⁵ together with the carbon to which they are attached form an optionally substituted heterocycloalkyl (e.g., a 3- to 6-membered heterocycloalkyl). In embodiments, R⁴ and R⁵ together with the carbon to which they are attached form an unsubstituted heterocycloalkyl (e.g., an unsubstituted 3- to 6-membered heterocycloalkyl).

In embodiments, R⁶ is hydrogen. In embodiments, R⁶ is an ester (e.g., OR¹⁸ as described herein). In embodiments, R⁶ is OR¹⁸, wherein R¹⁸ is C₁₋₆ alkyl.

In embodiments, R¹ is optionally substituted C₁₋₃ alkyl; and/or R³ is hydrogen, optionally substituted aryl, optionally substituted heteroaryl, optionally substituted cycloalkyl, optionally substituted heterocycloalkyl, carbonyl, or ether.

In embodiments, R¹ is unsubstituted C₁₋₃ alkyl (e.g., CH₃ or CH₂CH₃). In embodiments, R¹ is CH₃. In embodiments, R² is hydrogen. In embodiments, R⁴ and R⁵ are each hydrogen. In embodiments, R⁶ is OH.

In embodiments, each R¹ and R² is unsubstituted C₁₋₃ alkyl. In embodiments, each R¹ and R² is CH₃. In embodiments, R⁴ and R⁵ are each hydrogen. In embodiments, R⁶ is OH.

In embodiments, R² is unsubstituted C₁₋₃ alkyl (e.g., CH₃ or CH₂CH₃). In embodiments, R² is CH₃. In embodiments, R³ is hydrogen. In embodiments, R⁴ and R⁵ are each hydrogen. In embodiments, R⁶ is OH.

In embodiments, a compound has a structure according to Formula (I),

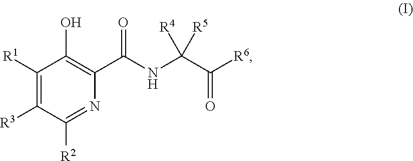

or a pharmaceutically acceptable salt thereof, wherein

R¹ is C₁₋₃ alkyl optionally substituted with OR⁷, halogen, or aryl, which is optionally substituted with halogen, and wherein R⁷ is C₁₋₃ alkyl optionally substituted with aryl, or R¹ is optionally substituted C₃₋₆ cycloalkyl or optionally substituted 3- to 6-membered heterocycloalkyl;

R² is hydrogen, halogen, CN, or C₁₋₃ alkyl optionally substituted with one or more halogens;

R³ is selected from the group consisting of:

hydrogen;

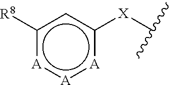

wherein X is a covalent bond, O, S, SO₂, C₁₋₄ alkylene, C₂₋₄ alkynylene, or C₂₋₄ heteroalkynylene; each A is independently N or CR⁹, R⁸ and R⁹ are independently hydrogen, halogen, OR¹⁰, or C₁₋₃ alkyl optionally substituted with one or more halogens, and R¹⁰ is C₁₋₃ alkyl or aryl;

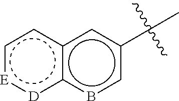

wherein B is N or CR¹¹, D is N, NH, or CR¹¹, E is N, CR¹¹, or CHR¹², and R¹¹ and R¹² are independently hydrogen or C₁₋₃ alkyl, and wherein the dashed circle represents the presence or absence of a conjugated system;

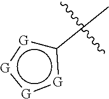

wherein each G is independently N, NH, NR¹³, or CR¹⁴; R¹³ is C₃₋₆ cycloalkyl, 3- to 6-membered heterocycloalkyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens, and R¹⁴ is hydrogen, halogen, C₃₋₆ cycloalkyl, 3- to 6-membered heterocycloalkyl, or C₁₋₃ alkyl;

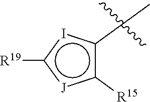

wherein I is O, S, or CH, J is N or CH, R¹⁵ is hydrogen, C₃₋₆ cycloalkyl, 3- to 6-membered heterocycloalkyl, or C₁₋₃ alkyl, and R¹⁹ is hydrogen, C₃₋₆ cycloalkyl, 3- to 6-membered heterocycloalkyl, or aryl; OR⁶ wherein R¹⁶ is aryl;

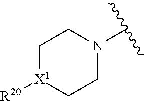

wherein X¹ is N or CH, and R²⁰ is optionally substituted aryl; and COR¹⁷ wherein R¹⁷ is aryl;

R⁴ and R⁵ are independently hydrogen, C₁₋₃ alkyl optionally substituted with one or more halogens, or R⁴ and R⁵ together with the carbon to which they are attached form a optionally substituted cycloalkyl or heterocycloalkyl; and

R⁶ is OH or OR¹⁸, wherein R¹⁸ is C₁₋₆ alkyl.

In embodiments, R¹ is unsubstituted C₁₋₃ alkyl. In embodiments, R¹ is CH₃, or CH₂CH₃.

In embodiments, R¹ is substituted C₁₋₃ alkyl. In embodiments, R¹ is C₁₋₃ alkyl substituted with OR⁷. In embodiments, R⁷ is unsubstituted C₁₋₃ alkyl. In embodiments, R⁷ is substituted C₁₋₃ alkyl. In embodiments, R⁷ is C₁₋₃ alkyl substituted with aryl. In embodiments, an aryl is a phenyl. In embodiments, R⁷ is C₁₋₃ alkyl substituted with phenyl. In embodiments, R¹ is C₁₋₃ alkyl substituted with OBn. In embodiments, R¹ is CH₂CH₂OBn.

In embodiments, R¹ is C₁₋₃ alkyl substituted with one or more halogens (e.g., F, Cl, Br or I). In embodiments, R¹ is difluoromethyl.

In embodiments, R¹ is C₁₋₃ alkyl substituted with aryl which is optionally substituted with halogen. In embodiments, the optionally substituted aryl is an optionally substituted phenyl. In embodiments, the aryl or phenyl is unsubstituted aryl or unsubstituted phenyl. In embodiments, the aryl or phenyl is substituted with one or more halogens. In embodiments, R¹ is

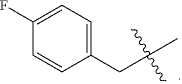 [SMILES: *Cc1ccc(F)cc1]

In embodiments, R¹ is optionally substituted C₃₋₆ cycloalkyl (e.g., optionally substituted cyclopropyl). In embodiments, R¹ is C₃₋₆ cycloalkyl (e.g., unsubstituted cyclopropyl). In embodiments, R¹ is substituted C₃₋₆ cycloalkyl (e.g., C₃₋₆ cycloalkyl comprising 1, 2, or 3 substituents).

In embodiments, R¹ is optionally substituted 3- to 6-membered heterocycloalkyl. In embodiments, R¹ is unsubstituted 3- to 6-membered heterocycloalkyl. In embodiments, R¹ is substituted 3- to 6-membered heterocycloalkyl (e.g., 3- to 6-membered heterocycloalkyl comprising 1, 2, or 3 substituents).

In embodiments, R² is hydrogen.

In embodiments, R² is CN.

In embodiments, R² is halogen. In embodiments, a halogen is F, Cl, Br, or I.

In embodiments, R² is unsubstituted C₁₋₃ alkyl. In embodiments, R² is CH₃.

In embodiments, R² is C₁₋₃ alkyl substituted with one or more halogens.

In embodiments, R³ is hydrogen. In embodiments, R³ is not hydrogen.

In embodiments, R³ is

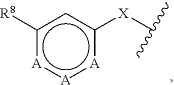

wherein

X is a covalent bond, O, S, SO₂, C₁₋₄ alkylene, C₂₋₄ alkynylene, or C₂₋₄ heteroalkynylene;

each A is independently N or CR⁹;

R⁸ and R⁹ are independently hydrogen, halogen, OR¹⁰, or C₁₋₃ alkyl optionally substituted with one or more halogens; and

R¹⁰ is C₁₋₃ alkyl or aryl.

In embodiments, R³ is unsubstituted phenyl, fluorophenyl, chlorophenyl, difluorophenyl, dichlorophenyl, or trifluoromethylphenyl.

In embodiments, R³ is

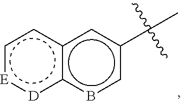

wherein

B is N or CR¹¹;

D is N, NH, or CR¹¹;

E is N, CR¹¹, or CHR¹²; and

R¹¹ and R¹² are independently hydrogen or C₁₋₃ alkyl; and wherein

the dashed circle represents the presence or absence of a conjugated system.

In embodiments, R³ is

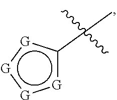

wherein

each G is independently N, NH, NR¹³, or CR¹⁴;

R¹³ is C₃₋₆ cycloalkyl, 3- to 6-membered heterocycloalkyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens; and

R¹⁴ is hydrogen, halogen, C₃₋₆ cycloalkyl, 3- to 6-membered heterocycloalkyl, or C₁₋₃ alkyl.

In embodiments, R³ is pyrrolyl, tetrazolyl, triazolyl, or pyrazolyl, optionally substituted by aryl or cycloalkyl. In embodiments, R³ (e.g., pyrrolyl, tetrazolyl, triazolyl, or pyrazolyl) is substituted by cyclopropyl, unsubstituted phenyl, fluorophenyl, chlorophenyl, difluorophenyl, dichlorophenyl, or trifluoromethylphenyl.

In embodiments, R³ is

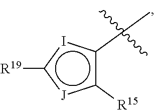

wherein

I is O, S, or CH;

J is N or CH;

R¹⁵ is hydrogen, C₃₋₆ cycloalkyl, 3- to 6-membered heterocycloalkyl, or C₁₋₃ alkyl; and

R¹⁹ is hydrogen, C₃₋₆ cycloalkyl, 3- to 6-membered heterocycloalkyl, or aryl.

In embodiments, R³ is OR¹⁶ wherein R¹⁶ is aryl. In embodiments, the aryl is a phenyl. In embodiments, R³ is OPh.

In embodiments, R³ is

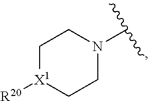

wherein

X¹ is N or CH; and

R²⁰ is optionally substituted aryl.

In embodiments, R³ is piperidinyl or piperazinyl, optionally substituted by cyclopropyl or aryl. In embodiments, R³ (e.g., piperidinyl or piperazinyl) is substituted by cyclopropyl, unsubstituted phenyl, fluorophenyl, chlorophenyl, difluorophenyl, dichlorophenyl, or trifluoromethylphenyl.

In embodiments, R³ is COR¹⁷ wherein R¹⁷ is aryl. In embodiments, the aryl is a phenyl. In embodiments, R³ is COPh.

In embodiments, R³ is

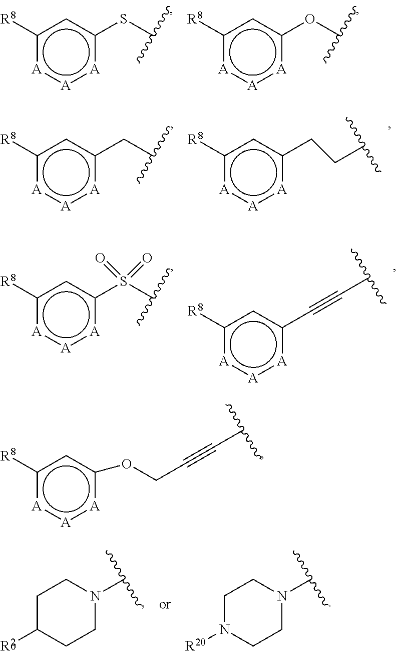

In embodiments, R⁴ and R⁵ are both hydrogen.

In embodiments, one of R⁴ and R⁵ is hydrogen, and the other is C₁₋₃ alkyl. In embodiments, the C₁₋₃ alkyl is unsubstituted. In embodiments, the C₁₋₃ alkyl is substituted with one or more halogens. In embodiments, the C₁₋₃ alkyl is CH₃.

In embodiments, R⁴ and R⁵ are both C₁₋₃ alkyl. In embodiments, the C₁₋₃ alkyl is unsubstituted. In embodiments, the C₁₋₃ alkyl is substituted with one or more halogens. In embodiments, the C₁₋₃ alkyl is CH₃.

In embodiments, R⁴ and R⁵ together with the carbon to which they are attached form a cycloalkyl or heterocycloalkyl. In embodiments, the cycloalkyl or heterocycloalkyl is unsubstituted. In embodiments, the cycloalkyl or heterocycloalkyl is unsubstituted (e.g., cycloalkyl or heterocycloalkyl comprising 1, 2, or 3 substituents). In embodiments, the cycloalkyl or heterocycloalkyl is a 3-membered ring. In embodiments, the cycloalkyl or heterocycloalkyl is a 4-membered ring. In embodiments, the heterocycloalkyl is an oxygen-containing heterocycloalkyl. In embodiments, the cycloalkyl or heterocycloalkyl is selected from the group consisting of cyclopropyl, cyclobutyl, and

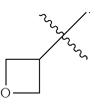 [SMILES: *C1COC1]

In embodiments, R⁶ is OH.

In embodiments, R⁶ is OR¹⁸, wherein R¹⁸ is C₁₋₆ alkyl.

In embodiments, R¹ is unsubstituted C₁₋₃ alkyl (e.g., CH₃ or CH₂CH₃). In embodiments, R¹ is CH₃. In embodiments, R² is hydrogen. In embodiments, R⁴ and R⁵ are each hydrogen. In embodiments, R⁶ is OH.

In embodiments, each R¹ and R² is unsubstituted C₁₋₃ alkyl. In embodiments, each R¹ and R² is CH₃. In embodiments, R⁴ and R⁵ are each hydrogen. In embodiments, R⁶ is OH.

In embodiments, R² is unsubstituted C₁₋₃ alkyl (e.g., CH₃ or CH₂CH₃). In embodiments, R² is CH₃. In embodiments, R³ is hydrogen. In embodiments, R⁴ and R⁵ are each hydrogen. In embodiments, R⁶ is OH.

In embodiments, R¹ is C₁₋₃ alkyl optionally substituted with OR⁷ or aryl, which is optionally substituted with halogen, and wherein R⁷ is C₁₋₃ alkyl optionally substituted with aryl; and/or R³ is selected from the group consisting of: hydrogen,

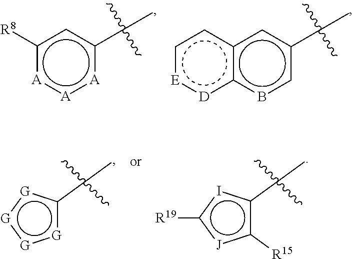

In embodiments, a compound of Formula (I) has the following structure,

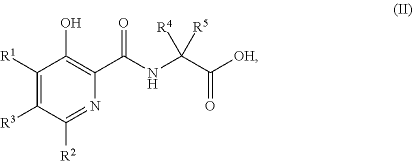

or a pharmaceutically acceptable salt thereof, wherein R¹, R², R³, R⁴, and R⁵ are as defined anywhere herein.

In embodiments, a compound of Formula (I) or Formula (II) has the following structure,

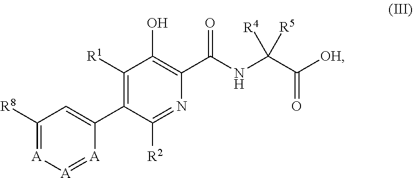

or a pharmaceutically acceptable salt thereof, wherein R¹, R², R⁴, and R⁵ are as defined anywhere herein, and wherein

each A is independently N or CR⁹;

R⁸ and R⁹ are independently hydrogen, halogen, OR¹⁰, or C₁₋₃ alkyl optionally substituted with one or more halogens; and

R¹⁰ is C₁₋₃ alkyl or aryl.

In embodiments, A is N. In embodiments, A is CR⁹. In embodiments, all three A groups are CR⁹. In embodiments, one A is CR⁹, and the other two A groups are N. In embodiments, one A is N, the other two A groups are CR⁹. In embodiments, all three A groups are N.

In embodiments, at least one of R⁸ and R⁹ is hydrogen. In embodiments, one of R⁸ and R⁹ is hydrogen.

In embodiments, none of R⁸ and R⁹ is hydrogen.

In embodiments, R⁸ is hydrogen.

In embodiments, R⁸ is halogen. In embodiments, a halogen is F, Cl, Br, or I. In embodiments, R⁸ is Cl.

In embodiments, R⁸ is OR¹⁰, wherein R¹⁰ is C₁₋₃ alkyl. In embodiments, R⁸ is OMe.

In embodiments, R⁸ is OR¹⁰, wherein R¹⁰ is aryl. In embodiments, the aryl is a phenyl. In embodiments, R⁸ is OPh.

In embodiments, R⁸ is unsubstituted C₁₋₃ alkyl. In embodiments, R⁸ is C₁₋₃ alkyl substituted with one or more halogens.

In embodiments, R⁹ is hydrogen.

In embodiments, R⁹ is halogen. In embodiments, a halogen is F, Cl, Br, or I. In embodiments, R⁹ is Cl.

In embodiments, R⁹ is OR¹⁰, wherein R¹⁰ is C₁₋₃ alkyl. In embodiments, R⁹ is OMe.

In embodiments, R⁹ is OR¹⁰, wherein R¹⁰ is aryl. In embodiments, the aryl is a phenyl. In embodiments, R⁹ is OPh.

In embodiments, R⁹ is unsubstituted C₁₋₃ alkyl. In embodiments, R⁹ is C₁₋₃ alkyl substituted with one or more halogens. In embodiments, R⁹ is CH₃. In embodiments, R⁹ is CF₃.

In embodiments, a compound of Formula (I), Formula (II), or Formula (III) has the following structure,

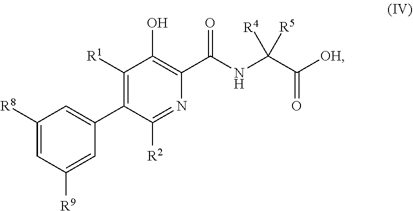

or a pharmaceutically acceptable salt thereof, wherein R¹, R², R⁴, R⁵, R⁸ and R⁹ are as defined anywhere herein.

In embodiments, a compound of Formula (I), Formula (II), or Formula (III) has the following structure,

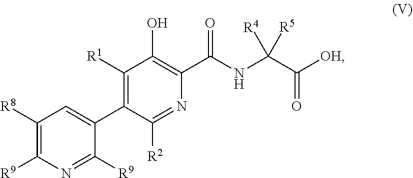

or a pharmaceutically acceptable salt thereof, wherein R¹, R², R⁴, R⁵, R⁸ and R⁹ are as defined anywhere herein.

In embodiments,

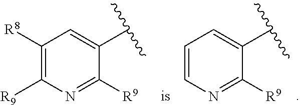

In embodiments, R⁹ is halogen, OR¹⁰, or C₁₋₃ alkyl optionally substituted with one or more halogens. In embodiments, R⁹ is halogen.

In embodiments,

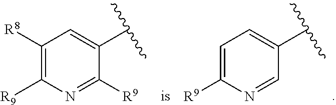

In embodiments, R⁹ is halogen, OR¹⁰, or C₁₋₃ alkyl optionally substituted with one or more halogens. In embodiments, R⁹ is halogen,

In embodiments,

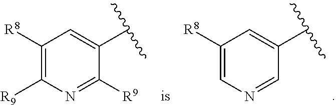

In embodiments, R⁸ is halogen, OR¹⁰, or C₁₋₃ alkyl optionally substituted with one or more halogens. In embodiments, R⁸ is halogen.

In embodiments, a compound of Formula (I) or Formula (II) has the following structure,

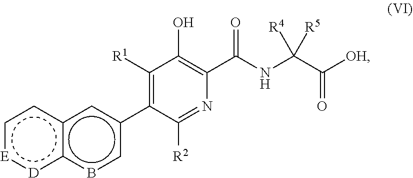

or a pharmaceutically acceptable salt thereof, wherein R¹, R², R⁴, and R⁵ are as defined anywhere herein, and wherein

B is N or CR¹¹;

D is N, NH, or CR¹¹;

E is N, CR¹¹, or CHR¹²; and

R¹¹ and R¹² are independently hydrogen or C₁₋₃ alkyl; and the dashed circle represents the presence or absence of a conjugated system.

In embodiments, the dashed circle is present, and R³ is

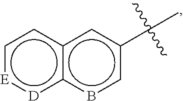

wherein B is N or CR¹¹; D is N, or CR¹¹; and E is N or CR¹².

In embodiments, D is CR¹¹, E is CR¹², and B is N.

In embodiments, B is CR¹¹, E is CR¹², and D is N.

In embodiments, B and D are both CR¹¹, and E is N.

In embodiments, B and D are both CR¹¹, and E is CR¹².

In embodiments, the dashed circle is not present, and R³ is

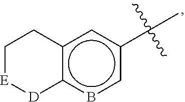

wherein B is N or CR¹¹; D is NH; and E is CHR¹².

In embodiments, B is CR¹¹, and E is CHR¹².

In embodiments, R¹¹ is hydrogen.

In embodiments, R¹¹ is C₁₋₃ alkyl. In embodiments, R¹¹ is CH₃.

In embodiments, R¹² is hydrogen.

In embodiments, R¹² is C₁₋₃ alkyl. In embodiments, R¹² is CH₃.

In embodiments, a compound of Formula (I), Formula (II), or Formula (VI) has the following structure,

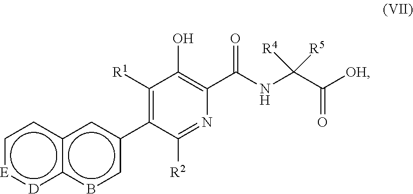

or a pharmaceutically acceptable salt thereof, wherein B, D, E, R¹, R², R⁴, and R⁵ are as defined anywhere herein.

In embodiments,

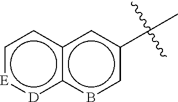

is

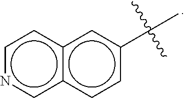

In embodiments,

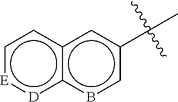

is

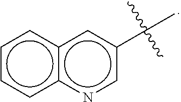

In embodiments

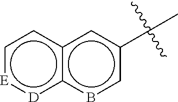

is

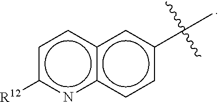

In embodiments, R¹² is hydrogen or C₁₋₃ alkyl. In embodiments, R¹² is hydrogen. In embodiments, R¹² is CH₃.

In embodiments,

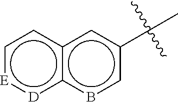

is

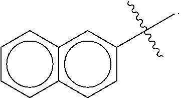

In embodiments, a compound of Formula (I), Formula (II), or Formula (VI) has the following structure,

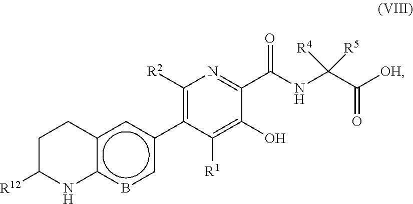

or a pharmaceutically acceptable salt thereof, wherein B, R¹, R², R⁴, R⁵ and R¹² are as defined anywhere herein.

In embodiments,

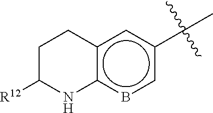

is

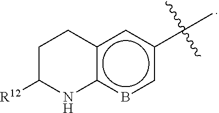

In embodiments, R¹² is C₁₋₃ alkyl. In embodiments, R¹² is CH₃.

In embodiments, a compound of Formula (I) or Formula (II) has the following structure,

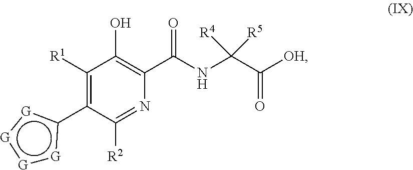

or a pharmaceutically acceptable salt thereof, wherein R¹, R², R⁴, and R⁵ are as defined anywhere herein, and wherein

each G is independently N, NH, NR¹³, or CR¹⁴;

R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens; and

R¹⁴ is hydrogen, halogen, cyclopropyl, or C₁₋₃ alkyl.

In embodiments, G is N. In embodiments, G is NH. In embodiments, G is NR¹³. In embodiments, G is CR¹⁴.

In embodiments, R¹³ is cyclopropyl.

In embodiments, R¹³ is unsubstituted aryl. In embodiments, R¹³ is aryl substituted with one or more halogens. In embodiments, R¹³ is aryl substituted with one or more optionally substituted C₁₋₃ alkyl (e.g., C₁₋₃ alkyl substituted with one or more halogens). In embodiments, an aryl is a phenyl group. In embodiments, R¹³ is unsubstituted phenyl. In embodiments, R¹³ is phenyl substituted with one or more halogens. In embodiments, R¹³ is phenyl substituted with one or more optionally substituted C₁₋₃ alkyl (e.g., C₁₋₃ alkyl substituted with one or more halogens). In embodiments, R¹³ is selected from the group consisting of p-trifluoromethylphenyl, m-fluorophenyl, p-fluorophenyl, p-chlorophenyl, 2,4-dichlorophenyl, and 3,5-dichlorophenyl.

In embodiments, R¹³ is heteroaryl. In embodiments, the heteroaryl is unsubstituted. In embodiments, the heteroaryl is substituted. In embodiments, the heteroaryl is a pyridyl. In embodiments, R¹³ is 2-pyridyl, 3-pyridyl, or 4-pyridyl.

In embodiments, R¹³ is unsubstituted heterocycloalkyl. In embodiments, R¹³ is heterocycloalkyl substituted with t-butyloxycarbonyl. In embodiments, the heterocycloalkyl is a 6-membered heterocycloalkyl. In embodiments, the heterocycloalkyl is a nitrogen-containing heterocycloalkyl. In embodiments, the heterocycloalkyl is an oxygen-containing heterocycloalkyl. In embodiments, R¹³ is

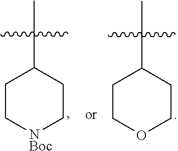 [SMILES: *C1CCN(C(=O)OC(C)(C)C)CC1 ; *C1CCOCC1]

In embodiments, R¹³ is unsubstituted C₁₋₄ alkyl. In embodiments, R¹³ is

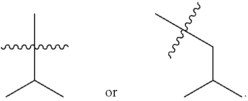

In embodiments, R¹³ is C₁₋₄ alkyl substituted with aryl. In embodiments, the aryl is unsubstituted. In embodiments, the aryl is substituted with one or more halogens. In embodiments, an aryl is a phenyl group. In embodiments, the phenyl is unsubstituted phenyl. In embodiments, the phenyl is substituted with one or more halogens. In embodiments, R¹³ is

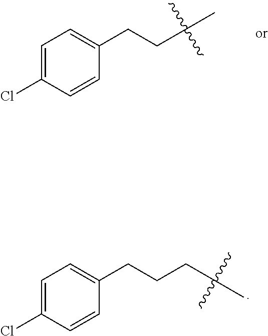 [SMILES: *CCc1ccc(Cl)cc1 ; *CCCc1ccc(Cl)cc1]

In embodiments, R¹⁴ is hydrogen.

In embodiments, R¹⁴ is halogen. In embodiments, a halogen is F, Cl, Br, or I. In embodiments, R¹⁴ is F.

In embodiments, R¹⁴ is cyclopropyl.

In embodiments, R¹⁴ is C₁₋₃ alkyl. In embodiments, R¹⁴ is CH₃.

In embodiments,

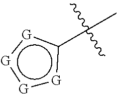

is

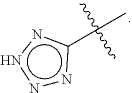

In embodiments,

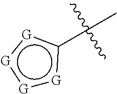

is

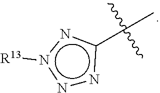

In embodiments, R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens. In embodiments, R¹³ is unsubstituted aryl. In embodiments, R¹³ is Ph. In embodiments, R¹³ is aryl substituted with one or more halogens. In embodiments, R¹³ is

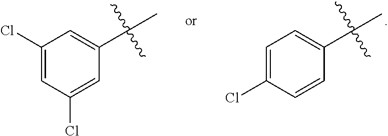 [SMILES: *c1cc(Cl)cc(Cl)c1 ; *c1ccc(Cl)cc1]

In embodiments, R¹³ is cyclopropyl.

In embodiments,

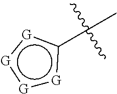

is

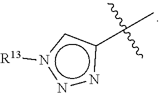

In embodiments, R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens. In embodiments, R¹³ is aryl optionally substituted with one or more halogens. In embodiments, R¹³ is C₁₋₄ alkyl substituted with aryl, which is substituted with one or more halogens. In embodiments, R¹³ is aryl optionally substituted with one or more halogens. In embodiments, R¹³ is Ph,

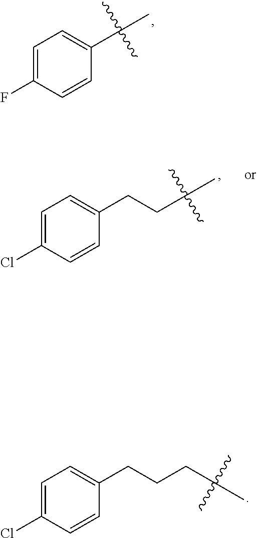 [SMILES: *c1ccc(F)cc1 ; *CCc1ccc(Cl)cc1 ; *CCCc1ccc(Cl)cc1]

In embodiments,

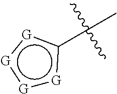

is

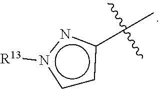

In embodiments,

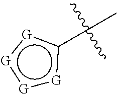

is

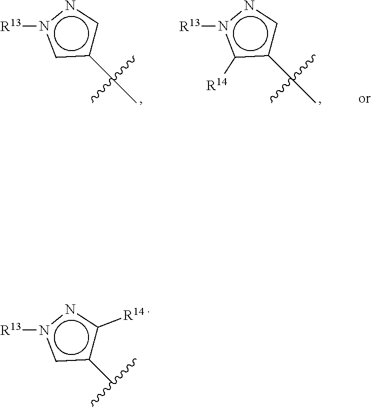

In embodiments, R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens. In embodiments, R¹³ is aryl. In embodiments, R¹³ is Ph. In embodiments, R¹³ is Ph,

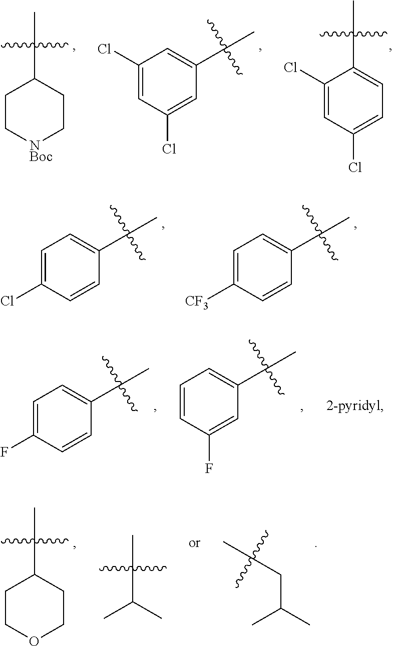

In embodiments, R¹⁴ is hydrogen, halogen, cyclopropyl, or C₁₋₃ alkyl. In embodiments, R¹⁴ is C₁₋₃ alkyl (e.g., methyl).

In embodiments,

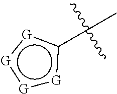

is

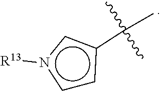

In embodiments, R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens. In embodiments, R¹³ is aryl. In embodiments, R¹³ is Ph.

In embodiments, a compound of Formula (I), Formula (II), or Formula (IX) has the following structure,

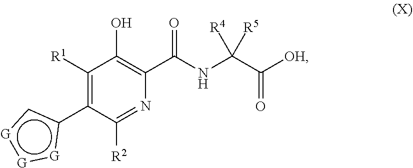

or a pharmaceutically acceptable salt thereof, wherein G, R¹, R², R⁴, and R⁵ are as defined anywhere herein.

In embodiments,

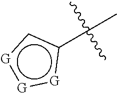

is

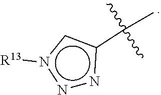

In embodiments, R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens. In embodiments, R¹³ is aryl optionally substituted with one or more halogens. In embodiments, R¹³ is C₁₋₄ alkyl substituted with aryl, which is substituted with one or more halogens. In embodiments, R¹³ is aryl optionally substituted with one or more halogens. In embodiments, R¹³ is Ph,

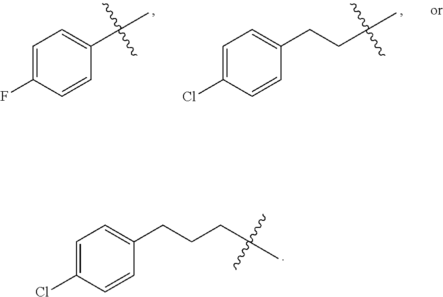 [SMILES: *c1ccc(F)cc1 ; *CCc1ccc(Cl)cc1 ; *CCCc1ccc(Cl)cc1]

In embodiments, a compound of Formula (I), Formula (II), Formula (IX), or Formula (X) has the following structure,

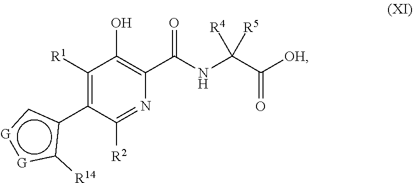

or a pharmaceutically acceptable salt thereof, wherein R¹, R², R⁴, R⁵, and R¹⁴ are as defined anywhere herein.

In embodiments, a compound of Formula (I), Formula (II), Formula (IX), Formula (X), or Formula (XI) has the following structure,

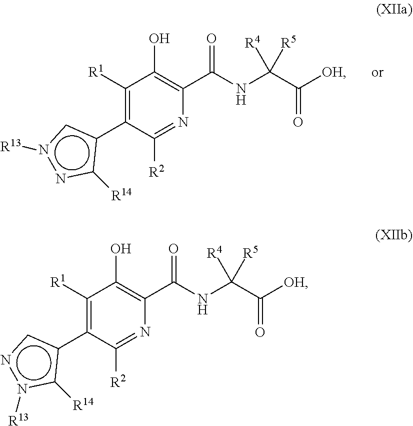

or a pharmaceutically acceptable salt thereof, wherein R¹, R², R⁴, R⁵, R¹³ and R¹⁴ are as defined anywhere herein.

In embodiments, R¹⁴ is halogen or C₁₋₃ alkyl. In embodiments, R¹⁴ is methyl. In embodiments, R¹⁴ is F.

In embodiments, R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens. In embodiments, R¹³ is aryl optionally substituted with one or more halogens. In embodiments, R¹³ is Ph, 3-flourophenyl, or 4-flourophenyl.

In embodiments, R¹⁴ is hydrogen and

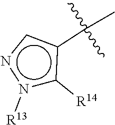

is

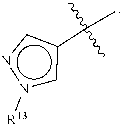

In embodiments, R¹⁴ is hydrogen and

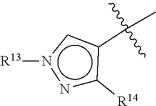

is

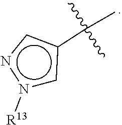

In embodiments, R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens. In embodiments, R¹³ is C₁₋₄ alkyl. In embodiments, R¹³ is heteroaryl. In embodiments, R¹³ is heterocycloalkyl. In embodiments, R¹³ is aryl substituted with one or more halogens. In embodiments, R¹³ is aryl substituted with optionally substituted C₁₋₃ alkyl (e.g., C₁₋₃ alkyl substituted with one or more halogens). In embodiments, R¹³ is Ph,

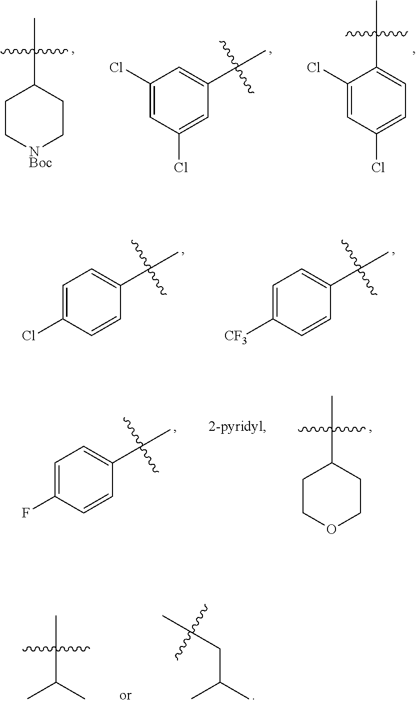

In embodiments, a compound of Formula (I), Formula (II), Formula (IX), Formula (X), Formula (XI), or Formula (XIIa) has the following structure,

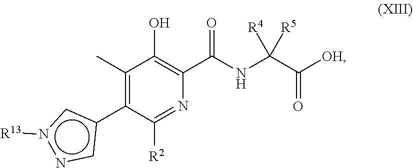

or a pharmaceutically acceptable salt thereof, wherein R², R⁴, R⁵, and R¹³ are as defined anywhere herein.

In embodiments, R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens. In embodiments, R¹³ is heterocycloalkyl substituted with t-butyloxycarbonyl. In embodiments, R¹³ is aryl. In embodiments, R¹³ is aryl substituted with one or more halogens. In embodiments, R¹³ is aryl substituted with optionally substituted C₁₋₃ alkyl (e.g., C₁₋₃ alkyl substituted with one or more halogens). In embodiments, R¹³ is Ph,

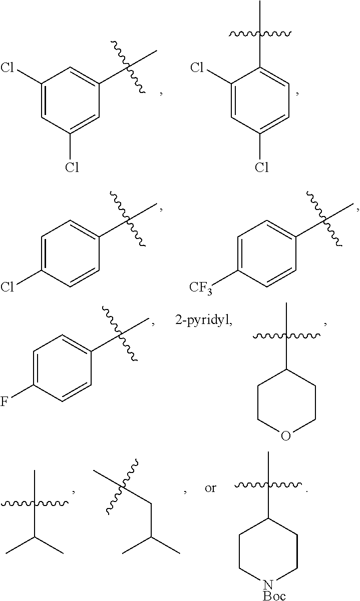

In embodiments, a compound of Formula (I), Formula (II), Formula (IX), Formula (X), or Formula (XI) has the following structure,

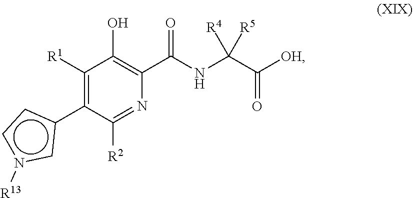

or a pharmaceutically acceptable salt thereof, wherein R¹, R², R⁴, R⁵, and R¹³ are as defined anywhere herein.

In embodiments, R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens. In embodiments, R¹³ is unsubstituted aryl. In embodiments, R¹³ is Ph.

In embodiments, a compound of Formula (I), Formula (II), or Formula (IX) has the following structure,

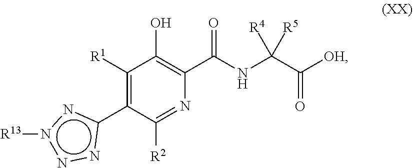

or a pharmaceutically acceptable salt thereof, wherein R¹, R², R⁴, R⁵, and R¹³ are as defined anywhere herein.

In embodiments, R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens. In embodiments, R¹³ is unsubstituted aryl. In embodiments, R¹³ is Ph. In embodiments, R¹³ is aryl substituted with one or more halogens. In embodiments, R¹³ is

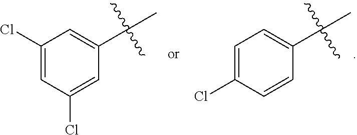 [SMILES: *c1cc(Cl)cc(Cl)c1 ; *c1ccc(Cl)cc1]

In embodiments, R¹³ is cyclopropyl.

In embodiments, a compound of Formula (I), Formula (II), or Formula (IX) has the following structure,

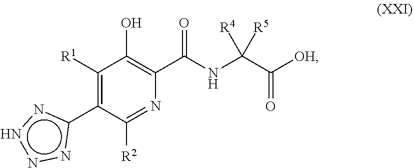

or a pharmaceutically acceptable salt thereof, wherein R¹, R², R⁴, and R⁵ are as defined anywhere herein.

In embodiments, a compound of Formula (I) or Formula (II) has the following structure,

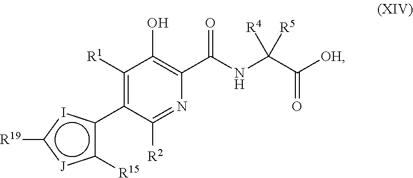

or a pharmaceutically acceptable salt thereof, wherein R¹, R², R⁴, and R⁵ are as defined anywhere herein, and wherein

I is O, S, or CH;

J is N or CH;

R¹⁵ is hydrogen or C₁₋₃ alkyl; and

R¹⁹ is hydrogen or aryl.

In embodiments, I is O. In embodiments, I is S. In embodiments, I is CH.

In embodiments, J is N. In embodiments, J is CH.

In embodiments, R¹⁵ is hydrogen.

In embodiments, R¹⁵ is C₁₋₃ alkyl. In embodiments, R¹⁵ is CH₃.

In embodiments, R¹⁹ is hydrogen.

In embodiments, R¹⁹ is aryl. In embodiments, R¹⁹ is phenyl.

In embodiments, a compound of Formula (I), (II) or (XIV) has the following structure,

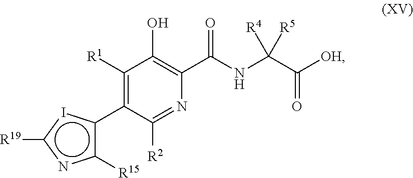

or a pharmaceutically acceptable salt thereof, wherein I, R¹, R², R⁴, R⁵, R¹⁵ and R¹⁹ are as defined anywhere herein.

In embodiments,

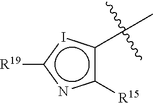

is

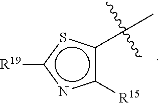

In embodiments, R¹⁹ is aryl. In embodiments, R¹⁹ is phenyl. In embodiments, R¹⁵ is hydrogen or C₁₋₃ alkyl. In embodiments, R¹⁵ is hydrogen or CH₃.

In embodiments,

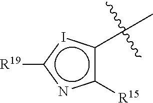

is

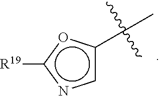

In embodiments, R¹⁹ is aryl. In embodiments, R¹⁹ is phenyl.

In embodiments, a compound of Formula (I) or Formula (II) has the following structure,

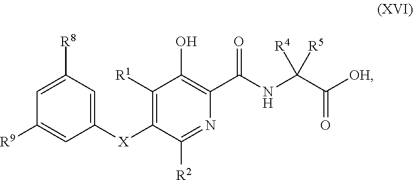

or a pharmaceutically acceptable salt thereof, wherein R¹, R², R⁴, R⁵, R⁸ and R⁹ are as defined anywhere herein, and wherein X is O, S, or SO₂.

In embodiments, X is O. In embodiments, X is S. In embodiments, X is In embodiments, X is SO₂.

In embodiments, a compound of Formula (I) or Formula (II) has the following structure,

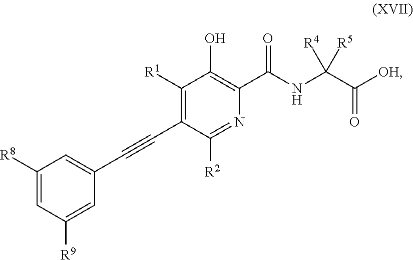

or a pharmaceutically acceptable salt thereof, wherein R¹, R², R⁴, R⁵, R⁸ and R⁹ are as defined anywhere herein.

In embodiments, a compound of Formula (I) or Formula (II) has the following structure,

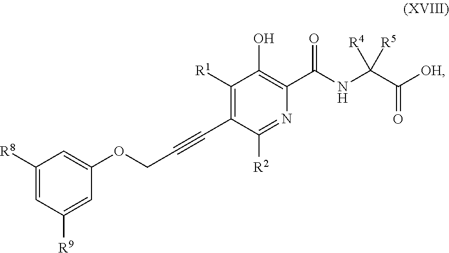

or a pharmaceutically acceptable salt thereof, wherein R¹, R², R⁴, R⁵, R⁸ and R⁹ are as defined anywhere herein.

In embodiments, a compound of Formula (I) or Formula (II) has the following structure,

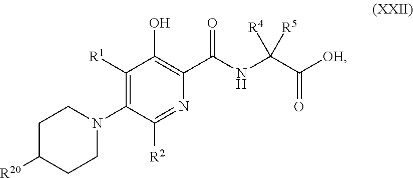

or a pharmaceutically acceptable salt thereof, wherein R¹, R², R⁴, and R⁵ are as defined anywhere herein, and wherein R²⁰ is optionally substituted aryl.

In embodiments, R²⁰ is substituted aryl (e.g., comprising 1, 2, or 3 substituents). In embodiments, R²⁰ is unsubstituted aryl. In embodiments, an aryl is a phenyl. In embodiments, R²⁰ is Ph.

In embodiments, a compound of Formula (I) or Formula (II) has the following structure,

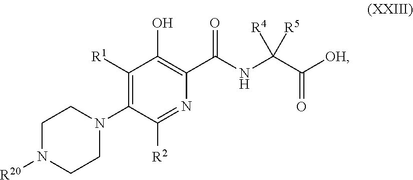

or a pharmaceutically acceptable salt thereof, wherein R¹, R², R⁴, and R⁵ are as defined anywhere herein, and wherein R²⁰ is optionally substituted aryl.

In embodiments, R²⁰ is substituted aryl (e.g., comprising 1, 2, or 3 substituents). In embodiments, R²⁰ is unsubstituted aryl. In embodiments, an aryl is a phenyl. In embodiments, R²⁰ is Ph.

Exemplary Compounds

In some embodiments, the PHD inhibitor compound is any one of Compounds 1-50 or a pharmaceutically acceptable salt thereof.

| Cmpd No. | Structure |
|---|---|
| 1 | 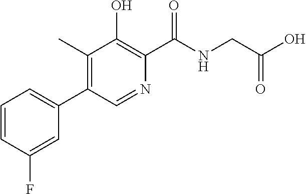 [SMILES: Cc1c(-c2cccc(F)c2)cnc(C(=O)NCC(=O)O)c1O] |
| 2 | 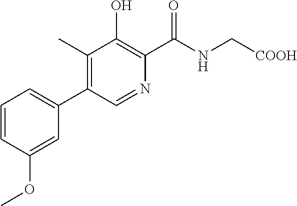 [SMILES: COc1cccc(-c2cnc(C(=O)NCC(=O)O)c(O)c2C)c1] |
| 3 | 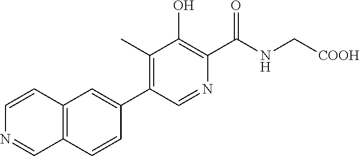 [SMILES: Cc1c(-c2ccc3cnccc3c2)cnc(C(=O)NCC(=O)O)c1O] |
| 4 | 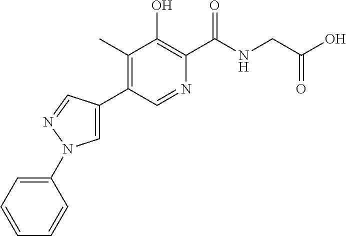 [SMILES: Cc1c(-c2cnn(-c3ccccc3)c2)cnc(C(=O)NCC(=O)O)c1O] |
| 5 | 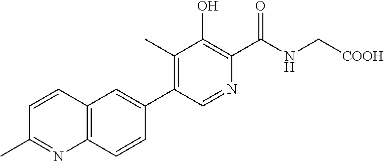 [SMILES: Cc1ccc2cc(-c3cnc(C(=O)NCC(=O)O)c(O)c3C)ccc2n1] |
| 6 | 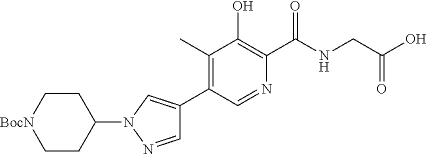 [SMILES: Cc1c(-c2cnn(C3CCN(C(=O)OC(C)(C)C)CC3)c2)cnc(C(=O)NCC(=O)O)c1O] |
| 7 | 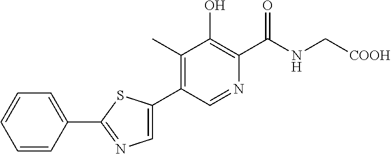 [SMILES: Cc1c(-c2cnc(-c3ccccc3)s2)cnc(C(=O)NCC(=O)O)c1O] |
| 8 | 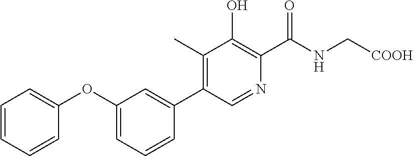 [SMILES: Cc1c(-c2cccc(Oc3ccccc3)c2)cnc(C(=O)NCC(=O)O)c1O] |
| 9 | 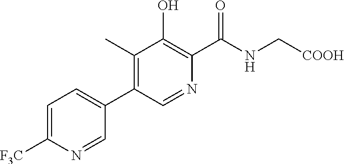 [SMILES: Cc1c(-c2ccc(C(F)(F)F)nc2)cnc(C(=O)NCC(=O)O)c1O] |
| 10 | 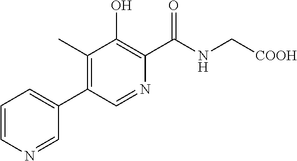 [SMILES: Cc1c(-c2cccnc2)cnc(C(=O)NCC(=O)O)c1O] |
| 11 | 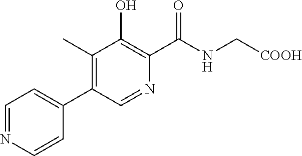 [SMILES: Cc1c(-c2ccncc2)cnc(C(=O)NCC(=O)O)c1O] |
| 12 | 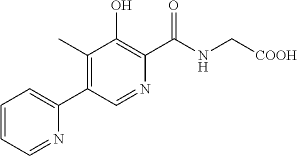 [SMILES: Cc1c(-c2ccccn2)cnc(C(=O)NCC(=O)O)c1O] |
| 13 | 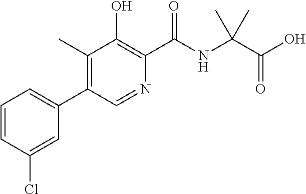 [SMILES: Cc1c(-c2cccc(Cl)c2)cnc(C(=O)NC(C)(C)C(=O)O)c1O] |
| 14 | 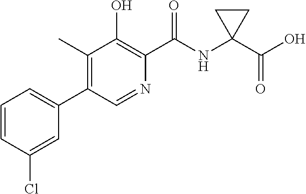 [SMILES: Cc1c(-c2cccc(Cl)c2)cnc(C(=O)NC2(C(=O)O)CC2)c1O] |
| 15 | 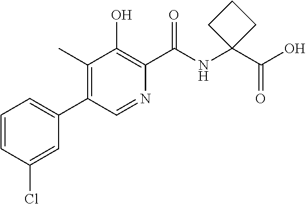 [SMILES: Cc1c(-c2cccc(Cl)c2)cnc(C(=O)NC2(C(=O)O)CCC2)c1O] |
| 16 | 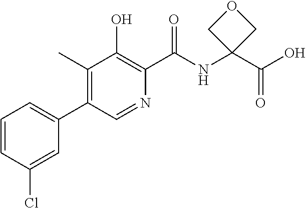 [SMILES: Cc1c(-c2cccc(Cl)c2)cnc(C(=O)NC2(C(=O)O)COC2)c1O] |
| 17 | 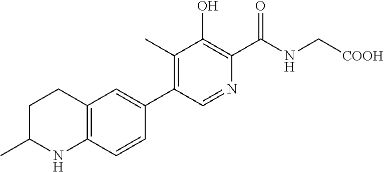 [SMILES: Cc1c(-c2ccc3c(c2)CCC(C)N3)cnc(C(=O)NCC(=O)O)c1O] |
| 18 | 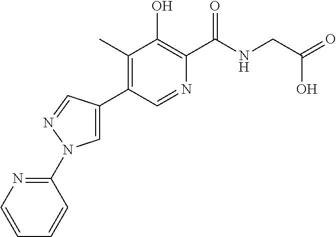 [SMILES: Cc1c(-c2cnn(-c3ccccn3)c2)cnc(C(=O)NCC(=O)O)c1O] |
| 19 | 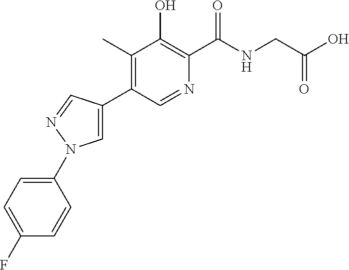 [SMILES: Cc1c(-c2cnn(-c3ccc(F)cc3)c2)cnc(C(=O)NCC(=O)O)c1O] |
| 20 | 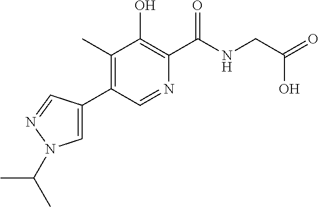 [SMILES: Cc1c(-c2cnn(C(C)C)c2)cnc(C(=O)NCC(=O)O)c1O] |
| 21 | 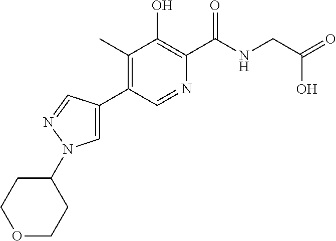 [SMILES: Cc1c(-c2cnn(C3CCOCC3)c2)cnc(C(=O)NCC(=O)O)c1O] |
| 22 | 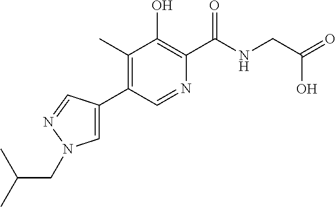 [SMILES: Cc1c(-c2cnn(CC(C)C)c2)cnc(C(=O)NCC(=O)O)c1O] |
| 23 | 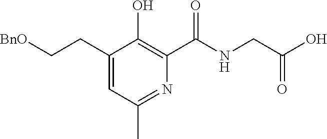 [SMILES: Cc1cc(CCOCc2ccccc2)c(O)c(C(=O)NCC(=O)O)n1] |
| 24 | 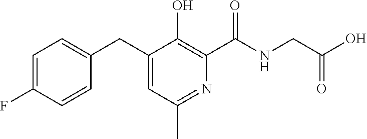 [SMILES: Cc1cc(Cc2ccc(F)cc2)c(O)c(C(=O)NCC(=O)O)n1] |
| 25 | 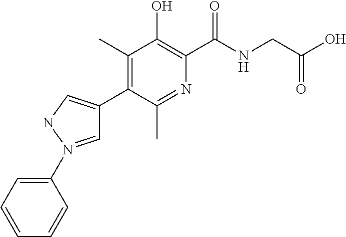 |
| 26 | 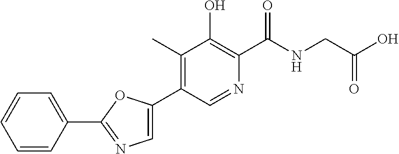 [SMILES: Cc1c(-c2cnc(-c3ccccc3)o2)cnc(C(=O)NCC(=O)O)c1O] |
| 27 | 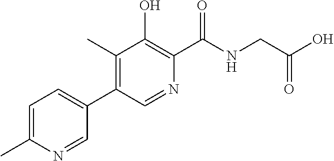 [SMILES: Cc1ccc(-c2cnc(C(=O)NCC(=O)O)c(O)c2C)cn1] |
| 28 | 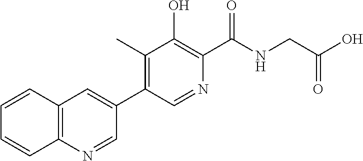 [SMILES: Cc1c(-c2cnc3ccccc3c2)cnc(C(=O)NCC(=O)O)c1O] |
| 29 | 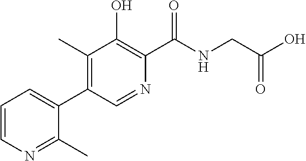 [SMILES: Cc1ncccc1-c1cnc(C(=O)NCC(=O)O)c(O)c1C] |
| 30 | 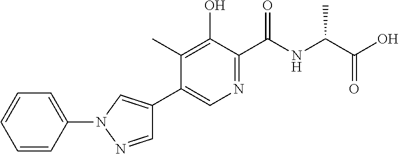 [SMILES: Cc1c(-c2cnn(-c3ccccc3)c2)cnc(C(=O)N[C@H](C)C(=O)O)c1O] |
| 31 | 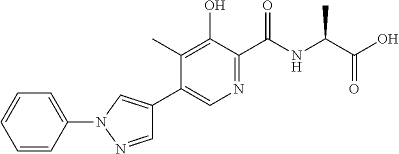 [SMILES: Cc1c(-c2cnn(-c3ccccc3)c2)cnc(C(=O)N[C@@H](C)C(=O)O)c1O] |
| 32 | 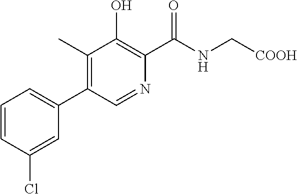 [SMILES: Cc1c(-c2cccc(Cl)c2)cnc(C(=O)NCC(=O)O)c1O] |
| 33 | 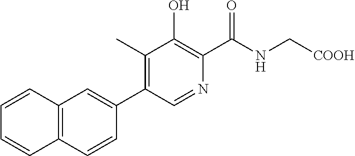 [SMILES: Cc1c(-c2ccc3ccccc3c2)cnc(C(=O)NCC(=O)O)c1O] |
| 34 | 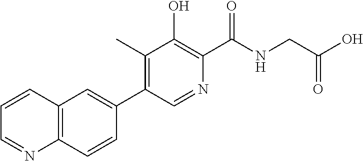 [SMILES: Cc1c(-c2ccc3ncccc3c2)cnc(C(=O)NCC(=O)O)c1O] |
| 35 | 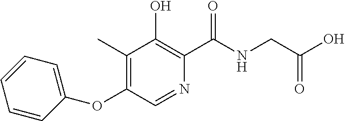 [SMILES: Cc1c(Oc2ccccc2)cnc(C(=O)NCC(=O)O)c1O] |
| 36 | 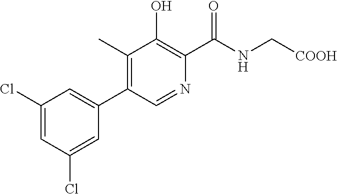 [SMILES: Cc1c(-c2cc(Cl)cc(Cl)c2)cnc(C(=O)NCC(=O)O)c1O] |
| 37 | 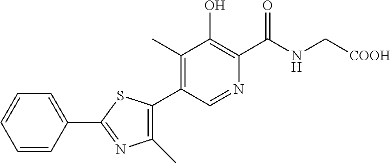 [SMILES: Cc1nc(-c2ccccc2)sc1-c1cnc(C(=O)NCC(=O)O)c(O)c1C] |
| 38 | 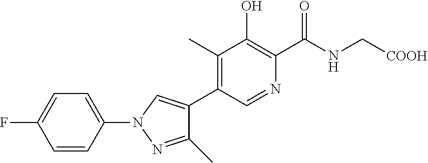 [SMILES: Cc1nn(-c2ccc(F)cc2)cc1-c1cnc(C(=O)NCC(=O)O)c(O)c1C] |
| 39 | 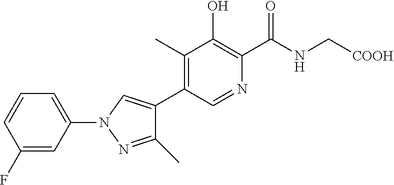 [SMILES: Cc1nn(-c2cccc(F)c2)cc1-c1cnc(C(=O)NCC(=O)O)c(O)c1C] |
| 40 | 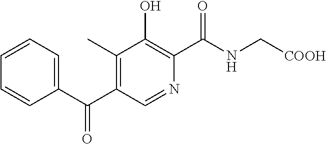 [SMILES: Cc1c(C(=O)c2ccccc2)cnc(C(=O)NCC(=O)O)c1O] |
| 41 | 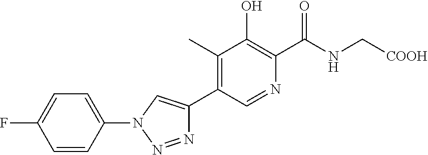 [SMILES: Cc1c(-c2cn(-c3ccc(F)cc3)nn2)cnc(C(=O)NCC(=O)O)c1O] |
| 42 | 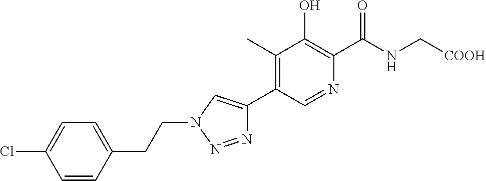 [SMILES: Cc1c(-c2cn(CCc3ccc(Cl)cc3)nn2)cnc(C(=O)NCC(=O)O)c1O] |
| 43 | 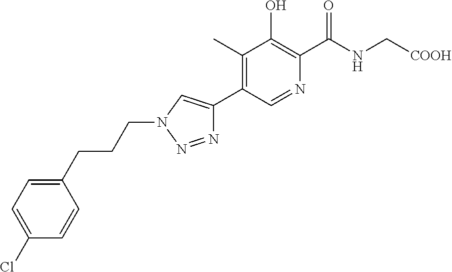 [SMILES: Cc1c(-c2cn(CCCc3ccc(Cl)cc3)nn2)cnc(C(=O)NCC(=O)O)c1O] |
| 44 | 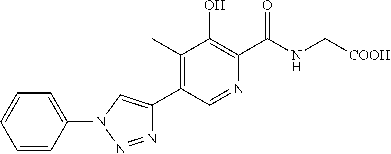 [SMILES: Cc1c(-c2cn(-c3ccccc3)nn2)cnc(C(=O)NCC(=O)O)c1O] |
| 45 | 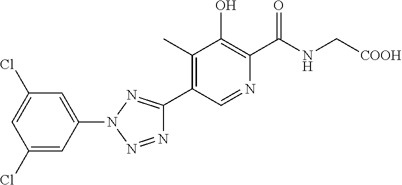 [SMILES: Cc1c(-c2nnn(-c3cc(Cl)cc(Cl)c3)n2)cnc(C(=O)NCC(=O)O)c1O] |
| 46 | 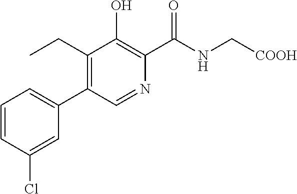 [SMILES: CCc1c(-c2cccc(Cl)c2)cnc(C(=O)NCC(=O)O)c1O] |
| 47 | 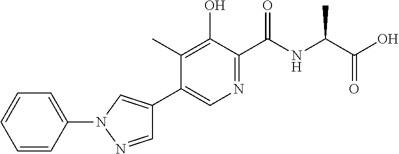 [SMILES: Cc1c(-c2cnn(-c3ccccc3)c2)cnc(C(=O)N[C@@H](C)C(=O)O)c1O] |
| 48 | 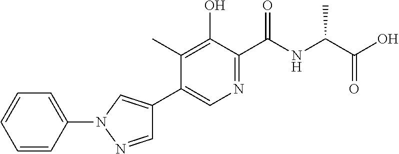 [SMILES: Cc1c(-c2cnn(-c3ccccc3)c2)cnc(C(=O)N[C@H](C)C(=O)O)c1O] |
| 49 | 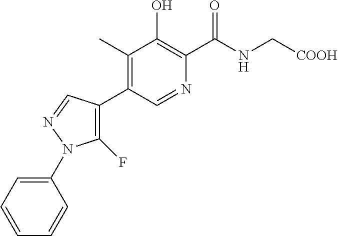 [SMILES: Cc1c(-c2cnn(-c3ccccc3)c2F)cnc(C(=O)NCC(=O)O)c1O] |
| 50 | 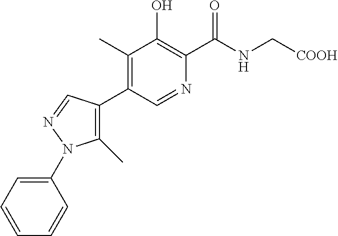 [SMILES: Cc1c(-c2cnn(-c3ccccc3)c2C)cnc(C(=O)NCC(=O)O)c1O] |

In some embodiments, the PHD inhibitor compound is any one of Compounds 51-70, or a pharmaceutically acceptable salt thereof.

| Cmpd No. | Structure |
|---|---|
| 51 | 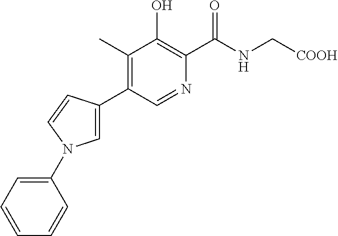 [SMILES: Cc1c(-c2ccn(-c3ccccc3)c2)cnc(C(=O)NCC(=O)O)c1O] |
| 52 | 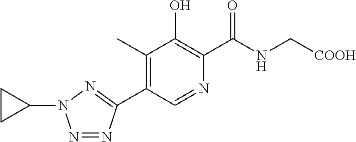 [SMILES: Cc1c(-c2nnn(C3CC3)n2)cnc(C(=O)NCC(=O)O)c1O] |
| 53 | 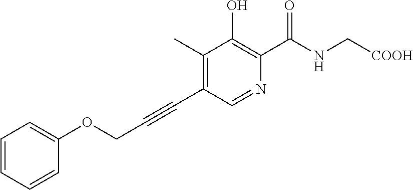 [SMILES: Cc1c(C#CCOc2ccccc2)cnc(C(=O)NCC(=O)O)c1O] |
| 54 | 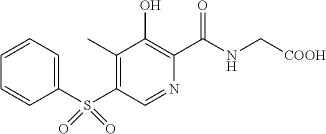 [SMILES: Cc1c(S(=O)(=O)c2ccccc2)cnc(C(=O)NCC(=O)O)c1O] |
| 55 | 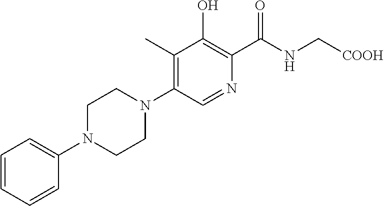 [SMILES: Cc1c(N2CCN(c3ccccc3)CC2)cnc(C(=O)NCC(=O)O)c1O] |
| 56 | 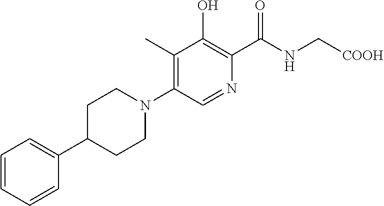 [SMILES: Cc1c(N2CCC(c3ccccc3)CC2)cnc(C(=O)NCC(=O)O)c1O] |
| 57 | 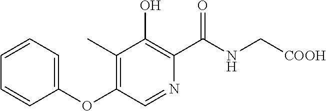 [SMILES: Cc1c(Oc2ccccc2)cnc(C(=O)NCC(=O)O)c1O] |
| 58 | 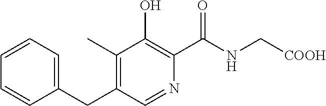 [SMILES: Cc1c(Cc2ccccc2)cnc(C(=O)NCC(=O)O)c1O] |
| 59 | 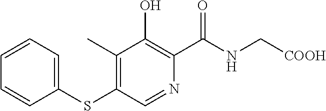 [SMILES: Cc1c(Sc2ccccc2)cnc(C(=O)NCC(=O)O)c1O] |
| 60 | 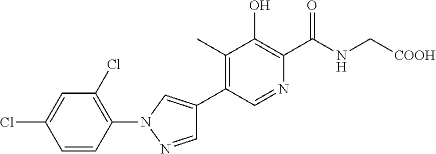 [SMILES: Cc1c(-c2cnn(-c3ccc(Cl)cc3Cl)c2)cnc(C(=O)NCC(=O)O)c1O] |
| 61 | 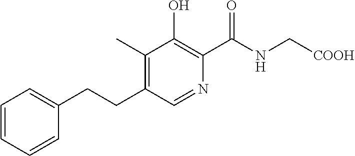 [SMILES: Cc1c(CCc2ccccc2)cnc(C(=O)NCC(=O)O)c1O] |
| 62 | 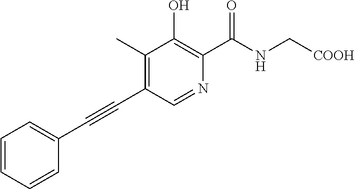 [SMILES: Cc1c(C#Cc2ccccc2)cnc(C(=O)NCC(=O)O)c1O] |
| 63 | 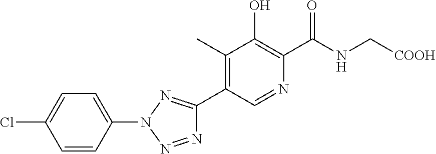 [SMILES: Cc1c(-c2nnn(-c3ccc(Cl)cc3)n2)cnc(C(=O)NCC(=O)O)c1O] |
| 64 | 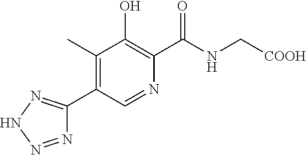 [SMILES: Cc1c(-c2nn[nH]n2)cnc(C(=O)NCC(=O)O)c1O] |
| 65 | 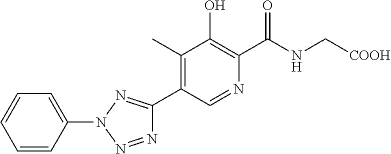 [SMILES: Cc1c(-c2nnn(-c3ccccc3)n2)cnc(C(=O)NCC(=O)O)c1O] |
| 66 | 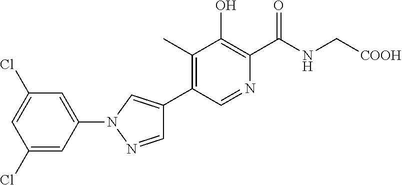 [SMILES: Cc1c(-c2cnn(-c3cc(Cl)cc(Cl)c3)c2)cnc(C(=O)NCC(=O)O)c1O] |
| 67 | 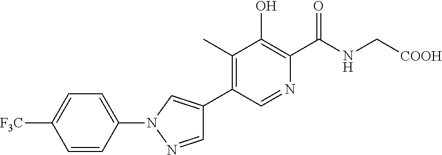 [SMILES: Cc1c(-c2cnn(-c3ccc(C(F)(F)F)cc3)c2)cnc(C(=O)NCC(=O)O)c1O] |
| 68 | 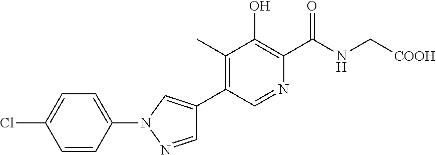 [SMILES: Cc1c(-c2cnn(-c3ccc(Cl)cc3)c2)cnc(C(=O)NCC(=O)O)c1O] |
| 69 | 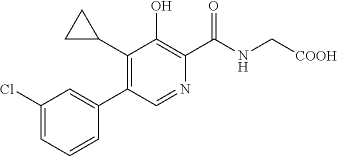 [SMILES: O=C(O)CNC(=O)c1ncc(-c2cccc(Cl)c2)c(C2CC2)c1O] |
| 70 | 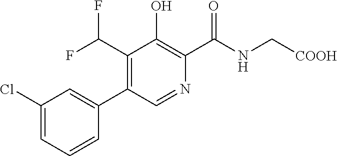 [SMILES: O=C(O)CNC(=O)c1ncc(-c2cccc(Cl)c2)c(C(F)F)c1O] |

Isotopologues

It should be understood that in the compounds described herein (e.g., a compound of any one of Formulas (I)-(XXIII) such as any one of compounds 1-70), the atoms may exhibit their natural isotopic abundances, or one or more of the atoms may be artificially enriched in a particular isotope having the same atomic number, but an atomic mass or mass number different from the atomic mass or mass number predominately found in nature. The present invention is meant to include all suitable isotopic variations of the compounds of the compounds described herein (e.g., a compound of any one of Formulas (I)-(XXIII) such as any one of compounds 1-70). For example, different isotopic forms of hydrogen (H) include protium (¹H), deuterium (²H), and tritium (³H). Protium is the predominant hydrogen isotope found in nature.

In some embodiments, one or more of the hydrogens of the compounds described herein (e.g., a compound of any one of Formulas (I)-(XXIII) such as any one of compounds 1-70) is replaced by a deuterium. Enriching for deuterium may afford certain therapeutic advantages, such as increasing in vivo half-life or reducing dosage requirements, or may provide a compound useful as a standard for characterization of biological samples. In some embodiments, one or more of the hydrogens of the compounds described herein (e.g., a compound of any one of Formulas (I)-(XXIII) such as any one of compounds 1-70) is replaced by tritium. Tritium is radioactive and may therefore provide for a radiolabeled compound, useful as a tracer in metabolic or kinetic studies.

Isotopic-enrichment of compounds disclosed herein (e.g., a compound of any one of Formulas (I)-(XXIII) such as any one of compounds 1-70), may be achieved without undue experimentation by conventional techniques well known to those skilled in the art or by processes analogous to those described in the Schemes and Examples herein using appropriate isotopically-enriched reagents and/or intermediates.

The term “isotopologue” refers to a species that has the same chemical structure and formula as a specific compound provided herein, with the exception of the positions of isotopic substitution and/or level of isotopic enrichment at one or more positions, e.g., hydrogen vs. deuterium. Thus, the term “compound,” as used herein, encompasses a collection of molecules having identical chemical structure, but also having isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound provided depends upon a number of factors including, but not limited to, the isotopic purity of deuterated reagents used to make the compound and the efficiency of incorporation of deuterium in the various synthesis steps used to prepare the compound.

When a position is designated as “H” or “hydrogen”, the position is understood to have hydrogen at its natural abundance isotopic composition. When a position is designated as “D” or “deuterium”, the position is understood to have deuterium at an abundance that is at least 3340 times greater than the natural abundance of deuterium, which is 0.015% (i.e., the term “D” or “deuterium” indicates at least 50.1% incorporation of deuterium).

In embodiments, a compound provided herein may have an isotopic enrichment factor for each deuterium present at a site designated as a potential site of deuteration on the compound of at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Synthesis of Compounds of the Inventions

The compounds described herein (e.g., a compound of any one of Formulas (I)-(XXIII) such as any one of compounds 1-70) can be prepared according to methods known in the art, including the exemplary syntheses of the Examples provided herein.

Abbreviations and acronyms used herein including the following:

| Term | Acronym |
|---|---|
| 4-Dimethylaminopyridine | DMAP |
| Acetyl | Ac |
| Aqueous | aq. |
| Benzotriazol-1-yl-oxytripyrrolidinophosphonium | PyBOP |
| hexafluorophosphate | |
| Benzyl | Bn |
| 1,1′-Bis(diphenylphosphino)ferrocene | dppf |
| tert-Butyloxycarbonyl | Boc |
| Broad singlet | brs |
| Dichloromethane | DCM |
| Dimethylsulfoxide | DMSO |
| Doublet | d |
| Electrospray ionization | ESI |
| Equivalent | eq |
| Ethyl acetate | EtOAc |
| Gram | g |
| Hexanes | Hex |
| High performance liquid chromatography | HPLC |
| Hour | hr |
| Isopropyl | i-Pr |
| Liquid chromatography-mass spectrometry | LCMS |
| Megahertz | MHz |
| meta-Chloroperoxybenzoic acid | m-CPBA |
| Methanol | MeOH |
| Milligram | mg |
| Milliliter | mL |
| Minute | min |
| Molarity | M |
| Multiplet | m |
| N,N-Diisopropylethylamine | DIPEA |
| N,N-Dimethylformamide | DMF |
| N,N-dimethylformamide dimethyl acetal | DMF-DMA |
| Normal | N |
| Nuclear magnetic resonance | NMR |
| Palladium on carbon | Pd/C |
| Pentet | p |
| Petroleum ether | PE |
| Phenyl | Ph |
| Preparatory | Prep |
| Quartet | q |
| Room temperature | RT |
| Singlet | s |
| Tetrahydrofuran | THF |
| Thin layer chromatography | TLC |
| Triethylamine | TEA |
| Trifluoroacetic acid | TFA |
| Trimethylsilyl | TMS |
| Triplet | t |

Compositions and Methods

The invention provides for use of a compound of any one of Formulas (I)-(XXIII), or a pharmaceutically acceptable salt thereof, for the manufacture of a medicament for use in treating various conditions or disorders as described herein. In one embodiment, a pharmaceutical composition is provided comprising at least one compound of any one of Formulas (I)-(XXIII), or a pharmaceutically acceptable salt thereof, and a pharmaceutically acceptable excipient or carrier. In various embodiments, the medicament or pharmaceutical composition can further comprise or be used in combination with at least one additional therapeutic agent.

The compounds of the present invention, or medicaments or compositions comprising the compounds, can be used to inhibit the activity of PHD. Inhibition of PHD may be of particular benefit in treating diseases including heart (e.g. ischemic heart disease, congestive heart failure, and valvular heart disease), lung (e.g., lung inflammation, pneumonia, acute lung injury, pulmonary hypertension, pulmonary fibrosis, and chronic obstructive pulmonary disease), respiratory (e.g., respiratory infection, acute respiratory distress syndrome), liver (e.g. acute liver failure and liver fibrosis and cirrhosis), and kidney (e.g. acute kidney injury and chronic kidney disease) disease, inflammatory bowel disease (IBD), ischemic reperfusion injury (e.g., stroke), and retinopathy of prematurity (ROP).

In one embodiment, the method of the invention comprises administering to a patient in need a therapeutically effective amount of a compound of any one of Formulas (I)-(XXIII), or a pharmaceutically acceptable salt thereof, or a pharmaceutical composition comprising one or more compounds of any one of Formulas (I)-(XXIII).

The invention is also directed to a method of inhibiting the activity of PHD. In one embodiment, the method comprises contacting PHD with an effective amount of one or more compounds selected from the group comprising compounds of any one of Formulas (I)-(XXIII), or a pharmaceutically acceptable salt thereof.

In still other embodiments, the compounds disclosed herein (e.g., a compound of any one of Formulas (I)-(XXIII) such as any one of compounds 1-70), or a pharmaceutically acceptable salt thereof, are useful for the treatment or prevention of anemia comprising treatment of anemic conditions associated with chronic kidney disease, polycystic kidney disease, aplastic anemia, autoimmune hemolytic anemia, bone marrow transplantation anemia, Churg-Strauss syndrome, Diamond Blackfan anemia, Fanconi's anemia, Felty syndrome, graft versus host disease, hematopoietic stem cell transplantation, hemolytic uremic syndrome, myelodysplastic syndrome, nocturnal paroxysmal hemoglobinuria, osteomyelofibrosis, pancytopenia, pure red-cell aplasia, purpura Schoenlein-Henoch, refractory anemia with excess of blasts, rheumatoid arthritis, Shwachman syndrome, sickle cell disease, thalassemia major, thalassemia minor, thrombocytopenic purpura, anemic or non-anemic patients undergoing surgery, anemia associated with or secondary to trauma, sideroblastic anemia, anemic secondary to other treatment including: reverse transcriptase inhibitors to treat HIV, corticosteroid hormones, cyclic cisplatin or non-cisplatin-containing chemotherapeutics, vinca alkaloids, mitotic inhibitors, topoisomerase II inhibitors, anthracyclines, alkylating agents, particularly anemia secondary to inflammatory, aging and/or chronic diseases. PHD inhibition may also be used to treat symptoms of anemia including chronic fatigue, pallor, and dizziness.

In other embodiments, the compounds disclosed herein (e.g., a compound of Formulas (I)-(XXIII) such as any one of compounds 1-70), or a pharmaceutically acceptable salt thereof, are useful for the treatment or prevention of diseases of metabolic disorders, including but not limited to diabetes and obesity.

In yet other embodiments, the compounds disclosed herein (e.g., a compound of Formulas (I)-(XXIII) such as any one of compounds 1-70), or a pharmaceutically acceptable salt thereof, are useful for the treatment or prevention of vascular disorders. These include but are not limited to hypoxic or wound healing related diseases requiring pro-angiogenic mediators for vasculogenesis, angiogenesis, and arteriogenesis

In still other embodiments, the compounds disclosed herein (e.g., a compound of Formulas (I)-(XXIII) such as any one of compounds 1-70), or a pharmaceutically acceptable salt thereof, are useful for the treatment or prevention of ischemia reperfusion injury. These include but are not limited to stroke, myocardial infarction, and acute kidney injury.

In other embodiments, the compounds disclosed herein (e.g., a compound of Formulas (I)-(XXIII) such as any one of compounds 1-70), or a pharmaceutically acceptable salt thereof, are useful in the treatment of inflammatory bowel disease. These include but are not limited to ulcerative colitis, and Crohn's disease.

In other embodiments, the compounds disclosed herein (e.g., a compound of Formulas (I)-(XXIII) such as any one of compounds 1-70), or a pharmaceutically acceptable salt thereof, are useful in the treatment of cancers, such as colorectal cancer.

In other embodiments, the compounds disclosed herein (e.g., a compound of Formulas (I)-(XXIII) such as any one of compounds 1-70), or a pharmaceutically acceptable salt thereof, are useful in the treatment of atherosclerosis.

In other embodiments, the compounds disclosed herein (e.g., a compound of Formulas (I)-(XXIII) such as any one of compounds 1-70), or a pharmaceutically acceptable salt thereof, are useful in the treatment of cardiovascular disease.

In other embodiments, the compounds disclosed herein (e.g., a compound of Formulas (I)-(XXIII) such as any one of compounds 1-70), or a pharmaceutically acceptable salt thereof, are useful in the treatment of a disease or condition of the eye. These include but are not limited to radiation retinopathy, retinopathy of prematurity (ROP), diabetic retinopathy, age-related macular degeneration, and ocular ischemia.

In other embodiments, the compounds disclosed herein (e.g., a compound of Formulas (I)-(XXIII) such as any one of compounds 1-70), or a pharmaceutically acceptable salt thereof, are useful in the treatment of a disease that is associated with hyperoxia.

In other embodiments, the compounds disclosed herein (e.g., a compound of Formulas (I)-(XXIII) such as any one of compounds 1-70), or a pharmaceutically acceptable salt thereof, are useful in the treatment of bronchopulmonary dysplasia (BPD).

In yet other embodiments, the compounds disclosed herein (e.g., a compound of Formulas (I)-(XXIII) such as any one of compounds 1-70), or a pharmaceutically acceptable salt thereof, are useful in the treatment of heart diseases. The conditions include but are not limited to postoperative myocardial ischemia in pancreatic surgery, myocardial injury after percutaneous coronary intervention (PCI), myocardial injury after non-cardiac surgery, perioperative myocardial ischemia in elective operation of abdominal aortic aneurysm, myocardial injury after PCI, myocardial damage in patients undergoing coronary artery bypass graft (CABG) surgery, Minimally invasive mitral valve (MIMV) repair or replacement, adult patient undergoing open heart surgery, chronic heart failure, NYHA class II-IV.

In other embodiments, the compounds disclosed herein (e.g., a compound of Formulas (I)-(XXIII) such as any one of compounds 1-70), or a pharmaceutically acceptable salt thereof, are useful in the treatment of lung diseases, such as lung inflammation, pneumonia, bronchitis, acute lung injury (ALI), pulmonary hypertension, pulmonary fibrosis, asthma, acute respiratory distress syndrome (ARDS), or chronic obstructive pulmonary disease. The conditions include but are not limited to lung injury during elective lung lobectomy, lung injury during coronary artery bypass graft surgery (CABG surgery), and lung transplantation.

In other embodiments, the compounds disclosed herein (e.g., a compound of Formulas (I)-(XXIII) such as any one of compounds 1-70), or a pharmaceutically acceptable salt thereof, are useful in the treatment of respiratory diseases. The conditions include but are not limited to respiratory infection, acute respiratory distress syndrome (ARDS), lung inflammation, pneumonia, and acute lung injury.

In other embodiments, the compounds disclosed herein (e.g., a compound of Formulas (I)-(XXIII) such as any one of compounds 1-70), or a pharmaceutically acceptable salt thereof, are useful in the treatment of liver disease. The conditions include but are not limited to non-alcoholic steatohepatitis (NASH).

In other embodiments, the compounds disclosed herein (e.g., a compound of Formulas (I)-(XXIII) such as any one of compounds 1-70), or a pharmaceutically acceptable salt thereof, are useful in the treatment of kidney disease. The conditions include but are not limited to contrast-induced acute kidney injury, stage III-IV chronic kidney disease undergoing planned coronary angiography, acute kidney injury in patients undergoing valvular heart surgery, non-dialysis dependent chronic kidney disease, chronic kidney disease patients initiating dialysis, non-dialysis dependent chronic kidney disease.

In other embodiments, the compounds disclosed herein (e.g., a compound of Formulas (I)-(XXIII) such as any one of compounds 1-70), or a pharmaceutically acceptable salt thereof, are useful in the treatment of injury to and/or failure of one or more organs (e.g., injury to and/or failure of lung, heart, liver, or kidney). The conditions include but are not limited to acute organ injury or organ failure, and induced organ dysfunction.

In other embodiments, the compounds disclosed herein (e.g., a compound of Formulas (I)-(XXIII) such as any one of compounds 1-70), or a pharmaceutically acceptable salt thereof, are useful in the treatment of respiratory viral (e.g., coronavirus) infection or pulmonary viral (e.g., coronavirus) infection.

In addition, the compounds disclosed herein (e.g., a compound of Formulas (I)-(XXIII) such as any one of compounds 1-70), or a pharmaceutically acceptable salt thereof, may be used in combination with additional active ingredients in the treatment of the above conditions. The additional compounds may be co-administered separately with the compounds disclosed herein (e.g., a compound of Formulas (I)-(XXIII) such as any one of compounds 1-70), or a pharmaceutically acceptable salt thereof, or included with an additional active ingredient in a pharmaceutical composition according to the invention. In an exemplary embodiment, additional active ingredients are those that are known or discovered to be effective in the treatment of conditions, disorders, or diseases mediated by PHD enzyme or that are active against another targets associated with the particular condition, disorder, or disease, such as an alternate PHD modulator. The combination may serve to increase efficacy (e.g., by including in the combination a compound potentiating the potency or effectiveness of a compound according to the invention), decrease one or more side effects, or decrease the required dose of the compound according to the invention.

The compounds of the invention are used, alone or in combination with one or more other active ingredients, to formulate pharmaceutical compositions of the invention. A pharmaceutical composition of the invention comprises: (a) an effective amount of the compounds disclosed herein (e.g., a compound of Formulas (I)-(XXIII) such as any one of compounds 1-70), or a pharmaceutically acceptable salt, pharmaceutically acceptable prodrug, or pharmaceutically active metabolite thereof; and (b) a pharmaceutically acceptable excipient.

A “pharmaceutically acceptable excipient” refers to a substance that is nontoxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols. Suitable excipients may also include antioxidants. Such antioxidants may be used in a pharmaceutical composition or in a storage medium to prolong the shelf-life of the drug product.

### Pharmaceutical Formulations and Routes of Administration

The compounds and compositions of the present invention can be delivered directly or in pharmaceutical compositions or medicaments along with suitable carriers or excipients, as is well known in the art. Present methods of treatment can comprise administration of an effective amount of a compound of the invention to a subject in need. In a preferred embodiment, the subject is a mammalian subject, and in a most preferred embodiment, the subject is a human subject.

An effective amount of such compound, composition, or medicament can readily be determined by routine experimentation, as can the most effective and convenient route of administration, and the most appropriate formulation. Various formulations and drug delivery systems are available in the art. See, e.g., Gennaro, A. R., ed. (1995) Remington's Pharmaceutical Sciences, supra.

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

Pharmaceutical dosage forms of a compound of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art and include those listed in various pharmacopoeias. See, e.g., the U.S. Pharmacopeia (USP), Japanese Pharmacopoeia (JP), European Pharmacopoeia (EP), and British pharmacopeia (BP); the U.S. Food and Drug.

Administration (www.fda.gov) Center for Drug Evaluation and Research (CEDR) publications, e.g., Inactive Ingredient Guide (1996); Ash and Ash, Eds. (2002) Handbook of Pharmaceutical Additives, Synapse Information Resources, Inc., Endicott NY; etc.) [0149]Pharmaceutical dosage forms of a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms, and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid, or a liquid formulation, for example a gel, a (micro-) emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol from pressurized packs, or a nebuliser, usually with the use of a propellant, e.g., halogenated carbons derived from methane and ethane, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compounds and compositions formulated for parenteral administration by injection are usually sterile and can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the compounds of the invention are well-known in the art and include water-based solutions containing a base, such as, for example, sodium hydroxide, to form an ionized compound; sucrose or sodium chloride as a tonicity agent; and a buffer, for example, a buffer that contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

A therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays. In certain some embodiments, a compound of the disclosure is formulated for oral administration. An exemplary dose of a compound of the disclosure in a pharmaceutical formulation for oral administration is from about 0.5 to about 10 mg/kg body weight of subject. In some embodiments, a pharmaceutical formulation comprises from about 0.7 to about 5.0 mg/kg body weight of subject, or alternatively, from about 1.0 to about 2.5 mg/kg body weight of subject. A typical dosing regimen for oral administration would be administration of the pharmaceutical formulation for oral administration three times per week, two times per week, once per week or daily.

An effective amount or a therapeutically effective amount or dose of an agent, e.g., a compound of the invention, refers to that amount of the agent or compound that results in amelioration of symptoms or a prolongation of survival in a subject. Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. Agents that exhibit high therapeutic indices are preferred.

The effective amount or therapeutically effective amount is the amount of the compound or pharmaceutical composition that will elicit the biological or medical response of a tissue, system, animal or human that is being sought by the researcher, veterinarian, medical doctor or other clinician. Dosages particularly fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects; i.e., the minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of compound or composition administered may be dependent on a variety of factors, including the sex, age, and weight of the subject being treated, the severity of the affliction, the manner of administration, and the judgment of the prescribing physician.

The present compounds and compositions may, if desired, be presented in a pack or dispenser device containing one or more unit dosage forms containing the active ingredient. Such a pack or device may, for example, comprise metal or plastic foil, such as a blister pack; or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein and are specifically contemplated.

Exemplification

### General Method

Most of chemicals were purchased from Sinopharm Chemical Reagent Co.(SCRC), Sigma-Aldrich, Alfa or other vendors.

¹H NMR or ¹⁹F NMR spectra were recorded on Bruker AVIII 400 or Bruker AVIII 500.

LCMS measurement was run on Agilent 1200 HPLC/6100 SQ System using the follow conditions:

Method A: Mobile Phase: A: Water (0.01% TFA) B: Acetonitrile (0.01% TFA); Gradient Phase: 5% B increase to 95% B within 1.4 min, 95% B with 1.6 min (total runtime:3 min); Flow Rate: 2.3 mL/min; Column: SunFire C18, 4.6*50 mm, 3.5 μm; Column Temperature: 50° C. Detectors: ADC ELSD, DAD(214 nm and 254 nm), ES-API.

Method B: Mobile Phase: A: Water (10 mM NH₄HCO₃) B: Acetonitrile; Gradient Phase: 5% to 95% B within 1.5 min, 95% B with 1.5 min (total runtime:3 min); Flow Rate: 2.0 mL/min; Column: XBridge C18,4.6*50 mm, 3.5 um; Column Temperature: 40° C. Detectors: ADC ELSD, DAD(214 nm and 254 nm), MSD (ES-API).

### General Scheme for Synthesis of a Compound of Formula (I)

Compounds of Formula (I) are prepared according to Scheme A using commercially available materials. The reaction of halogenated pyridines (Compound a) with an oxidant yields N-oxide pyridine compounds of Compound (b). Cyanation of Compound (b) furnishes Compound (c). Cross-coupling of Compound (c) and boronic acids yields Compound (e). Halogen displacement of Compound (e) using benzyl alcohol furnishes Compound (f). Nitrile hydrolysis of Compound (f) followed by amide formation with amino esters yields amides (Compound (i)). Deprotection of the benzyl group furnishes compounds of Formula (I), and the subsequent saponification of the ester furnishes Compound (j).

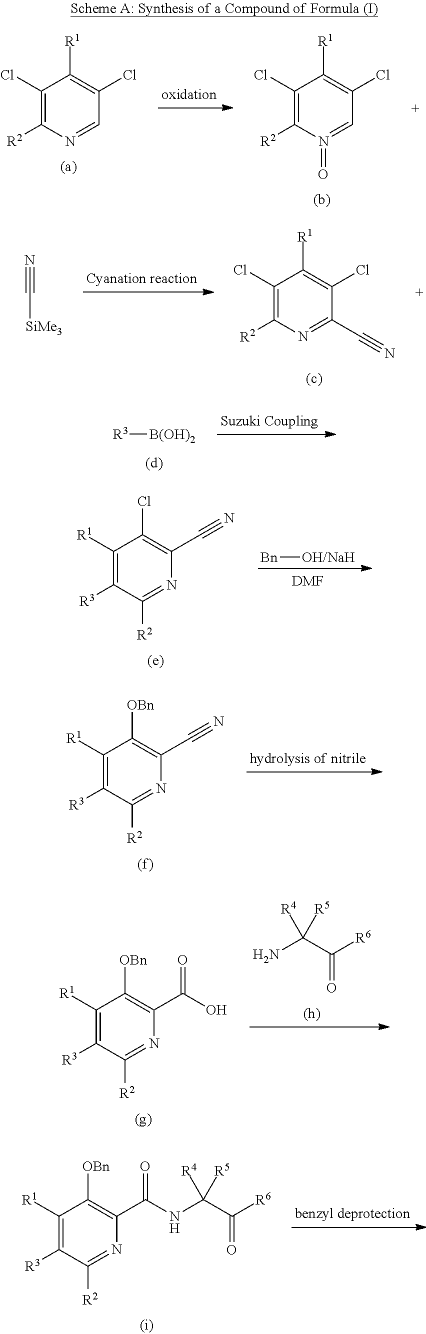

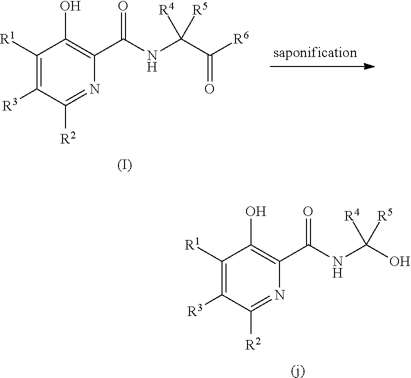

### Synthesis for Exemplary Compounds

### Example 1: Preparation of Compound 1

### 3,5-Dichloro-4-methylpyridine 1-oxide

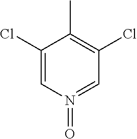

To a solution of 3,5-dichloro-4-methyl-pyridine (5.0 g, 30.8 mmol) in dichloromethane (70.0 mL) was added 3-chloroperoxybenzoic acid (8.12 g, 40.11 mmol, 85%) at 0° C. The mixture was stirred at room temperature for 18.0 h and potassium carbonate (4.42 g, 32.0 mmol) was added. The mixture was stirred for another 1 h and insoluble solid was filtered. The filtrate was concentrated to obtain 3,5-dichloro-4-methylpyridine 1-oxide (4.7 g, 26.4 mmol, 85.1% yield) as white solid. LC-MS: m/z=178.1 [M+H]⁺, retention time 1.47 min (Method A). The product was pure enough and used directly to the next step.

### 3,5-Dichloro-4-methylpicolinonitrile

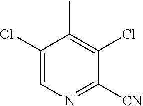

A mixture of 3,5-dichloro-4-methyl-pyridine 1-oxide (5.0 g, 28.4 mmol), trimethylsilyl cyanide (5.0 g, 40.3 mmol) and triethylamine (4.28 g, 42.3 mmol) in acetonitrile (90.0 mL) was stirred at 85° C. for 24.0 h. The mixture was diluted with water and extracted with ethyl acetate. The organic layer was washed with brine, dried over sodium sulfate and concentrated under reduced pressure. The crude product was purified by flash chromatography (petroleum ether/ethyl acetate=10/1) to obtain 3,5-dichloro-4-methylpicolinonitrile (4.96 g, 26.8 mmol, 94.5% yield) as yellow oil. LC-MS: m/z=187.2 [M+H]⁺, retention time 1.74 min (Method A).

### 3-Chloro-5-(3-fluorophenyl)-4-methylpicolinonitrile

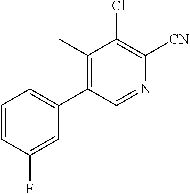 [SMILES: Cc1c(-c2cccc(F)c2)cnc(C#N)c1Cl]

To a solution of 3,5-dichloro-4-methylpicolinonitrile (500 mg, 2.67 mmol), (3-fluorophenyl)boronic acid (374 mg 2.67 mmol) and potassium carbonate (443 mg, 3.21 mmol) was added [1,1′-bis(diphenylphosphino)ferrocene]dichloropalladium(II) (78 mg, 0.11 mmol) in N,N-dimethylformamide/water (5.0 mL/0.5 mL). The mixture was stirred at 45° C. under nitrogen for 16.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=2/1) to afford 3-chloro-5-(3-fluorophenyl)-4-methylpicolinonitrile (400 mg, 1.63 mmol, 61% yield) as a yellow solid. LC-MS: m/z=247.1 [M+H]+, retention time=1.83 min (Method A).

### 3-(Benzyloxy)-5-(3-fluorophenyl)-4-methylpicolinonitrile

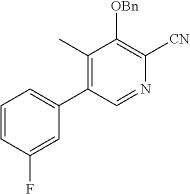 [SMILES: Cc1c(-c2cccc(F)c2)cnc(C#N)c1OCc1ccccc1]

To a solution of 3-chloro-5-(3-fluorophenyl)-4-methylpicolinonitrile (400.0 mg, 1.62 mmol) in N,N-dimethylformamide (10.0 mL) was added sodium hydride (78 mg, 1.94 mmol, 60% w/w dispersion in mineral oil) at 0° C. under nitrogen. The mixture was stirred at 0° C. for 10 min and then benzyl alcohol (210 mg, 1.94 mmol) was added. The solution was stirred at 0° C. for 1.0 h and diluted with ethyl acetate and water. The organic layer was washed with brine, dried over sodium sulfate and concentrated under reduced pressure. The crude product was purified by flash chromatography (petroleum ether/ethyl acetate=3/1) to give 3-(benzyloxy)-5-(3-fluorophenyl)-4-methylpicolinonitrile (378 mg, 1.18 mmol, 73% yield) as yellow solid. LC-MS: m/z=319.1 [M+H]+, retention time=2.21 min (Method A).

### 3-(Benzyloxy)-5-(3-fluorophenyl)-4-methylpicolinic acid

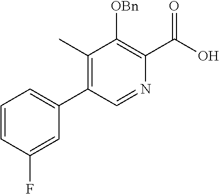 [SMILES: Cc1c(-c2cccc(F)c2)cnc(C(=O)O)c1OCc1ccccc1]

To a solution of 3-(benzyloxy)-5-(3-fluorophenyl)-4-methylpicolinonitrile (378 mg, 1.19 mmol) in ethanol (10.0 mL) was added 30% aqueous sodium hydroxide (5.0 mL). The mixture was stirred at 100° C. for 5.0 h, cooled and concentrated to remove ethanol. The resulting aqueous solution was acidified with 10% hydrochloric acid solution to pH=3˜4. The precipitate was filtered and dried to afford 3-(benzyloxy)-5-(3-fluorophenyl)-4-methylpicolinic acid (340 mg, 1.01 mmol, 85% yield) as white solid. LC-MS: m/z=338.1 [M+H]⁺, retention time 2.00 min (Method A). The product was pure enough and used directly to the next step.

### Ethyl (3-(benzyloxy)-5-(3-fluorophenyl)-4-methylpicolinoyl)glycinate

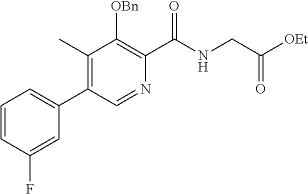 [SMILES: CCOC(=O)CNC(=O)c1ncc(-c2cccc(F)c2)c(C)c1OCc1ccccc1]

A mixture of 3-(benzyloxy)-5-(3-fluorophenyl)-4-methylpicolinic acid (170 mg, 0.50 mmol), ethyl glycinate hydrochloride (70 mg, 0.50 mmol), benzotriazole-1-yl-oxytripyrrolidinophosphonium hexafluorophosphate (288 mg, 0.55 mmol) and triethylamine (254 mg, 2.52 mmol) in dichloromethane (5.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/1) to obtain ethyl (3-(benzyloxy)-5-(3-fluorophenyl)-4-methylpicolinoyl)glycinate (200 mg, 0.47 mmol, 94% yield) as white solid. LC-MS: m/z=423.1 [M+H]⁺, retention time 2.15 min (Method A).

### Ethyl (5-(3-fluorophenyl)-3-hydroxy-4-methylpicolinoyl)glycinate

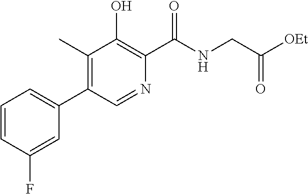 [SMILES: CCOC(=O)CNC(=O)c1ncc(-c2cccc(F)c2)c(C)c1O]

A mixture of ethyl (3-(benzyloxy)-5-(3-fluorophenyl)-4-methylpicolinoyl)glycinate (200 mg, 0.47 mmol) and 10% palladium on carbon (20.0 mg) in tetrahydrofuran (10.0 mL) was stirred under hydrogen atmosphere for 18.0 h. The insoluble solid was filtered and the filtrate was concentrated to give ethyl (3-hydroxy-4-methyl-5-(1-phenyl-1H-pyrazol-4-yl)picolinoyl)glycinate (150 mg, 0.45 mmol, 95% yield) as a yellow solid. LC-MS: m/z=333.1 [M+H]⁺, retention time 2.19 min (Method A). The product was pure enough and used directly to the next step.

### (5-(3-Fluorophenyl)-3-hydroxy-4-methylpicolinoyl)glycine

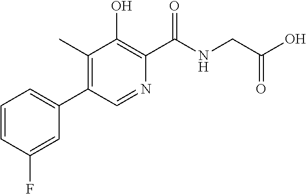 [SMILES: Cc1c(-c2cccc(F)c2)cnc(C(=O)NCC(=O)O)c1O]

To a solution of ethyl (3-hydroxy-4-methyl-5-(1-phenyl-1H-pyrazol-4-yl)picolinoyl) glycinate (150 mg, 0.45 mmol) in tetrahydrofuran/water (8.0 mL/2.0 mL) was added lithium hydroxide monohydrate (164 mg, 4.0 mmol). The mixture was stirred overnight and concentrated to remove tetrahydrofuran. The resulting aqueous solution was acidified with 10% hydrochloric acid solution to pH=3˜4. The precipitate was filtered, washed with water and dried to obtain (5-(3-fluorophenyl)-3-hydroxy-4-methylpicolinoyl)glycine (24.0 mg, 0.06 mmol, 13% yield) as white solid. LC-MS: m/z=305.1 [M+H]⁺, retention time 4.37 min (Method A). ¹HNMR (400 MHz, DMSO-d₆) δ 12.81 (s, 1H), 9.38 (t, J=5.9 Hz, 1H), 8.06 (s, 1H), 7.57 (dd, J=14.1, 7.8 Hz, 1H), 7.42-7.16 (m, 3H), 4.01 (d, J=6.1 Hz, 2H), 2.16 (s, 3H).

### Example 2: Preparation of Compound 2

### 3-Chloro-5-(3-methoxyphenyl)-4-methylpicolinonitrile

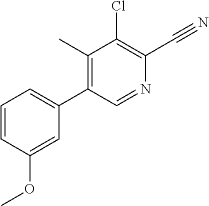 [SMILES: COc1cccc(-c2cnc(C#N)c(Cl)c2C)c1]

To a solution of 3,5-dichloro-4-methylpicolinonitrile (600 mg, 3.21 mmol), (3-methoxyphenyl)boronic acid (487.7 mg, 3.21 mmol) and potassium carbonate (531.34 mg, 3.85 mmol was added [1,1′-bis(diphenylphosphino)ferrocene]dichloropalladium(II) (117.27 mg, 0.05 mmol)) in N,N-dimethylformamide/water (2.0 mL/0.2 mL). The mixture was stirred at 45° C. under nitrogen for 12.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=2/1) to afford 3-chloro-5-(3-methoxyphenyl)-4-methylpicolinonitrile (500 mg, 1.94 mmol, 61% yield) as a yellow solid. LC-MS: m/z=259 [M+H]⁺, retention time 2.13 min (Method B).

### 3-(Benzyloxy)-5-(3-methoxyphenyl)-4-methylpicolinonitrile

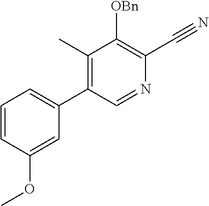 [SMILES: COc1cccc(-c2cnc(C#N)c(OCc3ccccc3)c2C)c1]

To a solution of 3-chloro-5-(3-methoxyphenyl)-4-methylpicolinonitrile (500 mg, 1.94 mmol) in N,N-dimethylformamide (10.0 mL) was added sodium hydride (100.8 mg, 2.52 mmol, 60% w/w dispersion in mineral oil) at 0° C. under nitrogen. The mixture was stirred at 0° C. for 10 min and then benzyl alcohol (172.1 mg, 2.52 mmol) was added. The solution was stirred at room temperature for 2.0 h and diluted with ethyl acetate and water. The organic layer was washed with brine, dried over sodium sulfate and concentrated under reduced pressure. The crude product was purified by flash chromatography (petroleum ether/ethyl acetate=3/1) to give 3-(benzyloxy)-5-(3-methoxyphenyl)-4-methylpicolinonitrile (350 mg, 1.06 mmol, 54.6% yield) as yellow solid. LC-MS: m/z=331.0 [M+H]⁺, retention time 1.91 min (Method A).

### 3-(Benzyloxy)-5-(3-methoxyphenyl)-4-methylpicolinic Acid

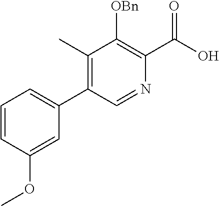 [SMILES: COc1cccc(-c2cnc(C(=O)O)c(OCc3ccccc3)c2C)c1]

To a solution of 3-(benzyloxy)-5-(3-methoxyphenyl)-4-methylpicolinonitrile (350 mg, 1.06 mmol in ethanol (10.0 mL) was added 30% aqueous sodium hydroxide (5.0 mL). The mixture was stirred at 100° C. for 3.0 h, cooled and concentrated to remove ethanol. The resulting aqueous solution was acidified with 10% hydrochloric acid solution to pH=3˜4. The precipitate was filtered and dried to afford 3-(benzyloxy)-5-(3-methoxyphenyl)-4-methylpicolinic acid (350 mg, 1.0 mmol, 94.61% yield) as white solid. LC-MS: m/z=350.0 [M+H]⁺, retention time 1.38 min (Method B). The product was pure enough and used directly to the next step.

### Ethyl (3-(benzyloxy)-5-(3-methoxyphenyl)-4-methylpicolinoyl)glycinate

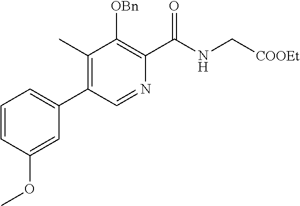 [SMILES: CCOC(=O)CNC(=O)c1ncc(-c2cccc(OC)c2)c(C)c1OCc1ccccc1]

A mixture of 3-(benzyloxy)-5-(3-methoxyphenyl)-4-methylpicolinic acid (180 mg, 0.52 mmol, ethyl glycinate hydrochloride (86.0 mg, 0.62 mmol), benzotriazole-1-yl-oxytripyrrolidinophosphonium hexafluorophosphate (321.8 mg, 0.62 mmol) and triethylamine (260.5 mg, 2.58 mmol) in dichloromethane (5.0 mL) was stirred at room temperature for 12.0 h. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/1) to obtain ethyl (3-(benzyloxy)-5-(3-methoxyphenyl)-4-methylpicolinoyl)glycinate (180 mg, 0.41 mmol, 79.76% yield) as white solid. LC-MS: m/z=435.0 [M+H]⁺, retention time 2.18 min (Method B).

### Ethyl (3-hydroxy-5-(3-methoxyphenyl)-4-methylpicolinoyl)glycinate

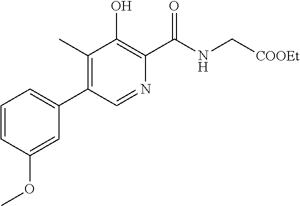 [SMILES: CCOC(=O)CNC(=O)c1ncc(-c2cccc(OC)c2)c(C)c1O]

A mixture of ethyl (3-(benzyloxy)-5-(3-methoxyphenyl)-4-methylpicolinoyl)glycinate (180 mg, 0.42 mmol) and 10% palladium on carbon (20.0 mg) in tetrahydrofuran (10.0 mL) was stirred under hydrogen atmosphere overnight. The insoluble solid was filtered and the filtrate was concentrated to give ethyl (3-hydroxy-5-(3-methoxyphenyl)-4-methylpicolinoyl)glycinate (180 mg, crude) as a yellow solid. LC-MS: m/z=345.0 [M+H]⁺, retention time 1.88 min (Method A). The product was pure enough and used directly to the next step.

### (3-Hydroxy-5-(3-methoxyphenyl)-4-methylpicolinoyl)glycine

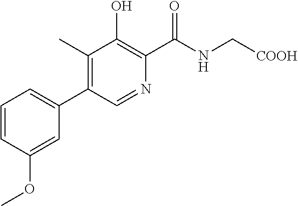 [SMILES: COc1cccc(-c2cnc(C(=O)NCC(=O)O)c(O)c2C)c1]

To a solution of ethyl (3-hydroxy-5-(3-methoxyphenyl)-4-methylpicolinoyl)glycinate (160 mg, 0.47 mmol) in tetrahydrofuran/water (10.0 mL/4.0 mL) was added sodium hydroxide (160 mg, 4.0 mmol). The mixture was stirred at 40° C. for 12.0 h and concentrated to remove tetrahydrofuran. The resulting aqueous solution was acidified with 10% hydrochloric acid solution to pH=3˜4. The precipitate was filtered, washed with water and dried to obtain (3-hydroxy-5-(3-methoxyphenyl)-4-methylpicolinoyl)glycine (62 mg, 0.20 mmol, 42% yield) as white solid. LC-MS: m/z=317.0 [M+H]⁺, retention time 4.40 min (Method A). ¹HNMR (400 MHz, DMSO-d₆) δ 12.78 (s, 1H), 9.37 (t, J=6.0 Hz, 1H), 8.05 (s, 1H), 7.44 (dd, J=10.1, 6.1 Hz, 1H), 7.03 (dt, J=7.4, 3.8 Hz, 1H), 6.99 (dd, J=3.7, 1.9 Hz, 2H), 4.02 (t, J=7.9 Hz, 2H), 3.82 (s, 3H), 2.17 (s, 3H).

### Example 3: Preparation of Compound 3

### 6-(4,4,5,5-Tetramethyl-1,3,2-dioxaborolan-2-yl)isoquinoline

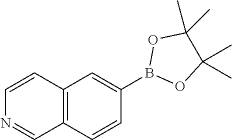 [SMILES: CC1(C)OB(c2ccc3cnccc3c2)OC1(C)C]

To a solution of 6-bromoisoquinoline (1.04 g, 5.0 mmol), bis(pinacolato)diboron (2.54 g, 10.0 mmol) and potassium acetate (1.96 g, 20.0 mmol) was added [1,1′-bis(diphenylphosphino)ferrocene]dichloropalladium(II) (365 mg, 0.5 mmol) in 1,4-dioxane (15.0 mL). The mixture was stirred at 90° C. under nitrogen for 2.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=2/1) to afford 6-(4,4,5,5-tetramethyl-1,3,2-dioxaborolan-2-yl)isoquinoline (0.79 g, 3.1 mmol, 62% yield) as white solid. LC-MS: m/z=256.0 [M+H]⁺, retention time 1.41 min (Method B).

### 3-Chloro-5-(isoquinolin-6-yl)-4-methylpicolinonitrile

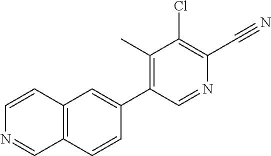 [SMILES: Cc1c(-c2ccc3cnccc3c2)cnc(C#N)c1Cl]

To a solution of 3,5-dichloro-4-methylpicolinonitrile (400 mg, 2.16 mmol), 6-(4,4,5,5-tetramethyl-1,3,2-dioxaborolan-2-yl)isoquinoline (550 mg, 2.16 mmol) and potassium carbonate (358 mg, 2.59 mmol) was added [1,1′-bis(diphenylphosphino) ferrocene]dichloropalladium(II) (146 mg, 0.2 mmol)) in N,N-dimethylformamide/water (5.0 mL/0.5 mL). The mixture was stirred at 45° C. under nitrogen overnight and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=1/1) to afford 3-chloro-5-(isoquinolin-6-yl)-4-methylpicolinonitrile (500 mg, 1.79 mmol, 83% yield) as a yellow solid. LC-MS: m/z=280.0 [M+H]⁺, retention time 1.58 min (Method B).

### 5-(Isoquinolin-6-yl)-3-((4-methoxybenzyl)oxy)-4-methylpicolinonitrile

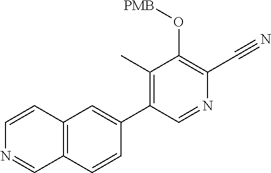

To a solution of 3-chloro-5-(isoquinolin-6-yl)-4-methylpicolinonitrile (500.0 mg, 1.79 mmol) in N,N-dimethylformamide (10.0 mL) was sodium hydride (86 mg, 2.15 mmol, 60% w/w dispersion in mineral oil) at 0° C. under nitrogen. The mixture was stirred at 0° C. for 10 min and then (4-methoxyphenyl)methanol (296 mg, 2.15 mmol) was added. The solution was stirred at 0° C. for 1.0 h and diluted with ethyl acetate and water. The organic layer was washed with brine, dried over sodium sulfate and concentrated under reduced pressure. The crude product was purified by flash chromatography (petroleum ether/ethyl acetate=1/1) to give 5-(isoquinolin-6-yl)-3-((4-methoxybenzyl)oxy)-4-methylpicolinonitrile (250 mg, 0.66 mmol, 37% yield) as yellow solid. LC-MS: m/z=382.1 [M+H]+, retention time=2.21 min (Method A).

### 3-Hydroxy-5-(isoquinolin-6-yl)-4-methylpicolinic Acid

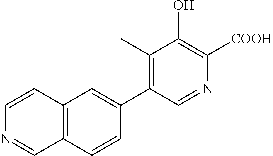 [SMILES: Cc1c(-c2ccc3cnccc3c2)cnc(C(=O)O)c1O]

To a solution of 5-(isoquinolin-6-yl)-3-((4-methoxybenzyl)oxy)-4-methylpicolinonitrile (250 mg, 0.66 mmol) in ethanol (10.0 mL) was added 30% aqueous sodium hydroxide (4.0 mL). The mixture was stirred at 100° C. for 5.0 h, cooled and concentrated to remove ethanol. The resulting aqueous solution was acidified with 10% hydrochloric acid solution to pH=3˜4. The precipitate was filtered and dried to afford 3-hydroxy-5-(isoquinolin-6-yl)-4-methylpicolinic acid (250 mg, crude) as white solid. LC-MS: m/z=401.1 [M+H]⁺, retention time 2.00 min (Method A). The product was pure enough and used directly to the next step.

### Ethyl (3-hydroxy-5-(isoquinolin-6-yl)-4-methylpicolinoyl)glycinate

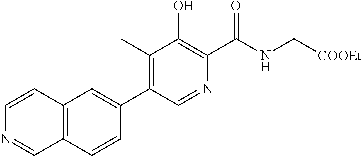 [SMILES: CCOC(=O)CNC(=O)c1ncc(-c2ccc3cnccc3c2)c(C)c1O]

A mixture of 3-hydroxy-5-(isoquinolin-6-yl)-4-methylpicolinic acid (250 mg, 0.62 mmol), ethyl glycinate hydrochloride (87 mg, 0.62 mmol), benzotriazole-1-yl-oxytripyrrolidinophosphonium hexafluorophosphate (390 mg, 0.75 mmol) and triethylamine (254 mg, 2.52 mmol) in dichloromethane (5.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/1) to obtain ethyl (3-hydroxy-5-(isoquinolin-6-yl)-4-methylpicolinoyl)glycinate (140 mg, 0.38 mmol, 43% yield) as white solid. LC-MS: m/z=366.1 [M+H]⁺, retention time 2.15 min (Method A).

### (3-Hydroxy-5-(isoquinolin-6-yl)-4-methylpicolinoyl)glycine

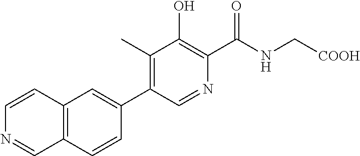 [SMILES: Cc1c(-c2ccc3cnccc3c2)cnc(C(=O)NCC(=O)O)c1O]

To a solution of ethyl (3-hydroxy-5-(isoquinolin-6-yl)-4-methylpicolinoyl)glycinate (140 mg, 0.38 mmol) in tetrahydrofuran/water (8.0 mL/2.0 mL) was added lithium hydroxide monohydrate (164 mg, 4.0 mmol). The mixture was stirred overnight and concentrated to remove tetrahydrofuran. The resulting aqueous solution was acidified with 10% hydrochloric acid solution to pH=3˜4. The precipitate was filtered, washed with water and purified by reverse prep-HPLC to obtain (3-hydroxy-5-(isoquinolin-6-yl)-4-methylpicolinoyl)glycine (72.6 mg, 0.22 mmol, 56% yield) as white solid. LC-MS: m/z=338.1 [M+H]⁺, retention time 2.28 min (Method A). ¹HNMR (500 MHz, DMSO-d₆) δ 12.88 (s, 1H), 9.71 (s, 1H), 9.45 (t, J=6.0 Hz, 1H), 8.68 (d, J=6.1 Hz, 1H), 8.47 (d, J=8.5 Hz, 1H), 8.27 (s, 1H), 8.24 (d, J=6.1 Hz, 1H), 8.19 (s, 1H), 7.95 (dd, J=8.5, 1.4 Hz, 1H), 4.04 (d, J=6.1 Hz, 2H), 2.20 (s, 3H).

### Example 4: Preparation of Compound 4

### 3-Chloro-4-methyl-5-(1-phenyl-1H-pyrazol-4-yl)picolinonitrile

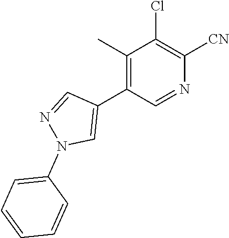 [SMILES: Cc1c(-c2cnn(-c3ccccc3)c2)cnc(C#N)c1Cl]

To a solution of 3,5-dichloro-4-methyl-pyridine-2-carbonitrile (550 mg, 2.96 mmol), 1-phenyl-4-(4,4,5,5-tetramethyl-1,3,2-dioxaborolan-2-yl)-1H-pyrazole (800 mg 2.96 mmol) and potassium carbonate (490 mg, 3.55 mmol) was added [1,1′-bis(diphenylphosphino)ferrocene]dichloropalladium(II) (86.5 mg, 0.12 mmol) in N,N-dimethylformamide/water (10.0 mL/1.0 mL). The mixture was stirred at 50° C. under nitrogen for 16.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=2/1) to afford 3-chloro-4-methyl-5-(1-phenyl-1H-pyrazol-4-yl)picolinonitrile (400 mg, 1.36 mmol, 46% yield) as yellow solid. LC-MS: m/z=295.3 [M+H]⁺, retention time=1.909 min (Method A).

### 3-(Benzyloxy)-4-methyl-5-(1-phenyl-1H-pyrazol-4-yl)picolinonitrile

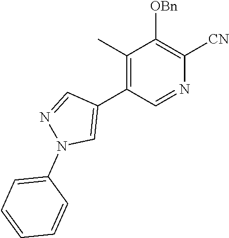 [SMILES: Cc1c(-c2cnn(-c3ccccc3)c2)cnc(C#N)c1OCc1ccccc1]

To a solution of 3-chloro-4-methyl-5-(1-phenyl-1H-pyrazol-4-yl)picolinonitrile (600.0 mg, 2.04 mmol) in N,N-dimethylformamide (10.0 mL) was sodium hydride (90 mg, 2.24 mmol, 60% w/w dispersion in mineral oil) at 0° C. under nitrogen. The mixture was stirred at 0° C. for 10 min and then benzyl alcohol (220.2 mg, 2.04 mmol) was added. The solution was stirred at 0° C. for 1.0 h and diluted with ethyl acetate and water. The organic layer was washed with brine, dried over sodium sulfate and concentrated under reduced pressure. The crude product was purified by flash chromatography (petroleum ether/ethyl acetate=3/1) to give 3-(benzyloxy)-4-methyl-5-(1-phenyl-1H-pyrazol-4-yl)picolinonitrile (300 mg, 0.82 mmol, 40.2% yield) as yellow solid. LC-MS: m/z=367.1 [M+H]⁺, retention time=2.20 min (Method A).

### 3-(Benzyloxy)-4-methyl-5-(1-phenyl-1H-pyrazol-4-yl)picolinic acid

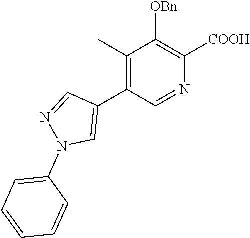 [SMILES: Cc1c(-c2cnn(-c3ccccc3)c2)cnc(C(=O)O)c1OCc1ccccc1]

To a solution of 3-(benzyloxy)-4-methyl-5-(1-phenyl-1H-pyrazol-4-yl)picolinonitrile (300 mg, 0.82 mmol) in ethanol (5.0 mL) was added 30% aqueous sodium hydroxide (5.0 mL). The mixture was stirred at 100° C. for 5.0 h, cooled and concentrated to remove ethanol. The resulting aqueous solution was acidified with 10% hydrochloric acid solution to pH=3˜4. The precipitate was filtered and dried to afford 3-(benzyloxy)-4-methyl-5-(1-phenyl-1H-pyrazol-4-yl)picolinic acid (290 mg, 0.75 mmol, 91.4% yield) as white solid. LC-MS: m/z=386.4 [M+H]⁺, retention time 1.74 min (Method A). The product was pure enough and used directly to the next step.

### Ethyl (3-(benzyloxy)-4-methyl-5-(1-phenyl-1H-pyrazol-4-yl)picolinoyl)glycinate

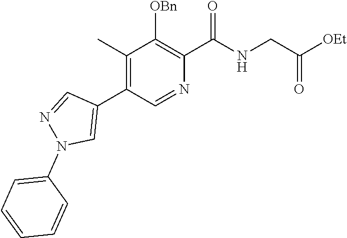 [SMILES: CCOC(=O)CNC(=O)c1ncc(-c2cnn(-c3ccccc3)c2)c(C)c1OCc1ccccc1]

A mixture of 3-(benzyloxy)-4-methyl-5-(1-phenyl-1H-pyrazol-4-yl)picolinic acid (290 mg, 0.75 mmol), ethyl glycinate hydrochloride (104 mg, 0.75 mmol), benzotriazole-1-yl-oxytripyrrolidinophosphonium hexafluorophosphate (430 mg, 0.83 mmol), triethylamine (380 mg, 3.75 mmol) in dichloromethane (8.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/1) to obtain ethyl (3-(benzyloxy)-4-methyl-5-(1-phenyl-1H-pyrazol-4-yl)picolinoyl)glycinate (200 mg, 0.42 mmol, 56.5% yield) as yellow solid. LC-MS: m/z=471.1 [M+H]⁺, retention time 2.13 min (Method A).

### Ethyl (3-hydroxy-4-methyl-5-(1-phenyl-1H-pyrazol-4-yl)picolinoyl)glycinate

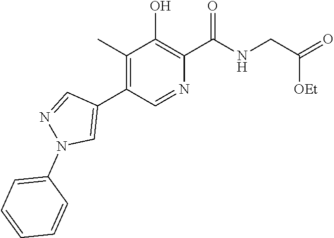 [SMILES: CCOC(=O)CNC(=O)c1ncc(-c2cnn(-c3ccccc3)c2)c(C)c1O]

A mixture of ethyl (3-(benzyloxy)-4-methyl-5-(1-phenyl-1H-pyrazol-4-yl)picolinoyl)glycinate (200 mg, 0.42 mmol) and 10% palladium on carbon (20.0 mg) in tetrahydrofuran (10.0 mL) was stirred under hydrogen atmosphere for 18 h. The insoluble solid was filtered and the filtrate was concentrated to give ethyl (3-hydroxy-4-methyl-5-(1-phenyl-1H-pyrazol-4-yl)picolinoyl)glycinate (150 mg, 0.39 mmol, 94% yield) as white solid. LC-MS: m/z=381.0 [M+H]⁺, retention time 2.16 min (Method A). The product was pure enough and used directly to the next step.

### (3-Hydroxy-4-methyl-5-(1-phenyl-1H-pyrazol-4-yl)picolinoyl)glycine

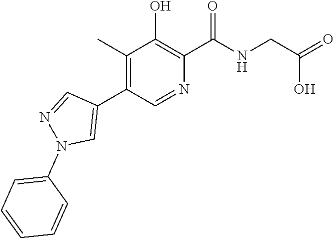 [SMILES: Cc1c(-c2cnn(-c3ccccc3)c2)cnc(C(=O)NCC(=O)O)c1O]

To a solution of ethyl (3-hydroxy-4-methyl-5-(1-phenyl-1H-pyrazol-4-yl)picolinoyl)glycinate (150 mg, 0.39 mmol) in tetrahydrofuran/water (6.0 mL/3.0 mL) was added lithium hydroxide monohydrate (164 mg, 4.0 mmol). The mixture was stirred overnight and concentrated to remove tetrahydrofuran. The resulting aqueous solution was acidified with 10% hydrochloric acid solution to pH=3˜4. The precipitate was filtered, washed with water and dried to obtain (3-hydroxy-4-methyl-5-(1-phenyl-1H-pyrazol-4-yl)picolinoyl)glycine (121.6 mg, 0.35 mmol, 88.5% yield) as white solid. LC-MS: m/z=353.1 [M+H]⁺, retention time 4.534 min (Method A). ¹HNMR (500 MHz, DMSO-d₆) δ 12.80 (s, 1H), 9.32 (t, J=6.1 Hz, 1H), 8.94 (s, 1H), 8.33 (s, 1H), 8.19 (s, 1H), 7.94 (d, J=7.8 Hz, 2H), 7.55 (t, J=7.9 Hz, 2H), 7.37 (t, J=7.4 Hz, 1H), 4.01 (d, J=6.1 Hz, 2H), 2.38 (s, 3H).

### Example 5: Preparation of Compound 5

### 2-Methyl-6-(4,4,5,5-tetramethyl-1,3,2-dioxaborolan-2-yl)quinoline

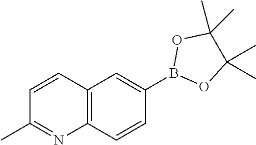

To a solution of 6-bromo-2-methylquinoline (2.0 g, 9.01 mmol), bis(pinacolato)diboron (2.6 g, 10.28 mmol) and potassium acetate (2.65 g, 27 mmol) was added [1,1′-bis(diphenylphosphino)ferrocene]dichloropalladium(II) (660 mg, 0.9 mmol) in 1,4-dioxane (15.0 mL). The mixture was stirred at 100° C. under nitrogen for 2.0 h and cooled to room temperature. Ethyl acetate and water were added to the solution, and the layers were separated. The organic layer was washed with brine, dried over sodium sulfate and concentrated to dryness. Crude 2-methyl-6-(4,4,5,5-tetramethyl-1,3,2-dioxaborolan-2-yl)quinoline (2.1 g, 7.81 mmol, 87.5% yield) was obtained. LC-MS: m/z=270.2 [M+H]⁺, retention time 2.08 min (Method B). The product was used directly to the next step.

### 3-Chloro-4-methyl-5-(2-methylquinolin-6-yl)picolinonitrile

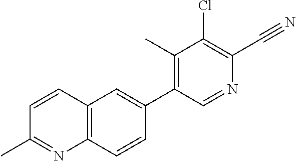 [SMILES: Cc1ccc2cc(-c3cnc(C#N)c(Cl)c3C)ccc2n1]

To a solution of 3,5-dichloro-4-methyl-pyridine-2-carbonitrile (1.2 g, 6.42 mmol), 2-methyl-6-(4,4,5,5-tetramethyl-1,3,2-dioxaborolan-2-yl)quinoline (1.73 g, 6.42 mmol) and potassium carbonate (1.16 g, 7.70 mmol) was added [1,1′-bis(diphenylphosphino)ferrocene]dichloropalladium(II) (234.5 mg, 0.32 mmol) in N,N-dimethylformamide/water (10.0 mL/1.0 mL). The mixture was stirred at 45° C. overnight under nitrogen and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=1/1) to afford 3-chloro-4-methyl-5-(2-methylquinolin-6-yl)picolinonitrile (900 mg, 3.07 mmol, 47.9% yield) as yellow solid. LC-MS: m/z=294.0 [M+H]⁺, retention time 1.61 min (Method A).

### 3-((4-Methoxybenzyl)oxy)-4-methyl-5-(2-methylquinolin-6-yl)picolinonitrile

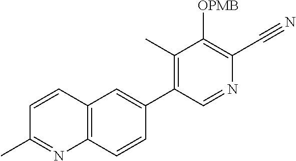

To a solution of 3-chloro-5-(isoquinolin-6-yl)-4-methylpicolinonitrile (500 mg, 1.71 mmol) in N,N-dimethylformamide (10.0 mL) was sodium hydride (95.6 mg, 2.39 mmol, 60% w/w dispersion in mineral oil) at 0° C. under nitrogen. The mixture was stirred at 0° C. for 10 min and then (4-methoxyphenyl)methanol (329.7 mg, 2.39 mmol) was added. The solution was stirred at 0° C. for 2.0 h and diluted with ethyl acetate and water. The organic layer was washed with brine, dried over sodium sulfate and concentrated under reduced pressure. The crude product was purified by flash chromatography (petroleum ether/ethyl acetate=2/1) to give 3-((4-methoxybenzyl)oxy)-4-methyl-5-(2-methylquinolin-6-yl)picolinonitrile (400 mg, 1.01 mmol, 59.2% yield) as yellow solid. LC-MS: m/z=396.0 [M+H]⁺, retention time 1.49 min (Method A).

### 3-Hydroxy-4-methyl-5-(2-methylquinolin-6-yl)picolinic acid

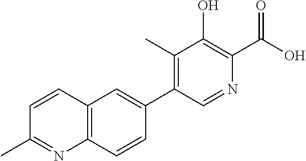 [SMILES: Cc1ccc2cc(-c3cnc(C(=O)O)c(O)c3C)ccc2n1]

To a solution of 3-((4-methoxybenzyl)oxy)-4-methyl-5-(2-methylquinolin-6-yl)picolinonitrile (400 mg, 0.47 mmol) in ethanol (10.0 mL) was added 30% aqueous sodium hydroxide (4.0 mL). The mixture was stirred at 100° C. for 5.0 h, cooled and concentrated to remove ethanol. The resulting aqueous solution was acidified with 10% hydrochloric acid solution to pH=3˜4. The precipitate was filtered and dried to afford 3-hydroxy-4-methyl-5-(2-methylquinolin-6-yl)picolinic acid (400 mg, crude) as white solid. LC-MS: m/z=295.2 [M+H]⁺, retention time 1.15 min (Method B). The product was pure enough and used directly to the next step.

### Ethyl (3-hydroxy-4-methyl-5-(2-methylquinolin-6-yl)picolinoyl)glycinate

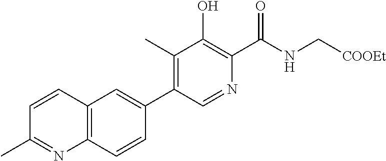 [SMILES: CCOC(=O)CNC(=O)c1ncc(-c2ccc3nc(C)ccc3c2)c(C)c1O]

A mixture of 3-hydroxy-4-methyl-5-(2-methylquinolin-6-yl)picolinic acid (200.0 mg, 0.68 mmol), ethyl glycinate hydrochloride (113.5 mg, 0.82 mmol), benzotriazole-1-yl-oxytripyrrolidinophosphonium hexafluorophosphate (424.5 mg, 0.82 mmol) and triethylamine (343.5 mg, 3.40 mmol) in dichloromethane (10.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/2) to obtain ethyl (3-hydroxy-4-methyl-5-(2-methylquinolin-6-yl)picolinoyl)glycinate (120 mg, 0.32 mmol, 47.1% yield) as white solid. LC-MS: m/z=380.1 [M+H]⁺, retention time 1.60 min (Method A).

### (3-Hydroxy-4-methyl-5-(2-methylquinolin-6-yl)picolinoyl)glycine

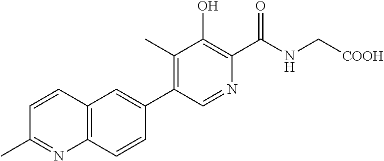 [SMILES: Cc1ccc2cc(-c3cnc(C(=O)NCC(=O)O)c(O)c3C)ccc2n1]

To a solution of ethyl (3-hydroxy-4-methyl-5-(2-methylquinolin-6-yl)picolinoyl)glycinate (120 mg, 0.33 mmol) in tetrahydrofuran/water (8.0 mL/2.0 mL) was added lithium hydroxide monohydrate (140 mg, 3.3 mmol). The mixture was stirred overnight and concentrated to remove tetrahydrofuran. The resulting aqueous solution was acidified with 10% hydrochloric acid solution to pH=3˜4. The precipitate was filtered, washed with water and purified by reverse prep-HPLC to obtain (3-hydroxy-4-methyl-5-(2-methylquinolin-6-yl)picolinoyl)glycine (5.9 mg, 0.017 mmol, 5.09%) as white solid. LC-MS: m/z=352.1 [M+H]⁺, retention time 2.05 min (Method B). ¹HNMR (400 MHz, DMSO-d₆) δ 9.04 (s, 1H), 8.32 (d, J=8.4 Hz, 1H), 8.15 (s, 1H), 8.03 (d, J=8.8 Hz, 2H), 7.78 (d, J=8.6 Hz, 1H), 7.50 (d, J=8.4 Hz, 1H), 3.72 (d, J=4.3 Hz, 2H), 2.70 (s, 3H), 2.20 (s, 3H).

### Example 6: Preparation of Compound 6

### Tert-butyl 4-(4-(5-chloro-6-cyano-4-methylpyridin-3-yl)-1H-pyrazol-1-yl)piperidine-1-carboxylate

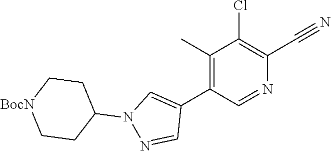 [SMILES: Cc1c(-c2cnn(C3CCN(C(=O)OC(C)(C)C)CC3)c2)cnc(C#N)c1Cl]

To a solution of 3,5-dichloro-4-methyl-pyridine-2-carbonitrile (561 mg, 3.0 mmol), tert-butyl 4-(4-(4,4,5,5-tetramethyl-1,3,2-dioxaborolan-2-yl)-1H-pyrazol-1-yl)piperidine-1-carboxylate (1.13 3.0 mmol) and potassium carbonate (497 mg, 3.6 mmol) was added [1,1′-bis(diphenylphosphino)ferrocene]dichloropalladium(II) (110 mg, 0.15 mmol) in N,N-dimethylformamide/water (10.0 mL/1.0 mL). The mixture was stirred at 50° C. under nitrogen for 16.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=2/1) to afford tert-butyl 4-(4-(5-chloro-6-cyano-4-methylpyridin-3-yl)-1H-pyrazol-1-yl)piperidine-1-carboxylate (400 mg, 1.0 mmol, 33% yield) as yellow solid. LC-MS: m/z=346.2[M−56]+, retention time=2.078 min (Method A).

### Tert-butyl 4-(4-(5-(benzyloxy)-6-cyano-4-methylpyridin-3-yl)-1H-pyrazol-1-yl)piperidine-1-carboxylate

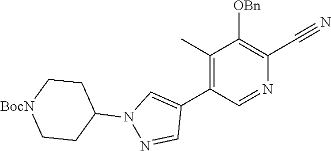 [SMILES: Cc1c(-c2cnn(C3CCN(C(=O)OC(C)(C)C)CC3)c2)cnc(C#N)c1OCc1ccccc1]

To a solution of tert-butyl 4-(4-(5-chloro-6-cyano-4-methylpyridin-3-yl)-1H-pyrazol-1-yl)piperidine-1-carboxylate (600 mg, 1.49 mmol) in N,N-dimethylformamide (10.0 mL) was sodium hydride (72 mg, 1.79 mmol, 60% w/w dispersion in mineral oil) at 0° C. under nitrogen. The mixture was stirred at 0° C. for 10 min and then benzyl alcohol (194 mg, 1.79 mmol) was added. The solution was stirred at 0° C. for 1.0 h and diluted with ethyl acetate and water. The organic layer was washed with brine, dried over sodium sulfate and concentrated under reduced pressure. The crude product was purified by flash chromatography (petroleum ether/ethyl acetate=3/1) to give tert-butyl 4-(4-(5-(benzyloxy)-6-cyano-4-methylpyridin-3-yl)-1H-pyrazol-1-yl)piperidine-1-carboxylate (330 mg, 0.70 mmol, 46% yield) as yellow solid. LC-MS: m/z=474.0 [M+H]⁺, retention time=2.21 min (Method A).

### 3-(Benzyloxy)-5-(1-(1-(tert-butoxycarbonyl)piperidin-4-yl)-1H-pyrazol-4-yl)-4-methylpicolinic Acid

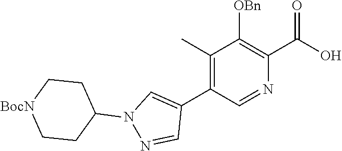 [SMILES: Cc1c(-c2cnn(C3CCN(C(=O)OC(C)(C)C)CC3)c2)cnc(C(=O)O)c1OCc1ccccc1]

To a solution of tert-butyl 4-(4-(5-(benzyloxy)-6-cyano-4-methylpyridin-3-yl)-1H-pyrazol-1-yl)piperidine-1-carboxylate (330 mg, 0.70 mmol) in ethanol (15.0 mL) was added 30% aqueous sodium hydroxide (5.0 mL). The mixture was stirred at 100° C. for 1.0 h, cooled and concentrated to remove ethanol. The resulting aqueous solution was acidified with 10% hydrochloric acid solution to pH=3˜4. The precipitate was filtered and dried to afford 3-(benzyloxy)-5-(1-(1-(tert-butoxycarbonyl)piperidin-4-yl)-1H-pyrazol-4-yl)-4-methylpicolinic acid (330 mg, 0.33 mmol, 96% yield) as white solid. LC-MS: m/z=493.2 [M+H]⁺, retention time 1.97 min (Method A). The product was pure enough and used directly to the next step.

### Tert-butyl 4-(4-(5-(benzyloxy)-6-((2-ethoxy-2-oxoethyl)carbamoyl)-4-methylpyridin-3-yl)-1H-pyrazol-1-yl)piperidine-1-carboxylate

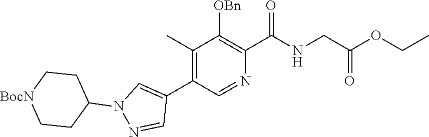 [SMILES: CCOC(=O)CNC(=O)c1ncc(-c2cnn(C3CCN(C(=O)OC(C)(C)C)CC3)c2)c(C)c1OCc1ccccc1]

A mixture of 3-(benzyloxy)-5-(1-(1-(tert-butoxycarbonyl)piperidin-4-yl)-1H-pyrazol-4-yl)-4-methylpicolinic acid (165 mg, 0.33 mmol), ethyl glycinate hydrochloride (47 mg, 0.33 mmol), benzotriazole-1-yl-oxytripyrrolidinophosphonium hexafluorophosphate (192 mg, 0.37 mmol) and triethylamine (167 mg, 1.67 mmol) in dichloromethane (10.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/1) to obtain tert-butyl 4-(4-(5-(benzyloxy)-6-((2-ethoxy-2-oxoethyl)carbamoyl)-4-methylpyridin-3-yl)-1H-pyrazol-1-yl)piperidine-1-carboxylate (165 mg, 0.29 mmol, 85% yield) as white solid. LC-MS: m/z=578.0 [M+H]⁺, retention time 2.14 min (Method A).

### Tert-butyl 4-(4-(6-((2-ethoxy-2-oxoethyl)carbamoyl)-5-hydroxy-4-methylpyridin-3-yl)-1H-pyrazol-1-yl)piperidine-1-carboxylate

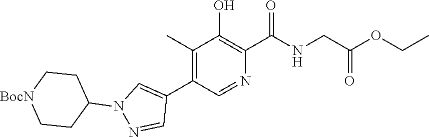 [SMILES: CCOC(=O)CNC(=O)c1ncc(-c2cnn(C3CCN(C(=O)OC(C)(C)C)CC3)c2)c(C)c1O]

A mixture of tert-butyl 4-(4-(5-(benzyloxy)-6-((2-ethoxy-2-oxoethyl)carbamoyl)-4-methylpyridin-3-yl)-1H-pyrazol-1-yl)piperidine-1-carboxylate (165 mg, 0.29 mmol) and 10% palladium on carbon (20.0 mg) in tetrahydrofuran (10.0 mL) was stirred under hydrogen atmosphere for 18.0 h. The insoluble solid was filtered and the filtrate was concentrated to give tert-butyl 4-(4-(6-((2-ethoxy-2-oxoethyl)carbamoyl)-5-hydroxy-4-methylpyridin-3-yl)-1H-pyrazol-1-yl)piperidine-1-carboxylate (135 mg, 0.27 mmol, 97% yield) as a yellow solid. LC-MS: m/z=460.1[M+H]⁺, retention time 2.19 min (Method A). The product was pure enough and used directly to the next step.

### (5-(1-(1-(Tert-butoxycarbonyl)piperidin-4-yl)-1H-pyrazol-4-yl)-3-hydroxy-4-methylpicolinoyl)glycine

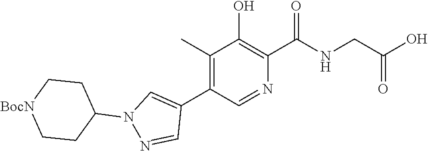 [SMILES: Cc1c(-c2cnn(C3CCN(C(=O)OC(C)(C)C)CC3)c2)cnc(C(=O)NCC(=O)O)c1O]

To a solution of tert-butyl 4-(4-(6-((2-ethoxy-2-oxoethyl)carbamoyl)-5-hydroxy-4-methylpyridin-3-yl)-1H-pyrazol-1-yl)piperidine-1-carboxylate (135 mg, 0.27 mmol) in tetrahydrofuran/water (8.0 mL/2.0 mL) was added lithium hydroxide monohydrate (164 mg, 4.0 mmol). The mixture was stirred overnight and concentrated to remove tetrahydrofuran. The resulting aqueous solution was acidified with 10% hydrochloric acid solution to pH=3˜4. The precipitate was filtered, washed with water and dried to obtain (5-(1-(1-(tert-butoxycarbonyl)piperidin-4-yl)-1H-pyrazol-4-yl)-3-hydroxy-4-methylpicol

inoyl)glycine (85.5 mg, 0.18 mmol, 67% yield) as white solid. LC-MS: m/z=502.1 [M+H]⁺, retention time 5.33 min (Method A). ¹HNMR (400 MHz, DMSO-d₆) δ 12.75 (s, 1H), 9.62-9.10 (m, 1H), 8.28 (s, 1H), 8.23 (s, 1H), 7.88 (s, 1H), 4.56-4.30 (m, 1H), 4.15-4.03 (m, 2H), 4.00 (d, J=6.1 Hz, 2H), 2.93 (br s, 2H), 2.29 (d, J=8.5 Hz, 3H), 2.14-2.00 (m, 2H), 1.92-1.77 (m, 2H), 1.43 (s, 9H).

### Example 7: Preparation of Compound 7

### 2-Phenyl-5-(tributylstannyl)thiazole

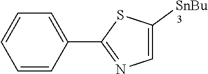

To a solution of 2-phenylthiazole (10.0 g, 62.03 mmol) in anhydrous tetrahydrofuran (200.0 mL) was added n-butyllithium (30.98 mL, 77.54 mmol, 2.5 M in hexane) at −78° C. under nitrogen. The mixture was stirred at −78° C. for 30 min and then chlorotributyltin (20.8 mL, 71.34 mmol) was added. The mixture was allowed to warm up to 0° C. and left stirring for another one hour. The reaction was quenched with saturated ammonium chloride solution and extracted twice with ethyl acetate. The organic layer was separated, washed with brine, dried over sodium sulfate and concentrated under reduced pressure. The crude product was purified by flash chromatography (petroleum ether/ethyl acetate=5/1) to afford 2-phenyl-5-(tributylstannyl)thiazole (27.7 g, 61.6 mmol, 99% yield) as a yellow solid. LC-MS: m/z=451.2 [M+H]⁺, retention time 2.30 min (Method A).

### 3-Chloro-4-methyl-5-(2-phenylthiazol-5-yl)picolinonitrile

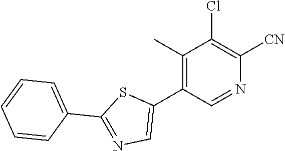 [SMILES: Cc1c(-c2cnc(-c3ccccc3)s2)cnc(C#N)c1Cl]

To a solution of 3,5-dibromo-4,6-dimethylpicolinonitrile (2.9 g, 15.55 mmol), 2-phenyl-5-(tributylstannyl)thiazole (5.0 g, 11.10 mmol), caesium fluoride (5.06 g, 33.31 mmol) and copper iodide (423 mg, 2.22 mmol) in N,N-dimethylformamide (20.0 mL) was added tetrakis(triphenylphosphine)palladium (1.28 g, 1.11 mmol). The mixture was stirred at 40° C. under nitrogen for 16.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=2/1) to afford 3-chloro-4-methyl-5-(2-phenylthiazol-5-yl)picolinonitrile (150 mg, 0.48 mmol, 10% yield) as green solid. LC-MS: m/z=312.1 [M+H]⁺, retention time=1.909 min (Method A).

### 3-Hydroxy-4-methyl-5-(2-phenylthiazol-5-yl)picolinonitrile

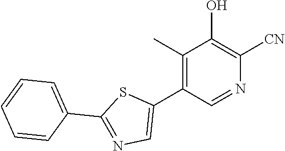 [SMILES: Cc1c(-c2cnc(-c3ccccc3)s2)cnc(C#N)c1O]

To a solution of 3-chloro-4-methyl-5-(2-phenylthiazol-5-yl)picolinonitrile (1.50 g, 4.81 mmol) and potassium carbonate (2.0 g, 14.43 mmol) in N,N-dimethylacetamide (10.0 mL) was added benzyl alcohol (780 mg, 7.22 mmol). The mixture was stirred at 120° C. for 2.0 d and concentrated. The residue was partitioned between ethyl acetate and water. The organic layer was washed with brine, dried over sodium sulfate and concentrated under reduced pressure. The crude product was purified by reverse prep-HPLC to obtain 3-hydroxy-4-methyl-5-(2-phenylthiazol-5-yl)picolinonitrile (200 mg, 0.68 mmol, 14% yield) as white solid. LC-MS: m/z=294.1 [M+H]⁺, retention time=1.40 min (Method A).

### 3-Hydroxy-4-methyl-5-(2-phenylthiazol-5-yl)picolinic Acid

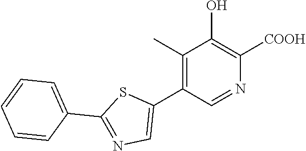 [SMILES: Cc1c(-c2cnc(-c3ccccc3)s2)cnc(C(=O)O)c1O]

To a solution of 3-hydroxy-4-methyl-5-(2-phenylthiazol-5-yl)picolinonitrile (200 mg, 0.68 mmol) in ethanol (10.0 mL) was added 30% aqueous sodium hydroxide (3.0 mL). The mixture was stirred at 100° C. for 3.0 h, cooled and concentrated to remove ethanol. The resulting aqueous solution was acidified with 10% hydrochloric acid solution to pH=3˜4. The precipitate was filtered and dried to afford 3-hydroxy-4-methyl-5-(2-phenylthiazol-5-yl)picolinic acid (200 mg, 0.64 mmol, 94% yield) as white solid. LC-MS: m/z=313.4 [M+H]⁺, retention time 1.74 min (Method A). The product was pure enough and used directly to the next step.

### Ethyl (3-hydroxy-4-methyl-5-(2-phenylthiazol-5-yl)picolinoyl)glycinate

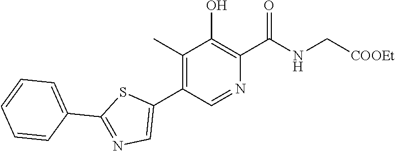 [SMILES: CCOC(=O)CNC(=O)c1ncc(-c2cnc(-c3ccccc3)s2)c(C)c1O]

A mixture of 3-hydroxy-4-methyl-5-(2-phenylthiazol-5-yl)picolinic acid (200 mg, 0.64 mmol), ethyl glycinate hydrochloride (150 mg, 1.08 mmol), benzotriazole-1-yl-oxytripyrrolidinophosphonium hexafluorophosphate (420 mg, 0.77 mmol) and triethylamine (380 mg, 3.75 mmol) in dichloromethane (20.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/2) to obtain ethyl (3-hydroxy-4-methyl-5-(2-phenylthiazol-5-yl)picolinoyl)glycinate (140 mg, 0.35 mmol, 55% yield) as white solid. LC-MS: m/z=398.1 [M+H]⁺, retention time 2.13 min (Method A).

### (3-Hydroxy-4-methyl-5-(2-phenylthiazol-5-yl)picolinoyl)glycine

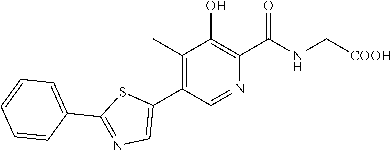 [SMILES: Cc1c(-c2cnc(-c3ccccc3)s2)cnc(C(=O)NCC(=O)O)c1O]

To a solution of ethyl (3-hydroxy-4-methyl-5-(2-phenylthiazol-5-yl)picolinoyl)glycinate (140 mg, 0.35 mmol) in tetrahydrofuran/water (8.0 mL/4.0 mL) was added lithium hydroxide monohydrate (164 mg, 4.0 mmol). The mixture was stirred at 40° C. overnight and concentrated to remove tetrahydrofuran. The resulting aqueous solution was acidified with 10% hydrochloric acid solution to pH=3˜4. The precipitate was filtered, washed with water and dried to obtain (3-hydroxy-4-methyl-5-(2-phenylthiazol-5-yl)picolinoyl)glycine (106.6 mg, 82% yield) as white solid. LC-MS: m/z=370.0 [M+H]⁺, retention time 4.92 min (Method A). ¹HNMR (400 MHz, DMSO-d₆) δ 12.91 (s, 1H), 9.41 (t, J=6.1 Hz, 1H), 8.31 (s, 1H), 8.21 (s, 1H), 8.12-7.87 (m, 2H), 7.78-7.38 (m, 3H), 4.02 (d, J=6.1 Hz, 2H), 2.38 (s, 3H).

### Example 8: Preparation of Compound 8

### 4,4,5,5-Tetramethyl-2-(3-phenoxyphenyl)-1,3,2-dioxaborolane

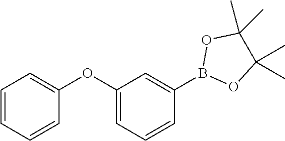 [SMILES: CC1(C)OB(c2cccc(Oc3ccccc3)c2)OC1(C)C]

To a solution of 1-bromo-3-phenoxybenzene (1.5 g, 6.02 mmol), bis(pinacolato)diboron (3.06 g, 12.04 mmol) and potassium acetate (2.36 g, 24.1 mmol) was added [1,1′-bis(diphenylphosphino)ferrocene]dichloropalladium(II) (438 mg, 0.6 mmol) in 1,4-dioxane (15.0 mL). The mixture was stirred at 90° C. under nitrogen for 2.0 h and cooled to room temperature. Ethyl acetate and water were added to the solution, and the layers were separated. The organic layer was washed with brine, dried over sodium sulfate and concentrated to dryness. Crude 4,4,5,5-tetramethyl-2-(3-phenoxyphenyl)-1,3,2-dioxaborolane (1.0 g, 3.37 mmol, 56.1% yield) was obtained. LC-MS: m/z=297.0 [M+H]⁺, retention time 2.41 min (Method B) The product was used directly to the next step.

### 3-Chloro-4-methyl-5-(3-phenoxyphenyl)picolinonitrile

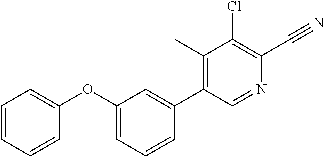 [SMILES: Cc1c(-c2cccc(Oc3ccccc3)c2)cnc(C#N)c1Cl]

To a solution of 3,5-dichloro-4-methyl-pyridine-2-carbonitrile (626 mg, 3.37 mmol), 4,4,5,5-tetramethyl-2-(3-phenoxyphenyl)-1,3,2-dioxaborolane (1.0 g, 3.37 mmol) and potassium carbonate (697 mg, 5.05 mmol) was added [1,1′-bis(diphenylphosphino)ferrocene]dichloropalladium(II) (123 mg, 0.17 mmol) in N,N-dimethylformamide/water (10.0 mL/1.0 mL). The mixture was stirred at 50° C. overnight under nitrogen and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=3/1) to afford 3-chloro-4-methyl-5-(3-phenoxyphenyl)picolinonitrile (450 mg, 1.41 mmol, 41.7% yield) as yellow solid. LC-MS: m/z=321.0 [M+H]⁺, retention time 2.30 min (Method A).

### 3-(Benzyloxy)-4-methyl-5-(3-phenoxyphenyl)picolinonitrile

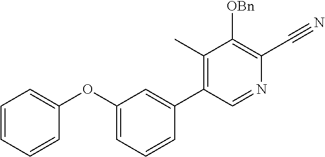 [SMILES: Cc1c(-c2cccc(Oc3ccccc3)c2)cnc(C#N)c1OCc1ccccc1]

To a solution of 3-chloro-4-methyl-5-(3-phenoxyphenyl)picolinonitrile (450 mg, 1.41 mmol) in N,N-dimethylformamide (10.0 mL) was sodium hydride (113 mg, 2.82 mmol, 60% w/w dispersion in mineral oil) at 0° C. under nitrogen. The mixture was stirred at 0° C. for 10 min and then benzyl alcohol (152 mg, 1.41 mmol) was added. The solution was stirred at 0° C. for 1.0 h and diluted with ethyl acetate and water. The organic layer was washed with brine, dried over sodium sulfate and concentrated under reduced pressure. The crude product was purified by flash chromatography (petroleum ether/ethyl acetate=2/1) to give 3-(benzyloxy)-4-methyl-5-(3-phenoxyphenyl)picolinonitrile (350 mg, 0.89 mmol, 63.3% yield) as yellow solid. LC-MS: m/z=352.0 [M+H]⁺, retention time 1.70 min (Method A).

### 3-(Benzyloxy)-4-methyl-5-(3-phenoxyphenyl)picolinic acid

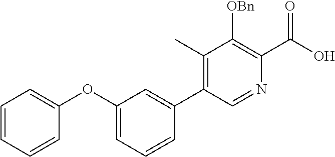 [SMILES: Cc1c(-c2cccc(Oc3ccccc3)c2)cnc(C(=O)O)c1OCc1ccccc1]

To a solution of 3-(benzyloxy)-4-methyl-5-(3-phenoxyphenyl)picolinonitrile (350 mg, 0.89 mmol) in ethanol (10.0 mL) was added 30% aqueous sodium hydroxide (4.0 mL). The mixture was stirred at 100° C. overnight, cooled and concentrated to remove ethanol. The resulting aqueous solution was acidified with 10% hydrochloric acid solution to pH=3˜4. The precipitate was filtered and dried to afford 3-(benzyloxy)-4-methyl-5-(3-phenoxyphenyl)picolinic acid (300 mg, 0.73 mmol, 82.0% yield) as white solid. LC-MS: m/z=393.0 (M+H)⁺, retention time 2.35 min (Method B). The product was pure enough and used directly to the next step.

### Ethyl (3-(benzyloxy)-4-methyl-5-(3-phenoxyphenyl)picolinoyl)glycinate

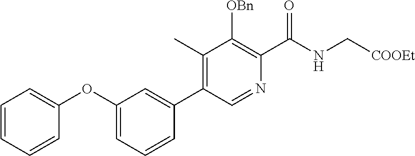 [SMILES: CCOC(=O)CNC(=O)c1ncc(-c2cccc(Oc3ccccc3)c2)c(C)c1OCc1ccccc1]

A mixture of 3-(benzyloxy)-4-methyl-5-(3-phenoxyphenyl)picolinic acid (300 mg, 0.73 mmol), ethyl glycinate hydrochloride (152 mg, 1.09 mmol), benzotriazole-1-yl-oxytripyrrolidinophosphonium hexafluorophosphate (456 mg, 0.88 mmol) and triethylamine (370 mg, 3.65 mmol) in dichloromethane (10.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/2) to obtain ethyl (3-(benzyloxy)-4-methyl-5-(3-phenoxyphenyl)picolinoyl)glycinate (210 mg, 0.42 mmol, 58.0% yield) as white solid. LC-MS: m/z=497.0 (M+H)⁺, retention time 2.26 min (Method A).

### Ethyl (3-hydroxy-4-methyl-5-(3-phenoxyphenyl)picolinoyl)glycinate

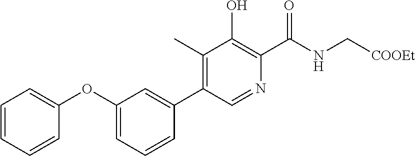 [SMILES: CCOC(=O)CNC(=O)c1ncc(-c2cccc(Oc3ccccc3)c2)c(C)c1O]

A mixture of ethyl (3-(benzyloxy)-4-methyl-5-(3-phenoxyphenyl)picolinoyl)glycinate (210 mg, 0.42 mmol) and 10% palladium on carbon (20.0 mg) in tetrahydrofuran (10.0 mL) was stirred at 40° C. under hydrogen atmosphere for 5.0 h. The insoluble solid was filtered and the filtrate was concentrated to give ethyl (3-hydroxy-4-methyl-5-(3-phenoxyphenyl)picolinoyl)glycinate (140 mg, 0.34 mmol, 81% yield) as a yellow solid. LC-MS: m/z=407.0 (M+H)⁺, retention time 2.32 min (Method A). The product was pure enough and used directly to the next step.

### (3-Hydroxy-4-methyl-5-(3-phenoxyphenyl)picolinoyl)glycine

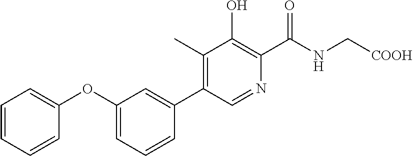 [SMILES: Cc1c(-c2cccc(Oc3ccccc3)c2)cnc(C(=O)NCC(=O)O)c1O]

To a solution of ethyl (3-hydroxy-4-methyl-5-(3-phenoxyphenyl)picolinoyl)glycinate (140 mg, 0.34 mmol) in tetrahydrofuran/water (8.0 mL/2.0 mL) was added lithium hydroxide monohydrate (164 mg, 4.0 mmol). The mixture was stirred overnight and concentrated to remove tetrahydrofuran. The resulting aqueous solution was acidified with 10% hydrochloric acid solution to pH=3˜4. The precipitate was filtered, washed with water and purified by reverse perp-HPLC to obtain (3-hydroxy-4-methyl-5-(3-phenoxyphenyl)picolinoyl)glycine (38.7 mg, 0.10 mmol, 30% yield) as white solid. LC-MS: m/z=379.0 [M+H]⁺, retention time 5.76 min (Method A). ¹HNMR (400 MHz, DMSO-d₆) δ 12.82 (s, 1H), 9.46-9.14 (m, 1H), 8.05 (s, 1H), 7.53 (t, J=8.0 Hz, 1H), 7.47-7.38 (m, 2H), 7.25-7.14 (m, 2H), 7.13-7.04 (m, 4H), 3.96 (d, J=6.0 Hz, 2H), 2.16 (s, 3H).

### Example 9: Preparation of Compound 9

### 5-Chloro-4-methyl-6′-(trifluoromethyl)-[3,3′-bipyridine]-6-carbonitrile

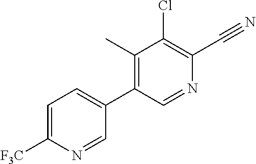 [SMILES: Cc1c(-c2ccc(C(F)(F)F)nc2)cnc(C#N)c1Cl]

To a solution of 3,5-dichloro-4-methyl-pyridine-2-carbonitrile (500.00 mg, 2.67 mmol), 5-(4,4,5,5-tetramethyl-1,3,2-dioxaborolan-2-yl)-2-(trifluoromethyl) pyridine (730.03 mg, 2.67 mmol) and potassium carbonate (443.41 mg, 3.21 mmol) was added [1,1′-bis(diphenylphosphino)ferrocene]dichloropalladium(II) (78.25 mg, 0.11 mmol) in N,N-dimethylformamide/water (10.0 mL/1.0 mL). The mixture was stirred at 50° C. overnight under nitrogen and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=5/1) to afford 5-chloro-4-methyl-6′-(trifluoromethyl)-[3,3′-bipyridine]-6-carbonitrile (577 mg, 1.94 mmol, 72% yield) as yellow solid. LC-MS: m/z=298 [M+H]⁺, retention time 2.052 min (Method A).

### 5-(Benzyloxy)-4-methyl-6′-(trifluoromethyl)-[3,3′-bipyridine]-6-carbonitrile

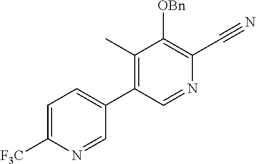 [SMILES: Cc1c(-c2ccc(C(F)(F)F)nc2)cnc(C#N)c1OCc1ccccc1]

To a solution of 5-chloro-4-methyl-6′-(trifluoromethyl)-[3,3′-bipyridine]-6-carbonitrile (500.00 mg, 1.68 mmol) in N,N-dimethylfonnamide (20.0 mL) was sodium hydride (80.8 mg, 2.02 mmol, 60% w/w dispersion in mineral oil) at 0° C. under nitrogen. The mixture was stirred at 0° C. for 10 min and then benzyl alcohol (217.97 mg, 2.02 mmol, 0.21 mL) was added. The solution was stirred at 0° C. for 50 min and diluted with ethyl acetate and water. The organic layer was washed with brine, dried over sodium sulfate and concentrated under reduced pressure. The crude product was purified by flash chromatography (petroleum ether/ethyl acetate=5/1) to give 5-(benzyloxy)-4-methyl-6′-(trifluoromethyl)-[3,3′-bipyridine]-6-carbonitrile (306 mg, 49% yield). LC-MS: m/z=370 [M+H]⁺, retention time 2.190 min (Method B).

### 5-(Benzyloxy)-4-methyl-6′-(trifluoromethyl)-[3,3′-bipyridine]-6-carboxylic Acid

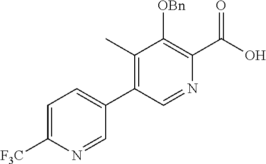 [SMILES: Cc1c(-c2ccc(C(F)(F)F)nc2)cnc(C(=O)O)c1OCc1ccccc1]

To a solution of 5-(benzyloxy)-4-methyl-6′-(trifluoromethyl)-[3,3′-bipyridine]-6-carbonitrile (306.00 mg, 0.83 mmol) in ethanol (10.0 mL) was added 30% aqueous sodium hydroxide (4.0 mL). The mixture was stirred at 100° C. for 3.0 h, cooled and concentrated to remove ethanol. The resulting aqueous solution was acidified with 10% hydrochloric acid solution to pH=3˜4. The precipitate was filtered and dried to afford 5-(benzyloxy)-4-methyl-6′-(trifluoromethyl)-[3,3′-bipyridine]-6-carboxylic acid (353 mg, crude) as white solid. LC-MS: m/z=389 [M+H]⁺, retention time 1.977 min (Method A). The product was pure enough and used directly to the next step.

### Ethyl (5-(benzyloxy)-4-methyl-6′-(trifluoromethyl)-[3,3′-bipyridine]-6-carbonyl) glycinate

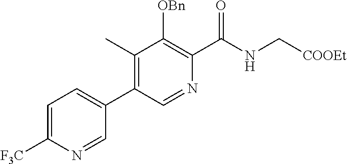 [SMILES: CCOC(=O)CNC(=O)c1ncc(-c2ccc(C(F)(F)F)nc2)c(C)c1OCc1ccccc1]

A mixture of 5-(benzyloxy)-4-methyl-6′-(trifluoromethyl)-[3,3′-bipyridine]-6-carboxylic acid (300.00 mg, 0.77 mmol), ethyl glycinate hydrochloride (107.83 mg, 0.77 mmol), benzotriazole-1-yl-oxytripyrrolidinophosphonium hexafluorophosphate (442.21 mg, 0.85 mmol) and triethylamine (390.85 mg, 3.86 mmol, 0.54 mL) in dichloromethane (20.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=2/1) to obtain ethyl (5-(benzyloxy)-4-methyl-6′-(trifluoromethyl)-[3,3′-bipyridine]-6-carbonyl) glycinate (257 mg, 0.54 mmol, 70% yield). LC-MS: m/z=474 [M+H]⁺, retention time 1.810 min (Method A).

### Ethyl (5-hydroxy-4-methyl-6′-(trifluoromethyl)-[3,3′-bipyridine]-6-carbonyl) glycinate

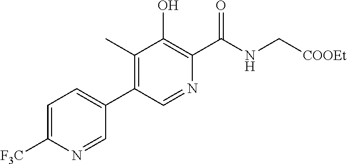 [SMILES: CCOC(=O)CNC(=O)c1ncc(-c2ccc(C(F)(F)F)nc2)c(C)c1O]

A mixture of ethyl (5-(benzyloxy)-4-methyl-6′-(trifluoromethyl)-[3,3′-bipyridine]-6-carbonyl) glycinate (230.00 mg, 0.49 mmol) and 10% palladium on carbon (20.0 mg) in tetrahydrofuran (10.0 mL) was stirred at room temperature under hydrogen atmosphere overnight. The insoluble solid was filtered and the filtrate was concentrated to give ethyl (5-hydroxy-4-methyl-6′-(trifluoromethyl)-[3,3′-bipyridine]-6-carbonyl) glycinate (230 mg, crude) as a yellow solid. LC-MS: m/z=384 [M+H]⁺, retention time 2.116 min (Method A). The product was pure enough and used directly to the next step.

### (5-Hydroxy-4-methyl-6′-(trifluoromethyl)-[3,3′-bipyridine]-6-carbonyl)glycine

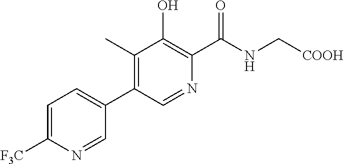 [SMILES: Cc1c(-c2ccc(C(F)(F)F)nc2)cnc(C(=O)NCC(=O)O)c1O]

To a solution of ethyl (5-hydroxy-4-methyl-6′-(trifluoromethyl)-[3,3′-bipyridine]-6-carbonyl) glycinate (200.00 mg, 0.52 mmol) in methanol/water (10.0 mL/2.0 mL) was added lithium hydroxide monohydrate (219 mg, 5.22 mmol). The mixture was stirred at 40° C. overnight and concentrated to remove methanol. The resulting aqueous solution was acidified with 10% hydrochloric acid solution to pH=3˜4. The precipitate was filtered, washed with water and purified by reverse perp-HPLC to obtain (5-hydroxy-4-methyl-6′-(trifluoromethyl)-[3,3′-bipyridine]-6-carbonyl)glycine (Formate) (48.2 mg, 0.14 mmol, 26% yield) as yellow solid. LC-MS: m/z=356 [M+H]⁺, retention time 4.420 min (Method A). ¹H NMR (500 MHz, DMSO-d₆) δ 12.88 (br, 2H), 9.43 (t, J=6.0 Hz, 1H), 8.90 (d, J=1.5 Hz, 1H), 8.24 (dd, J=8.5 Hz, J=2.0 Hz, 1H), 8.16 (s, 1H), 8.07 (d, J=7.5 Hz, 1H), 4.01 (d, J=6.0 Hz, 2H), 2.18 (s, 3H).

### Example 10: Preparation of Compound 10

### 5-Chloro-4-methyl-[3,3′-bipyridine]-6-carbonitrile

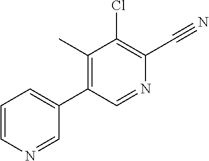 [SMILES: Cc1c(-c2cccnc2)cnc(C#N)c1Cl]

To a solution of 3,5-dichloro-4-methyl-pyridine-2-carbonitrile (500 mg, 2.67 mmol), pyridin-3-ylboronic acid (329 mg 2.67 mmol) and potassium carbonate (443 mg, 3.21 mmol) was added [1,1′-bis(diphenylphosphino)ferrocene]dichloropalladium(II) (78 mg, 0.11 mmol) in N,N-dimethylformamide/water (5.0 mL/0.5 mL)). The mixture was stirred at 45° C. overnight under nitrogen and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=1/1) to afford 5-chloro-4-methyl-[3,3′-bipyridine]-6-carbonitrile (300 mg, 1.31 mmol, 49% yield) as a yellow solid. LC-MS: m/z=230.1 [M+H]⁺, retention time=1.83 min (Method A).

### 5-(Benzyloxy)-4-methyl-[3,3′-bipyridine]-6-carbonitrile

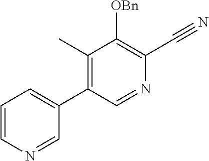 [SMILES: Cc1c(-c2cccnc2)cnc(C#N)c1OCc1ccccc1]

To a solution of 5-chloro-4-methyl-[3,3′-bipyridine]-6-carbonitrile (300.0 mg, 1.31 mmol) in N,N-dimethylformamide (10.0 mL) was sodium hydride (78 mg, 1.94 mmol, 60% w/w dispersion in mineral oil) at 0° C. under nitrogen. The mixture was stirred at 0° C. for 10 min and then benzyl alcohol (152 mg, 1.41 mmol) was added. The solution was stirred at 0° C. for 1.0 h and diluted with ethyl acetate and water. The organic layer was washed with brine, dried over sodium sulfate and concentrated under reduced pressure. The crude product was purified by flash chromatography (petroleum ether/ethyl acetate=3/1) to give 5-(benzyloxy)-4-methyl-[3,3′-bipyridine]-6-carbonitrile (300 mg, 0.99 mmol, 76% yield) as yellow solid. LC-MS: m/z=302.1 [M+H]⁺, retention time=2.21 min (Method A).

### 5-(Benzyloxy)-4-methyl-[3,3′-bipyridine]-6-carboxylic acid

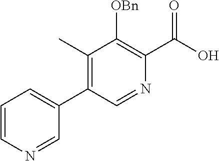 [SMILES: Cc1c(-c2cccnc2)cnc(C(=O)O)c1OCc1ccccc1]

To a solution of 5-(benzyloxy)-4-methyl-[3,3′-bipyridine]-6-carbonitrile (300 mg, 0.99 mmol) in ethanol (10.0 mL) was added 30% aqueous sodium hydroxide (4.0 mL). The mixture was stirred at 100° C. for 3.0 h, cooled and concentrated to remove ethanol. The resulting aqueous solution was acidified with 10% hydrochloric acid solution to pH=3˜4. The precipitate was filtered and dried to afford 5-(benzyloxy)-4-methyl-[3,3′-bipyridine]-6-carboxylic acid (300 mg, 0.94 mmol, 94% yield) as white solid. LC-MS: m/z=321.1 [M+H]⁺, retention time 2.00 min (Method A). The product was pure enough and used directly to the next step.

### Ethyl (5-(benzyloxy)-4-methyl-[3,3′-bipyridine]-6-carbonyl)glycinate

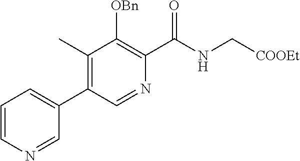 [SMILES: CCOC(=O)CNC(=O)c1ncc(-c2cccnc2)c(C)c1OCc1ccccc1]

A mixture of 5-(benzyloxy)-4-methyl-[3,3′-bipyridine]-6-carboxylic acid (300 mg, 0.94 mmol), ethyl glycinate hydrochloride (144 mg, 1.03 mmol), benzotriazole-1-yl-oxytripyrrolidinophosphonium hexafluorophosphate (536 mg, 1.03 mmol) and triethylamine (473 mg, 4.68 mmol) in dichloromethane (10.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/1) to obtain ethyl (5-(benzyloxy)-4-methyl-[3,3′-bipyridine]-6-carbonyl)glycinate (240 mg, 0.59 mmol, 63% yield) as white solid. LC-MS: m/z=406.1 [M+H]⁺, retention time 2.15 min (Method A).

### Ethyl (5-hydroxy-4-methyl-[3,3′-bipyridine]-6-carbonyl)glycinate

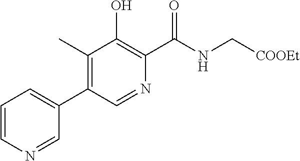 [SMILES: CCOC(=O)CNC(=O)c1ncc(-c2cccnc2)c(C)c1O]

A mixture of ethyl (5-(benzyloxy)-4-methyl-[3,3′-bipyridine]-6-carbonyl)glycinate (240 mg, 0.59 mmol) and 10% palladium on carbon (20.0 mg) in tetrahydrofuran (10.0 mL) was stirred at room temperature under hydrogen atmosphere for 18.0 h. The insoluble solid was filtered and the filtrate was concentrated to give ethyl (5-hydroxy-4-methyl-[3,3′-bipyridine]-6-carbonyl)glycinate (185 mg, 0.59 mmol, 99% yield) as white solid. LC-MS: m/z=316.1 [M+H]⁺, retention time 2.19 min (Method A). The product was pure enough and used directly to the next step.

### (5-Hydroxy-4-methyl-[3,3′-bipyridine]-6-carbonyl)glycine

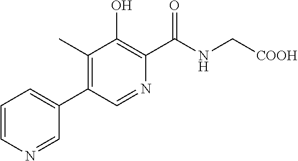 [SMILES: Cc1c(-c2cccnc2)cnc(C(=O)NCC(=O)O)c1O]

To a solution of ethyl (5-hydroxy-4-methyl-[3,3′-bipyridine]-6-carbonyl)glycinate (185 mg, 0.59 mmol) in tetrahydrofuran/water (8.0 mL/2.0 mL) was added lithium hydroxide monohydrate (164 mg, 4.0 mmol). The mixture was stirred overnight and concentrated to remove tetrahydrofuran. The resulting aqueous solution was acidified with 10% hydrochloric acid solution to pH=3˜4. The precipitate was filtered, washed with water and purified by reverse prep-HPLC to obtain (5-hydroxy-4-methyl-[3,3′-bipyridine]-6-carbonyl)glycine (Formate) (34.0 mg, 0.118 mmol, 20% yield) as white solid. LC-MS: m/z=288.1 [M+H]⁺, retention time 2.20 min (Method A). ¹HNMR (400 MHz, DMSO-d₆) δ 12.83 (s, 2H), 9.41 (t, J=6.1 Hz, 1H), 8.84-8.50 (m, 2H), 8.13 (s, 1H), 8.10 (s, 1H), 8.04-7.81 (m, 1H), 7.73-7.41 (m, 1H), 4.01 (d, J=6.1 Hz, 2H), 2.17 (s, 3H).

### Example 11: Preparation of Compound 11

### 5-Chloro-4-methyl-[3,4′-bipyridine]-6-carbonitrile

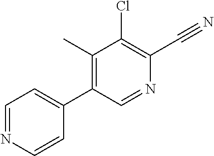 [SMILES: Cc1c(-c2ccncc2)cnc(C#N)c1Cl]

To a solution of 3,5-dichloro-4-methyl-pyridine-2-carbonitrile (500.00 mg, 2.67 mmol), pyridin-4-ylboronic acid (328.63 mg, 2.67 mmol) and potassium carbonate (443.41 mg, 3.21 mmol) was added [1,1′-bis(diphenylphosphino)ferrocene]dichloropalladium(II) (78.25 mg, 0.11 mmol) in N,N-dimethylformamide/water (10.0 mL/1.0 mL). The mixture was stirred at 50° C. for 16.0 h under nitrogen and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=1/1) to provide 5-chloro-4-methyl-[3,4′-bipyridine]-6-carbonitrile (292 mg, 1.27 mmol, 48% yield). LC-MS: m/z=230 [M+H]⁺, retention time 1.599 min (Method A).

### 5-(Benzyloxy)-4-methyl-[3,4′-bipyridine]-6-carbonitrile

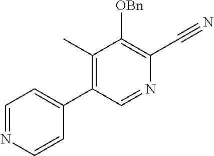 [SMILES: Cc1c(-c2ccncc2)cnc(C#N)c1OCc1ccccc1]

To a solution of 5-chloro-4-methyl-[3,4′-bipyridine]-6-carbonitrile (250.00 mg, 1.09 mmol) in N,N-dimethylformamide (10.0 mL) was sodium hydride (52.3 mg, 1.31 mmol, 60% w/w dispersion in mineral oil) at 0° C. under nitrogen. The mixture was stirred at 0° C. for 10 min and then benzyl alcohol (141.25 mg, 1.31 mmol, 0.14 mL) was added. The solution was stirred at 0° C. for 50 min and diluted with ethyl acetate and water. The organic layer was washed with brine, dried over sodium sulfate and concentrated under reduced pressure. The crude product was purified by flash chromatography (petroleum ether/ethyl acetate=1/1) to provide 5-(benzyloxy)-4-methyl-[3,4′-bipyridine]-6-carbonitrile (86 mg, 0.29 mmol, 26% yield). LC-MS: m/z=302 [M+H]⁺, retention time 1.895 min (Method B).

### 5-Hydroxy-4-methyl-[3,4′-bipyridine]-6-carboxylic acid

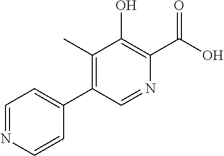 [SMILES: Cc1c(-c2ccncc2)cnc(C(=O)O)c1O]

To a solution of 35-(benzyloxy)-4-methyl-[3,4′-bipyridine]-6-carbonitrile (86.00 mg, 0.29 mmol) in ethanol (5.0 mL) was added 30% aqueous sodium hydroxide (4.0 mL). The mixture was stirred at 100° C. for 3.0 h, cooled and concentrated to remove ethanol. The resulting aqueous solution was acidified with 10% hydrochloric acid solution to pH=3˜4. The precipitate was filtered and dried to afford 5-hydroxy-4-methyl-[3,4′-bipyridine]-6-carboxylic acid (160 mg, crude) as white solid. LC-MS: m/z=231 [M+H]⁺, retention time 1.020 min (Method A). The product was pure enough and used directly to the next step.

### Ethyl (5-hydroxy-4-methyl-[3,4′-bipyridine]-6-carbonyl)glycinate

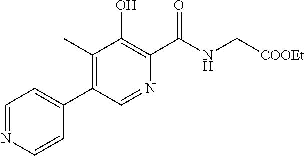 [SMILES: CCOC(=O)CNC(=O)c1ncc(-c2ccncc2)c(C)c1O]

A mixture of 5-hydroxy-4-methyl-[3,4′-bipyridine]-6-carboxylic acid (130.00 mg, 0.56 mmol), ethyl glycinate hydrochloride (78.82 mg, 0.56 mmol), benzotriazole-1-yl-oxytripyrrolidinophosphonium hexafluorophosphate (323.24 mg, 0.62 mmol and triethylamine (285.70 mg, 2.82 mmol, 0.4 mL in dichloromethane (10.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=3/2) to provide ethyl (5-hydroxy-4-methyl-[3,4′-bipyridine]-6-carbonyl)glycinate (36 mg, 0.114 mmol, 20% yield). LC-MS: m/z=316 [M+H]⁺, retention time 1.586 min (Method A).

### (5-Hydroxy-4-methyl-[3,4′-bipyridine]-6-carbonyl)glycine

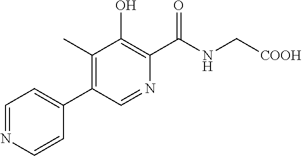 [SMILES: Cc1c(-c2ccncc2)cnc(C(=O)NCC(=O)O)c1O]

To a solution of ethyl (5-hydroxy-4-methyl-[3,4′-bipyridine]-6-carbonyl)glycinate (30.00 mg, 0.10 mmol) in methanl/water (4.0 mL/1.0 mL) was added lithium hydroxide monohydrate (22.79 mg, 0.95 mmol). The mixture was stirred at 40° C. for 16.0 h and concentrated to remove methanol. The resulting aqueous solution was acidified with 10% hydrochloric acid solution to pH=3˜4. The precipitate was filtered, washed with water and dried to afford (5-hydroxy-4-methyl-[3,4′-bipyridine]-6-carbonyl)glycine (13.2 mg, 46% yield) as white solid. LC-MS: m/z=288 [M+H]⁺, retention time 2.159 min (Method A). ¹H NMR (500 MHz, DMSO-d₆) δ 12.85 (br, 2H), 9.42 (s, 1H), 8.72 (dd, J=4.5 Hz, J=1.5 Hz, 2H), 8.08 (s, 1H), 7.51 (dd, J=4.0 Hz, J=1.5 Hz, 2H), 4.01 (d, J=6.0 Hz, 2H), 2.17 (s, 3H).

### Example 12: Preparation of Compound 12

### 5′-Chloro-4′-methyl-[2,3′-bipyridine]-6′-carbonitrile

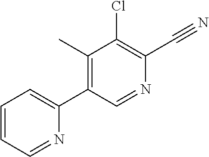 [SMILES: Cc1c(-c2ccccn2)cnc(C#N)c1Cl]

To a solution of 3,5-dibromo-4,6-dimethylpicolinonitrile (1.86 g, 10 mmol), 2-(tributylstannyl)pyridine (4.42 g, 12 mmol), caesium fluoride (302 mg, 2.0 mmol) and copper iodide (380 mg, 2.0 mmol) in N,N-dimethylformamide (15.0 mL) was added tetrakis(triphenylphosphine)palladium (116 mg, 0.1 mmol). The mixture was stirred at 50° C. under nitrogen for 16.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=10/1) to afford 5′-chloro-4′-methyl-[2,3′-bipyridine]-6′-carbonitrile (460 mg, 2.0 mmol, 20% yield) as yellow solid. LC-MS: m/z=230.0 [M+H]⁺, retention time=1.704 min (Method A).

### 5′-(Benzyloxy)-4′-methyl-[2,3′-bipyridine]-6′-carbonitrile

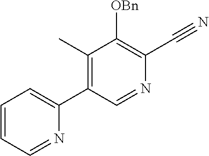 [SMILES: Cc1c(-c2ccccn2)cnc(C#N)c1OCc1ccccc1]

To a solution of 5′-chloro-4′-methyl-[2,3′-bipyridine]-6′-carbonitrile (160.0 mg, 0.70 mmol) in N,N-dimethylformamide (5.0 mL) was sodium hydride (33 mg, 0.84 mmol, 60% w/w dispersion in mineral oil) at 0° C. under nitrogen. The mixture was stirred at 0° C. for 10 min and then benzyl alcohol (90 mg, 0.84 mmol) was added. The solution was stirred at 0° C. for 1 h and diluted with ethyl acetate and water. The organic layer was washed with brine, dried over sodium sulfate and concentrated under reduced pressure. The crude product was purified by flash chromatography (petroleum ether/ethyl acetate=1/2) to give 5′-(benzyloxy)-4′-methyl-[2,3′-bipyridine]-6′-carbonitrile (120 mg, 0.40 mmol, 57% yield) as yellow solid. LC-MS: m/z=302.1 [M+H]⁺, retention time=2.21 min (Method A).

### 5′-Hydroxy-4′-methyl-[2,3′-bipyridine]-6′-carboxylic Acid

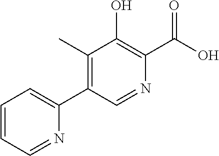 [SMILES: Cc1c(-c2ccccn2)cnc(C(=O)O)c1O]

To a solution of 5′-(benzyloxy)-4′-methyl-[2,3′-bipyridine]-6′-carbonitrile (120 mg, 0.40 mmol) in ethanol (10.0 mL) was added 30% aqueous sodium hydroxide (4.0 mL). The mixture was stirred at 100° C. for 5.0 h, cooled and concentrated to remove ethanol. The resulting aqueous solution was acidified with 10% hydrochloric acid solution to pH=3˜4. The precipitate was filtered and dried to afford 5′-hydroxy-4′-methyl-[2,3′-bipyridine]-6′-carboxylic acid (140 mg, crude) as white solid. LC-MS: m/z=321.1 [M+H]⁺, retention time 2.00 min (Method A). The product was pure enough and used directly to the next step.

### Ethyl (5′-hydroxy-4′-methyl-[2,3′-bipyridine]-6′-carbonyl)glycinate

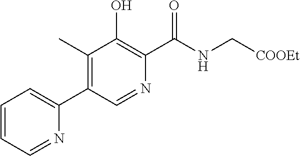 [SMILES: CCOC(=O)CNC(=O)c1ncc(-c2ccccn2)c(C)c1O]

A mixture of 5′-hydroxy-4′-methyl-[2,3′-bipyridine]-6′-carboxylic acid (140 mg, 0.44 mmol), ethyl glycinate hydrochloride (61 mg, 0.44 mmol), benzotriazole-1-yl-oxytripyrrolidinophosphonium hexafluorophosphate (250 mg, 0.48 mmol), triethylamine (254 mg, 2.52 mmol) in dichloromethane (5.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/2) to obtain ethyl (5′-hydroxy-4′-methyl-[2,3′-bipyridine]-6′-carbonyl)glycinate (50 mg, 0.16 mmol, 26% yield) as white solid. LC-MS: m/z=406.1 [M+H]⁺, retention time 2.15 min (Method A).

### (5′-Hydroxy-4′-methyl-[2,3′-bipyridine]-6′-carbonyl)glycine

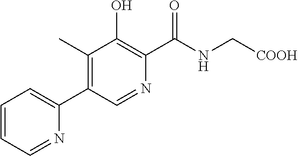 [SMILES: Cc1c(-c2ccccn2)cnc(C(=O)NCC(=O)O)c1O]

To a solution of ethyl (5′-hydroxy-4′-methyl-[2,3′-bipyridine]-6′-carbonyl)glycinate (50 mg, 0.16 mmol) in tetrahydrofuran/water (8.0 mL/2.0 mL) was added lithium hydroxide monohydrate (164 mg, 4.0 mmol). The mixture was stirred overnight and concentrated to remove tetrahydrofuran. The resulting aqueous solution was acidified with 10% hydrochloric acid solution to pH=3˜4. The precipitate was filtered, washed with water and purified by reverse Prep-HPLC to obtain (5′-hydroxy-4′-methyl-[2,3′-bipyridine]-6′-carbonyl)glycine (9.6 mg, 21% yield) as white solid. LC-MS: m/z=288.0 [M+H]⁺, retention time 2.54 min (Method A). ¹HNMR (400 MHz, DMSO-d₆) δ 12.82 (s, 2H), 9.42 (t, J=6.0 Hz, 1H), 8.76 (d, J=4.3 Hz, 1H), 8.21 (s, 1H), 7.99 (td, J=7.8, 1.7 Hz, 1H), 7.68 (d, J=7.8 Hz, 1H), 7.50 (dd, J=6.8, 4.9 Hz, 1H), 4.02 (d, J=6.2 Hz, 2H), 2.24 (s, 3H).

### Example 13: Preparation of Compound 13

### 3-Chloro-5-(3-chlorophenyl)-4-methylpicolinonitrile

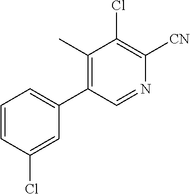 [SMILES: Cc1c(-c2cccc(Cl)c2)cnc(C#N)c1Cl]

To a solution of 3,5-dichloro-4-methylpicolinonitrile (500 mg, 2.67 mmol), (3-chlorophenyl)boronic acid (418 mg 2.67 mmol) and potassium carbonate (443 mg, 3.21 mmol) was added [1,1′-bis(diphenylphosphino)ferrocene]dichloropalladium(II) (78 mg, 0.11 mmol) in N,N-dimethylformamide/water (5.0 mL/0.5 mL). The mixture was stirred at 45° C. under nitrogen for 16.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=2/1) to afford 3-chloro-5-(3-chlorophenyl)-4-methylpicolinonitrile (640 mg, 2.43 mmol, 91% yield) as a yellow solid. LC-MS: m/z=264.1 [M+H]⁺, retention time=1.83 min (Method A).

### 3-(Benzyloxy)-5-(3-chlorophenyl)-4-methylpicolinonitrile

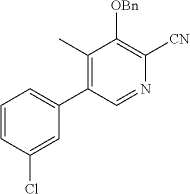 [SMILES: Cc1c(-c2cccc(Cl)c2)cnc(C#N)c1OCc1ccccc1]

To a solution of 3-chloro-5-(3-chlorophenyl)-4-methylpicolinonitrile (3.2 g, 12.16 mmol) in N,N-dimethylformamide (40.0 mL) was sodium hydride (584 mg, 14.60 mmol, 60% w/w dispersion in mineral oil) at 0° C. under nitrogen. The mixture was stirred at 0° C. for 10 min and then benzyl alcohol (1.58 g, 14.60 mmol) was added. The solution was stirred at 0° C. for 1.0 h and diluted with ethyl acetate and water. The organic layer was washed with brine, dried over sodium sulfate and concentrated under reduced pressure. The crude product was purified by flash chromatography (petroleum ether/ethyl acetate=3/1) to give 3-(benzyloxy)-5-(3-chlorophenyl)-4-methylpicolinonitrile (3.2 g, 9.56 mmol, 80% yield) as yellow solid. LC-MS: m/z=335.1 [M+H]⁺, retention time=2.21 min (Method A).

### 3-(Benzyloxy)-5-(3-chlorophenyl)-4-methylpicolinic acid

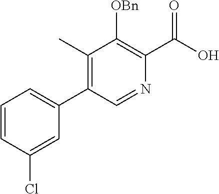 [SMILES: Cc1c(-c2cccc(Cl)c2)cnc(C(=O)O)c1OCc1ccccc1]

To a solution of 3-(benzyloxy)-5-(3-chlorophenyl)-4-methylpicolinonitrile (3.2 g, 9.56 mmol) in ethanol (60.0 mL) was added 30% aqueous sodium hydroxide (20.0 mL). The mixture was stirred at 100° C. for 5.0 h, cooled and concentrated to remove ethanol. The resulting aqueous solution was acidified with 10% hydrochloric acid solution to pH=3˜4. The precipitate was filtered and dried to afford 3-(benzyloxy)-5-(3-chlorophenyl)-4-methylpicolinic acid (2.1 g, 5.96 mmol, 64% yield) as white solid. LC-MS: m/z=354.1 [M+H]⁺, retention time 2.00 min (Method A). The product was pure enough and used directly to the next step.

### Methyl 2-(3-(benzyloxy)-5-(3-chlorophenyl)-4-methylpicolinamido)-2-methylpropanoate

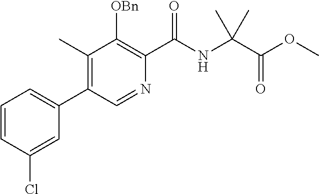 [SMILES: COC(=O)C(C)(C)NC(=O)c1ncc(-c2cccc(Cl)c2)c(C)c1OCc1ccccc1]

A mixture of 3-(benzyloxy)-5-(3-chlorophenyl)-4-methylpicolinic acid (100 mg, 0.28 mmol), methyl 2-amino-2-methylpropanoate hydrochloride (44 mg, 0.28 mmol), benzotriazole-1-yl-oxytripyrrolidinophosphonium hexafluorophosphate (162 mg, 0.31 mmol) and triethylamine (143 mg, 1.41 mmol) in dichloromethane (5.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/1) to obtain methyl 2-(3-(benzyloxy)-5-(3-chlorophenyl)-4-methylpicolinamido)-2-methylpropanoate (110 mg, 0.24 mmol, 87% yield) as white solid. LC-MS: m/z=453.1 [M+H]⁺, retention time 2.15 min (Method A).

### Methyl 2-(5-(3-chlorophenyl)-3-hydroxy-4-methylpicolinamido)-2-methylpropanoate

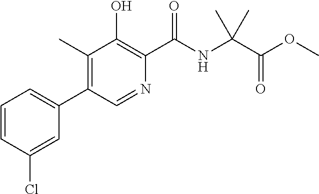 [SMILES: COC(=O)C(C)(C)NC(=O)c1ncc(-c2cccc(Cl)c2)c(C)c1O]

A mixture of methyl 2-(3-(benzyloxy)-5-(3-chlorophenyl)-4-methylpicolinamido)-2-methylpropanoate (500 mg, 1.10 mmol) and 10% palladium on carbon (50.0 mg) in tetrahydrofuran (10.0 mL) was stirred under hydrogen atmosphere for 18.0 h. The insoluble solid was filtered and the filtrate was concentrated to give methyl 2-(5-(3-chlorophenyl)-3-hydroxy-4-methylpicolinamido)-2-methylpropanoate (240 mg, 0.66 mmol, 60% yield) as white solid. LC-MS: m/z=363.1 [M+H]⁺, retention time 2.19 min (Method A). The product was pure enough and used directly to the next step.

### 2-(5-(3-Chlorophenyl)-3-hydroxy-4-methylpicolinamido)-2-methylpropanoic Acid

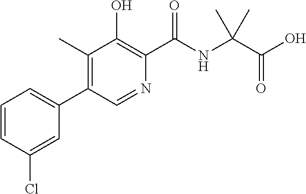 [SMILES: Cc1c(-c2cccc(Cl)c2)cnc(C(=O)NC(C)(C)C(=O)O)c1O]

To a solution of methyl 2-(5-(3-chlorophenyl)-3-hydroxy-4-methylpicolinamido)-2-methylpropanoate (240 mg, 0.66 mmol) in tetrahydrofuran/water (10.0 mL/2.0 mL) was added lithium hydroxide monohydrate (164 mg, 4.0 mmol). The mixture was stirred overnight and concentrated to remove tetrahydrofuran. The resulting aqueous solution was acidified with 10% hydrochloric acid solution to pH=3˜4. The precipitate was filtered, washed with water and dried to obtain 2-(5-(3-chlorophenyl)-3-hydroxy-4-methylpicolinamido)-2-methylpropanoic acid (176.3 mg, 0.51 mmol, 77% yield) as white solid. LC-MS: m/z=349.0 [M+H]⁺, retention time 5.36 min (Method A). ¹HNMR (400 MHz, DMSO-d₆) δ 12.93 (br s, 1H), 12.70 (s, 1H), 9.07 (s, 1H), 8.04 (s, 1H), 7.67-7.45 (m, 3H), 7.47-7.18 (m, 1H), 2.14 (s, 3H), 1.59 (s, 6H).

### Example 14: Preparation of Compound 14

### Ethyl 1-(3-(benzyloxy)-5-(3-chlorophenyl)-4-methylpicolinamido)cyclopropane-1-carboxylate

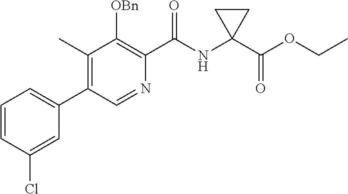 [SMILES: CCOC(=O)C1(NC(=O)c2ncc(-c3cccc(Cl)c3)c(C)c2OCc2ccccc2)CC1]

A mixture of 3-(benzyloxy)-5-(3-chlorophenyl)-4-methylpicolinic acid (Intermediate from Example 13) (100 mg, 0.28 mmol), ethyl 1-aminocyclopropane-1-carboxylate hydrochloride (43 mg, 0.28 mmol), benzotriazole-1-yl-oxytripyrrolidinophosphonium hexafluorophosphate (162 mg, 0.31 mmol) and triethylamine (143 mg, 1.41 mmol) in dichloromethane (5.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/1) to obtain ethyl 1-(3-(benzyloxy)-5-(3-chlorophenyl)-4-methylpicolinamido)cyclopropane-1-carboxylate (110 mg, 0.24 mmol, 85% yield) as white solid. LC-MS: m/z=465.1 [M+H]⁺, retention time 2.15 min (Method A).

### Ethyl 1-(5-(3-chlorophenyl)-3-hydroxy-4-methylpicolinamido)cyclopropane-1-carboxylate

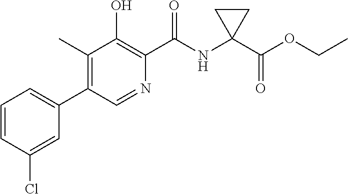 [SMILES: CCOC(=O)C1(NC(=O)c2ncc(-c3cccc(Cl)c3)c(C)c2O)CC1]

A mixture of ethyl 1-(3-(benzyloxy)-5-(3-chlorophenyl)-4-methylpicolinamido)cyclopropane-1-carboxylate (500 mg, 1.08 mmol) and 10% palladium on carbon (50.0 mg) in tetrahydrofuran (10.0 mL) was stirred under hydrogen atmosphere for 18.0 h. The insoluble solid was filtered and the filtrate was concentrated to give ethyl 1-(5-(3-chlorophenyl)-3-hydroxy-4-methylpicolinamido)cyclopropane-1-carboxylate (250 mg, 0.67 mmol, 62% yield) as white solid. LC-MS: m/z=375.1 [M+H]⁺, retention time 2.19 min (Method A). The product was pure enough and used directly to the next step.

### 1-(5-(3-Chlorophenyl)-3-hydroxy-4-methylpicolinamido)cyclopropane-1-carboxylic acid

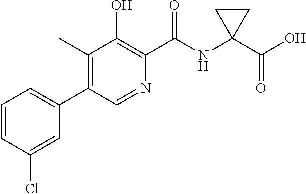 [SMILES: Cc1c(-c2cccc(Cl)c2)cnc(C(=O)NC2(C(=O)O)CC2)c1O]

To a solution of ethyl 1-(5-(3-chlorophenyl)-3-hydroxy-4-methylpicolinamido)cyclopropane-1-carboxylate (250 mg, 0.67 mmol) in tetrahydrofuran/water (10.0 mL/2.0 mL) was added lithium hydroxide monohydrate (164 mg, 4.0 mmol). The mixture was stirred overnight and concentrated to remove tetrahydrofuran. The resulting aqueous solution was acidified with 10% hydrochloric acid solution to pH=3˜4. The precipitate was filtered, washed with water and dried to obtain 1-(5-(3-chlorophenyl)-3-hydroxy-4-methylpicolinamido)cyclopropane-1-carboxylic acid (133.0 mg, 0.38 mmol, 58% yield) as white solid. LC-MS: m/z=347.0 [M+H]⁺, retention time 4.93 min (Method A). ¹HNMR (400 MHz, DMSO-d₆) δ 12.93 (s, 1H), 12.57 (s, 1H), 9.71 (s, 1H), 8.02 (s, 1H), 7.67-7.46 (m, 3H), 7.45-7.36 (m, 1H), 2.14 (s, 3H), 1.44 (dd, J=7.8, 4.6 Hz, 2H), 1.25 (dd, J=7.9, 4.6 Hz, 2H).

### Example 15: Preparation of Compound 15

### Methyl 1-(3-(benzyloxy)-5-(3-chlorophenyl)-4-methylpicolinamido)cyclobutane-1-carboxylate

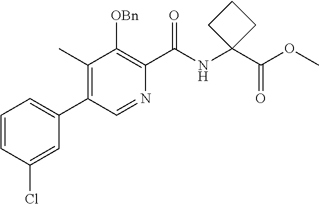 [SMILES: COC(=O)C1(NC(=O)c2ncc(-c3cccc(Cl)c3)c(C)c2OCc2ccccc2)CCC1]

A mixture of 3-(benzyloxy)-5-(3-chlorophenyl)-4-methylpicolinic acid (Intermediate from Example 13) (400 mg, 1.13 mmol), methyl 1-aminocyclobutane-1-carboxylate hydrochloride (187 mg, 1.13 mmol), benzotriazole-1-yl-oxytripyrrolidinophosphonium hexafluorophosphate (647 mg, 1.24 mmol) and triethylamine (571 mg, 5.65 mmol) in dichloromethane (15.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/1) to obtain methyl 1-(3-(benzyloxy)-5-(3-chlorophenyl)-4-methylpicolinamido)cyclobutane-1-carboxylate (500 mg, 1.08 mmol, 95% yield) as white solid. LC-MS: m/z=465.1 [M+H]⁺, retention time 2.15 min (Method A).

### Methyl 1-(5-(3-chlorophenyl)-3-hydroxy-4-methylpicolinamido)cyclobutane-1-carboxylate

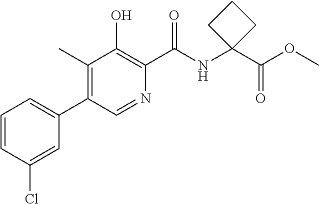 [SMILES: COC(=O)C1(NC(=O)c2ncc(-c3cccc(Cl)c3)c(C)c2O)CCC1]

A mixture of methyl 1-(3-(benzyloxy)-5-(3-chlorophenyl)-4-methylpicolinamido)cyclobutane-1-carboxylate (500 mg, 1.08 mmol) and 10% palladium on carbon (50.0 mg) in tetrahydrofuran (10.0 mL) was stirred under hydrogen atmosphere for 18.0 h. The insoluble solid was filtered and the filtrate was concentrated to give methyl 1-(5-(3-chlorophenyl)-3-hydroxy-4-methylpicolinamido)cyclobutane-1-carboxylate (250 mg, 0.67 mmol, 62% yield) as white solid. LC-MS: m/z=375.1 [M+H]⁺, retention time 2.19 min (Method A). The product was pure enough and used directly to the next step.

### 1-(5-(3-Chlorophenyl)-3-hydroxy-4-methylpicolinamido)cyclobutane-1-carboxylic acid

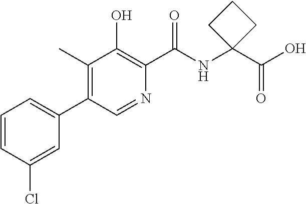 [SMILES: Cc1c(-c2cccc(Cl)c2)cnc(C(=O)NC2(C(=O)O)CCC2)c1O]

To a solution of methyl 1-(5-(3-chlorophenyl)-3-hydroxy-4-methylpicolinamido)cyclobutane-1-carboxylate (250 mg, 0.67 mmol) in tetrahydrofuran/water (10.0 mL/2.0 mL) was added lithium hydroxide monohydrate (164 mg, 4.0 mmol). The mixture was stirred overnight and concentrated to remove tetrahydrofuran. The resulting aqueous solution was acidified with 10% hydrochloric acid solution to pH=3˜4. The precipitate was filtered, washed with water and dried to obtain 1-(5-(3-chlorophenyl)-3-hydroxy-4-methylpicolinamido)cyclobutane-1-carboxylic acid (93.0 mg, 0.26 mmol, 39% yield) as white solid. LC-MS: m/z=361.0 [M+H]⁺, retention time 5.37 min (Method A). ¹HNMR (400 MHz, DMSO-d₆) δδ 12.80 (s, 1H), 12.69 (br s, 1H), 9.63 (s, 1H), 8.05 (s, 1H), 7.75-7.44 (m, 3H), 7.49-7.26 (m, 1H), 2.76-2.38 (m, 6H), 2.14 (s, 3H), 2.03-1.85 (m, 2H).

### Example 16: Preparation of Compound 16

### Methyl 3-(3-(benzyloxy)-5-(3-chlorophenyl)-4-methylpicolinamido)oxetane-3-carboxylate

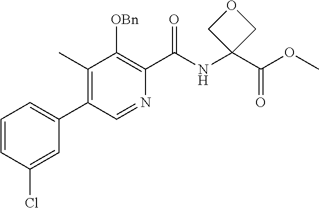 [SMILES: COC(=O)C1(NC(=O)c2ncc(-c3cccc(Cl)c3)c(C)c2OCc2ccccc2)COC1]

A mixture of 3-(benzyloxy)-5-(3-chlorophenyl)-4-methylpicolinic acid (Intermediate from Example 13) (450 mg, 1.27 mmol), methyl 3-aminooxetane-3-carboxylate hydrochloride (213 mg, 1.27 mmol), benzotriazole-1-yl-oxytripyrrolidinophosphonium hexafluorophosphate (728 mg, 1.40 mmol), triethylamine (571 mg, 5.65 mmol) in dichloromethane (15.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/1) to obtain methyl 3-(3-(benzyloxy)-5-(3-chlorophenyl)-4-methylpicolinamido)oxetane-3-carboxylate (500 mg, 1.07 mmol, 84% yield) as white solid. LC-MS: m/z=467.1 [M+H]⁺, retention time 2.15 min (Method A).

### Methyl 3-(5-(3-chlorophenyl)-3-hydroxy-4-methylpicolinamido)oxetane-3-carboxylate

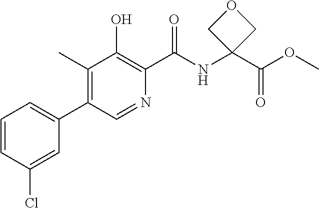 [SMILES: COC(=O)C1(NC(=O)c2ncc(-c3cccc(Cl)c3)c(C)c2O)COC1]

A mixture of methyl 3-(3-(benzyloxy)-5-(3-chlorophenyl)-4-methylpicolinamido)oxetane-3-carboxylate (500 mg, 1.07 mmol) and 10% palladium on carbon (50.0 mg) in tetrahydrofuran (10.0 mL) was stirred under hydrogen atmosphere for 18 h. The insoluble solid was filtered and the filtrate was concentrated to give methyl 3-(5-(3-chlorophenyl)-3-hydroxy-4-methylpicolinamido)oxetane-3-carboxylate (300 mg, 0.80 mmol, 74% yield) as white solid. LC-MS: m/z=377.1 [M+H]⁺, retention time 2.19 min (Method A). The product was pure enough and used directly to the next step.

### 3-(5-(3-Chlorophenyl)-3-hydroxy-4-methylpicolinamido)oxetane-3-carboxylic Acid

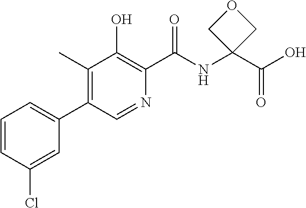 [SMILES: Cc1c(-c2cccc(Cl)c2)cnc(C(=O)NC2(C(=O)O)COC2)c1O]

To a solution of methyl 3-(5-(3-chlorophenyl)-3-hydroxy-4-methylpicolinamido)oxetane-3-carboxylate (300 mg, 0.80 mmol) in tetrahydrofuran/water (10.0 mL/2.0 mL) was added lithium hydroxide monohydrate (164 mg, 4.0 mmol). The mixture was stirred overnight and concentrated to remove tetrahydrofuran. The resulting aqueous solution was acidified with 10% hydrochloric acid solution to pH=3˜4. The precipitate was filtered, washed with water and dried to obtain 3-(5-(3-chlorophenyl)-3-hydroxy-4-methylpicolinamido)oxetane-3-carboxylic acid (71.4 mg, 0.20 mmol, 25% yield) as white solid. LC-MS: m/z=363.0 [M+H]⁺, retention time 4.85 min (Method A). ¹HNMR (400 MHz, DMSO-d₆) δ 12.53 (s, 1H), 10.18 (s, 1H), 8.08 (s, 1H), 7.58-7.53 (m, 3H), 7.45-7.37 (m, 1H), 4.94-4.80 (m, 4H), 2.14 (s, 3H).

### Example 17: Preparation of Compound 17

### 3-(Benzyloxy)-4-methyl-5-(2-methylquinolin-6-yl)picolinonitrile

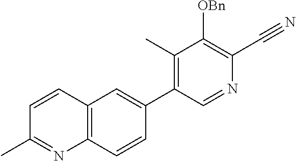 [SMILES: Cc1ccc2cc(-c3cnc(C#N)c(OCc4ccccc4)c3C)ccc2n1]

To a solution of 3-chloro-4-methyl-5-(3-phenoxyphenyl)picolinonitrile (Intermediate from Example 8) (700 mg, 2.39 mmol) in N,N-dimethylformamide (10.0 mL) was sodium hydride (114.80 mg, 2.87 mmol, 60% w/w dispersion in mineral oil) at 0° C. under nitrogen. The mixture was stirred at 0° C. for 10 min and then benzyl alcohol (309.6 mg, 2.87 mmol) was added. The solution was stirred at 0° C. for 1.0 h and diluted with ethyl acetate and water. The organic layer was washed with brine, dried over sodium sulfate and concentrated under reduced pressure. The crude product was purified by flash chromatography (petroleum ether/ethyl acetate=4/1) to give 3-(benzyloxy)-4-methyl-5-(2-methylquinolin-6-yl)picolinonitrile (400 mg, 1.09 mmol, 45.87% yield). LC-MS: m/z=366.0 [M+H]⁺, retention time 1.72 min (Method A).

### 3-(Benzyloxy)-4-methyl-5-(2-methylquinolin-6-yl)picolinic Acid

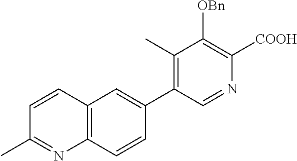 [SMILES: Cc1ccc2cc(-c3cnc(C(=O)O)c(OCc4ccccc4)c3C)ccc2n1]

To a solution of 3-(benzyloxy)-4-methyl-5-(2-methylquinolin-6-yl)picolinonitrile (400 mg, 1.09 mmol) in ethanol (10.0 mL) was added 30% aqueous sodium hydroxide (8.0 mL). The mixture was stirred at 100° C. for 3.0 h, cooled and concentrated to remove ethanol. The resulting aqueous solution was acidified with 10% hydrochloric acid solution to pH=3˜4. The precipitate was filtered and dried to afford 3-(benzyloxy)-4-methyl-5-(2-methylquinolin-6-yl)picolinic acid (350 mg, 0.91 mmol, 75.32% yield) as white solid. LC-MS: m/z=385.0 [M+H]⁺, retention time 1.51 min (Method A). The product was pure enough and used directly to the next step.

### Ethyl (3-(benzyloxy)-4-methyl-5-(2-methylquinolin-6-yl)picolinoyl)glycinate

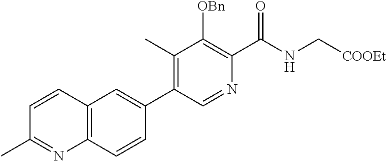 [SMILES: CCOC(=O)CNC(=O)c1ncc(-c2ccc3nc(C)ccc3c2)c(C)c1OCc1ccccc1]

A mixture of 3-(benzyloxy)-4-methyl-5-(2-methylquinolin-6-yl)picolinic acid (350 mg, 0.91 mmol), ethyl glycinate hydrochloride (73.84 mg, 0.53 mmol), benzotriazole-1-yl-oxytripyrrolidinophosphonium hexafluorophosphate (276.25 mg, 0.53 mmol) and triethylamine (223.57 mg, 2.21 mmol) in dichloromethane (5.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/2) to obtain ethyl (3-(benzyloxy)-4-methyl-5-(2-methylquinolin-6-yl)picolinoyl)glycinate (150 mg, 0.32 mmol, 72.69% yield) as white solid. LC-MS: m/z=470.0 [M+H]⁺, retention time 2.04 min (Method B).

### Ethyl (3-hydroxy-4-methyl-5-(2-methyl-1,2,3,4-tetrahydroquinolin-6-yl)picolinoyl)glycinate

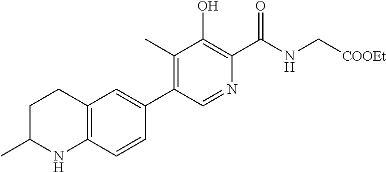 [SMILES: CCOC(=O)CNC(=O)c1ncc(-c2ccc3c(c2)CCC(C)N3)c(C)c1O]

A mixture of ethyl (3-(benzyloxy)-4-methyl-5-(2-methylquinolin-6-yl)picolinoyl)glycinate (150 mg, 0.32 mmol) and 10% palladium on carbon (20.0 mg) in tetrahydrofuran (10.0 mL) was stirred at room temperature under hydrogen atmosphere overnight. The insoluble solid was filtered and the filtrate was concentrated to get ethyl (3-hydroxy-4-methyl-5-(2-methyl-1,2,3,4-tetrahydroquinolin-6-yl)picolinoyl)glycinate (120 mg, 0.31 mmol, 97.91% yield) as a yellow solid. LC-MS: m/z=384.3 [M+H]⁺, retention time 2.27 min (Method B). The product was pure enough and used directly to the next step.

### (3-Hydroxy-4-methyl-5-(2-methyl-1,2,3,4-tetrahydroquinolin-6-yl)picolinoyl)glycine

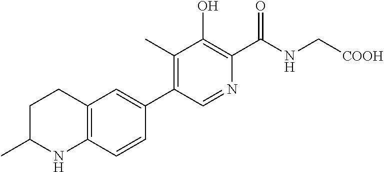 [SMILES: Cc1c(-c2ccc3c(c2)CCC(C)N3)cnc(C(=O)NCC(=O)O)c1O]

To a solution of ethyl (3-hydroxy-4-methyl-5-(2-methyl-1,2,3,4-tetrahydroquinolin-6-yl)picolinoyl)glycinate (100 mg, 0.26 mmol) in tetrahydrofuran/water (8.0 mL/2.0 mL) was added lithium hydroxide monohydrate (164 mg, 4.0 mmol). The mixture was stirred overnight and concentrated to remove tetrahydrofuran. The resulting aqueous solution was acidified with 10% hydrochloric acid solution to pH=3˜4. The precipitate was filtered, washed with water and purified by reverse perp-HPLC to obtain (3-hydroxy-4-methyl-5-(2-methyl-1,2,3,4-tetrahydroquinolin-6-yl)picolinoyl)glycine (Formate) (17.9 mg, 0.05 mmol, 19.39%) as white solid. LC-MS: m/z=356.1 [M+H]⁺, retention time 3.91 min (Method A). ¹HNMR (400 MHz, DMSO-d₆) δ 12.73 (s, 1H), 9.17 (s, 1H), 8.17 (s, 1H), 7.97 (s, 1H), 6.94 (d, J=6.3 Hz, 2H), 6.56 (d, J=8.7 Hz, 1H), 5.93 (br s, 1H), 3.95 (d, J=5.5 Hz, 2H), 2.82-2.60 (m, 3H), 2.58-2.53 (m, 1H), 2.18 (s, 3H), 1.93-1.81 (m, 1H), 1.56-1.37 (m, 1H), 1.17 (d, J=6.1 Hz, 3H).

### Example 18: Preparation of Compound 18

### 2-(4-Bromo-1H-pyrazol-1-yl)pyridine

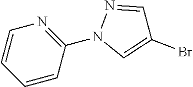 [SMILES: Brc1cnn(-c2ccccn2)c1]

To a solution of 2-(1H-pyrazol-1-yl)pyridine (1.0 g, 6.89 mmol) in acetic acid (20 mL) was added bromine (3302.54 mg, 20.67 mmol, 1.06 mL) dropwise. The mixture was stirred for 30 min at room temperature. The reaction was diluted with water and extracted twice with ethyl acetate. The organic layer was separated, washed with brine, dried over sodium sulfate and concentrated. The crude 2-(4-bromo-1H-pyrazol-1-yl)pyridine (1.35 g, crude) was obtained as yellow solid. LC-MS: m/z=224 [M+H]⁺, retention time 1.941 min (Method B). The product was used directly to the next step.

### 2-(4-(4,4,5,5-Tetramethyl-1,3,2-dioxaborolan-2-yl)-1H-pyrazol-1-yl)pyridine

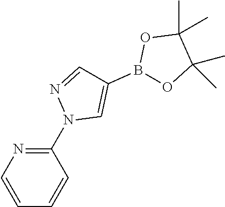 [SMILES: CC1(C)OB(c2cnn(-c3ccccn3)c2)OC1(C)C]

To a solution of 2-(4-bromo-1H-pyrazol-1-yl)pyridine (1.2 g, 5.36 mmol), bis(pinacolato)diboron (6.8 g, 26.78 mmol) and potassium acetate (2.63 g, 26.78 mmol) was added [1,1′-bis(diphenylphosphino)ferrocene]dichloropalladium(II) (391.88 mg, 0.54 mmol) in 1,4-dioxane (15.0 mL). The mixture was stirred at 90° C. under nitrogen for 16.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=10/1) to provide 2-(4-(4,4,5,5-tetramethyl-1,3,2-dioxaborolan-2-yl)-1H-pyrazol-1-yl)pyridine (1.38 g, 5.09 mmol, 95% yield). LC-MS: m/z=272 [M+H]⁺, retention time 2.000 min (Method B).

### 3-Chloro-4-methyl-5-(1-(pyridin-2-yl)-1H-pyrazol-4-yl)picolinonitrile

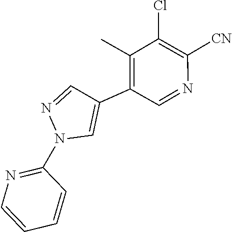 [SMILES: Cc1c(-c2cnn(-c3ccccn3)c2)cnc(C#N)c1Cl]

To a solution of 3,5-dichloro-4-methylpicolinonitrile (862.2 mg, 4.61 mmol), 2-(4-(4,4,5,5-tetramethyl-1,3,2-dioxaborolan-2-yl)-1H-pyrazol-1-yl)pyridine (1.25 g, 4.61 mmol) and potassium carbonate (764.6 mg, 5.53 mmol) was added [1,1′-bis(diphenylphosphino)ferrocene]dichloropalladium(II) (134.9 mg, 0.18 mmol in N,N-dimethylformamide/water (10.0 mL/1.0 mL). The mixture was stirred at 45° C. under nitrogen for 16.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=5/1) to provide the 3-chloro-4-methyl-5-(1-(pyridin-2-yl)-1H-pyrazol-4-yl)picolinonitrile (342 mg, 1.16 mmol, 25% yield). LC-MS: m/z=296 [M+H]⁺, retention time 2.090 min (Method A).

### 3-(Benzyloxy)-4-methyl-5-(1-(pyridin-2-yl)-1H-pyrazol-4-yl)picolinonitrile

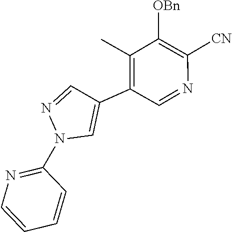 [SMILES: Cc1c(-c2cnn(-c3ccccn3)c2)cnc(C#N)c1OCc1ccccc1]

To a solution of 3-chloro-4-methyl-5-(1-(pyridin-2-yl)-1H-pyrazol-4-yl)picolinonitrile (300.0 mg, 1.01 mmol) in N,N-dimethylformamide (10.0 mL) was sodium hydride (48.8 mg, 1.22 mmol, 60% w/w dispersion in mineral oil) at 0° C. under nitrogen. The mixture was stirred at 0° C. for 10 min and then benzyl alcohol (131.6 mg, 1.22 mmol, 0.127 mL) was added. The solution was stirred at 0° C. for 50 min and diluted with ethyl acetate and water. The organic layer was washed with brine, dried over sodium sulfate and concentrated under reduced pressure. The crude product was purified by flash chromatography (petroleum ether/ethyl acetate=5/1) to provide 3-(benzyloxy)-4-methyl-5-(1-(pyridin-2-yl)-1H-pyrazol-4-yl)picolinonitrile (82 mg, 0.22 mmol, 22% yield). LC-MS: m/z=368 [M+H]⁺, retention time 2.138 min (Method B).

### 3-(Benzyloxy)-4-methyl-5-(1-(pyridin-2-yl)-1H-pyrazol-4-yl)picolinic Acid

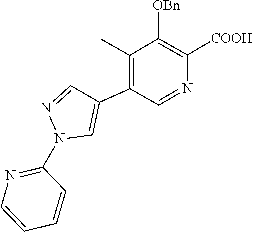 [SMILES: Cc1c(-c2cnn(-c3ccccn3)c2)cnc(C(=O)O)c1OCc1ccccc1]

To a solution of 3-(benzyloxy)-4-methyl-5-(1-(pyridin-2-yl)-1H-pyrazol-4-yl)picolinonitrile (70.0 mg, 0.19 mmol) in ethanol (3.0 mL) was added 30% aqueous sodium hydroxide (1.0 mL). The mixture was stirred at 100° C. for 5.0 h, cooled and concentrated to remove ethanol. The resulting aqueous solution was acidified with 10% hydrochloric acid solution to pH=3˜4. The precipitate was filtered and dried to afford 3-(benzyloxy)-4-methyl-5-(1-(pyridin-2-yl)-1H-pyrazol-4-yl)picolinic acid (72 mg, crude) as white solid. LC-MS: m/z=387 [M+H]⁺, retention time 1.290 min (Method B). The product was pure enough and used directly to the next step.

### Ethyl (3-(benzyloxy)-4-methyl-5-(1-(pyridin-2-yl)-1H-pyrazol-4-yl)picolinoyl) glycinate

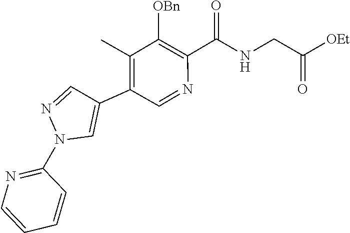 [SMILES: CCOC(=O)CNC(=O)c1ncc(-c2cnn(-c3ccccn3)c2)c(C)c1OCc1ccccc1]

A mixture of 3-(benzyloxy)-4-methyl-5-(1-(pyridin-2-yl)-1H-pyrazol-4-yl)picolinic acid (60. 0 mg, 0.16 mmol), ethyl glycinate hydrochloride (26.41 mg, 0.19 mmol), benzotriazole-1-yl-oxytripyrrolidinophosphonium hexafluorophosphate (88.89 mg, 0.17 mmol) and triethylamine (78.56 mg, 0.78 mmol) in dichloromethane (5.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=3/2) to provide ethyl (3-(benzyloxy)-4-methyl-5-(1-(pyridin-2-yl)-1H-pyrazol-4-yl)picolinoyl) glycinate (70 mg, 0.15 mmol, 93% yield). LC-MS: m/z=472 [M+H]⁺, retention time 2.028 min (Method B).

### Ethyl (3-hydroxy-4-methyl-5-(1-(pyridin-2-yl)-1H-pyrazol-4-yl)picolinoyl) glycinate

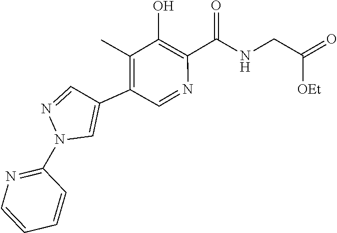 [SMILES: CCOC(=O)CNC(=O)c1ncc(-c2cnn(-c3ccccn3)c2)c(C)c1O]

A mixture of ethyl (3-(benzyloxy)-4-methyl-5-(1-(pyridin-2-yl)-1H-pyrazol-4-yl)picolinoyl) glycinate (60.0 mg, 0.13 mmol) and 10% palladium on carbon (20.0 mg) in tetrahydrofuran (10.0 mL) was stirred under hydrogen atmosphere for 5.0 h. The insoluble solid was filtered and the filtrate was concentrated to give ethyl (3-hydroxy-4-methyl-5-(1-(pyridin-2-yl)-1H-pyrazol-4-yl)picolinoyl) glycinate (50 mg, crude) as white solid. LC-MS: m/z=382 [M+H]⁺, retention time 2.127 min (Method A). The product was pure enough and used directly to the next step.

### (3-Hydroxy-4-methyl-5-(1-(pyridin-2-yl)-1H-pyrazol-4-yl)picolinoyl)glycine

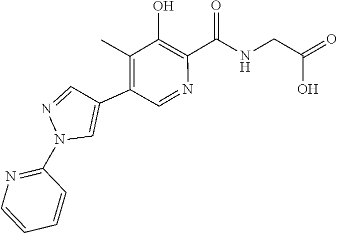 [SMILES: Cc1c(-c2cnn(-c3ccccn3)c2)cnc(C(=O)NCC(=O)O)c1O]

To a solution of ethyl (3-hydroxy-4-methyl-5-(1-(pyridin-2-yl)-1H-pyrazol-4-yl)picolinoyl) glycinate (50.0 mg, 0.13 mmol) in tetrahydrofuran/water (10.0 mL/2.0 mL) was added lithium hydroxide monohydrate (55.0 mg, 1.31 mmol). The mixture was stirred overnight and concentrated to remove tetrahydrofuran. The resulting aqueous solution was acidified with 10% hydrochloric acid solution to pH=3˜4. The precipitate was filtered, washed with water and purified by reverse prep-HPLC to obtain (3-hydroxy-4-methyl-5-(1-(pyridin-2-yl)-1H-pyrazol-4-yl)picolinoyl)glycine (Formate) (28.5 mg, 0.08 mmol, 62% yield) as white solid. LC-MS: m/z=354 [M+H]⁺, retention time 4.255 min (Method A). ¹H NMR (500 MHz, DMSO-d₆) δ 12.84 (br, 1H), 9.27 (br, 1H), 9.01 (s, 1H), 8.53 (d, J=4.0 Hz, 1H), 8.35 (s, 1H), 8.27 (s, 1H), 8.08-8.00 (m, 2H), 7.44-7.41 (m, 1H), 3.97 (d, J=6.0 Hz, 2H), 2.37 (s, 3H).

### Example 19: Preparation of Compound 19

### 4-Bromo-1-(4-fluorophenyl)-1H-pyrazole

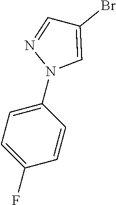 [SMILES: Fc1ccc(-n2cc(Br)cn2)cc1]

A mixture of 4-bromo-1H-pyrazole (1.47 g, 10.0 mmol), 1-fluoro-4-iodobenzene (2.44 g, 11.0 mmol), cesium carbonate (6.50 g, 20.0 mmol), copper iodide (380 mg, 2.0 mmol) and N,N′-dimethyl-1,2-ethanediamine (176 mg, 2.0 mmol) in acetonitrile (20.0 mL) was stirred at 80° C. overnight in a sealed tube. The solution was cooled to room temperature and filtered. Ethyl acetate and water were added to the solution, and the layers were separated. The organic layer was washed with brine, dried over sodium sulfate and concentrated to dryness. The residue was purified by flash chromatography (petroleum ether/ethyl acetate=5/1) to afford 4-bromo-1-(4-fluorophenyl)-1H-pyrazole (1.30 g, 5.39 mmol, 53.9% yield) as yellow solid. LC-MS: m/z=243.0 [M+H]⁺, retention time 2.01 min (Method A).

### 1-(4-Fluorophenyl)-4-(4,4,5,5-tetramethyl-1,3,2-dioxaborolan-2-yl)-1H-pyrazole

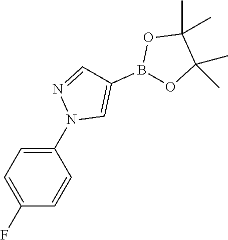 [SMILES: CC1(C)OB(c2cnn(-c3ccc(F)cc3)c2)OC1(C)C]

To a solution of 4-bromo-1-(4-fluorophenyl)-1H-pyrazole (1.30 g, 5.39 mmol), bis(pinacolato)diboron (1.21 g, 4.78 mmol) and potassium acetate (2.11 g, 21.6 mmol) was added [1,1′-bis(diphenylphosphino)ferrocene]dichloropalladium(II) (395 mg, 0.54 mmol) in 1,4-dioxane (15.0 mL). The mixture was stirred at 90° C. under nitrogen for 16.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=4/1) to provide 1-(4-fluorophenyl)-4-(4,4,5,5-tetramethyl-1,3,2-dioxaborolan-2-yl)-1H-pyrazole (1.21 g, 4.20 mmol, 77.9% yield). LC-MS: m/z=289.1 [M+H]⁺, retention time 2.14 min (Method A).

### 3-Chloro-5-(1-(4-fluorophenyl)-1H-pyrazol-4-yl)-4-methylpicolinonitrile

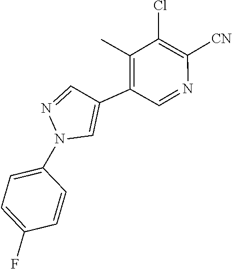 [SMILES: Cc1c(-c2cnn(-c3ccc(F)cc3)c2)cnc(C#N)c1Cl]

To a solution of 3,5-dichloro-4-methylpicolinonitrile (785 mg, 4.20 mmol), 1-(4-fluorophenyl)-4-(4,4,5,5-tetramethyl-1,3,2-dioxaborolan-2-yl)-1H-pyrazole (1.21 g, 4.20 mmol) and potassium carbonate (869.4 mg, 6.30 mmol) was added [1,1′-bis(diphenylphosphino)ferrocene]dichloropalladium(II) (307 mg, 0.42 mmol in N,N-dimethylformamide/water (10.0 mL/1.0 mL). The mixture was stirred at 50° C. under nitrogen for 16.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=2/1) to provide 3-chloro-5-(1-(4-fluorophenyl)-1H-pyrazol-4-yl)-4-methylpicolinonitrile (180 mg, 0.58 mmol, 13.8% yield). LC-MS: m/z=313.0 [M+H]⁺, retention time 2.13 min (Method A).

### 3-(Benzyloxy)-5-(1-(4-fluorophenyl)-1H-pyrazol-4-yl)-4-methylpicolinonitrile

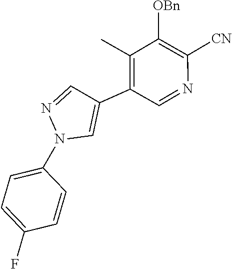 [SMILES: Cc1c(-c2cnn(-c3ccc(F)cc3)c2)cnc(C#N)c1OCc1ccccc1]

To a solution of 3-chloro-5-(1-(4-fluorophenyl)-1H-pyrazol-4-yl)-4-methylpicolinonitrile (180 mg, 0.58 mmol) in N,N-dimethylformamide (5.0 mL) was sodium hydride (27.8 mg, 0.70 mmol, 60% w/w dispersion in mineral oil) at 0° C. under nitrogen. The mixture was stirred at 0° C. for 10 min and then benzyl alcohol (62.6 mg, 0.58 mmol) was added. The solution was stirred at 0° C. for 1.0 h and diluted with ethyl acetate and water. The organic layer was washed with brine, dried over sodium sulfate and concentrated under reduced pressure. The crude product was purified by flash chromatography (petroleum ether/ethyl acetate=5/1) to provide 3-(benzyloxy)-5-(1-(4-fluorophenyl)-1H-pyrazol-4-yl)-4-methylpicolinonitrile (35 mg, 0.09 mmol, 15.7% yield). LC-MS: m/z=385.1 [M+H]⁺, retention time 2.194 min (Method A).

### 3-(Benzyloxy)-5-(1-(4-fluorophenyl)-1H-pyrazol-4-yl)-4-methylpicolinic acid

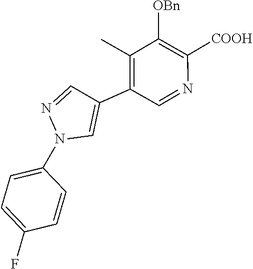 [SMILES: Cc1c(-c2cnn(-c3ccc(F)cc3)c2)cnc(C(=O)O)c1OCc1ccccc1]

To a solution of 3-(benzyloxy)-5-(1-(4-fluorophenyl)-1H-pyrazol-4-yl)-4-methylpicolinonitrile (35 mg, 0.09 mmol) in ethanol (3.0 mL) was added 30% aqueous sodium hydroxide (1.0 mL). The mixture was stirred at 100° C. for 5.0 h, cooled and concentrated to remove ethanol. The resulting aqueous solution was acidified with 10% hydrochloric acid solution to pH=3˜4. The precipitate was filtered and dried to afford 3-(benzyloxy)-5-(1-(4-fluorophenyl)-1H-pyrazol-4-yl)-4-methylpicolinic acid (32 mg, crude) as white solid. LC-MS: m/z=404.1 [M+H]⁺, retention time 2.014 min (Method A). The product was pure enough and used directly to the next step.

### Ethyl (3-(benzyloxy)-5-(1-(4-fluorophenyl)-1H-pyrazol-4-yl)-4-methylpicolinoyl)glycinate

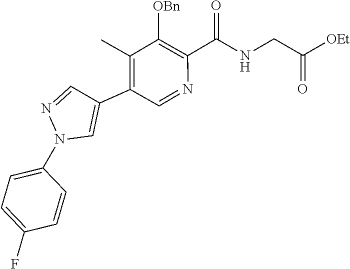 [SMILES: CCOC(=O)CNC(=O)c1ncc(-c2cnn(-c3ccc(F)cc3)c2)c(C)c1OCc1ccccc1]

A mixture of 3-(benzyloxy)-5-(1-(4-fluorophenyl)-1H-pyrazol-4-yl)-4-methylpicolinic acid (32 mg, crude), ethyl glycinate hydrochloride (13.9 mg, 0.1 mmol), benzotriazole-1-yl-oxytripyrrolidinophosphonium hexafluorophosphate (62.4 mg, 0.12 mmol) and triethylamine (50.5 mg, 0.5 mmol) in dichloromethane (5.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/1) to provide ethyl (3-(benzyloxy)-5-(1-(4-fluorophenyl)-1H-pyrazol-4-yl)-4-methylpicolinoyl)glycinate (40 mg, 0.08 mmol, 91% yield). LC-MS: m/z=489.1 [M+H]⁺, retention time 2.128 min (Method A).

### Ethyl (5-(1-(4-fluorophenyl)-1H-pyrazol-4-yl)-3-hydroxy-4-methylpicolinoyl)glycinate

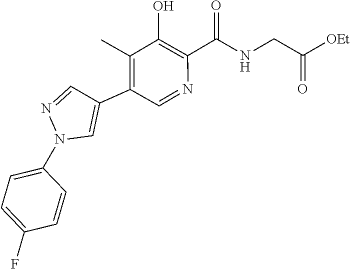 [SMILES: CCOC(=O)CNC(=O)c1ncc(-c2cnn(-c3ccc(F)cc3)c2)c(C)c1O]

A mixture of ethyl (3-(benzyloxy)-5-(1-(4-fluorophenyl)-1H-pyrazol-4-yl)-4-methylpicolinoyl)glycinate (40 mg, 0.08 mmol) and 10% palladium on carbon (10.0 mg) in tetrahydrofuran (5.0 mL) was stirred under hydrogen atmosphere overnight. The insoluble solid was filtered and the filtrate was concentrated to give ethyl (5-(1-(4-fluorophenyl)-1H-pyrazol-4-yl)-3-hydroxy-4-methylpicolinoyl)glycinate (30 mg, 0.08 mmol, 100% yield) as yellow solid. LC-MS: m/z=399.0 [M+H]⁺, retention time 2.15 min (Method A). The product was pure enough and used directly to the next step.

### (5-(1-(4-Fluorophenyl)-1H-pyrazol-4-yl)-3-hydroxy-4-methylpicolinoyl)glycine

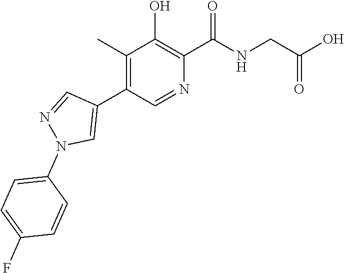 [SMILES: Cc1c(-c2cnn(-c3ccc(F)cc3)c2)cnc(C(=O)NCC(=O)O)c1O]

To a solution of ethyl (5-(1-(4-fluorophenyl)-1H-pyrazol-4-yl)-3-hydroxy-4-methylpicolinoyl)glycinate (30 mg, 0.08 mmol) in tetrahydrofuran/water (5.0 mL/1.0 mL) was added lithium hydroxide monohydrate (42 mg, 1.0 mmol). The mixture was stirred overnight and concentrated to remove tetrahydrofuran. The resulting aqueous solution was acidified with 10% hydrochloric acid solution to pH=3˜4. The precipitate was filtered, washed with water and dried to afford (5-(1-(4-fluorophenyl)-1H-pyrazol-4-yl)-3-hydroxy-4-methylpicolinoyl)glycine (20.1 mg, 0.05 mmol, 67.9% yield) as white solid. LC-MS: m/z=371.0 [M+H]⁺, retention time 4.641 min (Method A). ¹H NMR (500 MHz, DMSO-d₆) δ 12.88 (s, 1H), 9.21 (s, 1H), 8.91 (s, 1H), 8.32 (s, 1H), 8.20 (s, 1H), 8.07-7.84 (m, 2H), 7.53-7.28 (m, 2H), 3.92 (d, J=5.7 Hz, 2H), 2.37 (s, 3H).

### Example 20: Preparation of Compound 20

### 4-Bromo-1-isopropyl-1H-pyrazole

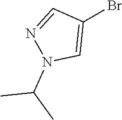

To a solution of 4-bromo-1H-pyrazole (1.47 g, 10.0 mmol) in N,N-dimethylformamide (10.0 mL) was sodium hydride (480 mg, 12.0 mmol, 60% w/w dispersion in mineral oil) at 0° C. under nitrogen. The mixture was stirred at 0° C. for 20 min and then 2-iodopropane (1.7 g, 10.0 mmol) was added. The solution was stirred at room temperature for 18.0 h and diluted with ethyl acetate and water. The organic layer was washed with brine, dried over sodium sulfate and concentrated under reduced pressure. The crude product was purified by flash chromatography (petroleum ether/ethyl acetate=10/1) to give 4-bromo-1-isopropyl-1H-pyrazole (1.47 g, 7.8 mmol, 78% yield). LC-MS: m/z=189.0 [M+H]⁺, retention time 1.90 min (Method B).

### 1-Isopropyl-4-(4,4,5,5-tetramethyl-1,3,2-dioxaborolan-2-yl)-1H-pyrazole

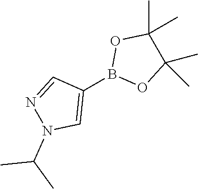 [SMILES: CC(C)n1cc(B2OC(C)(C)C(C)(C)O2)cn1]

To a solution of 4-bromo-1-isopropyl-1H-pyrazole (1.47 g, 7.8 mmol) bis(pinacolato)diboron (3.96 g, 15.6 mmol) and potassium acetate (3.06, 31.2 mmol) was added [1,1′-bis(diphenylphosphino)ferrocene]dichloropalladium(II) (570 mg, 0.78 mmol) in 1,4-dioxane (15.0 mL). The mixture was stirred at 90° C. under nitrogen for 16.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=5/1) to give 1-isopropyl-4-(4,4,5,5-tetramethyl-1,3,2-dioxaborolan-2-yl)-1H-pyrazole (1.18 g, 5.0 mmol, 64.1% yield). LC-MS: m/z=237.0[M+H]⁺, retention time 1.96 min (Method B).

### 3-Chloro-5-(1-isopropyl-1H-pyrazol-4-yl)-4-methylpicolinonitrile

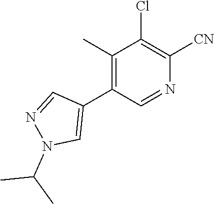 [SMILES: Cc1c(-c2cnn(C(C)C)c2)cnc(C#N)c1Cl]

To a solution of 3,5-dichloro-4-methylpicolinonitrile (1.5 g, 8.02 mmol), 1-isopropyl-4-(4,4,5,5-tetramethyl-1,3,2-dioxaborolan-2-yl)-1H-pyrazole (1.9 g 8.02 mmol) and potassium carbonate (1.3 g, 9.63 mmol) was added [1,1′-bis(diphenylphosphino)ferrocene]dichloropalladium(II) (235 mg, 0.32 mmol) in N,N-dimethylformamide/water (15.0 mL/1.5 mL). The mixture was stirred at 50° C. under nitrogen overnight and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=2/1) to afford 3-chloro-5-(1-isopropyl-1H-pyrazol-4-yl)-4-methylpicolinonitrile (580 mg, 2.22 mmol, 29% yield) as a yellow solid. LC-MS: m/z=261.1 [M+H]⁺, retention time=1.83 min (Method A).

### 3-(Benzyloxy)-5-(1-isopropyl-1H-pyrazol-4-yl)-4-methylpicolinonitrile

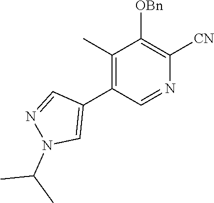 [SMILES: Cc1c(-c2cnn(C(C)C)c2)cnc(C#N)c1OCc1ccccc1]

To a solution of 3-chloro-5-(1-isopropyl-1H-pyrazol-4-yl)-4-methylpicolinonitrile (580.0 mg, 2.22 mmol) in N,N-dimethylformamide (5.0 mL) was sodium hydride (108 mg, 2.67 mmol, 60% w/w dispersion in mineral oil) at 0° C. under nitrogen. The mixture was stirred at 0° C. for 10 min and then benzyl alcohol (288 mg, 2.67 mmol) was added. The solution was stirred at 0° C. for 50 min and diluted with ethyl acetate and water. The organic layer was washed with brine, dried over sodium sulfate and concentrated under reduced pressure. The crude product was purified by flash chromatography (petroleum ether/ethyl acetate=3/1) to afford 3-(benzyloxy)-5-(1-isopropyl-1H-pyrazol-4-yl)-4-methylpicolinonitrile (180 mg, 0.54 mmol, 24% yield) as yellow solid. LC-MS: m/z=333.1 [M+H]+, retention time=2.21 min (Method A).

### 3-(Benzyloxy)-5-(1-isopropyl-1H-pyrazol-4-yl)-4-methylpicolinic acid

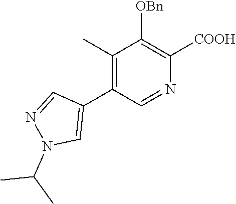 [SMILES: Cc1c(-c2cnn(C(C)C)c2)cnc(C(=O)O)c1OCc1ccccc1]

To a solution of 3-(benzyloxy)-5-(1-isopropyl-1H-pyrazol-4-yl)-4-methylpicolinonitrile (180 mg, 0.54 mmol) in ethanol (5.0 mL) was added 30% aqueous sodium hydroxide (1.0 mL). The mixture was stirred at 100° C. for 5.0 h, cooled and concentrated to remove ethanol. The resulting aqueous solution was acidified with 10% hydrochloric acid solution to pH=3˜4. The precipitate was filtered and dried to afford 3-(benzyloxy)-5-(1-isopropyl-1H-pyrazol-4-yl)-4-methylpicolinic acid (100 mg, 0.28 mmol, 53% yield) as white solid. LC-MS: m/z=352.1 [M+H]⁺, retention time 2.00 min (Method A). The product was pure enough and used directly to the next step.

### Ethyl (3-(benzyloxy)-5-(1-isopropyl-1H-pyrazol-4-yl)-4-methylpicolinoyl)glycinate

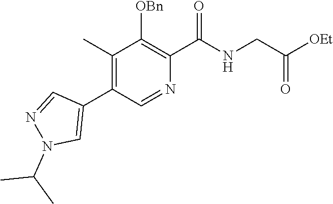 [SMILES: CCOC(=O)CNC(=O)c1ncc(-c2cnn(C(C)C)c2)c(C)c1OCc1ccccc1]

A mixture of 3-(benzyloxy)-5-(1-isopropyl-1H-pyrazol-4-yl)-4-methylpicolinic acid (100 mg, 0.28 mmol), ethyl glycinate hydrochloride (40 mg, 0.28 mmol), benzotriazole-1-yl-oxytripyrrolidinophosphonium hexafluorophosphate (178 mg, 0.34 mmol), triethylamine (254 mg, 2.52 mmol) in dichloromethane (5.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/1) to obtain ethyl (3-(benzyloxy)-5-(1-isopropyl-1H-pyrazol-4-yl)-4-methylpicolinoyl)glycinate (100 mg, 0.18 mmol, 81% yield) as white solid. LC-MS: m/z=437.1 [M+H]⁺, retention time 2.15 min (Method A).

### Ethyl (3-hydroxy-5-(1-isopropyl-1H-pyrazol-4-yl)-4-methylpicolinoyl)glycinate

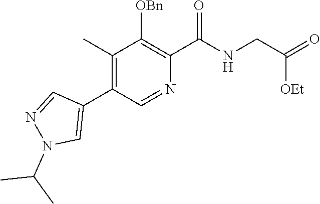 [SMILES: CCOC(=O)CNC(=O)c1ncc(-c2cnn(C(C)C)c2)c(C)c1OCc1ccccc1]

A mixture of ethyl (3-(benzyloxy)-5-(1-isopropyl-1H-pyrazol-4-yl)-4-methylpicolinoyl)glycinate (100 mg, 0.18 mmol) and 10% palladium on carbon (20.0 mg) in tetrahydrofuran (10.0 mL) was stirred under hydrogen atmosphere for 18 h. The insoluble solid was filtered and the filtrate was concentrated to provide ethyl (3-hydroxy-5-(1-isopropyl-1H-pyrazol-4-yl)-4-methylpicolinoyl)glycinate (80 mg, crude) as yellow solid. LC-MS: m/z=347.1 [M+H]⁺, retention time 2.19 min (Method A). The product was pure enough and used directly to the next step.

### (3-Hydroxy-5-(1-isopropyl-1H-pyrazol-4-yl)-4-methylpicolinoyl)glycine

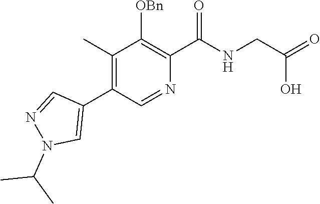 [SMILES: Cc1c(-c2cnn(C(C)C)c2)cnc(C(=O)NCC(=O)O)c1OCc1ccccc1]

To a solution of ethyl (3-hydroxy-5-(1-isopropyl-1H-pyrazol-4-yl)-4-methylpicolinoyl)glycinate (80 mg, crude) in tetrahydrofuran/water (8.0 mL/2.0 mL) was added lithium hydroxide monohydrate (164 mg, 4.0 mmol). The mixture was stirred overnight and concentrated to remove tetrahydrofuran. The resulting aqueous solution was acidified with 10% hydrochloric acid solution to pH=3˜4. The precipitate was filtered, washed with water and dried to obtain (3-hydroxy-5-(1-isopropyl-1H-pyrazol-4-yl)-4-methylpicolinoyl)glycine (45.7 mg, 62% yield) as white solid. LC-MS: m/z=319.1 [M+H]⁺, retention time 3.81 min (Method A). ¹HNMR (400 MHz, DMSO-d₆) δδ 12.75 (s, 1H), 9.24 (t, J=6.0 Hz, 1H), 8.23 (s, 2H), 7.86 (s, 1H), 4.58 (dt, J=13.3, 6.6 Hz, 1H), 3.99 (d, J=6.1 Hz, 2H), 2.31 (s, 3H), 1.48 (d, J=6.7 Hz, 6H).

### Example 21: Preparation of Compound 21

### 4-bromo-1-(tetrahydro-2H-pyran-4-yl)-1H-pyrazole

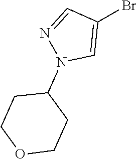 [SMILES: Brc1cnn(C2CCOCC2)c1]

To a solution of 4-bromo-1H-pyrazole (1.47 g, 10.0 mmol) in N,N-dimethylformamide (10.0 mL) was sodium hydride (480 mg, 12.0 mmol, 60% w/w dispersion in mineral oil) at 0° C. under nitrogen. The mixture was stirred at 0° C. for 20 min and then tetrahydro-2H-pyran-4-yl methanesulfonate (1.8 g, 10.0 mmol) was added. The solution was stirred at 10° C. for 2.0 h and diluted with ethyl acetate and water. The organic layer was washed with brine, dried over sodium sulfate and concentrated under reduced pressure. The crude product was purified by flash chromatography (petroleum ether/ethyl acetate=10/1) to give 4-bromo-1-(tetrahydro-2H-pyran-4-yl)-1H-pyrazole (1.16 g, 5.0 mmol, 50% yield). LC-MS: m/z=231.0 [M+H]⁺, retention time 1.76 min (Method B).

### 1-(Tetrahydro-2H-pyran-4-yl)-4-(4,4,5,5-tetramethyl-1,3,2-dioxaborolan-2-yl)-1H-pyrazole

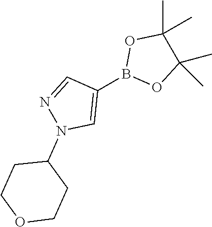 [SMILES: CC1(C)OB(c2cnn(C3CCOCC3)c2)OC1(C)C]

To a solution of 4-bromo-1-(tetrahydro-2H-pyran-4-yl)-1H-pyrazole (2.8 g, 12.10 mmol), bis(pinacolato)diboron (3.7 g, 14.50 mmol) and potassium acetate (3.6 g, 36.30 mmol) was added [1,1′-bis(diphenylphosphino)ferrocene]dichloropalladium(II) (443 mg, 0.61 mmol) in 1,4-dioxane (15.0 mL). The mixture was stirred at 90° C. under nitrogen for 16.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=5/1) to afford 1-(tetrahydro-2H-pyran-4-yl)-4-(4,4,5,5-tetramethyl-1,3,2-dioxaborolan-2-yl)-1H-pyrazole (2.5 g, 8.99 mmol, 74% yield) as a white solid. LC-MS: m/z=279.1 [M+H]⁺, retention time=1.83 min (Method A).

### 3-Chloro-4-methyl-5-(1-(tetrahydro-2H-pyran-4-yl)-1H-pyrazol-4-yl)picolinonitrile

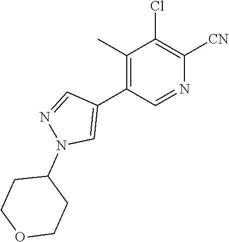 [SMILES: Cc1c(-c2cnn(C3CCOCC3)c2)cnc(C#N)c1Cl]

To a solution of 3,5-dichloro-4-methylpicolinonitrile (1.51 g, 8.09 mmol), 1-(tetrahydro-2H-pyran-4-yl)-4-(4,4,5,5-tetramethyl-1,3,2-dioxaborolan-2-yl)-1H-pyrazole (2.25 g 8.09 mmol) and potassium carbonate (1.34 g, 9.71 mmol) was added [1,1′-bis(diphenylphosphino)ferrocene]dichloropalladium(II) (237 mg, 0.32 mmol) in N,N-dimethylformamide/water (6.0 mL/0.6 mL). The mixture was stirred at 50° C. under nitrogen for 16.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=2/1) to afford 3-chloro-4-methyl-5-(1-(tetrahydro-2H-pyran-4-yl)-1H-pyrazol-4-yl)picolinonitrile (1.0 g, 3.31 mmol, 37% yield) as a yellow solid. LC-MS: m/z=303.1 [M+H]+, retention time=1.83 min (Method A).

### 3-(Benzyloxy)-4-methyl-5-(1-(tetrahydro-2H-pyran-4-yl)-1H-pyrazol-4-yl)picolinonitrile

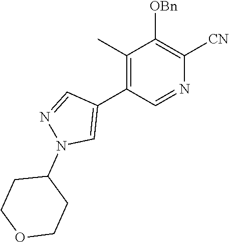 [SMILES: Cc1c(-c2cnn(C3CCOCC3)c2)cnc(C#N)c1OCc1ccccc1]

To a solution of 3-chloro-4-methyl-5-(1-(tetrahydro-2H-pyran-4-yl)-1H-pyrazol-4-yl)picolinonitrile (900.0 mg, 2.97 mmol) in N,N-dimethylformamide (10.0 mL) was sodium hydride (143 mg, 3.57 mmol, 60% w/w dispersion in mineral oil) at 0° C. under nitrogen. The mixture was stirred at 0° C. for 10 min and then benzyl alcohol (386 mg, 3.57 mmol) was added. The solution was stirred at 0° C. for 1.0 h and diluted with ethyl acetate and water. The organic layer was washed with brine, dried over sodium sulfate and concentrated under reduced pressure. The crude product was purified by flash chromatography (petroleum ether/ethyl acetate=3/1) to give 3-(benzyloxy)-4-methyl-5-(1-(tetrahydro-2H-pyran-4-yl)-1H-pyrazol-4-yl)picolinonitrile (100 mg, 0.27 mmol, 8% yield) as yellow solid. LC-MS: m/z=375.1 [M+H]+, retention time=2.21 min (Method A).

### 3-(Benzyloxy)-4-methyl-5-(1-(tetrahydro-2H-pyran-4-yl)-1H-pyrazol-4-yl)picolinic acid

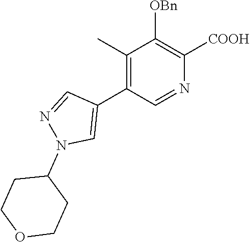 [SMILES: Cc1c(-c2cnn(C3CCOCC3)c2)cnc(C(=O)O)c1OCc1ccccc1]

To a solution of 3-(benzyloxy)-4-methyl-5-(1-(tetrahydro-2H-pyran-4-yl)-1H-pyrazol-4-yl)picolinonitrile (100 mg, 0.27 mmol) in ethanol (5.0 mL) was added 30% aqueous sodium hydroxide (1.5 mL). The mixture was stirred at 100° C. for 5.0 h, cooled and concentrated to remove ethanol. The resulting aqueous solution was acidified with 10% hydrochloric acid solution to pH=3˜4. The precipitate was filtered and dried to afford 3-(benzyloxy)-4-methyl-5-(1-(tetrahydro-2H-pyran-4-yl)-1H-pyrazol-4-yl)picolinic acid (90 mg, 0.23 mmol, 86% yield) as white solid. LC-MS: m/z=394.1 [M+H]⁺, retention time 2.00 min (Method A). The product was pure enough and used directly to the next step.

### Ethyl (3-(benzyloxy)-4-methyl-5-(1-(tetrahydro-2H-pyran-4-yl)-1H-pyrazol-4-yl)picolinoyl)glycinate

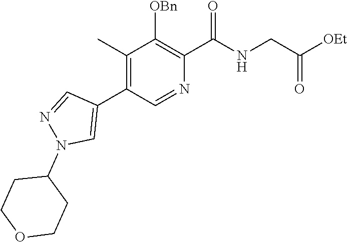 [SMILES: CCOC(=O)CNC(=O)c1ncc(-c2cnn(C3CCOCC3)c2)c(C)c1OCc1ccccc1]

A mixture of 3-(benzyloxy)-4-methyl-5-(1-(tetrahydro-2H-pyran-4-yl)-1H-pyrazol-4-yl)picolinic acid (90 mg, 0.23 mmol), ethyl glycinate hydrochloride (32 mg, 0.23 mmol), benzotriazole-1-yl-oxytripyrrolidinophosphonium hexafluorophosphate (143 mg, 0.27 mmol), triethylamine (254 mg, 2.52 mmol) in dichloromethane (5.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/1) to obtain ethyl (3-(benzyloxy)-4-methyl-5-(1-(tetrahydro-2H-pyran-4-yl)-1H-pyrazol-4-yl)picolinoyl)glycinate (90 mg, 0.19 mmol, 82% yield) as white solid. LC-MS: m/z=479.1 [M+H]⁺, retention time 2.15 min (Method A).

### Ethyl (3-hydroxy-4-methyl-5-(1-(tetrahydro-2H-pyran-4-yl)-1H-pyrazol-4-yl)picolinoyl)glycinate

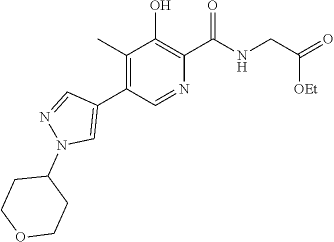 [SMILES: CCOC(=O)CNC(=O)c1ncc(-c2cnn(C3CCOCC3)c2)c(C)c1O]

A mixture of ethyl (3-(benzyloxy)-4-methyl-5-(1-(tetrahydro-2H-pyran-4-yl)-1H-pyrazol-4-yl)picolinoyl)glycinate (90 mg, 0.19 mmol) and 10% palladium on carbon (20.0 mg) in tetrahydrofuran (10.0 mL) was stirred under hydrogen atmosphere for 18 h. The insoluble solid was filtered and the filtrate was concentrated to give ethyl (3-hydroxy-4-methyl-5-(1-(tetrahydro-2H-pyran-4-yl)-1H-pyrazol-4-yl)picolinoyl)glycinate (70 mg, 0.18 mmol, 96% yield) as yellow solid. LC-MS: m/z=389.1 [M+H]⁺, retention time 2.19 min (Method A). The product was pure enough and used directly to the next step.

### (3-Hydroxy-4-methyl-5-(1-(tetrahydro-2H-pyran-4-yl)-1H-pyrazol-4-yl)picolinoyl)glycine

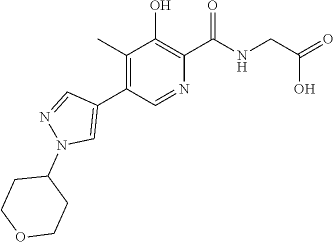 [SMILES: Cc1c(-c2cnn(C3CCOCC3)c2)cnc(C(=O)NCC(=O)O)c1O]

To a solution of ethyl (3-hydroxy-4-methyl-5-(1-(tetrahydro-2H-pyran-4-yl)-1H-pyrazol-4-yl)picolinoyl)glycinate (70 mg, 0.18 mmol) in tetrahydrofuran/water (8.0 mL/2.0 mL) was added lithium hydroxide monohydrate (164 mg, 4.0 mmol). The mixture was stirred overnight and concentrated to remove tetrahydrofuran. The resulting aqueous solution was acidified with 10% hydrochloric acid solution to pH=3˜4. The precipitate was filtered, washed with water and purified by reverse prep-HPLC to obtain (3-hydroxy-4-methyl-5-(1-(tetrahydro-2H-pyran-4-yl)-1H-pyrazol-4-yl)picolinoyl)glycine (10.8 mg, 0.03 mmol, 17% yield) as white solid. LC-MS: m/z=361.1 [M+H]⁺, retention time 3.45 min (Method A). ¹HNMR (500 MHz, DMSO-d₆) δ 12.76 (s, 2H), 9.25 (t, J=6.0 Hz, 1H), 8.28 (s, 1H), 8.23 (s, 1H), 7.89 (s, 1H), 4.69-4.32 (m, 1H), 3.98 (d, J=6.2 Hz, 4H), 3.58-3.43 (m, 3H), 2.31 (s, 3H), 2.07-1.93 (m, 4H).

### Example 22: Preparation of Compound 22

### 4-Bromo-1-isobutyl-1H-pyrazole

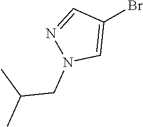

To a solution of 4-bromo-1H-pyrazole (1.0 g, 6.80 mmol) and 1-bromo-2-methylpropane (1118.68 mg, 8.16 mmol)in N,N-dimethylformamide (10.0 mL) was added potassium carbonate (2.82 g, 20.41 mmol). The mixture was stirred at 90° C. for 18.0 h and diluted with ethyl acetate and water. The organic layer was washed with brine, dried over sodium sulfate and concentrated under reduced pressure. The crude product was purified by flash chromatography (petroleum ether/ethyl acetate=20/1) to provide 4-bromo-1-isobutyl-1H-pyrazole (1.15 g, 5.69 mmol, 83% yield). LC-MS: m/z=203 [M+H]⁺, retention time 1.861 min (Method B).

### 1-Isobutyl-4-(4,4,5,5-tetramethyl-1,3,2-dioxaborolan-2-yl)-1H-pyrazole

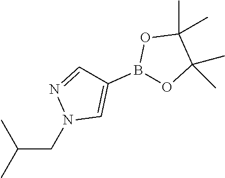 [SMILES: CC(C)Cn1cc(B2OC(C)(C)C(C)(C)O2)cn1]

To a solution of 4-bromo-1-isobutyl-1H-pyrazole (1.1 g, 5.42 mmol) bis(pinacolato)diboron (6.88 g, 27.08 mmol) and potassium acetate (1.59 g, 16.25 mmol) was added [1,1′-bis(diphenylphosphino)ferrocene]dichloropalladium(II) (396 mg, 0.54 mmol) in 1,4-dioxane (15.0 mL). The mixture was stirred at 90° C. under nitrogen for 16.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=10/1) to provide 1-isobutyl-4-(4,4,5,5-tetramethyl-1,3,2-dioxaborolan-2-yl)-1H-pyrazole (1.1 g, 4.39 mmol, 81% yield). LC-MS: m/z=251 [M+H]⁺, retention time 2.047 min (Method A).

### 3-Chloro-5-(1-isobutyl-1H-pyrazol-4-yl)-4-methylpicolinonitrile

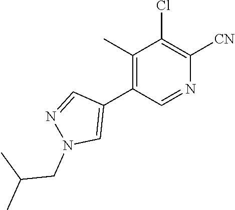 [SMILES: Cc1c(-c2cnn(CC(C)C)c2)cnc(C#N)c1Cl]

To a solution of 3,5-dichloro-4-methylpicolinonitrile (411.2 mg, 2.20 mmol), 1-isobutyl-4-(4,4,5,5-tetramethyl-1,3,2-dioxaborolan-2-yl)-1H-pyrazole (550.0 mg, 2.20 mmol) and potassium carbonate (364.7 mg, 2.64 mmol) was added [1,1′-bis(diphenylphosphino)ferrocene]dichloropalladium(II) (64.4 mg, 0.09 mmol) in N,N-dimethylformamide/water (10.0 mL/1.0 mL). The mixture was stirred at 50° C. under nitrogen overnight and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=3/1) to provide 3-chloro-5-(1-isobutyl-1H-pyrazol-4-yl)-4-methylpicolinonitrile (732 mg, 2.67 mmol, 87% yield). LC-MS: m/z=275 [M+H]⁺, retention time 2.065 min (Method A).

### 3-(Benzyloxy)-5-(1-isobutyl-1H-pyrazol-4-yl)-4-methylpicolinonitrile

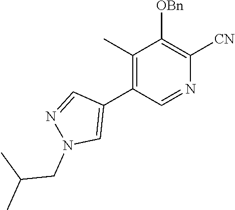 [SMILES: Cc1c(-c2cnn(CC(C)C)c2)cnc(C#N)c1OCc1ccccc1]

To a solution of 3-chloro-5-(1-isobutyl-1H-pyrazol-4-yl)-4-methylpicolinonitrile (650.00 mg, 2.37 mmol) in N,N-dimethylformamide (10.0 mL) was sodium hydride (113.6 mg, 2.84 mmol, 60% w/w dispersion in mineral oil) at 0° C. under nitrogen. The mixture was stirred at 0° C. for 10 min and then benzyl alcohol (307.0 mg, 2.84 mmol, 0.3 mL) was added. The solution was stirred at 0° C. for 50 min and diluted with ethyl acetate and water. The organic layer was washed with brine, dried over sodium sulfate and concentrated under reduced pressure. The crude product was purified by flash chromatography (petroleum ether/ethyl acetate=3/1) to provide 3-(benzyloxy)-5-(1-isobutyl-1H-pyrazol-4-yl)-4-methylpicolinonitrile (105 mg, 0.30 mmol, 13% yield). LC-MS: m/z=347 [M+H]⁺, retention time 2.089 min (Method B).

### 3-(Benzyloxy)-5-(1-isobutyl-1H-pyrazol-4-yl)-4-methylpicolinic acid

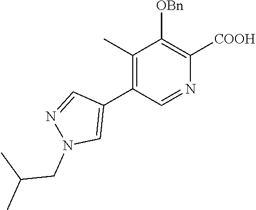 [SMILES: Cc1c(-c2cnn(CC(C)C)c2)cnc(C(=O)O)c1OCc1ccccc1]

To a solution of 3-(benzyloxy)-5-(1-isobutyl-1H-pyrazol-4-yl)-4-methylpicolinonitrile (95.0 mg, 0.27 mmol) in ethanol (3.0 mL) was added 30% aqueous sodium hydroxide (1.0 mL). The mixture was stirred at 100° C. for 5.0 h, cooled and concentrated to remove ethanol. The resulting aqueous solution was acidified with 10% hydrochloric acid solution to pH=3˜4. The precipitate was filtered and dried to afford 3-(benzyloxy)-5-(1-isobutyl-1H-pyrazol-4-yl)-4-methylpicolinic acid (90 mg, crude) as white solid. LC-MS: m/z=366 [M+H]⁺, retention time 1.391 min (Method B). The product was pure enough and used directly to the next step.

### Ethyl (3-(benzyloxy)-5-(1-isobutyl-1H-pyrazol-4-yl)-4-methylpicolinoyl)glycinate

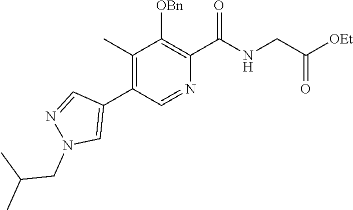 [SMILES: CCOC(=O)CNC(=O)c1ncc(-c2cnn(CC(C)C)c2)c(C)c1OCc1ccccc1]

A mixture of 3-(benzyloxy)-5-(1-isobutyl-1H-pyrazol-4-yl)-4-methylpicolinic acid (90.00 mg, 0.25 mmol), ethyl glycinate hydrochloride (41.7 mg, 0.30 mmol), benzotriazole-1-yl-oxytripyrrolidinophosphonium hexafluorophosphate (140.98 mg, 0.27 mmol) and triethylamine (124.6 mg, 1.23 mmol) in dichloromethane (10.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/1) to provide ethyl (3-(benzyloxy)-5-(1-isobutyl-1H-pyrazol-4-yl)-4-methylpicolinoyl)glycinate (30 mg, 0.067 mmol, 27% yield). LC-MS: m/z=451 [M+H]⁺, retention time 2.008 min (Method B).

### Ethyl (3-hydroxy-5-(1-isobutyl-1H-pyrazol-4-yl)-4-methylpicolinoyl)glycinate

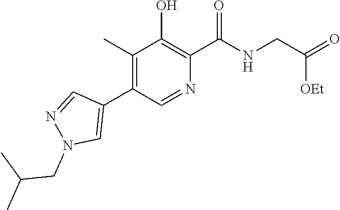 [SMILES: CCOC(=O)CNC(=O)c1ncc(-c2cnn(CC(C)C)c2)c(C)c1O]

A mixture of ethyl (3-(benzyloxy)-5-(1-isobutyl-1H-pyrazol-4-yl)-4-methylpicolinoyl) glycinate (25.0 mg, 0.06 mmol) and 10% palladium on carbon (20.0 mg) in tetrahydrofuran (10.0 mL) was stirred under hydrogen atmosphere for 18.0 h. The insoluble solid was filtered and the filtrate was concentrated to provide ethyl (3-hydroxy-5-(1-isobutyl-1H-pyrazol-4-yl)-4-methylpicolinoyl)glycinate (30.0 mg, crude) as yellow solid. LC-MS: m/z=361 [M+H]⁺, retention time 1.966 min (Method B). The product was pure enough and used directly to the next step.

### (3-Hydroxy-5-(1-isobutyl-1H-pyrazol-4-yl)-4-methylpicolinoyl)glycine

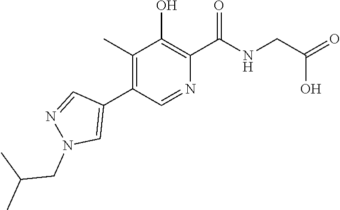 [SMILES: Cc1c(-c2cnn(CC(C)C)c2)cnc(C(=O)NCC(=O)O)c1O]

To a solution of ethyl (3-hydroxy-5-(1-isobutyl-1H-pyrazol-4-yl)-4-methylpicolinoyl) glycinate (25.00 mg, 0.07 mmol) in tetrahydrofuran/water (8.0 mL/2.0 mL) was added lithium hydroxide monohydrate (164 mg, 4.0 mmol). The mixture was stirred overnight and concentrated to remove tetrahydrofuran. The resulting aqueous solution was acidified with 10% hydrochloric acid solution to pH=3˜4. The precipitate was purified by reverse Prep-HPLC to provide (3-hydroxy-5-(1-isobutyl-1H-pyrazol-4-yl)-4-methylpicolinoyl)glycine (Formate) (6.4 mg, 0.02 mmol, 27% yield) as yellow solid. LC-MS: m/z=333 [M+H]⁺, retention time 4.188 min (Method A). ¹H NMR (400 MHz, DMSO-d₆) δ 12.75 (s, 1H), 9.27-9.25 (m, 1H), 8.22 (s, 1H), 8.20 (s, 1H), 7.87 (s, 1H), 4.00 (s, 2H), 3.98 (d, J=2.4 Hz, 2H), 2.30 (s, 3H), 2.19-2.15 (m, 1H), 0.88 (d, J=6.8 Hz, 6H).

### Example 23: Preparation of Compound 23

### Methyl 3-oxohept-6-enoate

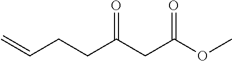

To a suspension of sodium hydride (1.89 g, 47.30 mmol, 60% w/w dispersion in mineral oil) in anhydrous tetrahydrofuran (120 mL) was added methyl 3-oxobutanoate (3.23 g, 27.82 mmol) at 0° C. The solution was stirred at 0° C. for 30 min and n-butyllithium (17.8 mL, 44.52 mmol, 2.5 M in n-hexane) was added. After stirring for 30 min, 3-bromoprop-1-ene (3.70 g, 30.61 mmol) was added. The reaction mixture was warmed up to 20° C. and stirred for another 2.0 h. The reaction was quenched by addition of saturated aqueous ammonium chloride solution and extracted with ethyl acetate. The organic layer was washed with brine, dried over sodium sulfate and concentrated to dryness. The residue was purified by flash chromatography (petroleum ether/ethyl acetate=100/3) to afford methyl 3-oxohept-6-enoate (2.74 g, 17.6 mmol, 63% yield) as yellow oil. LC-MS: m/z=157 [M+H]⁺, retention time 1.446 min (Method B).

### Methyl 4-(2-(benzyloxy)ethyl)-3-oxohept-6-enoate

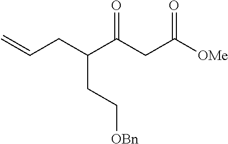 [SMILES: C=CCC(CCOCc1ccccc1)C(=O)CC(=O)OC]

To a solution of methyl 3-oxohept-6-enoate (800.0 mg, 5.12 mmol) in anhydrous tetrahydrofuran (15.0 mL) was added freshly lithium diisopropylamide (11.2 mmol, 5.63 mL, 2.0 M in n-hexane) at 0° C. The mixture was stirred at 0° C. for 15 min and ((2-bromoethoxy)methyl) benzene (1.32 g, 6.15 mmol) was added. The mixture was allowed to warm up to 20° C. and left stirring for 3.0 h. The reaction was quenched with saturated ammonium chloride solution and extracted twice with ethyl acetate. The organic layer was separated, washed with brine, dried over sodium sulfate and concentrated under reduced pressure. The crude product was purified by flash chromatography (petroleum ether/ethyl acetate=10/1) to afford methyl 4-(2-(benzyloxy)ethyl)-3-oxohept-6-enoate (729 mg, 2.51 mmol, 49% yield) as yellow oil. LC-MS: m/z=313 [M+Na]+, retention time 2.080 min (Method A).

### Methyl 2,2-diazido-4-(2-(benzyloxy)ethyl)-3-oxohept-6-enoate

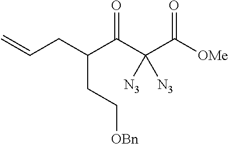 [SMILES: C=CCC(CCOCc1ccccc1)C(=O)C(N=[N+]=[N-])(N=[N+]=[N-])C(=O)OC]

A mixture of methyl 4-(2-(benzyloxy)ethyl)-3-oxohept-6-enoate (700.0 mg, 2.41 mmol), sodium azide (626.9 mg, 9.64 mmol), sodium hydrocarbonate (607.6 mg, 7.23 mmol) and iodine (1.25 g, 4.94 mmol) in dimethylsulfoxide/water (30.0 mL/15.0 mL) was stirred at room temperature for 16.0 h. The reaction was quenched with saturated ammonium chloride solution and extracted ethyl acetate. The organic layer was separated, washed with brine, dried over sodium sulfate and concentrated under reduced pressure. The crude product was purified by flash chromatography (petroleum ether/ethyl acetate=10/1) to afford methyl 2,2-diazido-4-(2-(benzyloxy)ethyl)-3-oxohept-6-enoate (330 mg, 0.89 mmol, 37% yield). LC-MS: m/z=395 [M+Na]⁺, retention time 2.314 min (Method A).

### Methyl 4-(2-(benzyloxy)ethyl)-3-hydroxy-6-methylpicolinate

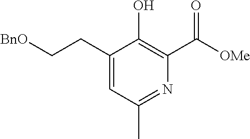 [SMILES: COC(=O)c1nc(C)cc(CCOCc2ccccc2)c1O]

A mixture of methyl 2,2-diazido-4-(2-(benzyloxy)ethyl)-3-oxohept-6-enoate (300.0 mg, 0.81 mmol) in toluene (5.0 mL) was stirred at 110° C. for 16.0 h in a sealed tube. The solution was cooled and concentrated to give dryness. The resulting residue was purified by flash chromatography (petroleum ether/ethyl acetate=3/1) to provide the methyl 4-(2-(benzyloxy)ethyl)-3-hydroxy-6-methylpicolinate (65 mg, 0.22 mmol, 27% yield). LC-MS: m/z=302 [M+H]⁺, retention time 1.991 min (Method A).

### 4-(2-(Benzyloxy)ethyl)-3-hydroxy-6-methylpicolinic Acid

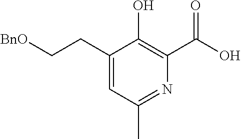 [SMILES: Cc1cc(CCOCc2ccccc2)c(O)c(C(=O)O)n1]

To a solution of methyl 4-(2-(benzyloxy)ethyl)-3-hydroxy-6-methylpicolinate (60.0 mg, 0.20 mmol) in methanol/water (4.0 mL/1.0 mL) was added lithium hydroxide monohydrate (83.6 mg, 1.99 mmol). The mixture was stirred overnight and concentrated to remove methanol. The resulting aqueous solution was acidified by adding 10% hydrochloric acid (5.0 mL) and extracted ethyl acetate twice. The organic layer was separated, washed with brine, dried over sodium sulfate and concentrated under reduced pressure. The crude 4-(2-(benzyloxy)ethyl)-3-hydroxy-6-methylpicolinic acid (55 mg, 0.19 mmol, 96% yield) was obtained as yellow solid. LC-MS: m/z=288 [M+H]⁺, retention time 1.626 min (Method A). The crude product was used to the next step.

### Ethyl (4-(2-(benzyloxy)ethyl)-3-hydroxy-6-methylpicolinoyl)glycinate

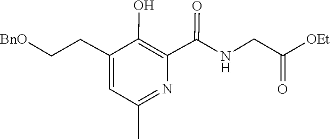 [SMILES: CCOC(=O)CNC(=O)c1nc(C)cc(CCOCc2ccccc2)c1O]

A mixture of 4-(2-(benzyloxy)ethyl)-3-hydroxy-6-methylpicolinic acid (50.00 mg, 0.17 mmol), ethyl glycinate hydrochloride (21.53 mg, 0.21 mmol), benzotriazole-1-yl-oxytripyrrolidinophosphonium hexafluorophosphate (99.62 mg, 0.19 mmol) and triethylamine (88.05 mg, 0.87 mmol) in dichloromethane (10.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=4/1) to provide ethyl (4-(2-(benzyloxy)ethyl)-3-hydroxy-6-methylpicolinoyl)glycinate (48 mg, 0.13 mmol, 76% yield) as white solid. LC-MS: m/z=373 [M+H]⁺, retention time 2.179 min (Method A).

### (4-(2-(Benzyloxy)ethyl)-3-hydroxy-6-methylpicolinoyl)glycine

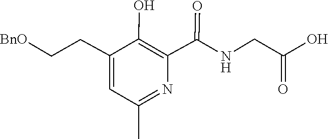 [SMILES: Cc1cc(CCOCc2ccccc2)c(O)c(C(=O)NCC(=O)O)n1]

To a solution of ethyl (4-(2-(benzyloxy)ethyl)-3-hydroxy-6-methylpicolinoyl)glycinate (48.0 mg, 0.13 mmol) in methanol/water (4.0 mL/1.0 mL) was added lithium hydroxide monohydrate (54.2 mg, 1.29 mmol). The mixture was stirred overnight and concentrated to remove methanol. The resulting aqueous solution was acidified with 10% hydrochloric acid solution to pH=3˜4. The precipitate was purified by reverse Prep-HPLC to provide (4-(2-(benzyloxy)ethyl)-3-hydroxy-6-methylpicolinoyl)glycine (Formate) (29.7 mg, 0.09 mmol, 66% yield) as red solid. LC-MS: m/z=345 [M+H]⁺, retention time 4.596 min (Method A). ¹H NMR (500 MHz, DMSO-d₆) δ 12.75 (br, 1H), 12.40 (s, 1H), 9.11 (t, J=6.0 Hz, 1H), 7.34-7.31 (m, 3H), 7.28-7.25 (m, 3H), 4.48 (s, 2H), 3.99 (d, J=6.0 Hz, 2H), 3.69 (t, J=6.5 Hz, 2H), 2.87 (t, J=6.5 Hz, 2H), 2.42 (s, 3H).

### Example 24: Preparation of Compound 24

### Methyl 4-(4-fluorobenzyl)-3-oxohept-6-enoate

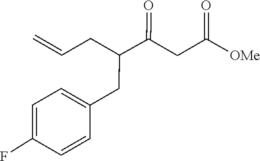 [SMILES: C=CCC(Cc1ccc(F)cc1)C(=O)CC(=O)OC]

To a solution of methyl 3-oxohept-6-enoate (Intermediate for Example 22) (500.0 mg, 3.20 mmol) in anhydrous tetrahydrofuran (20.0 mL) was added freshly lithium diisopropylamide (7.04 mmol, 3.52 mL, 2.0 M in n-hexane) at 0° C. The mixture was stirred at 0° C. for 30 min and 1-(bromomethyl)-4-fluorobenzene (726.2 mg, 3.84 mmol) was added. The mixture was allowed to warm up to 20° C. and left stirring for 3.0 h. The reaction was quenched with saturated ammonium chloride solution and extracted twice with ethyl acetate. The organic layer was separated, washed with brine, dried over sodium sulfate and concentrated under reduced pressure. The crude product was purified by flash chromatography (petroleum ether/ethyl acetate=10/1) to give methyl 4-(4-fluorobenzyl)-3-oxohept-6-enoate (650 mg, 2.46 mmol, 77% yield) as yellow oil. LC-MS: m/z=265 [M+H]⁺, retention time 1.934 min (Method B).

### Methyl 2,2-diazido-4-(4-fluorobenzyl)-3-oxohept-6-enoate

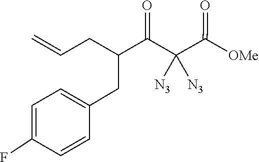 [SMILES: C=CCC(Cc1ccc(F)cc1)C(=O)C(N=[N+]=[N-])(N=[N+]=[N-])C(=O)OC]

A mixture of methyl 4-(4-fluorobenzyl)-3-oxohept-6-enoate (500.0 mg, 1.89 mmol), sodium azide (369.2 mg, 5.68 mmol), sodium hydrocarbonate (476.7 mg, 5.68 mmol) and iodine (984.3 mg, 3.88 mmol) in dimethylsulfoxide/water (20.0 mL/10.0 mL) was stirred at room temperature for 16.0 h. The reaction was quenched with saturated ammonium chloride solution and extracted ethyl acetate. The organic layer was separated, washed with brine, dried over sodium sulfate and concentrated under reduced pressure. The crude product was purified by flash chromatography (petroleum ether/ethyl acetate=10/1) to provide methyl 2,2-diazido-4-(4-fluorobenzyl)-3-oxohept-6-enoate (310 mg, 0.89 mmol, 47% yield) as yellow solid. LC-MS: m/z=369 [M+Na]+, retention time 2.279 min (Method A).

### Methyl 4-(4-fluorobenzyl)-3-hydroxy-6-methylpicolinate

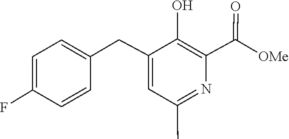 [SMILES: COC(=O)c1nc(C)cc(Cc2ccc(F)cc2)c1O]

A mixture of methyl 2,2-diazido-4-(4-fluorobenzyl)-3-oxohept-6-enoate (300.0 mg, 0.87 mmol) in toluene (5.0 mL) was stirred at 110° C. for 2.0 h in a sealed tube. The solution was cooled and concentrated to give dryness. The resulting residue was purified by flash chromatography (petroleum ether/ethyl acetate=10/3) to provide methyl 4-(4-fluorobenzyl)-3-hydroxy-6-methylpicolinate (64 mg, 0.23 mmol, 27% yield) as yellow oil. LC-MS: m/z=276 [M+H]⁺, retention time 2.006 min (Method A).

### 4-(4-Fluorobenzyl)-3-hydroxy-6-methylpicolinic Acid

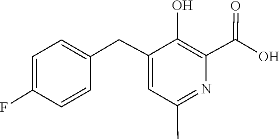 [SMILES: Cc1cc(Cc2ccc(F)cc2)c(O)c(C(=O)O)n1]

To a solution of methyl 4-(4-fluorobenzyl)-3-hydroxy-6-methylpicolinate (60.0 mg, 0.22 mmol) in methanol/water (4.0 mL/1.0 mL) was added lithium hydroxide monohydrate (91.5 mg, 2.18 mmol). The mixture was stirred overnight and concentrated to remove methanol. The resulting aqueous solution was acidified by adding 10% hydrochloric acid (5.0 mL) and extracted ethyl acetate twice. The organic layer was separated, washed with brine, dried over sodium sulfate and concentrated under reduced pressure. The crude 4-(4-fluorobenzyl)-3-hydroxy-6-methylpicolinic acid (34 mg, 0.13 mmol, 59% yield) as yellow solid. LC-MS: m/z=262 [M+H]⁺, retention time 1.626 min (Method A). The crude product was used to the next step.

### Ethyl (4-(4-fluorobenzyl)-3-hydroxy-6-methylpicolinoyl)glycinate

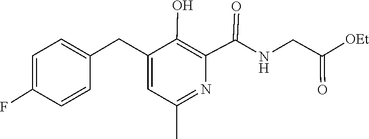 [SMILES: CCOC(=O)CNC(=O)c1nc(C)cc(Cc2ccc(F)cc2)c1O]

A mixture of 4-(4-fluorobenzyl)-3-hydroxy-6-methylpicolinic acid (30.0 mg, 0.11 mmol), ethyl glycinate hydrochloride (14.21 mg, 0.14 mmol), benzotriazole-1-yl-oxytripyrrolidinophosphonium hexafluorophosphate (65.73 mg, 0.13 mmol) and triethylamine (58.10 mg, 0.57 mmol) in dichloromethane (3.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=85/15) to provide ethyl (4-(4-fluorobenzyl)-3-hydroxy-6-methylpicolinoyl)glycinate (26 mg, 0.08 mmol, 68% yield) as yellow solid. LC-MS: m/z=347 [M+H]⁺, retention time 2.180 min (Method A).

### (4-(4-Fluorobenzyl)-3-hydroxy-6-methylpicolinoyl)glycine

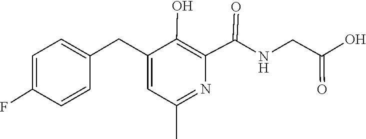 [SMILES: Cc1cc(Cc2ccc(F)cc2)c(O)c(C(=O)NCC(=O)O)n1]

To a solution of ethyl (4-(4-fluorobenzyl)-3-hydroxy-6-methylpicolinoyl)glycinate (26.00 mg, 0.08 mmol) in methanol/water (4.0 mL/1.0 mL) was added lithium hydroxide monohydrate (31.53 mg, 0.75 mmol). The mixture was stirred overnight and concentrated to remove methanol. The resulting aqueous solution was acidified with 10% hydrochloric acid solution to pH=3˜4. The precipitate was purified by reverse Prep-HPLC to provide (4-(4-fluorobenzyl)-3-hydroxy-6-methylpicolinoyl) glycine (Formate) (11.2 mg, 0.04 mmol, 47% yield) as white solid. LC-MS: m/z=319 [M+H]⁺, retention time 4.616 min (Method A). ¹H NMR (400 MHz, DMSO-d₆) δ 12.85 (br, 1H), 12.47 (s, 1H), 9.15-9.11 (m, 1H), 7.32-7.25 (m, 3H), 7.11 (t, J=8.8 Hz, 2H), 3.98 (d, J=6.4 Hz, 2H), 3.93 (s, 2H), 2.41 (s, 3H).

### Example 25: Preparation of Compound 25

### 3,5-Dibromo-2,4-dimethylpyridine 1-oxide

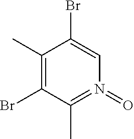

To a solution of 3,5-dibromo-2,4-dimethylpyridine (400 mg, 1.51 mmol) in dichloromethane (10.0 mL) was added 3-chloroperoxybenzoic acid (400 mg, 1.96 mmol, 85%) at 0° C. The mixture was stirred at room temperature for 18.0 h and potassium carbonate (400 mg, 3.20 mmol) was added. The mixture was stirred for another 1 h and insoluble solid was filtered. The filtrate was concentrated to obtain 3,5-dibromo-2,4-dimethylpyridine 1-oxide (400 mg, 1.43 mmol, 94% yield) as white solid. LC-MS: m/z=281.1 [M+H]⁺, retention time 1.47 min (Method A). The product was pure enough and used directly to the next step.

### 3,5-Dibromo-4,6-dimethylpicolinonitrile

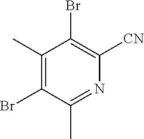

A mixture of 3,5-dibromo-2,4-dimethylpyridine 1-oxide (400 mg, 1.42 mmol), trimethylsilyl cyanide (2.0 mL) and triethylamine (2.0 mL) in acetonitrile (10.0 mL) was stirred at 85° C. for 24.0 h. The mixture was diluted with water and extracted with ethyl acetate. The organic layer was washed with brine, dried over sodium sulfate and concentrated under reduced pressure. The crude product was purified by flash chromatography (petroleum ether/ethyl acetate=10/1) to obtain 3,5-dibromo-4,6-dimethylpicolinonitrile (240 mg, 0.69 mmol, 46% yield) as yellow oil. LC-MS: m/z=291.2 [M+H]⁺, retention time 1.74 min (Method A).

### 3-Bromo-4,6-dimethyl-5-(1-phenyl-1H-pyrazol-4-yl)picolinonitrile

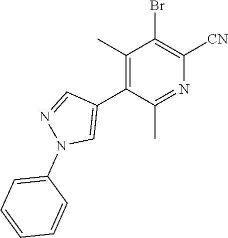 [SMILES: Cc1nc(C#N)c(Br)c(C)c1-c1cnn(-c2ccccc2)c1]

To a solution of 3,5-dibromo-4,6-dimethylpicolinonitrile (600 mg, 2.07 mmol), 1-phenyl-4-(4,4,5,5-tetramethyl-1,3,2-dioxaborolan-2-yl)-1H-pyrazole (559 mg 2.07 mmol) and potassium carbonate (343 mg, 2.48 mmol) was added [1,1′-bis(diphenylphosphino)ferrocene]dichloropalladium(II) (239 mg, 0.21 mmol) in N,N-dimethylformamide/water (3.0 mL/0.3 mL). The mixture was stirred at 45° C. under nitrogen for 16.0 h and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=2/1) to afford 3-bromo-4,6-dimethyl-5-(1-phenyl-1H-pyrazol-4-yl)picolinonitrile (95 mg, 0.27 mmol, 32% yield) as yellow solid. LC-MS: m/z=354.3 [M+H]+, retention time=1.909 min (Method A).

### 3-Hydroxy-4,6-dimethyl-5-(1-phenyl-1H-pyrazol-4-yl)picolinonitrile

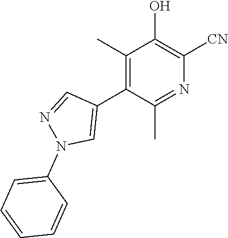 [SMILES: Cc1nc(C#N)c(O)c(C)c1-c1cnn(-c2ccccc2)c1]

A mixture of 3-bromo-4,6-dimethyl-5-(1-phenyl-1H-pyrazol-4-yl)picolinonitrile (90.0 mg, 0.25 mmol), potassium carbonate (106 mg, 0.76 mmol) and benzyl alcohol (270 mg, 2.5 mmol) in N,N-dimethylacetamide (3.0 mL) was stirred at 120° C. for 72.0 h. The mixture was cooled and evaporated to give dryness. The resulting residue was purified by reverse prep-HPLC to obtain 3-hydroxy-4,6-dimethyl-5-(1-phenyl-1H-pyrazol-4-yl)picolinonitrile (20 mg, 0.07 mmol, 27% yield) as white solid. LC-MS: m/z=291.1 [M+H]+, retention time=1.40 min (Method A).

### 3-Hydroxy-4,6-dimethyl-5-(1-phenyl-1H-pyrazol-4-yl)picolinic Acid

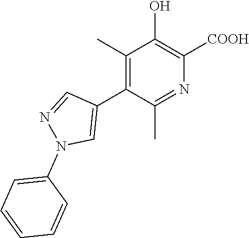 [SMILES: Cc1nc(C(=O)O)c(O)c(C)c1-c1cnn(-c2ccccc2)c1]

To a solution of 3-hydroxy-4,6-dimethyl-5-(1-phenyl-1H-pyrazol-4-yl)picolinonitrile (20 mg, 0.07 mmol) in ethanol (5.0 mL) was added 30% aqueous sodium hydroxide (1.0 mL). The mixture was stirred at 100° C. for 3.0 h, cooled and concentrated to remove ethanol. The resulting aqueous solution was acidified with 10% hydrochloric acid solution to pH=3˜4. The precipitate was filtered and dried to afford 3-hydroxy-4,6-dimethyl-5-(1-phenyl-1H-pyrazol-4-yl)picolinic acid (25 mg, crude) as white solid. LC-MS: m/z=310.4 [M+H]⁺, retention time 1.74 min (Method A). The product was pure enough and used directly to the next step.

### Ethyl (3-hydroxy-4,6-dimethyl-5-(1-phenyl-1H-pyrazol-4-yl)picolinoyl)glycinate

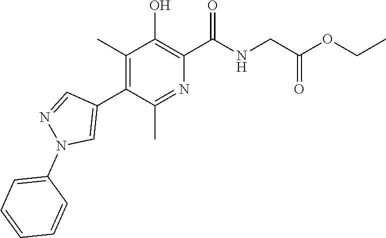 [SMILES: CCOC(=O)CNC(=O)c1nc(C)c(-c2cnn(-c3ccccc3)c2)c(C)c1O]

A mixture of 3-hydroxy-4,6-dimethyl-5-(1-phenyl-1H-pyrazol-4-yl)picolinic acid (25 mg, 0.08 mmol), ethyl glycinate hydrochloride (17 mg, 0.12 mmol), benzotriazole-1-yl-oxytripyrrolidinophosphonium hexafluorophosphate (50 mg, 0.10 mmol) and triethylamine (380 mg, 3.75 mmol) in dichloromethane (8.0 mL) was stirred at room temperature overnight. The reaction was quenched with water and extracted with dichloromethane. The organic layer was separated, washed with brine, dried over sodium sulfate and concentrated under reduced pressure. The crude product was purified by flash chromatography (petroleum ether/ethyl acetate=1/2) to obtain ethyl (3-hydroxy-4,6-dimethyl-5-(1-phenyl-1H-pyrazol-4-yl)picolinoyl)glycinate (20 mg, 0.05 mmol, 63% yield) as white solid. LC-MS: m/z=395.1 [M+H]⁺, retention time 2.13 min (Method A).

### (3-Hydroxy-4,6-dimethyl-5-(1-phenyl-1H-pyrazol-4-yl)picolinoyl)glycine

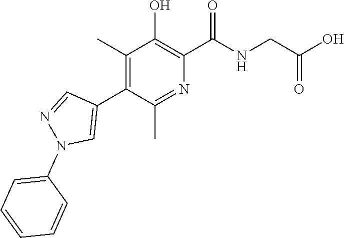 [SMILES: Cc1nc(C(=O)NCC(=O)O)c(O)c(C)c1-c1cnn(-c2ccccc2)c1]

To a solution of ethyl (3-hydroxy-4,6-dimethyl-5-(1-phenyl-1H-pyrazol-4-yl)picolinoyl)glycinate (20 mg, 0.05 mmol) in methanol/water (6.0 mL/3.0 mL) was added lithium hydroxide monohydrate (164 mg, 4.0 mmol). The mixture was stirred overnight and concentrated to remove methanol. The resulting aqueous solution was acidified with 10% hydrochloric acid solution to pH=3˜4. The precipitate was filtered, washed with water and dried to obtain (3-hydroxy-4,6-dimethyl-5-(1-phenyl-1H-pyrazol-4-yl)picolinoyl)glycine (6.4 mg, 0.017 mmol, 36% yield) as white solid. LC-MS: m/z=367.0 [M+H]⁺, retention time 4.81 min (Method A). ¹HNMR (400 MHz, DMSO-d₆) δ 12.49 (s, 1H), 9.18 (t, J=6.0 Hz, 1H), 8.73 (s, 1H), 8.01-7.81 (m, 3H), 7.54 (t, J=7.9 Hz, 2H), 7.35 (t, J=7.4 Hz, 1H), 4.02 (d, J=6.1 Hz, 2H), 2.39 (s, 3H), 2.12 (s, 3H).

### Example 26: Preparation of Compound 26

### 3,5-dichloro-4-methylpyridine 1-oxide

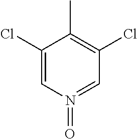

To a solution of 3,5-dichloro-4-methylpyridine (9.4 g, 57.7 mmol) in DCM (150 mL) at RT was added m-CPBA (12.9 g, 74.7 mmol) portion wise over 2 mins. The reaction was stirred at RT overnight. After the reaction was completed as indicated by TLC analysis, the reaction was added K₂CO₃ (12 g, 87 mmol) in one portion and stirred at RT for about 2 hrs. After the resulting suspension was filtered, the filtrate was concentrated to dryness. The residue was slurried in a mixed solvent (PE:EtOAc=50:1, 50 mL) to afford 7.4 g of the title compound. LC-MS (ESI+): m/z 178(M+H)⁺;

### 3,5-dichloro-4-methylpicolinonitrile

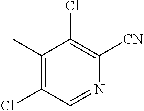

To a solution of 3,5-dichloro-4-methylpyridine 1-oxide (8 g, 44.94 mmol) in MeCN (150 mL) at room temperature was added TMS-CN (9 g, 89.88 mmol) and TEA (9.4 mL). The reaction was refluxed overnight. After the reaction was completed as indicated by TLC analysis, the reaction was quenched with brine (150 mL) and extracted with EA (150 mL×3). The combined organic phase was washed with brine, dried with anhydrous Na₂SO₄ (60 g), filtered and concentrated in vacuo. The residue was purified by silica column chromatography (PE:EtOAc=10:1) to give 6.8 g of the title compound. ¹H-NMR (300 MHz, CDCl₃) δ 8.52 (s, 1H), 2.57 (s, 3H)).

### 3-chloro-4-methyl-5-(4,4,5,5-tetramethyl-1,3,2-dioxaborolan-2-yl)picolinonitrile

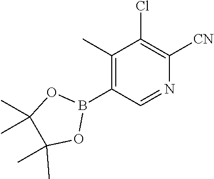 [SMILES: Cc1c(B2OC(C)(C)C(C)(C)O2)cnc(C#N)c1Cl]

Under nitrogen protection, a mixture of 3,5-dichloro-4-methylpicolinonitrile (3.72 g, 20 mmol), B₂Pin₂ (7.62 g, 30 mmol), Pd(dppf)Cl₂ (1.4 g, 2 mmol) and KOAc (5.88 g, 60 mmol) in dioxane (100 mL) was stirred at 100° C. overnight. After the reaction was completed as indicated by TLC, the resulting suspension was filtered. The filter cake was washed with EtOAc (200 mL), the combined filtrates were concentrated to dryness. The resulting residue was dissolved in an aqueous NaOH solution (60 mL,1N) and stirred for half hr. The aqueous solution was washed with EtOAc (100 mL). After separation, the organic phase was treated with an aqueous NaOH solution (30 mL,1N) again. After separation, the combined aqueous phase was acidified to pH=4˜5 with a diluted HCl solution (2N) and a large amount of solid was precipitated. The suspension was filtered to give 407 mg of the title compound. ¹H-NMR (300 MHz, CDCl₃) δ 8.68 (s, 1H), 2.61 (s, 3H), 1.34 (s, 12H).

### 3-chloro-4-methyl-5-(2-phenyloxazol-5-yl)picolinonitrile

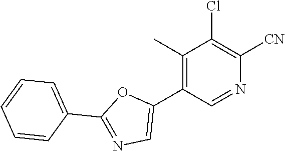 [SMILES: Cc1c(-c2cnc(-c3ccccc3)o2)cnc(C#N)c1Cl]

Under nitrogen protection, a mixture of 3-chloro-4-methyl-5-(4,4,5,5-tetramethyl-1,3,2-dioxaborolan-2-yl)picolinonitrile (270 mg, 0.97 mmol), 5-bromo-2-phenyloxazole (280 mg, 1.2 mmol), Pd(PPh₃)₄(138 mg, 0.12 mmol) and K₃PO₄·H₂O (958 g, 3.6 mmol) in dioxane (7 mL) and H₂O (0.4 mL) was stirred at 1000° C. overnight. After the reaction was completed as indicated by TLC analysis, the reaction was quenched with H₂O (10 mL) and extracted with DCM (25 mL×3). The combined organic phase was washed with brine, dried with anhydrous Na₂SO₄ (30 g), filtered and concentrated in vacuo. The residue was purified by silica column chromatography (DCM) to give 250 mg of the title compound. LC-MS (ESI+): m/z 296 (M+H)⁺;

### 3-(benzyloxy)-4-methyl-5-(2-phenyloxazol-5-yl)picolinonitrile

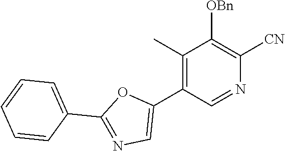 [SMILES: Cc1c(-c2cnc(-c3ccccc3)o2)cnc(C#N)c1OCc1ccccc1]

To a solution of 3-chloro-4-methyl-5-(2-phenyloxazol-5-yl)picolinonitrile (250 mg, 0.85 mmol) in THF (12.5 mL) at RT was added BnOH (184 mg, 1.7 mmol) and t-BuOK (184 mg, 1.7 mmol). The reaction was stirred at 400° C. for 3 hr. After the reaction was completed as indicated by TLC analysis, the reaction was quenched with H₂O (20 mL) and extracted with DCM (10 mL×3). The combined organic phase was washed with brine, dried with anhydrous Na₂SO₄ (5 g), filtered and concentrated in vacuo. The residue was purified by silica column chromatography (PE/EtOAc=5/1) to give 157 mg of the title compound. LC-MS (ESI+): m/z 368 (M+H)⁺; ¹H-NMR (300 MHz, CDCl₃) δ 8.84 (s, 1H), 8.14-8.11 (m, 2H), 7.54-7.50 (m, 6H), 7.44-7.26 (m, 3H), 2.25 (s, 2H), 2.44 (s, 3H).

### 3-(benzyloxy)-4-methyl-5-(2-phenyloxazol-5-yl)picolinic Acid

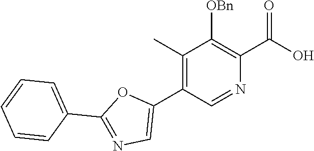 [SMILES: Cc1c(-c2cnc(-c3ccccc3)o2)cnc(C(=O)O)c1OCc1ccccc1]

To a solution of 3-(benzyloxy)-4-methyl-5-(2-phenyloxazol-5-yl)picolinonitrile (167 mg, 0.45 mmol) in EtOH (15 mL) at RT was added an aqueous NaOH solution (1.6 mL, 30 wt %). The reaction was stirred at reflux temperature overnight. After the reaction was completed as indicated by LCMS analysis, the reaction was cooled to RT and acidified to pH 4-5 with a diluted HCl solution. A large amount of solid was precipitated. The suspension was filtered to give 158 mg of the title compound. LC-MS (ESI+): m/z 387 (M+H)⁺;

### Methyl (3-(benzyloxy)-4-methyl-5-(2-phenyloxazol-5-yl)picolinoyl)glycinate

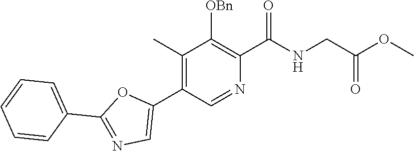 [SMILES: COC(=O)CNC(=O)c1ncc(-c2cnc(-c3ccccc3)o2)c(C)c1OCc1ccccc1]

To a solution of 3-(benzyloxy)-4-methyl-5-(2-phenyloxazol-5-yl)picolinic acid (158 mg, 0.41 mmol) in DMF (5 mL) at RT was added methyl glycinate hydrochloride (62 mg, 0.49 mmol), DIEA (212 mg, 1.64 mmol) and HATU (234 mg, 0.45 mmol). The reaction was stirred at RT overnight. After the reaction was completed as indicated by TLC analysis, the reaction was quenched with H₂O (20 mL) and extracted with DCM (10 mL×3). The combined organic phase was washed with brine, dried with anhydrous Na₂SO₄ (5 g), filtered and concentrated in vacuo. The residue was purified by silica column chromatography (PE/DCM=1/1) to give 115 mg of the title compound. LC-MS (ESI+): m/z 458 (M+H)⁺; ¹H-NMR (300 MHz, CDCl₃) δ 8.77 (s, 1H), 8.37 (brs, 1H), 8.14-8.11 (m, 2H), 7.55-7.49 (m, 6H), 7.42-7.35 (m, 3H), 5.13 (s, 2H), 4.29 (d, J=6 Hz, 2H), 3.81 (s, 3H), 2.44 (s, 3H).

### Methyl (3-hydroxy-4-methyl-5-(2-phenyloxazol-5-yl)picolinoyl)glycinate

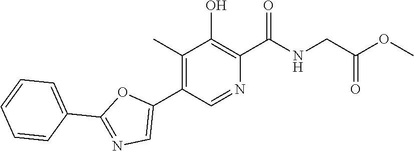 [SMILES: COC(=O)CNC(=O)c1ncc(-c2cnc(-c3ccccc3)o2)c(C)c1O]

To a solution of methyl (3-(benzyloxy)-4-methyl-5-(2-phenyloxazol-5-yl)picolinoyl)glycinate (167 mg, 0.45 mmol) in MeOH (6 mL) and DCM (3 mL) at RT was added Pd/C (10 wt %, 34 mg). The reaction was stirred under atmospheric hydrogen pressure from a balloon for 45 mins. After the reaction was completed as indicated by TLC analysis, the reaction was filtered through a package of Celite. The filtrate was concentrated in vacuo to give 80 mg of the title compound. ¹H-NMR (300 MHz, CDCl₃) δ 12.27 (s, 1H), 8.47 (s, 2H), 8.14-8.11 (m, 2H), 7.51 (d, J=3.6 Hz, 4H), 4.27 (d, J=6 Hz, 2H), 3.83 (s, 3H), 2.50 (s, 3H).

### (3-hydroxy-4-methyl-5-(2-phenyloxazol-5-yl)picolinoyl)glycine

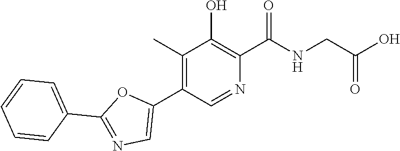 [SMILES: Cc1c(-c2cnc(-c3ccccc3)o2)cnc(C(=O)NCC(=O)O)c1O]

To a solution of methyl (3-hydroxy-4-methyl-5-(2-phenyloxazol-5-yl)picolinoyl)glycinate (80 mg, 0.22 mmol) in THF (5 mL) and H₂O (2 mL) at RT was added LiOH. H₂O (92 mg, 2.2 mmol). The reaction was stirred at 50° C. for 2 hr. After the reaction was completed as indicated by TLC analysis, the reaction was acidified to pH 4˜5 with a diluted HCl solution (1N). A large amount of solid was precipitated. The suspension was filtered to give 45 mg of the title compound. HPLC purity was 99.1%; LC-MS (ESI+): m/z 354 (M+H)⁺; ¹H-NMR (300 MHz, DMSO-d₆) δ 12.95 (s, 1H), 12.85 (brs, 1H), 9.38 (t, J=6.0 Hz, 1H), 8.61 (s, 1H), 8.15-8.12 (m, 2H), 7.95 (s, 1H), 7.64-7.58 (m, 3H), 4.03 (d, J=6.0 Hz, 2H), 2.67 (s, 3H).

### Example 27: Preparation of Compound 27

### 5-chloro-4,6′-dimethyl-[3,3′-bipyridine]-6-carbonitrile

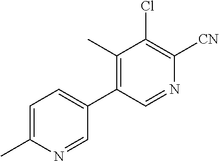 [SMILES: Cc1ccc(-c2cnc(C#N)c(Cl)c2C)cn1]

The compound was synthesized according to the procedure for the preparation of 3-chloro-4-methyl-5-(2-phenyloxazol-5-yl)picolinonitrile using 5-bromo-2-methylpyridine. LC-MS (ESI+): m/z 244 (M+H)⁺.

### 5-(benzyloxy)-4,6′-dimethyl-[3,3′-bipyridine]-6-carbonitrile

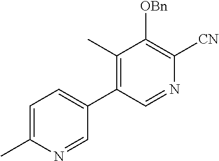 [SMILES: Cc1ccc(-c2cnc(C#N)c(OCc3ccccc3)c2C)cn1]

The compound was synthesized according to the procedure for the preparation of 3-(benzyloxy)-4-methyl-5-(2-phenyloxazol-5-yl)picolinonitrile. LC-MS (ESI+): m/z 316 (M+H)⁺; ¹H-NMR (300 MHz, CDCl₃) δ 8.45 (d, J=1.8 Hz, 1H), 8.30 (s, 1H), 7.55-7.48 (m, 3H), 7.55-7.42 (m, 3H), 7.37-7.28 (m, 1H), 5.28 (s, 2H), 2.65 (s, 3H), 2.17 (s, 3H).

### 5-(benzyloxy)-4,6′-dimethyl-[3,3′-bipyridine]-6-carboxylic Acid

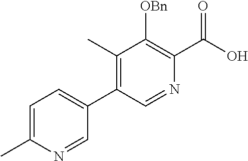 [SMILES: Cc1ccc(-c2cnc(C(=O)O)c(OCc3ccccc3)c2C)cn1]

The compound was synthesized according to the procedure for the preparation of 3-(benzyloxy)-4-methyl-5-(2-phenyloxazol-5-yl)picolinic acid. LC-MS (ESI+): m/z 335 (M+H)⁺; ¹H-NMR (300 MHz, DMSO-d₆) δ 13.45 (brs, 1H), 8.53 (d, J=1.8 Hz, 1H), 8.28 (s, 1H), 7.81 (dd, J=7.8, 2.1 Hz, 1H), 5.03 (s, 2H), 2.55 (s, 3H), 2.15 (s, 3H).

### methyl (5-(benzyloxy)-4,6′-dimethyl-[3,3′-bipyridine]-6-carbonyl)glycinate

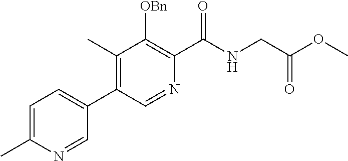

The compound was synthesized according to the procedure for the preparation of Methyl (3-(benzyloxy)-4-methyl-5-(2-phenyloxazol-5-yl)picolinoyl)glycinate. LC-MS (ESI+): m/z 406 (M+H)⁺;

### methyl (5-hydroxy-4,6′-dimethyl-[3,3′-bipyridine]-6-carbonyl)glycinate

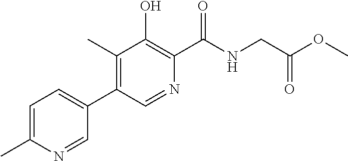

The compound was synthesized according to the procedure for the preparation of Methyl (3-hydroxy-4-methyl-5-(2-phenyloxazol-5-yl)picolinoyl)glycinate. LC-MS (ESI+): m/z 316 (M+H)⁺; ¹H-NMR (300 MHz, CDCl₃) δ 12.16 (s, 1H), 8.49 (d, J=1.8 Hz, 2H), 7.96 (s, 1H), 7.56 (dd, J=8.1 Hz, 2.1 Hz, 1H), 7.29 (s, 1H), 4.26 (d, J=5.7 Hz, 2H), 3.82 (s, 3H), 2.64 (s, 3H), 2.23 (s, 3H).

### (5-hydroxy-4,6′-dimethyl-[3,3′-bipyridine]-6-carbonyl)glycine

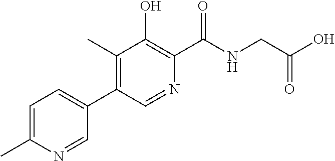 [SMILES: Cc1ccc(-c2cnc(C(=O)NCC(=O)O)c(O)c2C)cn1]

The compound was synthesized according to the procedure for the preparation of (3-hydroxy-4-methyl-5-(2-phenyloxazol-5-yl)picolinoyl)glycine. LC-MS (ESI+): m/z 302 (M+H)⁺; ¹H-NMR (300 MHz, DMSO-d₆) δ 12.80 (s, 1H), 9.38 (t, J=6 Hz, 1H), 8.53 (d, J=1.8 Hz, 1H), 7.82-7.79 (m, 1H), 7.42 (d, J=8.1 Hz, 1H), 4.01 (d, J=6.6 Hz, 2H), 2.55 (s, 3H), 2.16 (s, 3H).

### Example 28: Preparation of Compound 28

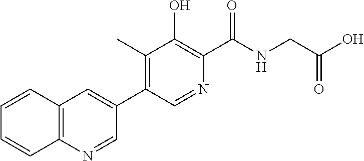 [SMILES: Cc1c(-c2cnc3ccccc3c2)cnc(C(=O)NCC(=O)O)c1O]

The compound was synthesized according to the procedure for the preparation of (3-hydroxy-4-methyl-5-(2-phenyloxazol-5-yl)picolinoyl)glycine. LC-MS (ESI+): m/z 338 (M+H)⁺; ¹H-NMR (300 MHz, DMSO-d₆) δ 12.86 (s, 1H), 12.80 (brs, 1H), 9.45 (t, J=6.Hz, 1H), 9.00 (d, J=2.1 Hz, 1H), 8.53 (s, 1H), 8.23 (s, 1H), 8.10 (t, J=8.1 Hz, 2H), 7.88-7.73 (m, 1H), 7.71-7.68 (m, 1H), 4.03 (d, J=6 Hz, 2H), 2.50 (s, 3H).

### Example 29: Preparation of Compound 29

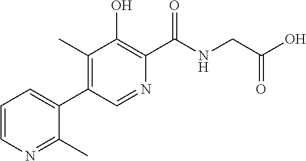 [SMILES: Cc1ncccc1-c1cnc(C(=O)NCC(=O)O)c(O)c1C]

The compound was synthesized according to the procedure for the preparation of (3-hydroxy-4-methyl-5-(2-phenyloxazol-5-yl)picolinoyl)glycine. LC-MS (ESI+): m/z 302 (M+H)⁺; ¹H-NMR (300 MHz, DMSO-d₆) δ 12.80 (s, 1H), 9.39 (t, J=6.5 Hz, 1H), 8.55 (dd, J=4.9, 1.8 Hz, 1H), 7.99 (s, 1H), 7.62 (dd, J=7.6, 1.8 Hz, 1H), 7.36 (dd, J=7.7, 4.9 Hz, 1H), 4.00 (d, J=6.0 Hz, 2H), 2.25 (s, 3H), 1.96 (s, 3H).

### Example 30: Preparation of Compound 30

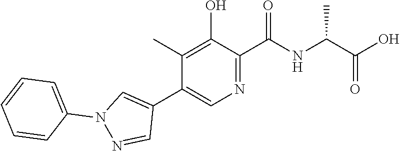 [SMILES: Cc1c(-c2cnn(-c3ccccc3)c2)cnc(C(=O)N[C@H](C)C(=O)O)c1O]

The compound was synthesized according to the procedure for the preparation of (3-hydroxy-4-methyl-5-(2-phenyloxazol-5-yl)picolinoyl)glycine. LC-MS (ESI+): m/z 367 (M+H)⁺; ¹H-NMR (300 MHz, DMSO-d₆) δ 12.88 (brs, 1H), 12.78 (s, 1H), 9.17 (d, J=7.7 Hz, 1H), 8.92 (s, 1H), 8.32 (s, 1H), 8.18 (s, 1H), 7.94 (d, J=8.0 Hz, 2H), 7.55 (t, J=7.8 Hz, 2H), 7.37 (t, J=7.4 Hz, 1H), 4.51 (t, J=7.2 Hz, 1H), 2.37 (s, 3H), 1.47 (d, J=7.2 Hz, 3H).

### Example 31: Preparation of Compound 31

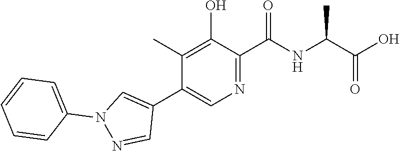 [SMILES: Cc1c(-c2cnn(-c3ccccc3)c2)cnc(C(=O)N[C@@H](C)C(=O)O)c1O]

The compound was synthesized according to the procedure for the preparation of (3-hydroxy-4-methyl-5-(2-phenyloxazol-5-yl)picolinoyl)glycine. LC-MS (ESI+): m/z 367 (M+H)⁺; ¹H-NMR (300 MHz, DMSO-d₆) δ 12.90 (brs, 1H), 12.77 (s, 1H), 9.17 (d, J=7.6 Hz, 1H), 8.93 (s, 1H), 8.33 (s, 1H), 8.18 (s, 1H), 7.94 (d, J=8.0 Hz, 2H), 7.55 (t, J=7.8 Hz, 2H), 7.37 (t, J=7.4 Hz, 1H), 4.51 (t, J=7.4 Hz, 1H), 2.37 (s, 3H), 1.47 (d, J=7.2 Hz, 3H).

### Example 32: Preparation of Compound 32

### 3,5-dichloro-4-methylpyridine 1-oxide

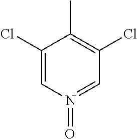

3,5-dichloro-4-methyl-pyridine (5 g, 30.86 mmol) was dissolved in DCM (70 mL) and cooled to 0° C. m-CPBA (7.85 g, 40.11 mmol) was added lot wise at 0° C. The reaction mixture was stirred at RT overnight. LCMS indicated complete conversion to desired product (MS/178). K₂CO₃ (4.427 g, 32.08 mmol) was added. The turbid reaction mixture was stirred for 1 h. The white precipitate was filtered through a pad of Celite, filter cake was rinsed with DCM (50 mL). The filtrate was concentrated to give a white solid (5.0 g). LC-MS (ESI+): m/z 179.0 (M+H)⁺.

### 3,5-dichloro-4-methylpicolinonitrile

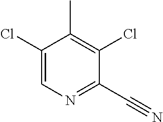

A mixture of 3,5-dichloro-4-methyl-pyridine 1-oxide (5 g, 28.087 mmol), TMS-cyanide (5 g, 50.38 mmol) and triethylamine (5.8 mL, 42.29 mmol) in acetonitrile (90 mL) was heated to reflux (85° C.) for 7 h. The reaction was cooled to RT and stirred overnight. The reaction was quenched with aqueous NaHCO₃ solution (50 mL). The mixture was diluted with ethyl acetate (100 mL). The layers were separated, organic layer was washed with brine (50 mL), dried over Na₂SO₄ and concentrated. The crude was purified by column (12 g column, 0-100% ethyl acetate in hexanes) afford pale brown liquid (4.09 g). LC-MS (ESI+): m/z 186.0 (M+H)⁺.

### 3-chloro-5-(3-chlorophenyl)-4-methylpicolinonitrile

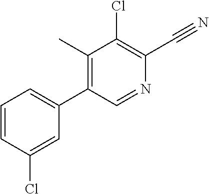 [SMILES: Cc1c(-c2cccc(Cl)c2)cnc(C#N)c1Cl]

Pd(dppf)Cl₂ (0.178 g, 0.243 mmol) was added to a stirred mixture of 3,5-dichloro-4-methyl-pyridine-2-carbonitrile (1.2 g, 6.416 mmol), (3-chlorophenyl) boronic acid (1 g, 6.416 mmol) and K₂CO₃ ((1.06 g, 7.699 mmol) in DMF (10 mL) and 1 mL water at rt. The resulting mixture was heated to 45° C. under nitrogen for 17 h. TLC (30% EtOAc in hexanes) indicated 90% consumption of starting material occurred. The reaction mixture was diluted with water (20 mL) and ethyl acetate (30 mL), stirred well. The layers were separated, organic layer was washed with brine (20 mL), dried over Na₂SO₄ and concentrated. The crude was purified by column (24 g column, 0-50% ethyl acetate in hexanes) afford product white solid. 1H-NMR indicated a 3:8 mixture of regioisomers. The mixture was subjected to the next reaction. LC-MS (ESI+): m/z 263.0 (M+H)⁺.

### 3-(benzyloxy)-5-(3-chlorophenyl)-4-methylpicolinonitrile

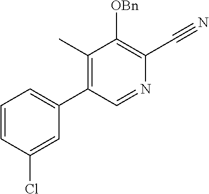 [SMILES: Cc1c(-c2cccc(Cl)c2)cnc(C#N)c1OCc1ccccc1]

4-chloro-6-(3-chlorophenyl)-5-methyl-pyridine-3-carbonitrile (1.35 g, 5.130 mmol) was dissolved in DMF (10 mL), reaction mixture cooled to 0° C. NaH (60% suspension in mineral oil, 246 mg, 6.156 mmol) was added. After 3 minutes, benzyl alcohol (0.637 mL, 6.156 mmol) was added drop wise at 0° C. The red reaction was stirred at 0° C. After 50 min, TLC (10% ethyl acetate in hexanes) showed 90% conversion. The reaction was quenched with water (20 mL). The mixture was extracted with ethyl acetate (30 mL). The organic layer was dried over Na₂SO₄ and concentrated. The crude was purified by column (20 g, 0-40% ethyl acetate in hexanes), two regioisomers (from previous reaction) were separated. Major product (1.16 g, 67%). The major product displayed a strong NOE between 8.40 ppm and 7.57-7.42 ppm, indicating desired product. LC-MS (ESI+): m/z 335.0 (M+H)⁺.

### 3-(benzyloxy)-5-(3-chlorophenyl)-4-methylpicolinic acid

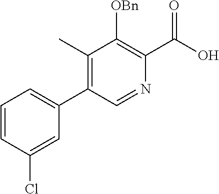 [SMILES: Cc1c(-c2cccc(Cl)c2)cnc(C(=O)O)c1OCc1ccccc1]

A mixture of 4-benzyloxy-6-(3-chlorophenyl)-5-methyl-pyridine-3-carbonitrile (1.1 g, 3.3 mmol) in ethanol (11 mL) and 30% aqueous NaOH solution (14 mL) was heated to 100° C. for 3 h. LCMS indicated complete conversion to desired high polar. The reaction was cooled to rt. Ethanol was evaporated under reduced pressure. The aqueous residue was acidified with conc HCl (pH-2), pale brown solid was crashed out. The solid was separated by filtration, washed with water and dried in air, affording a pale brown solid (1.2 g, 100%). The crude material was used in the next reaction. LC-MS (ESI+): m/z 354.0 (M+H)⁺.

### ethyl (3-(benzyloxy)-5-(3-chlorophenyl)-4-methylpicolinoyl)glycinate

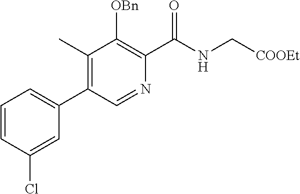 [SMILES: CCOC(=O)CNC(=O)c1ncc(-c2cccc(Cl)c2)c(C)c1OCc1ccccc1]

PyBOP (323 mg, 0.621 mmol) and triethylamine (0.39 mL, 2.862 mmol) were added to a stirred mixture of 4-benzyloxy-6-(3-chlorophenyl)-5-methyl-pyridine-3-carboxylic acid (200 mg, 0.565 mmol) and ethyl 2-aminoacetate; hydrochloride (79 mg, 0.565 mmol) in DCM (5 mL) at rt. The resulting mixture was stirred at RT for 2 h. LCMS indicated complete consumption of starting material occurred. The reaction was concentrated directly. The crude was purified by column (0-100% ethyl acetate in hexanes), affording white solid (201 mg, 81%). LC-MS (ESI+): m/z 439.0 (M+H)⁺.

### ethyl (5-(3-chlorophenyl)-3-hydroxy-4-methylpicolinoyl)glycinate

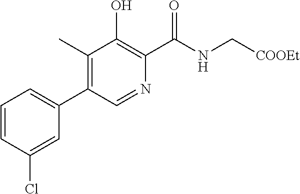 [SMILES: CCOC(=O)CNC(=O)c1ncc(-c2cccc(Cl)c2)c(C)c1O]

A stirred mixture of ethyl 2-[[4-benzyloxy-6-(3-chlorophenyl)-5-methyl-pyridine-3-carbonyl]amino]acetate (201 mg, 0.458 mmol) and 10% Pd-carbon (5 mg) in methanol (1 mL) and ethyl acetate (1 mL) was degassed with hydrogen for 2 min and then stirred at RT under hydrogen atmosphere for 12 h. LCMS indicated complete conversion to desired product. The reaction was filtered through a pad of Celite, rinsed with ethyl acetate (15 mL). The filtrate was concentrated. The crude product (151 mg, 0.429 mmol) was subjected to the next reaction without further purification. LC-MS (ESI+): m/z 349.0 (M+H)⁺.

### (5-(3-chlorophenyl)-3-hydroxy-4-methylpicolinoyl)glycine

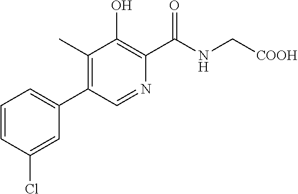 [SMILES: Cc1c(-c2cccc(Cl)c2)cnc(C(=O)NCC(=O)O)c1O]

Aqueous 1N NaOH (0.5 mL) was added to a stirred solution of ethyl 2-[[5-(3-chlorophenyl)-3-hydroxy-4-methyl-pyridine-2-carbonyl]amino]acetate (132 mg, 0.378 mmol) in THF (1.5 mL) and methanol (1.5 mL) at rt. The reaction was stirred at 40° C. for 1.5 h. The reaction was concentrated directly to remove THF and methanol. The residue was acidified by aq. 1 N HCl (0.6 mL). The precipitate was filtered, washed with water, and dried in air. The crude was purified by prep HPLC (30-80% MeCN in water over 25 min). LC-MS (ESI+): m/z 321.0 (M+H)⁺; 1H NMR (400 MHz, DMSO) δ 12.79 (s, 2H), 9.38 (t, J=6.1 Hz, 1H), 8.04 (d, J=4.7 Hz, 1H), 7.60-7.46 (m, 3H), 7.46-7.35 (m, 1H), 4.00 (d, J=6.2 Hz, 2H), 2.14 (s, 3H).

### Example 33: Preparation of Compound 33

### 3-chloro-4-methyl-5-(naphthalen-2-yl)picolinonitrile

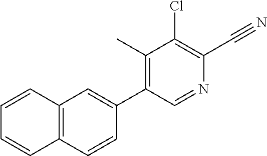 [SMILES: Cc1c(-c2ccc3ccccc3c2)cnc(C#N)c1Cl]

Pd(dppf)Cl₂ (0.032 g, 0.0248 mmol) was added to a stirred mixture of 3,5-dichloro-4-methyl-pyridine-2-carbonitrile (0.4 g, 2.14 mmol), 2-naphthylboronic acid (368 mg, 2.14 mmol) and K₂CO₃ ((354 mg, 2.57 mmol) in DMF (8 mL) and water (0.8 mL) at rt. The resulting mixture was heated to 45° C. under nitrogen for 17 h. TLC (30% ethyl acetate in hexanes) indicated complete consumption of starting material occurred. The reaction was diluted with ethyl acetate (20 mL) and water (15 mL). The layers were separated. The organic layer was washed with brine (10 mL), dried over Na₂SO₄ and concentrated. The crude was purified by column (24 g column, 0-50% ethyl acetate in hexanes), affording a mixture of two regioisomers as a white solid (520 mg, 87%). The mixture was subjected to the next reaction. LC-MS (ESI+): m/z 279.0 (M+H)⁺.

### 3-(benzyloxy)-4-methyl-5-(naphthalen-2-yl)picolinonitrile

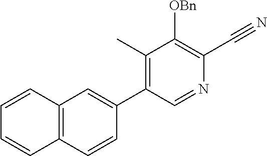 [SMILES: Cc1c(-c2ccc3ccccc3c2)cnc(C#N)c1OCc1ccccc1]

3-chloro-4-methyl-5-(2-naphthyl)pyridine-2-carbonitrile (520 mg, 1.87 mmol) was dissolved in DMF (10 mL) and cooled to 0° C. NaH (60% suspension in mineral oil, 90 mg, 2.24 mmol) was added at 0° C. and stirred for 3 min. Benzyl alcohol (0.23 mL, 2.24 mmol) was added at 0° C. The reaction mixture was warmed to RT and stirred for 30 min. The reaction was quenched with aq. NH₄Cl solution (50 mL) and the mixture was extracted with ethyl acetate (20 mL×3). The organic layer was washed with brine (20 mL), dried over Na₂SO₄ and concentrated. The crude was purified by column (20 g, 0-50% ethyl acetate in hexanes), affording the product (344 mg, 52%) as white solid. LC-MS (ESI+): m/z 351.0 (M+H)⁺.

### 3-(benzyloxy)-4-methyl-5-(naphthalen-2-yl)picolinic acid

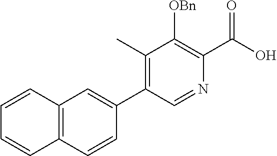 [SMILES: Cc1c(-c2ccc3ccccc3c2)cnc(C(=O)O)c1OCc1ccccc1]

A mixture of 3-benzyloxy-4-methyl-5-(2-naphthyl)pyridine-2-carbonitrile (344 mg, 0.98 mmol) and 30% aqueous NaOH solution (4.1 mL, 9 mmol) in ethanol (3.4 mL) was heated to 100° C. for 3 h. The reaction progress was monitored by LCMS. The reaction was concentrated directly to remove ethanol. The mixture was acidified with concentrated HCl (4 mL). The solid was removed by filtration, washed with water and dried in air to afford a yellow solid (360 mg, 100%). The crude was subjected to the next reaction without further purification. LC-MS (ESI+): m/z 370.0 (M+H)⁺.

### ethyl (3-(benzyloxy)-4-methyl-5-(naphthalen-2-yl)picolinoyl)glycinate

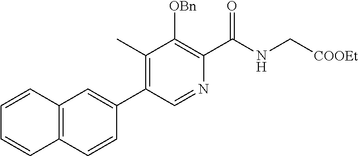 [SMILES: CCOC(=O)CNC(=O)c1ncc(-c2ccc3ccccc3c2)c(C)c1OCc1ccccc1]

Triethylamine (0.7 mL, 5.01 mmol) was added to a mixture of 3-benzyloxy-4-methyl-5-(2-naphthyl)pyridine-2-carboxylic acid (185 mg, 0.50 mmol), ethyl 2-aminoacetate hydrochloride (0.7512 mmol) and PyBOP (390 mg, 0.7512 mmol) in DCM (5 mL). The reaction mixture was stirred at rt. The reaction progress was monitored by LCMS. The reaction was concentrated directly and the crude material was purified by column (4 g column, 0-100% ethyl acetate in hexanes), affording the product as a viscus oil (132 mg, 58%). LC-MS (ESI+): m/z 455.0 (M+H)⁺.

### ethyl (3-hydroxy-4-methyl-5-(naphthalen-2-yl)picolinoyl)glycinate

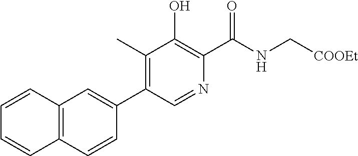 [SMILES: CCOC(=O)CNC(=O)c1ncc(-c2ccc3ccccc3c2)c(C)c1O]

A mixture of ethyl 2-[[3-benzyloxy-4-methyl-5-(2-naphthyl)pyridine-2-carbonyl]amino]acetate (132 mg, 0.2904 mmol) and 10% Pd on carbon (5 mg) in EtOAc (1 mL) and methanol (1 mL) was degassed with hydrogen for 3 min and then stirred under hydrogen at RT for 6 h. The reaction was diluted with ethyl acetate (20 mL) and filtered. The filtrate was concentrated to afford crude product (100 mg, 100%) as a brown oil. The crude was used in the next reaction without further purification. LC-MS (ESI+): m/z 365.0 (M+H)⁺.

### (3-hydroxy-4-methyl-5-(naphthalen-2-yl)picolinoyl)glycine

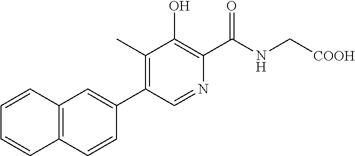 [SMILES: Cc1c(-c2ccc3ccccc3c2)cnc(C(=O)NCC(=O)O)c1O]

A mixture of crude ethyl 2-[[3-hydroxy-4-methyl-5-(2-naphthyl)pyridine-2-carbonyl]amino]acetate (105 mg, 0.2882 mmol) and aqueous 1 N NaOH (1 mL) in THF (1 mL) and methanol (1 mL) was heated to 45° C. for 1 h. LCMS indicated complete consumption of starting material occurred. The reaction was concentrated directly. The residue was acidified by 1 N HCl and the precipitate was filtered. The solid was dissolved in DMSO and purified by prep-HPLC (40-85% MeCN in water over 25 min), affording the product as a white solid (52 mg, 52%). LC-MS (ESI+): m/z 337.0 (M+H)⁺; 1H NMR (400 MHz, DMSO) δ 12.81 (s, 2H), 9.38 (t, J=5.9 Hz, 1H), 8.15 (s, 1H), 8.10-7.95 (m, 4H), 7.64-7.54 (m, 3H), 4.01 (d, J=6.0 Hz, 2H), 2.21 (s, 3H).

### Example 34: Preparation of Compound 34

### 3-chloro-4-methyl-5-(quinolin-6-yl)picolinonitrile

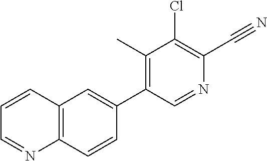 [SMILES: Cc1c(-c2ccc3ncccc3c2)cnc(C#N)c1Cl]

Pd(dppf)Cl₂ (0.032 g, 0.0248 mmol) was added to a stirred mixture of 3,5-dichloro-4-methyl-pyridine-2-carbonitrile (0.4 g, 2.14 mmol), 6-quinolylboronic acid (402 mg, 2.35 mmol) and K₂CO₃ ((354 mg, 2.57 mmol) in DMF (8 mL) and 0.8 mL water at rt. The resulting mixture was heated to 45° C. under nitrogen for 15 h. The reaction was diluted with ethyl acetate (20 mL) and filtered. The filtrate was concentrated and the resulting crude residue was purified by column (12 g, 0-100% ethyl acetate in hexanes) afford mixture of products (360 mg, 60%, mixture of Regio-isomers). LC-MS (ESI+): m/z 280.0 (M+H)⁺.

### 3-((4-methoxybenzyl)oxy)-4-methyl-5-(quinolin-6-yl)picolinonitrile

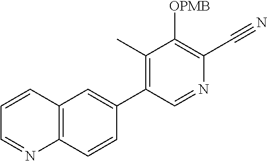

Sodium hydride (60% suspension in mineral oil, 107.68 mg, 2.69 mmol) was added to a stirred solution of 3-chloro-4-methyl-5-(6-quinolyl)pyridine-2-carbonitrile (502 mg, 1.79 mmol), (4-methoxyphenyl)methanol (0.33 mL, 2.69 mmol) in DMF (8.97 mL) at rt. The resulting mixture was stirred at rt. After 2 h, the reaction was quenched with water (20 mL) at 0° C. The mixture was extracted with ethyl acetate (20 mL). The organic layer was washed with brine, dried over Na₂SO₄ and concentrated. The crude was purified by column (0-100% ethyl acetate in hexanes), affording the major product (610 mg, 89%) as a brown liquid. LC-MS (ESI+): m/z 382.0 (M+H)⁺.

### 3-hydroxy-4-methyl-5-(quinolin-6-yl)picolinic Acid

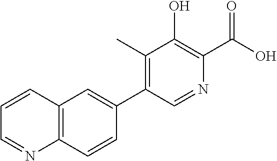 [SMILES: Cc1c(-c2ccc3ncccc3c2)cnc(C(=O)O)c1O]

A mixture of 3-[(4-methoxyphenyl)methoxy]-4-methyl-5-(6-quinolyl)pyridine-2-carbonitrile (610 mg, 1.6 mmol) and aq. 30% NaOH (6.67 mL, 50.03 mmol) in ethanol (6.1 mL) was heated to 100° C. for 3 h. The reaction was concentrated directly to remove ethanol. The mixture was cooled to 0° C. and acidified with concentrated HCl. The yellow solid was separated by filtration. The solid was dissolved in methanol (20 mL) and concentrated to give a yellow solid (160 mg). LCMS indicated MS/281. The crude was used in the next reaction without further purification. LC-MS (ESI+): m/z 281.0 (M+H)⁺.

### ethyl (3-hydroxy-4-methyl-5-(quinolin-6-yl)picolinoyl)glycinate

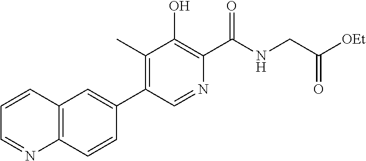 [SMILES: CCOC(=O)CNC(=O)c1ncc(-c2ccc3ncccc3c2)c(C)c1O]

Triethylamine (0.79 mL, 5.71 mmol) was added to a stirred mixture of 3-hydroxy-4-methyl-5-(6-quinolyl)pyridine-2-carboxylic acid (160 mg, 0.57 mmol), PyBOP (356 mg, 0.69 mmol) and ethyl 2-aminoacetate;hydrochloride (96 mg, 0.69 mmol) in DCM (10 mL) at rt. The reaction mixture was stirred overnight at rt. The reaction was concentrated directly. The crude residue was purified by column (4 g, 0-100% ethyl acetate in hexanes), affording a yellow foaming solid (58 mg, 28%). LC-MS (ESI+): m/z 366.0 (M+H)⁺.

### (3-hydroxy-4-methyl-5-(quinolin-6-yl)picolinoyl)glycine

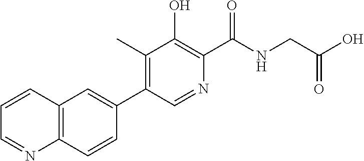 [SMILES: Cc1c(-c2ccc3ncccc3c2)cnc(C(=O)NCC(=O)O)c1O]

Aq. 1 N NaOH (0.4 mL, 0.40 mmol) was added to a stirred solution of ethyl 2-[[3-hydroxy-4-methyl-5-(6-quinolyl)pyridine-2-carbonyl]amino]acetate (58 mg, 0.160 mmol) in THF (1 mL) at rt. The resulting reaction was stirred at RT for 2 h. LCMS indicated complete consumption of starting material had occurred. The reaction mixture was concentrated directly to remove THF. The residue was acidified with aq. 1 N HCl (0.5 mL) and the yellow precipitate was filtered, washing with water. The solid was suspended in DMSO, sonicated for 10 min. The DMSO solution was filtered, yielding a white solid, which was further triturated in ethyl acetate. Further filtration afforded a white solid (22 mg, 40%). LC-MS (ESI+): m/z 338.0 (M+H)⁺; 1H NMR (400 MHz, DMSO) δ 12.84 (s, 1H), 9.42 (t, J=6.1 Hz, 1H), 8.97 (dd, J=4.2, 1.7 Hz, 1H), 8.45 (d, J=7.7 Hz, 1H), 8.17 (s, 1H), 8.13 (d, J=8.7 Hz, 1H), 8.09 (d, J=1.9 Hz, 1H), 7.84 (dd, J=8.7, 2.0 Hz, 1H), 7.61 (dd, J=8.3, 4.2 Hz, 1H), 4.01 (d, J=6.1 Hz, 2H), 2.21 (s, 3H).

### Example 35: Preparation of Compound 35

### 3,5-bis(benzyloxy)-4-methylpicolinonitrile

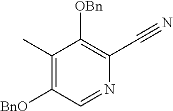 [SMILES: Cc1c(OCc2ccccc2)cnc(C#N)c1OCc1ccccc1]

Phenylmethanol (2.55 mL, 24.59 mmol) was added dropwise to a stirred suspension of NaH (60% suspension in mineral oil, 984 mg, 24.59 mmol) in DMF (21.38 mL) at 0° C. The reaction mixture was stirred for 15 min at 0° C. 3,5-dichloro-4-methyl-pyridine-2-carbonitrile (2 g, 10.69 mmol) in DMF (2 mL) was added at 0° C. The resulting red reaction mixture was stirred at RT for 1 h. LCMS indicated complete conversion to desired product mass (MS/331). The reaction was cooled to 0° C. and quenched with water (50 mL). The precipitate was filtered. The solid was purified by column (0-100% ethyl acetate in hexanes), affording a white solid (3.5 g, 99%). LC-MS (ESI+): m/z 331.0 (M+H)⁺.

### 3,5-bis(benzyloxy)-4-methylpicolinic Acid

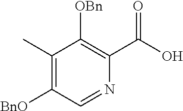 [SMILES: Cc1c(OCc2ccccc2)cnc(C(=O)O)c1OCc1ccccc1]

A mixture of 3,5-dibenzyloxy-4-methyl-pyridine-2-carbonitrile (3.5 g, 10.59 mmol) and 30% aq. NaOH (45 mL, 10.59 mmol) in ethanol (35 mL) was heated to 100° C. for 2 h. The reaction was concentrated directly to remove ethanol. The residue was cooled to 0° C., acidified with concentrated HCl (pH-3). The brown precipitate was filtered, resulting in a solid, which was washed with water (20 mL) and dried under vacuum, affording pale brown solid (3.2 g, 86%). The crude was used in the next reaction without further purification. LC-MS (ESI+): m/z 350.0 (M+H)⁺.

### ethyl (3,5-bis(benzyloxy)-4-methylpicolinoyl)glycinate

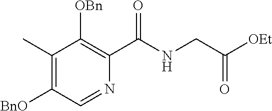 [SMILES: CCOC(=O)CNC(=O)c1ncc(OCc2ccccc2)c(C)c1OCc1ccccc1]

PyBOP (5.72 g, 10.99 mmol) was added to a suspension of 3,5-dibenzyloxy-4-methyl-pyridine-2-carboxylic acid (3.2 g, 9.16 mmol) in DCM (18.3 mL) at rt. The mixture was cooled to 0° C., triethylamine (6.36 mL, 45.8 mmol) was added. After 5 min, ethyl 2-aminoacetate;hydrochloride (1.92 g, 13.74 mmol) was added at 0° C. The resulting reaction mixture was stirred overnight at rt. The reaction was concentrated directly and the crude residue was purified by column (20 g, 0-100% ethyl acetate in hexanes), affording a brown liquid (3.6 g, 90%). LC-MS (ESI+): m/z 435.0 (M+H)⁺.

### ethyl (3,5-dihydroxy-4-methylpicolinoyl)glycinate

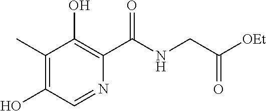 [SMILES: CCOC(=O)CNC(=O)c1ncc(O)c(C)c1O]

A mixture of ethyl 2-[(3,5-dibenzyloxy-4-methyl-pyridine-2-carbonyl)amino]acetate (3.6 g, 8.29 mmol) and 10% Pd on Carbon (0.26 mg, 8.29 mmol) in THF (20.7 mL) and Methanol (20.7 mL) was degassed with hydrogen for 5 min and stirred under hydrogen pressure for 3 h. LCMS indicated complete conversion to MS/255. The reaction mixture was filtered, rinsed with methanol, and the filtrate was concentrated to afford a white solid (2.1 g, 97%). The crude product was subjected to the next reaction without further purification. LC-MS (ESI+): m/z 255.0 (M+H)⁺.

### ethyl (3-hydroxy-4-methyl-5-phenoxypicolinoyl)glycinate

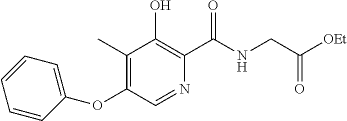 [SMILES: CCOC(=O)CNC(=O)c1ncc(Oc2ccccc2)c(C)c1O]

A mixture of ethyl 2-[(3,5-dihydroxy-4-methyl-pyridine-2-carbonyl)amino]acetate (42 mg, 0.17 mmol), iodobenzene (33.7 mg, 0.17 mmol) and K₂CO₃ (68.3 mg, 0.50 mmol) in DMF (2 mL) was heated to 90° C. for 48 h. The reaction was cooled to rt, diluted with DCM (10 mL) and filtered. The filtrate was concentrated. The crude was purified by column (4 g, 0-100% ethyl acetate in hexanes), affording a brown oil (6 mg, 11%). LC-MS (ESI+): m/z 331.0 (M+H)⁺.

### (3-hydroxy-4-methyl-5-phenoxypicolinoyl)glycine

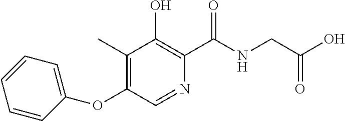 [SMILES: Cc1c(Oc2ccccc2)cnc(C(=O)NCC(=O)O)c1O]

Aq. 1 N NaOH (0.5 mL, 12.5 mmol) was added to a stirred solution of ethyl 2-[(3-hydroxy-4-methyl-5-phenoxy-pyridine-2-carbonyl)amino]acetate (6 mg, 0.02 mmol) in THF (2 mL) at rt. The resulting reaction mixture was stirred at RT for 2 h. The reaction was concentrated and acidified with 5 M HCl (0.1 mL). The mixture was concentrated. The crude residue was dissolved in DMSO (1 mL) and purified by prep-HPLC, affording a white solid (2.5 mg, 42%). LCMS (ESI+): m/z 303.0 (M+H)⁺; 1H NMR (400 MHz, DMSO) δ 12.92 (s, 2H), 9.12 (s, 1H), 7.76 (s, 1H), 7.45-7.31 (m, 2H), 7.16 (t, J=7.4 Hz, 1H), 7.09-6.96 (m, 2H), 3.89 (d, J=5.4 Hz, 2H), 2.10 (s, 3H).

### Example 36: Preparation of Compound 36

### ethyl (3-hydroxy-4-methyl-5-(((trifluoromethyl)sulfonyl)oxy)picolinoyl)glycinate

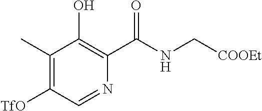 [SMILES: CCOC(=O)CNC(=O)c1ncc(OS(=O)(=O)C(F)(F)F)c(C)c1O]

Triethylamine (1.34 mL, 9.68 mmol) and 1,1,1-trifluoro-N-phenyl-N-(trifluoromethylsulfonyl)methanesulfonamide (3.17 g, 8.87 mmol) were added sequentially to a stirred suspension of ethyl 2-[(3,5-dihydroxy-4-methyl-pyridine-2-carbonyl)amino]acetate (2.05 g, 8.06 mmol) in ethanol (26.9 mL) at rt. The resulting reaction mixture was stirred overnight at 32° C. The reaction mixture turned to a clear brown homogeneous solution. TLC (30% ethyl acetate in hexanes) indicated complete conversion. The reaction was concentrated directly, and crude was purified by column (12 g, 0-10% MeOH in DCM), affording a brown liquid. Due to persistent impurities, the product was again purified by column (12 g, 0-70% ethyl acetate in hexanes) to afford a pale-yellow liquid. LC-MS (ESI+): m/z 387.0 (M+H)⁺.

### ethyl (3-(methoxymethoxy)-4-methyl-5-((((trifluoromethyl)sulfonyl)oxy)picolinoyl)glycinate

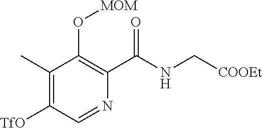

DIPEA (2.52 mL, 14.24 mmol) and chloro(methoxy)methane (0.65 mL, 8.54 mmol) were added sequentially to a stirred solution of ethyl 2-[[3-hydroxy-4-methyl-5-(trifluoromethylsulfonyloxy)pyridine-2-carbonyl]amino]acetate (2.2 g, 5.7 mmol) in DCM (32.4 mL) at 0° C. The resulting reaction mixture was stirred at 0° C. for 15 min and then warmed to RT for 1 h. TLC (30% ethyl acetate in hexanes) indicated complete consumption of starting material occurred. The reaction was quenched with cold water (20 mL). The organic layer was separated, dried over Na₂SO₄ and concentrated. The crude was purified by column (12 g, 0-100% ethyl acetate in hexanes), affording a white solid (1.5 g, 62%). LC-MS (ESI+): m/z 431.0 (M+H)⁺.

### ethyl (5-(3,5-dichlorophenyl)-3-(methoxymethoxy)-4-methylpicolinoyl)glycinate

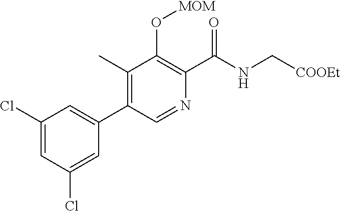

A mixture of (3,5-dichlorophenyl)boronic acid (33.3 mg, 0.17 mmol), ethyl 2-[[3-(methoxymethoxy)-4-methyl-5-(trifluoromethylsulfonyloxy)pyridine-2-carbonyl]amino]acetate (50 mg, 0.12 mmol), and Pd(dppf)Cl₂ (8.5 mg, 0.01 mmol) in 1,4-dioxane (2 mL) and water (0.5 mL) was heated to 85° C. for 20 h. LCMS indicated presence of desired product (MS/428). The reaction was diluted with methanol (10 mL) and filtered. The filtrate was concentrated. The crude product was purified by column (0-100% ethyl acetate in hexanes), affording the expected product along with the deprotected product. The two products were combined and concentrated to afford 23 mg. The mixture was subjected to the next reaction. LC-MS (ESI+): m/z 428.0 (M+H)⁺.

### (5-(3,5-dichlorophenyl)-3-hydroxy-4-methylpicolinoyl)glycine

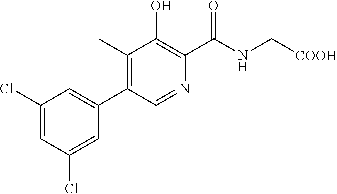 [SMILES: Cc1c(-c2cc(Cl)cc(Cl)c2)cnc(C(=O)NCC(=O)O)c1O]

TFA (0.1 mL, 0.05 mmol) was added to a stirred solution of ethyl 2-[[5-(3,5-dichlorophenyl)-3-(methoxymethoxy)-4-methyl-pyridine-2-carbonyl]amino]acetate (23 mg, 0.05 mmol) in DCM (2 mL) at rt. The resulting mixture was stirred overnight at rt. LC-MS indicated complete conversion had occurred. The reaction was concentrated directly to dryness. The residue was dissolved in THF (2 mL) and treated with aq. 1 N NaOH (0.2 mL, 0.05 mmol) at RT for 3 h. LCMS indicated complete conversion. The reaction was concentrated directly, acidified with 5 N HCl (0.11 mL), and concentrated to dryness. The residue was dissolved in DMSO (1 mL) and filtered. The filtrate was purified by HPLC to afford a white solid (7.6 mg, 36%). LC-MS (ESI+): m/z 356.0 (M+H)⁺; 1H NMR (400 MHz, DMSO) δ 12.83 (s, 1H), 9.36 (s, 1H), 8.06 (s, 1H), 7.77 (dd, J=5.2, 3.1 Hz, 2H), 7.45 (dd, J=8.3, 2.1 Hz, 1H), 3.96 (d, J=5.7 Hz, 2H), 2.14 (s, 3H).

### Example 37: Preparation of Compound 37

### ethyl (3-(methoxymethoxy)-4-methyl-5-(4-methyl-2-phenylthiazol-5-yl)picolinoyl)glycinate

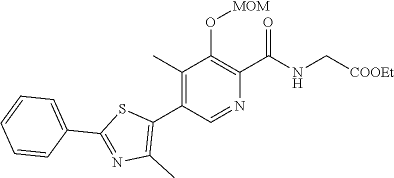

A mixture of 4-methyl-2-phenyl-5-(4,4,5,5-tetramethyl-1,3,2-dioxaborolan-2-yl)thiazole (162.8 mg, 0.54 mmol), ethyl 2-[[3-(methoxymethoxy)-4-methyl-5-(trifluoromethylsulfonyloxy)pyridine-2-carbonyl]amino]acetate (155.1 mg, 0.36 mmol), Pd(dppf)Cl₂ (26.4 mg, 0.04 mmol) and K₃PO₄ (114.7 mg, 0.54 mmol) in 1,4-dioxane (3 mL) and water (1 mL) was heated to 85° C. for 16 h. The reaction was cooled to rt, filtered and rinsed with ethyl acetate. The filtrate was concentrated and the crude residue was purified by column (4 g, 0-100% ethyl acetate in hexanes), affording a pale brown oil (64 mg, 39%). LC-MS (ESI+): m/z 456.0 (M+H)⁺.

### (3-hydroxy-4-methyl-5-(4-methyl-2-phenylthiazol-5-yl)picolinoyl)glycine

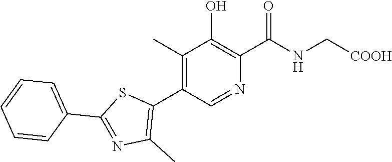 [SMILES: Cc1nc(-c2ccccc2)sc1-c1cnc(C(=O)NCC(=O)O)c(O)c1C]

1 N NaOH (0.6 mL, 15 mmol) was added to a stirred solution of ethyl 2-[[3-(methoxymethoxy)-4-methyl-5-(4-methyl-2-phenyl-thiazol-5-yl)pyridine-2-carbonyl]amino]acetate (64 mg, 0.14 mmol) in THF (3 mL) and methanol (1 mL) at RT and resulting reaction mixture was stirred at RT for 30 min. LCMS indicated complete ester hydrolysis occurred (MS/428). The reaction was acidified with 1 N HCl (0.8 mL) and stirred for 30 min. The reaction was concentrated directly to remove THF and methanol. The precipitate was filtered. The solid was purified by prep-HPLC (30-85% MeCN in water over 25 min), affording a white solid (11 mg, 20%). LC-MS (ESI+): m/z 384.0 (M+H)⁺; 1H NMR (400 MHz, CD3OD) δ 8.08 (s, 1H), 8.00-7.90 (m, 1H), 7.55-7.46 (m, 2H), 7.55-7.46 (m, 3H), 4.16 (s, 2H), 2.31 (s, 3H), 2.22 (s, 3H).

### Example 38: Preparation of Compound 38

### ethyl (5-(1-(4-fluorophenyl)-3-methyl-1H-pyrazol-4-yl)-3-(methoxymethoxy)-4-methylpicolinoyl)glycinate

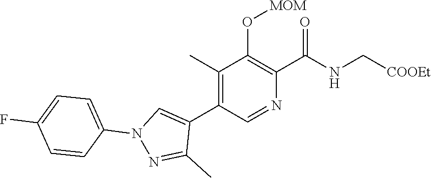

A mixture of ethyl 2-[[3-(methoxymethoxy)-4-methyl-5-(trifluoromethylsulfonyloxy)pyridine-2-carbonyl]amino]acetate (200 mg, 0.46 mmol), 1-(4-fluorophenyl)-3-methyl-4-(4,4,5,5-tetramethyl-1,3,2-dioxaborolan-2-yl)pyrazole (281 mg, 0.93 mmol), Pd(dppf)Cl₂ (34 mg, 0.05 mmol) and K₃PO₄ (148 mg, 0.70 mmol) in 1,4-dioxane (3 mL) and water (1 mL) was heated to 80° C. overnight. The reaction was cooled to rt, diluted with ethyl acetate (20 mL), filtered through a pad of Celite. The filtrate was concentrated, crude was purified by column (0-100% ethyl acetate in hexanes), affording a brown residue (80 mg, 38%). LC-MS (ESI+): m/z 384.0 (M+H)⁺.

### (5-(1-(4-fluorophenyl)-3-methyl-1H-pyrazol-4-yl)-3-hydroxy-4-methylpicolinoyl)glycine

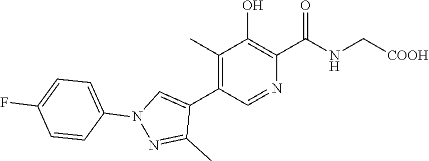 [SMILES: Cc1nn(-c2ccc(F)cc2)cc1-c1cnc(C(=O)NCC(=O)O)c(O)c1C]

Ethyl 2-[[5-[1-(4-fluorophenyl)-3-methyl-pyrazol-4-yl]-3-(methoxymethoxy)-4-methyl-pyridine-2-carbonyl]amino]acetate (80 mg, 0.18 mmol) was suspended in methanol (1 mL) and 5 N HCl (0.1 mL, 0.50 mmol) was added. The reaction mixture was stirred at RT for 1 h. The reaction was concentrated directly. The crude was dissolved in THF (3 mL) and 1 N NaOH (1.5 mL, 37.5 mmol) was added. The reaction mixture was stirred overnight at rt. The reaction was acidified by aq. 5 N HCl and concentrated directly. The crude was dissolved in DMSO (1.5 mL), filtered and purified by prep-HPLC, affording an off-white solid (8 mg, 12%). LC-MS (ESI+): m/z 385.0 (M+H)⁺; 1H NMR (400 MHz, DMSO) δ 12.77 (s, 1H), 9.34 (t, J=6.0 Hz, 1H), 8.72 (s, 1H), 8.09 (s, 1H), 7.73 (dd, J=9.0, 1.3 Hz, 2H), 7.54 (dd, J=14.8, 8.0 Hz, 1H), 7.24-7.05 (m, 1H), 3.98 (d, J=6.1 Hz, 2H), 2.22 (s, 3H), 2.18 (s, 3H).

### Example 39: Preparation of Compound 39

### ethyl (5-(1-(3-fluorophenyl)-3-methyl-1H-pyrazol-4-yl)-3-(methoxymethoxy)-4-methylpicolinoyl)glycinate

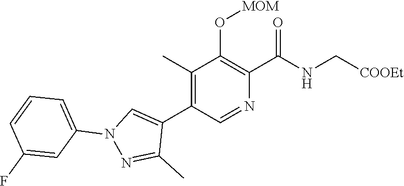

A mixture of ethyl 2-[[3-(methoxymethoxy)-4-methyl-5-(trifluoromethylsulfonyloxy)pyridine-2-carbonyl]amino]acetate (200 mg, 0.46 mmol), 1-(3-fluorophenyl)-3-methyl-4-(4,4,5,5-tetramethyl-1,3,2-dioxaborolan-2-yl)pyrazole (281 mg, 0.93 mmol), Pd(dppf)Cl₂ (34 mg, 0.05 mmol) and K₃PO₄ (296 mg, 1.40 mmol) in 1,4-dioxane (3 mL) and water (1 mL) was heated to 80° C. overnight. The reaction was cooled to rt, diluted with ethyl acetate (20 mL), and filtered through a pad of Celite. The filtrate was concentrated and the crude residue was purified by column (0-100% ethyl acetate in hexanes), affording a brown oil (110 mg, 59%); LC-MS (ESI+): m/z 457.0 (M+H)⁺.

### (5-(1-(3-fluorophenyl)-3-methyl-1H-pyrazol-4-yl)-3-hydroxy-4-methylpicolinoyl)glycine

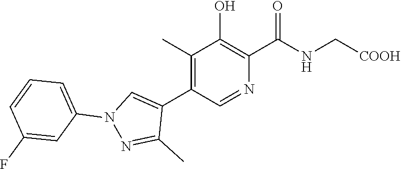 [SMILES: Cc1nn(-c2cccc(F)c2)cc1-c1cnc(C(=O)NCC(=O)O)c(O)c1C]

5 N HCl (0.1 mL, 0.24 mmol) was added to a stirred solution of ethyl 2-[[5-[1-(3-fluorophenyl)-3-methyl-pyrazol-4-yl]-3-(methoxymethoxy)-4-methyl-pyridine-2-carbonyl]amino]acetate (110 mg, 0.24 mmol) in methanol (2 mL) at rt. The reaction mixture was stirred at RT for 1 h. The reaction was concentrated to dryness. The crude residue was dissolved in THF (3 mL) and aq. 1 N NaOH (1.5 mL, 37.5 mmol) was added at rt. The resulting reaction mixture was stirred overnight at rt. The reaction was acidified by 5 N HCl and concentrated directly. The crude product was dissolved in DMSO (1.5 mL), filtered, and purified by prep-HPLC, affording a white solid (8 mg, 8.4%). LC-MS (ESI+): m/z 385.0 (M+H)⁺; 1H NMR (400 MHz, DMSO) δ 12.75 (s, 1H), 9.35 (t, J=6.1 Hz, 1H), 8.64 (s, 1H), 8.08 (s, 1H), 7.89 (ddd, J=6.9, 5.2, 2.8 Hz, 2H), 7.39-7.31 (m, 2H), 4.00 (d, J=6.1 Hz, 2H), 2.21 (s, 3H), 2.18 (s, 3H).

### Example 40: Preparation of Compound 40

### ethyl (3-(methoxymethoxy)-4-methyl-5-(1-phenylvinyl)picolinoyl)glycinate

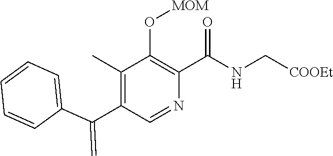

A mixture of 1-phenylvinylboronic acid (77.4 mg, 0.52 mmol), ethyl 2-[[3-(methoxymethoxy)-4-methyl-5-(trifluoromethylsulfonyloxy)pyridine-2-carbonyl]amino]acetate (150 mg, 0.35 mmol), Pd(dppf)Cl₂ (25.5 mg, 0.03 mmol), K₃PO₄ (222 mg, 1.05 mmol) and water (0.03 mL, 1.74 mmol) in 1,4-dioxane (3 mL) was heated to 80° C. overnight. The reaction was cooled to rt, diluted with ethyl acetate (20 mL), and filtered. The filtrate was concentrated. The resulting crude residue was purified by column (0-100% ethyl acetate in hexanes), affording a brown oil (32 mg, 24%); LC-MS (ESI+): m/z 385.0 (M+H)⁺.

### ethyl (5-benzoyl-3-hydroxy-4-methylpicolinoyl)glycinate

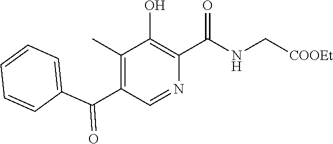 [SMILES: CCOC(=O)CNC(=O)c1ncc(C(=O)c2ccccc2)c(C)c1O]

To a stirring mixture of ethyl (3-(methoxymethoxy)-4-methyl-5-(1-phenylvinyl)picolinoyl)glycinate (32 mg, 0.09 mmol) and NaIO₄ (201 mg, 0.94 mmol) in THF (2 mL), acetone (1 mL) and aater (1 mL) at RT was added 2.5% OsO4 in tBuOH (0.12 mL). The resulting reaction mixture was stirred at RT for 3 days. The reaction was diluted with ethyl acetate (20 mL), filtered, andrinsed with ethyl acetate (20 mL). The filtrate was washed with brine (15 mL), dried over Na₂SO₄ and concentrated. The crude product (32 mg) was subjected to the next reaction without further purification; LC-MS (ESI+): m/z 343.0 (M+H)⁺.

### (5-benzoyl-3-hydroxy-4-methylpicolinoyl)glycine

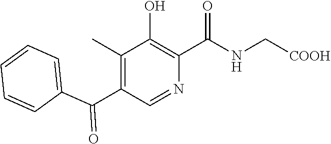 [SMILES: Cc1c(C(=O)c2ccccc2)cnc(C(=O)NCC(=O)O)c1O]

Aq. 1 N NaOH (0.5 mL, 12.5 mmol) was added to a stirred solution of ethyl 2-[(5-benzoyl-3-hydroxy-4-methyl-pyridine-2-carbonyl)amino]acetate (32 mg, 0.090 mmol) in THF (2 mL) at rt. The reaction mixture was stirred for 1 h at rt. The reaction was acidified with 5 N HCl (0.11 mL) and concentrated directly. The crude was dissolved in DMSO (1 mL) and purified by prep-HPLC, affording a white solid (7 mg, 21% over 2 steps). LC-MS (ESI+): m/z 315.0 (M+H)⁺; 1H NMR (400 MHz, DMSO) δ 12.90 (s, 1H), 12.81 (bs, 1H), 9.49 (t, J=6.1 Hz, 1H), 8.11 (s, 1H), 7.83-7.77 (m, 2H), 7.73 (dd, J=10.5, 4.3 Hz, 1H), 7.57 (t, J=7.8 Hz, 2H), 4.01 (d, J=6.2 Hz, 2H), 2.07 (s, 3H).

### Example 41: Preparation of Compound 41

### ethyl (3-(methoxymethoxy)-4-methyl-5-((trimethylsilyl)ethynyl)picolinoyl)glycinate

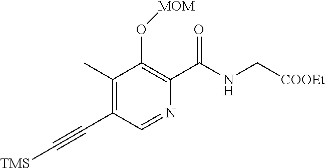

Triethylamine (0.81 mL, 5.81 mmol) and ethynyl(trimethyl)silane (0.32 mL, 2.32 mmol) were added to a stirred mixture of ethyl 2-[[3-(methoxymethoxy)-4-methyl-5-(trifluoromethylsulfonyloxy)pyridine-2-carbonyl]amino]acetate (500 mg, 1.16 mmol), Pd(PPh₃)₂Cl₂ (81.6 mg, 0.12 mmol) and CuI (44.3 mg, 0.23 mmol) in THF (11.6 mL) at rt. The resulting reaction mixture was stirred for 2 h at rt. The reaction mixture was diluted with ethyl acetate (25 mL) and filtered through a pad of Celite. The filtrate was concentrated. The crude residue was dissolved in ethyl acetate (25 mL), washed with water (10 mL), brine (10 mL), dried over Na₂SO₄, and concentrated. The crude product was purified by column (12 g, 0-70% ethyl acetate in hexanes), affording a brown oil (421 mg, 97%); LC-MS (ESI+): m/z 378.0 (M+H)⁺.

### ethyl (5-ethynyl-3-(methoxymethoxy)-4-methylpicolinoyl)glycinate

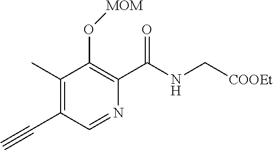

K₂CO₃ (15.4 mg, 0.11 mmol) was added to a stirred solution of ethyl 2-[[3-(methoxymethoxy)-4-methyl-5-(2-trimethylsilylethynyl)pyridine-2-carbonyl]amino]acetate (421 mg, 1.11 mmol) in methanol (5.56 mL) at 0° C. The resulting reaction mixture was stirred at 0° C. for 30 min. The reaction was diluted with ethyl acetate (10 mL) and filtered. The filtrate was concentrated and thecrude residue was purified by column (4 g, 0-100% ethyl acetate in hexanes), affording a colorless oil (265 mg, 77%); LC-MS (ESI+): m/z 307.0 (M+H)⁺.

### ethyl (5-(1-(4-fluorophenyl)-1H-1,2,3-triazol-4-yl)-3-hydroxy-4-methylpicolinoyl)glycinate

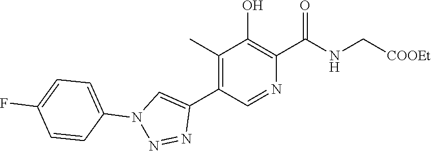 [SMILES: CCOC(=O)CNC(=O)c1ncc(-c2cn(-c3ccc(F)cc3)nn2)c(C)c1O]

Sodium ascorbate (5.3 mg, 0.03 mmol) and CuSO4 (2.1 mg, 0.01 mmol) were added to a stirred mixture of methyl 2-[[5-ethynyl-3-(methoxymethoxy)-4-methyl-pyridine-2-carbonyl]amino]acetate (39.1 mg, 0.13 mmol), (4-fluorophenyl) azide in tert-butylmethyl ether (0.27 mL, 0.130 mmol) in tert-butanol (1 mL), methanol (1 mL) and water (0.5 mL) at rt. The resulting reaction mixture was stirred at RT overnight. LCMS indicated complete conversion to MS/386. The reaction was diluted with ethyl acetate (15 mL) and filtered. The filtrate was concentrated and the resulting crude residue was purified by column (4 g, 0-100% ethyl acetate in hexanes), affording a white solid (50 mg, 69%); LC-MS (ESI+): m/z 400.0 (M+H)⁺.

### (5-(1-(4-fluorophenyl)-1H-1,2,3-triazol-4-yl)-3-hydroxy-4-methylpicolinoyl)glycine

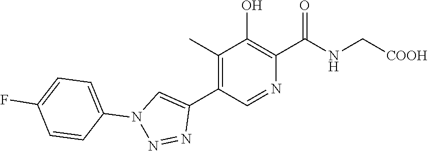 [SMILES: Cc1c(-c2cn(-c3ccc(F)cc3)nn2)cnc(C(=O)NCC(=O)O)c1O]

Aq. 1 N NaOH (0.41 mL, 0.41 mmol) was added to a stirred mixture of methyl 2-[[5-[1-(4-fluorophenyl)triazol-4-yl]-3-hydroxy-4-methyl-pyridine-2-carbonyl]amino]acetate (18.5 mg, 0.05 mmol), in THF (3 mL) and methanol (1 mL) at rt. The resulting white turbid mixture was stirred at RT for 1 h. LCMS indicated complete consumption of starting material occurred. The reaction was concentrated directly, and residue was acidified with 1 N HCl (0.6 mL). The precipitate was filtered and washed with water. The solid was dried under high vacuum to afford a white solid (16 mg, 85%). LC-MS (ESI+): m/z 371.0 (M+H)⁺; 1H NMR (500 MHz, DMSO) δ 12.88 (s, 1H), 12.80 (s, 1H), 9.40 (t, J=6.1 Hz, 1H), 9.27 (s, 1H), 8.57 (s, 1H), 8.08-8.00 (m, 2H), 7.58-7.45 (m, 2H), 4.01 (d, J=6.1 Hz, 2H), 2.47 (s, 3H).

### Example 42: Preparation of Compound 42

### ethyl (5-(1-(4-chlorophenethyl)-1H-1,2,3-triazol-4-yl)-3-hydroxy-4-methylpicolinoyl)glycinate

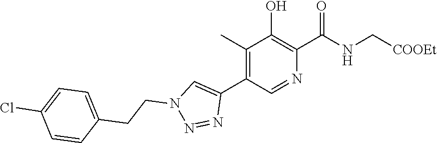 [SMILES: CCOC(=O)CNC(=O)c1ncc(-c2cn(CCc3ccc(Cl)cc3)nn2)c(C)c1O]

A mixture of ethyl 2-[[5-ethynyl-3-(methoxymethoxy)-4-methyl-pyridine-2-carbonyl]amino]acetate (50 mg, 0.16 mmol), 2-(4-chlorophenyl)ethylimino-imino-ammonium (59.6 mg, 0.33 mmol), CuSO₄ (2.6 mg, 0.02 mmol) and sodium ascorbate (12.9 mg, 0.07 mmol) in methanol (1 mL), tert-butanol (1 mL) and water (0.5 mL) was stirred overnight at rt. The reaction mixture was diluted with 50% MeOH in DCM (20 mL) and filtered. The filtrate was concentrated. The crude product (145 mg) was subjected to the next reaction without purification. LC-MS (ESI+): m/z 444.0 (M+H)⁺.

### (5-(1-(4-chlorophenethyl)-1H-1,2,3-triazol-4-yl)-3-hydroxy-4-methylpicolinoyl)glycine

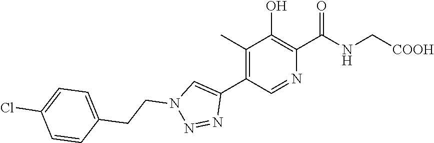 [SMILES: Cc1c(-c2cn(CCc3ccc(Cl)cc3)nn2)cnc(C(=O)NCC(=O)O)c1O]

Aq. 1 N NaOH (0.24 mL, 6.03 mmol) was added to a stirred solution of ethyl 2-[[5-[11-[2-(4-chlorophenyl)ethyl]triazol-4-yl]-3-hydroxy-4-methyl-pyridine-2-carbonyl]amino]acetate (70 mg, 0.16 mmol) in THF (3 mL) at rt. The reaction mixture was stirred for 1 h at rt. The reaction was quenched with 1 N HCl (0.52 mL). The mixture was concentrated directly to dryness. The solid was dissolved in DMSO (1 mL) and purified by prep-HPLC, affording the desired product as white solid (8 mg, 12%). LC-MS (ESI+): m/z 416.0 (M+H)⁺; ¹H NMR (400 MHz, DMSO) δ 8.23 (s, 1H), 8.01 (s, 1H), 7.19-7.12 (m, 2H), 7.03 (d, J=8.5 Hz, 2H), 4.63 (t, J=7.0 Hz, 2H), 4.02 (s, 2H), 3.19-3.13 (m, J=7.0 Hz, 2H), 2.16 (s, 3H).

### Example 43: Preparation of Compound 43

### ethyl (5-(1-(3-(4-chlorophenyl)propyl)-1H-1,2,3-triazol-4-yl)-3-hydroxy-4-methylpicolinoyl)glycinate

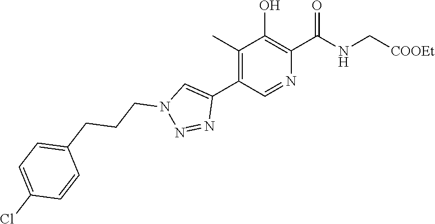 [SMILES: CCOC(=O)CNC(=O)c1ncc(-c2cn(CCCc3ccc(Cl)cc3)nn2)c(C)c1O]

A mixture of ethyl 2-[[5-ethynyl-3-(methoxymethoxy)-4-methyl-pyridine-2-carbonyl]amino]acetate (50 mg, 0.16 mmol), 3-(4-chlorophenyl)propylimino-imino-ammonium (64.2 mg, 0.33 mmol), CuSO4 (2.6 mg, 0.02 mmol) and sodium ascorbate (12.9 mg, 0.07 mmol) in tert-butanol (1 mL), methanol (1 mL) and water (0.50 mL) was stirred overnight at rt. The reaction was diluted with DCM (20 mL) and filtered. The filtrate was concentrated and the resulting crude product (150 mg) was subjected to the next reaction without purification; LC-MS (ESI+): m/z 458.0 (M+H)⁺.

### (5-(1-(3-(4-chlorophenyl)propyl)-1H-1,2,3-triazol-4-yl)-3-hydroxy-4-methylpicolinoyl)glycine

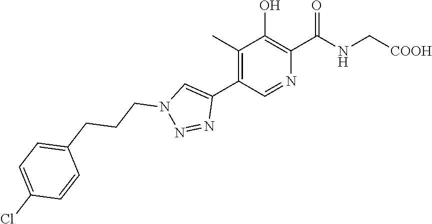 [SMILES: Cc1c(-c2cn(CCCc3ccc(Cl)cc3)nn2)cnc(C(=O)NCC(=O)O)c1O]

Aq. 1 N NaOH (0.5 mL, 12.5 mmol) was added to a stirred solution of ethyl 2-[[5-[1-[3-(4-chlorophenyl)propyl]triazol-4-yl]-3-hydroxy-4-methyl-pyridine-2-carbonyl]amino]acetate (75 mg, 0.16 mmol) in THF (3 mL) at rt. The reaction mixture was stirred for 1 h. LCMS indicated complete conversion occurred. The reaction was quenched with 1 N HCl (0.52 mL). The mixture was concentrated to dryness. The solid was dissolved in DMSO (1 mL) and purified by prep-HPLC, affording a white solid (36 mg, 49%). LC-MS (ESI+): m/z 430.0 (M+H)⁺; ¹H NMR (400 MHz, DMSO) δ 12.84 (s, 1H), 9.37 (t, J=6.1 Hz, 1H), 8.66 (s, 1H), 8.50 (s, 1H), 7.38-7.30 (m, 2H), 7.26 (d, J=8.4 Hz, 2H), 4.46 (t, J=7.1 Hz, 2H), 3.99 (d, J=6.1 Hz, 2H), 2.67-2.58 (m, 2H), 2.38 (s, 3H), 2.26-2.15 (m, 2H).

### Example 44: Preparation of Compound 44

### ethyl (3-hydroxy-4-methyl-5-(1-phenyl-1H-1,2,3-triazol-4-yl)picolinoyl)glycinate

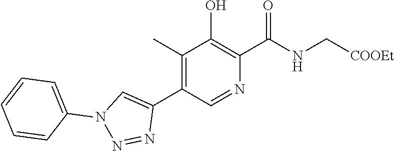 [SMILES: CCOC(=O)CNC(=O)c1ncc(-c2cn(-c3ccccc3)nn2)c(C)c1O]

CuSO₄ (2.8 mg, 0.02 mmol) and sodium ascorbate (14.0 mg, 0.07 mmol) were added to a stirred mixture of ethyl 2-[[5-ethynyl-3-(methoxymethoxy)-4-methyl-pyridine-2-carbonyl]amino]acetate (54 mg, 0.18 mmol) and 0.5 M phenyl azide in tert-butyl methyl ether (0.71 mL, 0.350 mmol) in tert-butanol (2 mL), methanol (2 mL) and water (1 mL) at rt. The resulting reaction mixture was stirred overnight at rt. LCMS indicated formation of product (MS/382). The reaction was diluted with DCM (20 mL), filtered, and the filtrate was concentrated. The resulting crude residue was concentrated by column (4 g, 0-100% ethyl acetate in hexanes), affording a white solid (46 mg, 68%). LCMS indicated product is a mixture of the methyl and ethyl esters (MS/368 and/MS/382).

### (3-hydroxy-4-methyl-5-(1-phenyl-1H-1,2,3-triazol-4-yl)picolinoyl)glycine

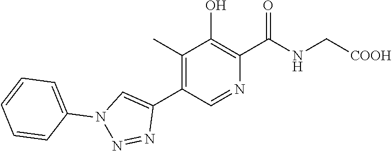 [SMILES: Cc1c(-c2cn(-c3ccccc3)nn2)cnc(C(=O)NCC(=O)O)c1O]

Aq. 1 N NaOH (1 mL, 25 mmol) was added to a stirred solution of ethyl 2-[[3-hydroxy-4-methyl-5-(1-phenyltriazol-4-yl)pyridine-2-carbonyl]amino]acetate (46 mg, 0.12 mmol) in THF (3 mL) and methanol (1 mL) at rt. The resulting reaction mixture was stirred for 2 h at rt. The reaction was quenched with aq. 1 N HCl (1.2 mL) and concentrated under reduced pressure. The crude was suspended in water (10 mL) and the formed solid was separated by filtration. The solid was suspended in ethyl acetate (2 mL), sonicated, solid was separated by filtration, affording an off white solid (35 mg, 78%). LC-MS (ESI+): m/z 354.0 (M+H)⁺; ¹H NMR (400 MHz, DMSO) δ 12.89 (s, 1H), 12.82 (bs, 1H), 9.43 (t, J=6.1 Hz, 1H), 9.30 (s, 1H), 8.58 (s, 1H), 8.00 (d, J=7.6 Hz, 2H), 7.65 (t, J=7.9 Hz, 2H), 7.54 (t, J=7.4 Hz, 1H), 4.01 (d, J=6.1 Hz, 2H), 2.47 (s, 3H).

### Example 45: Preparation of Compound 45

### ethyl (3-hydroxy-4-methyl-5-vinylpicolinoyl)glycinate

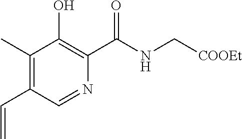 [SMILES: C=Cc1cnc(C(=O)NCC(=O)OCC)c(O)c1C]

A mixture of ethyl 2-[[3-(2-methoxyethoxymethoxy)-4-methyl-5-(trifluoromethylsulfonyloxy)pyridine-2-carbonyl]amino]acetate (300 mg, 0.63 mmol), tributyl(vinyl)stannane (0.22 mL, 0.76 mmol), LiCl (80.4 mg, 1.9 mmol) and Pd(PPh₃)₂Cl₂ (22.5 mg, 0.03 mmol) in DMF (5 mL) was heated to 69° C. for 2 h. The reaction was cooled to RT and left to stir at RT for three days. The reaction was concentrated and resulting crude residue was purified by column (4 g, 0-100% ethyl acetate in hexanes), affording the product as a brown oil (152 mg, 91%); LC-MS (ESI+): m/z 265.0 (M+H)⁺.

### ethyl (5-formyl-3-hydroxy-4-methylpicolinoyl)glycinate

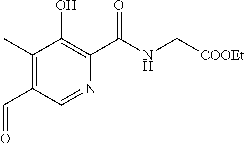 [SMILES: CCOC(=O)CNC(=O)c1ncc(C=O)c(C)c1O]

A solution of 2.5 M OsO₄ in tert-butanol (0.2 mL) was added to a stirred mixture of ethyl 2-[(3-hydroxy-4-methyl-5-vinyl-pyridine-2-carbonyl)amino]acetate (152 mg, 0.58 mmol), pyridine (0.1 mL, 0.58 mmol) and NaIO₄ (615 mg, 2.88 mmol) in acetone (1 mL), THF (2 mL) and water (1 mL) at rt. The resulting reaction mixture was stirred for 24 h at rt. The reaction was diluted with ethyl acetate (20 mL) and filtered. The filtrate was concentrated and the resulting crude product was purified by column (0-100% ethyl acetate in hexanes), affording a white solid (120 mg, 78%); LC-MS (ESI+): m/z 267.0 (M+H)⁺.

### ethyl (5-(2-(3,5-dichlorophenyl)-2H-tetrazol-5-yl)-3-hydroxy-4-methylpicolinoyl)glycinate

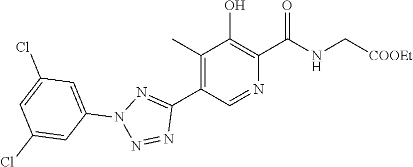 [SMILES: CCOC(=O)CNC(=O)c1ncc(-c2nnn(-c3cc(Cl)cc(Cl)c3)n2)c(C)c1O]

Ethyl 2-[(5-formyl-3-hydroxy-4-methyl-pyridine-2-carbonyl)amino]acetate (120 mg, 0.45 mmol) and 4-methylbenzenesulfonohydrazide (100 mg, 0.54 mmol) in DCM (10 mL) was stirred at RT for 30 min (a clear solution formed). The reaction was concentrated directly and the crude residue was dissolved in pyridine (4 mL) and cooled to −5° C. [(E)-(3,5-dichlorophenyl)azo]-tetrafluoro-boron (235 mg, 0.90 mmol) in ethanol (4 mL) and water (2.5 mL) was added dropwise at −5 to 0° C. The resulting reaction mixture was stirred at RT over night. The reaction was diluted with water (20 mL) and extracted with ethyl acetate (20 mL×2). The organic layer was concentrated and the crude residue was purified by column (4 g, 0-100% ethyl acetate in DCM), affording a red solid (94 mg, 46%); LC-MS (ESI+): m/z 452.0 (M+H)⁺.

### (5-(2-(3,5-dichlorophenyl)-2H-tetrazol-5-yl)-3-hydroxy-4-methylpicolinoyl)glycine

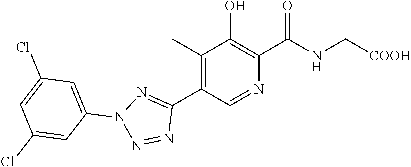 [SMILES: Cc1c(-c2nnn(-c3cc(Cl)cc(Cl)c3)n2)cnc(C(=O)NCC(=O)O)c1O]

Aq. 1 N NaOH (0.5 mL, 12.5 mmol) was added to a stirred solution of ethyl 2-[[5-[2-(3,5-dichlorophenyl)tetrazol-5-yl]-3-hydroxy-4-methyl-pyridine-2-carbonyl]amino]acetate (94 mg, 0.21 mmol) in THF (3 mL). The reaction was stirred at RT for 30 min. Methanol (1 mL) was added and the reaction mixture was stirred further at RT for 30 min. LCMS indicated complete conversion. The reaction was concentrated directly. The crude residue was acidified with 1 N HCl and the resulting precipitate was filtered. The solid was suspended in ethanol (10 mL), heated to 90° C. for 30 min, cooled to rt, and collected by filtration. The pure solid was dried under vacuum, affording a white solid (9.5 mg, 10%). LC-MS (ESI+): m/z 424.0 (M+H)⁺; ¹H NMR (400 MHz, DMSO) δ 13.02 (s, 1H), 12.86 (bs, 1H), 9.54 (t, J=5.7 Hz, 1H), 8.77 (s, 1H), 8.27 (d, J=1.8 Hz, 2H), 7.98 (t, J=1.8 Hz, 1H), 4.01 (d, J=6.0 Hz, 2H), 2.59 (s, 3H).

### Example 46: Preparation of Compound 46

### 3,5-dichloro-4-ethylpyridine 1-oxide

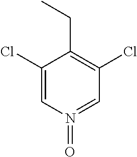

3-Chlorobenzenecarboperoxoic acid (1.41 g, 8.2 mmol) was dissolved in DCM (20 mL) and added dropwise to a stirred solution of 3,5-dichloro-4-ethyl-pyridine (1.11 g, 6.31 mmol) in DCM (15.8 mL) at rt. The resulting mixture was stirred overnight at rt. The reaction was quenched with solid K₂CO₃ (1.13 g, 8.2 mmol) and stirred for 1 h. The precipitate was filtered and rinsed with DCM (25 mL). The filtrate was concentrated to afford white solid (1.2 g, 99%). The crude was subjected to the next reaction without further purification; LC-MS (ESI+): m/z 193.0 (M+H)⁺.

### 3,5-dichloro-4-ethylpicolinonitrile

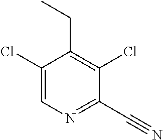

A mixture of 3,5-dichloro-4-ethyl-pyridine 1-oxide (1.2 g, 6.25 mmol), triethylamine (1.3 mL, 9.37 mmol) and trimethylsilylformonitrile (1.17 mL, 9.37 mmol) in MeCN (20 mL) was heated to 80° C. overnight. The reaction was cooled to RT and quenched with aqueous NaHCO₃ solution (10 mL). The mixture was diluted with ethyl acetate (20 mL). The layers were separated and the organic layer was washed with brine (10 mL), dried over Na2SO4 and concentrated. The crude was purified by column (12 g column, 0-50% ethyl acetate in hexanes), affording a pale brown liquid (1.03 g, 82%); LC-MS (ESI+): m/z 202.0 (M+H)⁺.

### 3-chloro-5-(3-chlorophenyl)-4-ethylpicolinonitrile

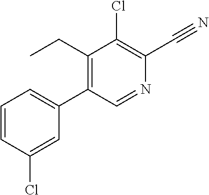 [SMILES: CCc1c(-c2cccc(Cl)c2)cnc(C#N)c1Cl]

A mixture of 3,5-dichloro-4-ethyl-pyridine-2-carbonitrile (490 mg, 2.44 mmol), (3-chlorophenyl)boronic acid (381 mg, 2.44 mmol), Pd(dppf)Cl₂ (89 mg, 0.12 mmol) and K₂CO₃ (504 mg, 3.66 mmol) in DMF (5 mL) and water (0.50 mL) was degassed with nitrogen and heated to 45° C. overnight. The reaction was diluted with ethyl acetate (20 mL), filtered through a pad of Celite, and rinsed with ethyl acetate (20 mL). The filtrate was concentrated. The crude product was purified by column (12 g, 0-100% ethyl acetate in hexanes), affording a pale brown oil (670 mg, 99%); LC-MS (ESI+): m/z 278.0 (M+H)⁺.

### 5-(3-chlorophenyl)-4-ethyl-3-((4-methoxybenzyl)oxy)picolinonitrile

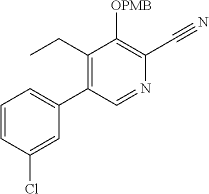

A suspension of 60% sodium hydride in mineral oil (193.4 mg, 4.83 mmol) was added to an ice-cold solution of (4-methoxyphenyl)methanol (0.6 mL, 4.83 mmol) and 3-chloro-5-(3-chlorophenyl)-4-ethyl-pyridine-2-carbonitrile (670 mg, 2.42 mmol) in DMF (7 mL) at 0° C. The resulting reaction mixture was stirred for 2 h. The reaction was quenched with cold water (20 mL). The mixture was extracted with ethyl acetate (20 mL×3). The combined organic layer was dried over Na₂SO₄ and concentrated. The crude product was purified by column (12 g, 0-60% ethyl acetate in hexanes), affording a white foaming solid (560 mg, 61%); LC-MS (ESI+): m/z 379.0 (M+H)⁺.

### 5-(3-chlorophenyl)-4-ethyl-3-((4-methoxybenzyl)oxy)picolinic acid

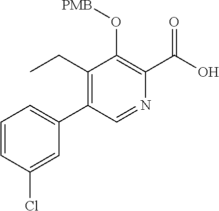

A mixture of 5-(3-chlorophenyl)-4-ethyl-3-[(4-methoxyphenyl)methoxy]pyridine-2-carbonitrile (560 mg, 1.48 mmol) and aq. 30% NaOH (6.27 mL, 47.02 mmol) in ethanol (5 mL) was heated to 100° C. for 2 h. The reaction was cooled to RT and concentrated directly to remove ethanol. The reaction mixture was acidified with 6 N HCl and the resulting precipitate was filtered. The solid was dissolved in methanol and concentrated to afford a brown residue (610 mg). The crude was subjected to the next reaction without further purification. LC-MS (ESI+): m/z 398.0 (M+H)⁺.

### ethyl (5-(3-chlorophenyl)-4-ethyl-3-((4-methoxybenzyl)oxy)picolinoyl)glycinate

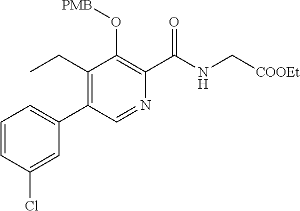

Triethylamine (0.52 mL, 3.77 mmol) was added to a stirred mixture of 5-(3-chlorophenyl)-4-ethyl-3-[(4-methoxyphenyl)methoxy]pyridine-2-carboxylic acid (300 mg, 0.75 mmol), ethyl 2-aminoacetate hydrochloride (210 mg, 1.51 mmol) and PyBOP (470 mg, 0.90 mmol) in DCM (5 mL) at rt. The resulting reaction mixture was stirred at RT for 3 days. The reaction was concentrated, and the crude residue was purified by column (4 g, 0-100% ethyl acetate in hexanes), affording a pale brown liquid (240 mg, 65%); LC-MS (ESI+): m/z 483.0 (M+H)⁺.

### (5-(3-chlorophenyl)-4-ethyl-3-hydroxypicolinoyl)glycine

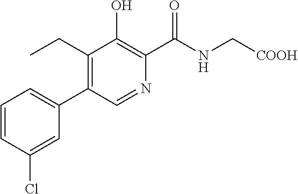 [SMILES: CCc1c(-c2cccc(Cl)c2)cnc(C(=O)NCC(=O)O)c1O]

Concentrated HCl (0.1 mL, 1.2 mmol) was added to a stirred solution of ethyl 2-[[5-(3-chlorophenyl)-4-ethyl-3-[(4-methoxyphenyl)methoxy]pyridine-2-carbonyl]amino]acetate (140 mg, 0.29 mmol) in ethanol (1.5 mL) at rt. The resulting reaction mixture was stirred for 2 h at rt. LCMS indicated complete PMB deprotection occurred. The reaction was concentrated directly under reduced pressure. The crude was dissolved in THF (1 mL) and methanol (1 mL) and treated with aq. 1 N NaOH (1.5 mL) for 2 h at rt. LCMS indicated complete conversion to the desired product. The reaction was concentrated directly to remove THF and methanol. The aqueous solution was acidified with 1 N HCl (1.52 mL) and the resulting precipitate was filtered. The crude solid was dissolved in DMSO (2 mL) and purified by prep-HPLC (30-85% Acetonitrile in water over 25 min), affording a white solid (62 mg, 60%). LC-MS (ESI+): m/z 335.0 (M+H)+; ¹H NMR (500 MHz, DMSO) δ 12.84 (s, 2H), 9.39 (s, 1H), 8.02 (s, 1H), 7.60-7.53 (m, 2H), 7.50 (d, J=1.0 Hz, 1H), 7.41-7.35 (m, 1H), 4.01 (d, J=6.0 Hz, 2H), 2.59-2.53 (q, J=5.4 Hz, 2H), 1.05 (t, J=7.5 Hz, 3H).

### Example 47: Preparation of Compound 47

### (3-hydroxy-4-methyl-5-(1-phenyl-1H-pyrazol-4-yl)picolinoyl)-L-alanine

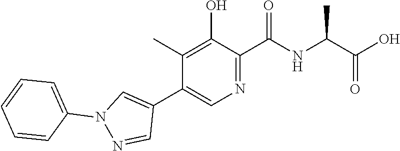 [SMILES: Cc1c(-c2cnn(-c3ccccc3)c2)cnc(C(=O)N[C@@H](C)C(=O)O)c1O]

The compound was synthesized according to the procedure for the preparation of (3-hydroxy-4-methyl-5-(2-phenyloxazol-5-yl)picolinoyl)glycine. LC-MS (ESI+): m/z 367 (M+H)+; 1H-NMR (300 MHz, DMSO-d6) δ 12.90 (brs, 1H), 12.77 (s, 1H), 9.17 (d, J=7.6 Hz, 1H), 8.93 (s, 1H), 8.33 (s, 1H), 8.18 (s, 1H), 7.94 (d, J=8.0 Hz, 2H), 7.55 (t, J=7.8 Hz, 2H), 7.37 (t, J=7.4 Hz, 1H), 4.51 (t, J=7.4 Hz, 1H), 2.37 (s, 3H), 1.47 (d, J=7.2 Hz, 3H).

### Example 48: Preparation of Compound 48

### (3-hydroxy-4-methyl-5-(1-phenyl-1H-pyrazol-4-yl)picolinoyl)-D-alanine

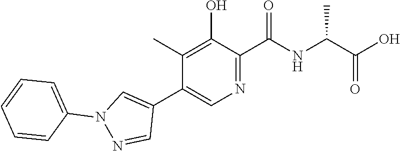 [SMILES: Cc1c(-c2cnn(-c3ccccc3)c2)cnc(C(=O)N[C@H](C)C(=O)O)c1O]

The compound was synthesized according to the procedure for the preparation of (3-hydroxy-4-methyl-5-(2-phenyloxazol-5-yl)picolinoyl)glycine. LC-MS (ESI+): m/z 367 (M+H)+; 1H-NMR (300 MHz, DMSO-d6) δ 12.88 (brs, 1H), 12.78 (s, 1H), 9.17 (d, J=7.7 Hz, 1H), 8.92 (s, 1H), 8.32 (s, 1H), 8.18 (s, 1H), 7.94 (d, J=8.0 Hz, 2H), 7.55 (t, J=7.8 Hz, 2H), 7.37 (t, J=7.4 Hz, 1H), 4.51 (t, J=7.2 Hz, 1H), 2.37 (s, 3H), 1.47 (d, J=7.2 Hz, 3H).

### Example 49: Preparation of Compound 49

### ethyl (5-(5-fluoro-1-phenyl-1H-pyrazol-4-yl)-3-((2-methoxyethoxy)methoxy)-4-methylpicolinoyl)glycinate

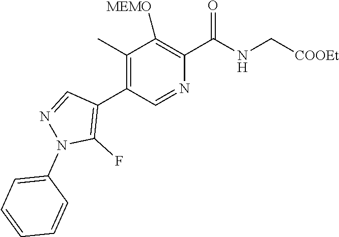

A mixture of ethyl 2-[[3-(2-methoxyethoxymethoxy)-4-methyl-5-(trifluoromethylsulfonyloxy)pyridine-2-carbonyl]amino]acetate (200 mg, 0.4200 mmol), 5-fluoro-1-phenyl-4-(4,4,5,5-tetramethyl-1,3,2-dioxaborolan-2-yl)pyrazole (240 mg, 0.830 mmol), Pd(dppf)Cl₂.DCM (34.4 mg, 0.040 mmol) and K₃PO₄ (268 mg, 1.26 mmol) in 1,4-Dioxane (3 mL) and Water (0.2000 mL) was degassed with Nitrogen and heated to 95° C. overnight. LCMS indicated MS/487. The reaction was cooled to rt. The reaction was diluted with methanol (10 mL) and filtered, filtrate was concentrated, crude was purified by column (4 g, 0-100% ethyl acetate in hexanes) afford brown oil (60 mg, 39%). LC-MS (ESI+): m/z 487.0 (M+H)⁺.

### (5-(5-fluoro-1-phenyl-1H-pyrazol-4-yl)-3-hydroxy-4-methylpicolinoyl)glycine

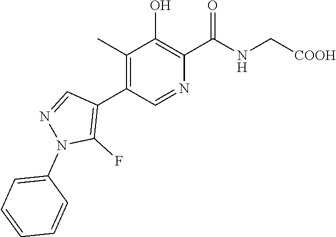 [SMILES: Cc1c(-c2cnn(-c3ccccc3)c2F)cnc(C(=O)NCC(=O)O)c1O]

4 M HCl in dioxane (0.2 mL, 0.1200 mmol) was added to a stirred solution of ethyl 2-[[5-(5-fluoro-1-phenyl-pyrazol-4-yl)-3-(2-methoxyethoxymethoxy)-4-methyl-pyridine-2-carbonyl]amino]acetate (60 mg, 0.120 mmol) in Ethanol (2 mL) at rt. The resulting reaction mixture was stirred at rt for 1 h. LCMS indicated complete MEM deprotection occurred. The reaction was concentrated directly, crude was dissolved in THF (2 mL) and Methanol (1 mL) and added aq. 1 N NaOH (0.5 mL). The reaction was stirred at rt for 2 h. LCMS indicated complete ester hydrolysis occurred. The reaction was concentrated directly, acidified by aq. 1 N HCl, precipitate was filtered. The solid was dissolved in DMSO and purified by prep-HPLC afford off-white solid (11 mg, 23%). LC-MS (ESI+): m/z 371.0 (M+H)⁺; 1H NMR (400 MHz, DMSO) δ 12.80 (s, 2H), 9.36 (t, J=6.2 Hz, 1H), 8.21 (s, 1H), 8.06 (d, J=3.1 Hz, 1H), 7.73 (d, J=8.3 Hz, 2H), 7.59 (t, J=7.9 Hz, 2H), 7.47 (t, J=7.4 Hz, 1H), 4.00 (d, J=6.1 Hz, 2H), 2.29 (d, J=1.1 Hz, 3H).

### Example 50: Preparation of Compound 50

### ethyl (3-hydroxy-4-methyl-5-(5-methyl-1-phenyl-1H-pyrazol-4-yl)picolinoyl)glycinate

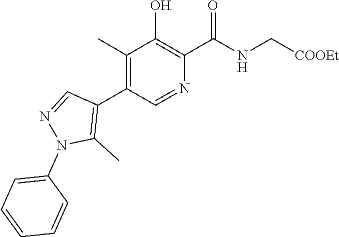 [SMILES: CCOC(=O)CNC(=O)c1ncc(-c2cnn(-c3ccccc3)c2C)c(C)c1O]

A mixture of ethyl 2-[[3-(2-methoxyethoxymethoxy)-4-methyl-5-(trifluoromethylsulfonyloxy)pyridine-2-carbonyl]amino]acetate (309 mg, 0.650 mmol), 5-methyl-1-phenyl-4-(4,4,5,5-tetramethyl-1,3,2-dioxaborolan-2-yl)pyrazole (250 mg, 0.880 mmol), Pd(dppf)Cl₂ (47.68 mg, 0.070 mmol) and K₃PO₄ (276.6 mg, 1.3 mmol) in 1,4-Dioxane (5 mL) and Water (0.25 mL) was heated to 90° C. overnight. The reaction was cooled to rt, diluted with ethyl acetate (10 mL) and aq. 1 N HCl (5 mL). The organic layer was separated. The organic layer was dried over Na₂SO₄ and concentrated. The crude was dissolved in ethanol (10 mL), treated with 4 N HCl in dioxane (0.3 mL) for 2 h. LCMS indicated MEM deprotection occurred. The reaction was concentrated directly. The crude was purified by column (4 g, 0-100% ethyl acetate in hexanes) afford yellow solid (40 mg, 16% over 2 steps). LC-MS (ESI+): m/z 395.0 (M+H)⁺.

### (3-hydroxy-4-methyl-5-(5-methyl-1-phenyl-1H-pyrazol-4-yl)picolinoyl)glycine

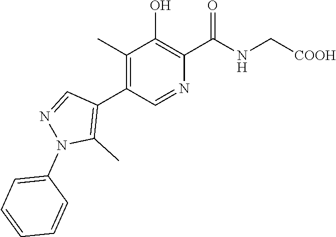 [SMILES: Cc1c(-c2cnn(-c3ccccc3)c2C)cnc(C(=O)NCC(=O)O)c1O]

Aq 1 N NaOH (0.5 mL) was added to a stirred solution of ethyl 2-[[3-hydroxy-4-methyl-5-(5-methyl-4-phenyl-pyrazol-1-yl)pyridine-2-carbonyl]amino]acetate (40 mg, 0.100 mmol) in THF (5 mL) and Methanol (2 mL) at rt. The resulting reaction mixture was stirred for 2 days at rt and heated to 45° C. for 2 h. The reaction was acidified with aq. 1 N HCl and concentrated directly. The crude was purified by Prep-HPLC by dissolving in DMSO (2 mL) afford off-white solid (20 mg, 52%). LC-MS (ESI+): m/z 367.0 (M+H)⁺; 1H NMR (400 MHz, DMSO) δ 12.73 (s, 2H), 9.33 (t, J=6.1 Hz, 1H), 8.07 (s, 1H), 7.83 (s, 1H), 7.65-7.53 (m, 4H), 7.51-7.38 (m, 1H), 4.00 (d, J=6.2 Hz, 2H), 2.26 (s, 2H), 2.20 (s, 2H).

### Example 51: Preparation of Compound 51

### Preparation of Compound 51 (AKE-7595)

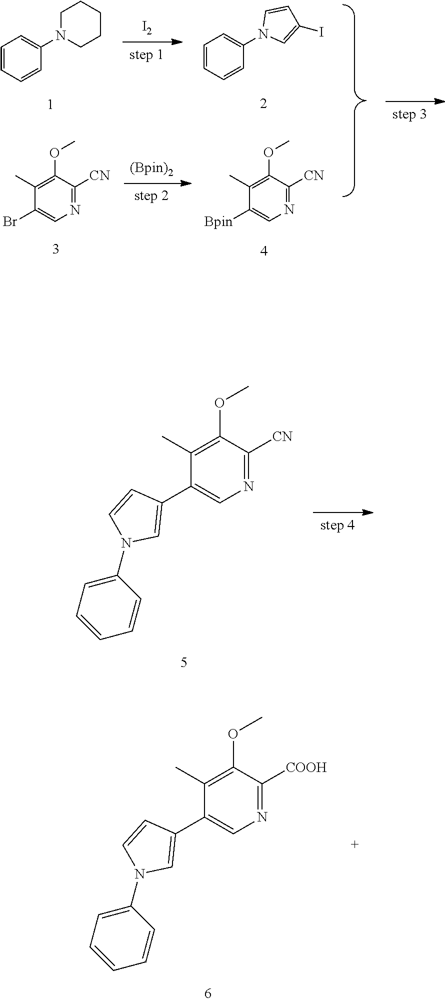

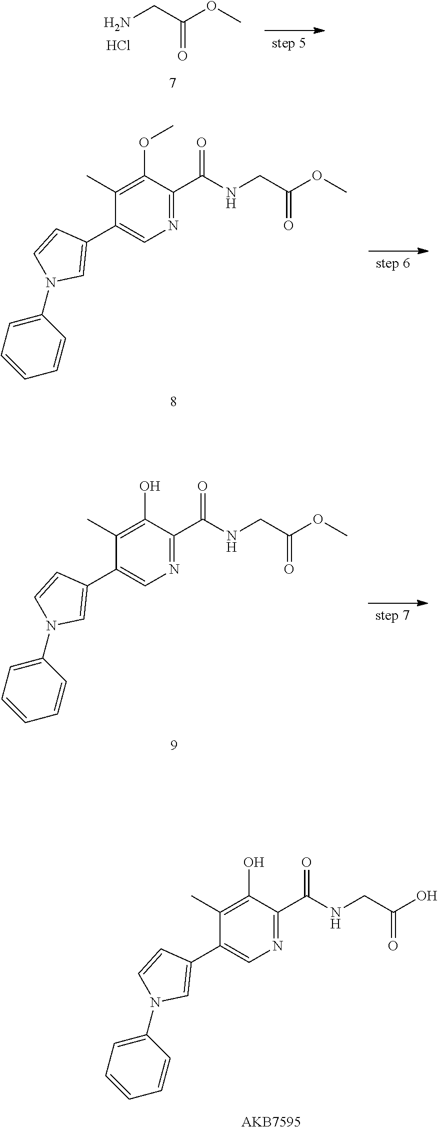

Step 1:

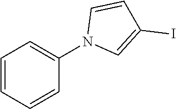 [SMILES: Ic1ccn(-c2ccccc2)c1]

A solution of 1-phenylpiperidine (1 g, 6.2 mmol), Cu(OAc)₂ (2.25 g, 12.40 mmol), 12 (1575 mg, 6.20 mmol) and DMAP (758 mg, 6.20 mmol) in ACN (20 mL) was stirred at 80° C. under oxygen atmosphere for 5.5 hrs. After the reaction was completed as indicated by TLC, the reaction was cooled to rt, quenched with a saturated aqueous NH4Cl solution (250 mL) and extracted with EtOAc (100 mL×3). The combined organic phase was dried, filtered and concentrated. The residue was purified by column chromatography (EA: PE=1:50) to give 74 mg of the title compound. ¹H-NMR (300 MHz, CDCl₃) (7.48-7.22 (m, 5H), 7.15 (d, J=1.8 Hz, 1H), 6.95 (dd, J=3.0, 1.8 Hz, 1H), 6.43 (d, J=3.0 Hz, 1H).

Step 2:

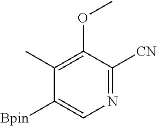

Under nitrogen protection, a solution of 5-bromo-3-methoxy-4-methylpicolinonitrile (160 mg, 0.71 mmol), B₂Pin₂ (269 mg, 1.06 mmol), KOAc (138 mg, 1.41 mmol) and Pd(dppf)Cl₂(26 mg, 0.035 mmol) in dioxane (10 mL) was stirred at 100° C. for about 6 hrs. After the most of 5-bromo-3-methoxy-4-methylpicolinonitrile was consumed as indicated by TLC analysis, the reaction was concentrated directly and the residue was purified by column chromatography to give 185 mg of the title compound as white solid. LC-MS (ESI+): m/z 275(M+H)⁺.

Step 3:

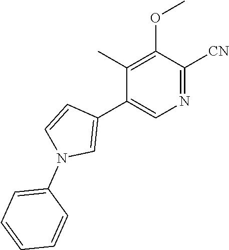 [SMILES: COc1c(C#N)ncc(-c2ccn(-c3ccccc3)c2)c1C]

Under nitrogen protection, a solution of 3-methoxy-4-methyl-5-(4,4,5,5-tetramethyl-1,3,2-dioxaborolan-2-yl)picolinonitrile (185 mg, 0.68 mmol), 3-iodo-1-phenyl-1H-pyrrole (294 mg, 1.09 mmol), Pd(dppf)Cl₂ (25 mg, 0.034 mmol), K₃PO₄·3H₂O (540 mg, 2.026 mmol) in dioxane (15 mL) and H₂O (3 mL) was stirred at 75° C. for about 3 hrs. After the reaction was completed as indicated by TLC analysis, the reaction was concentrated to dryness directly and the residue was purified by column chromatography to give 48 mg of the title compound as yellow oil. LC-MS (ESI+): m/z 289(M+H)⁺.

Step 4:

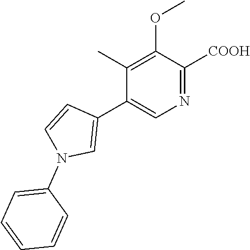 [SMILES: COc1c(C(=O)O)ncc(-c2ccn(-c3ccccc3)c2)c1C]

The compound was synthesized according to the step 5 of Preparation of AKB-7588 (Compound 57). LC-MS (ESI+): m/z 309(M+H)⁺;

Step 5:

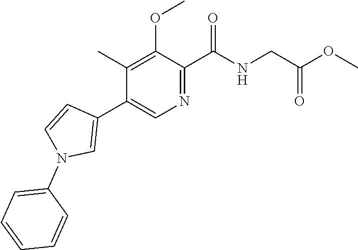 [SMILES: COC(=O)CNC(=O)c1ncc(-c2ccn(-c3ccccc3)c2)c(C)c1OC]

The compound was synthesized according to the step 6 of Preparation of AKB-7588 (Compound 57). LC-MS (ESI+): m/z 380(M+H)⁺;

Step 6:

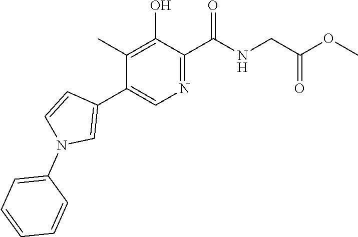 [SMILES: COC(=O)CNC(=O)c1ncc(-c2ccn(-c3ccccc3)c2)c(C)c1O]

The compound was synthesized according to the step 7 of Preparation of AKB-7588 (Compound 57). LC-MS (ESI+): m/z 366(M+H)⁺.

Step 7:

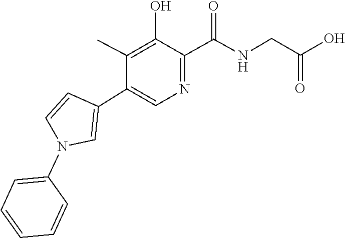 [SMILES: Cc1c(-c2ccn(-c3ccccc3)c2)cnc(C(=O)NCC(=O)O)c1O]

The compound was synthesized according to the step 8 of Preparation of AKB-7588 (Compound 57). LC-MS (ESI+): m/z 352 (M+H)⁺; ¹H-NMR (300 MHz, DMSO-d₆) δ 12.82 (brs, 1H), 12.78 (s, 1H), 9.25 (t, J=6.0 Hz, 1H), 8.29 (s, 1H), 7.75 (d, J=3.0 Hz, 1H), 7.70 (m, 2H), 7.60-7.50 (m, 3H), 7.40-7.32 (m, 1H), 6.88 (s, 1H), 4.00 (d, J=6.0 Hz, 2H), 2.40 (s, 3H).

### Example 52: Preparation of Compound 52

### Preparation of Compound 52 (AKE-7562)

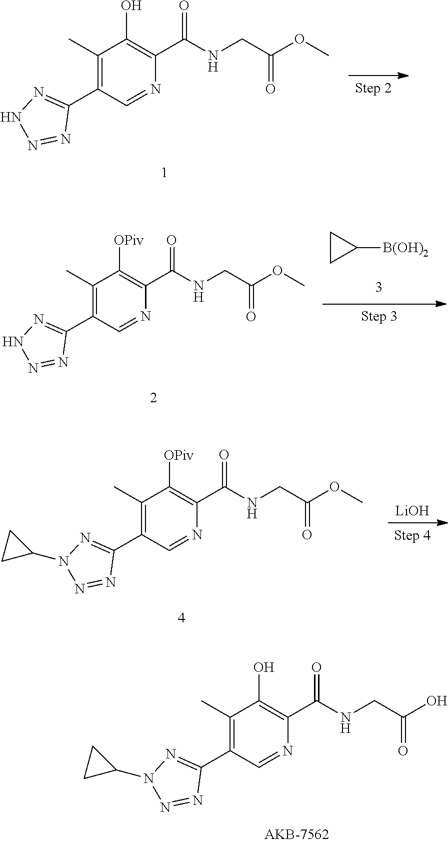

Step 1:

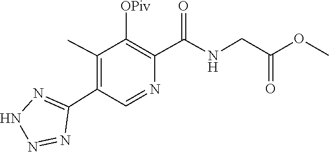

A solution of methyl (3-hydroxy-4-methyl-5-(2H-tetrazol-5-yl)picolinoyl)glycinate (69 mg, 0.24 mmol) and PivCl (34 mg, 0.28 mmol) in THF was added TEA (36 mg, 0.36 mmol) in one portion. The resulting mixture was stirred at rt for about 6 hrs. After the reaction was completed as indicated by TLC analysis, the reaction was concentrated to dryness directly for column purification. 70 mg of the title compound was obtained as oil. LC-MS (ESI+): m/z 377 (M+H)⁺.

Step 2:

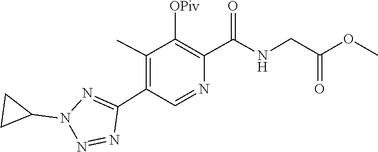

A suspension of 2-((2-methoxy-2-oxoethyl)carbamoyl)-4-methyl-5-(2H-tetrazol-5-yl)pyridin-3-yl pivalate (30 mg, 0.08 mmol), cyclopropylboronic acid (14 mg, 0.16 mmol), Cu(OAc)₂ (8 mg, 0.04 mmol), 2,2′-bipyridine (19 mg, 0.12 mmol) and Na₂CO₃ (21 mg, 0.20 mmol) in DCE (0.75 mL) was stirred under reflux for about 36 hrs. After the reaction was completed as indicated by LCMS analysis, the reaction was concentrated to dryness directly. The residue was purified by column chromatography (DCM: PE=1:1 to 1:0) to give 10 mg of the title compound. LC-MS (ESI+): m/z 417 (M+H)⁺.

Step 3:

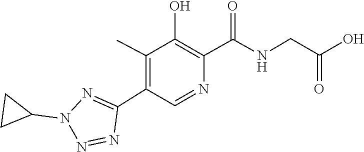 [SMILES: Cc1c(-c2nnn(C3CC3)n2)cnc(C(=O)NCC(=O)O)c1O]

The compound was synthesized according to the step 12 of Preparation of AKB-7560 (Compound 65). LC-MS (ESI+): m/z 319 (M+H)⁺; ¹H-NMR (300 MHz, DMSO-d₆) δ 12.95 (brs, 1H), 12.88 (brs, 1H), 9.54 (t, J=6.3 Hz, 1H), 8.63 (s, 1H), 4.52 (m, 1H), 4.02 (d, J=6.3 Hz, 2H), 2.52 (s, 3H), 1.50-1.40 (m, 2H), 1.40-1.33 (m, 2H).

### Example 53: Preparation of Compound 54

### Preparation of Compound 54 (AKE-7590)

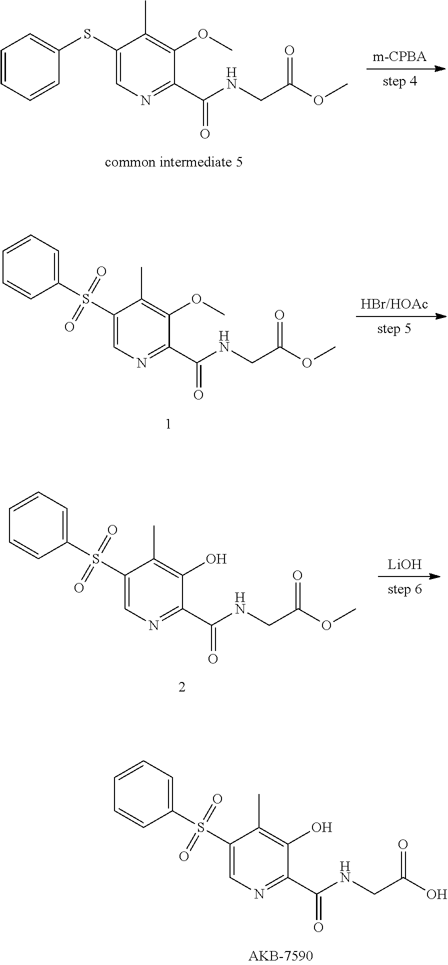

Step 4:

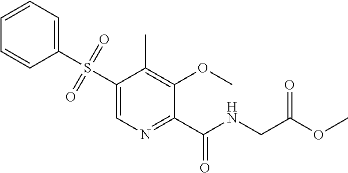 [SMILES: COC(=O)CNC(=O)c1ncc(S(=O)(=O)c2ccccc2)c(C)c1OC]

To a solution of methyl (3-methoxy-4-methyl-5-(phenylthio)picolinoyl)glycinate (180 mg, 0.52 mmol) in DCM (5 mL) at rt was added m-CPBA (269 mg, 1.56 mmol) portion wise over 5 min. The reaction was stirred at rt for about 5 hrs. After the reaction was completed as indicated by TLC analysis, the resulting mixture was diluted with water (10 mL) and extracted with DCM (10 mL×2). The combined organic phases were dried with Na₂SO₄ (10 g), filtered and concentrated. The residue was purified by flash silica-gel chromatography (EA: Hex=1:2 to 1:1) to give 190 mg of the title compound. LC-MS (ESI+): m/z 379 (M+H)⁺; ¹H-NMR (300 MHz, CDCl₃) (9.03 (s, 1H), 8.20 (brs, 1H), 7.98-7.88 (m, 2H), 7.70-7.50 (m, 3H), 4.25 (d, J=6.3 Hz, 2H), 3.89 (s, 3H), 3.80 (s, 3H), 2.46 (s, 3H).

Step 5:

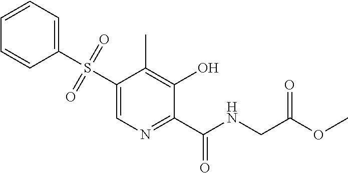 [SMILES: COC(=O)CNC(=O)c1ncc(S(=O)(=O)c2ccccc2)c(C)c1O]

The compound was synthesized according to the step 4 of Preparation of AKB-7589 (Compound 59). LC-MS (ESI+): m/z 365 (M+H)⁺.

Step 6:

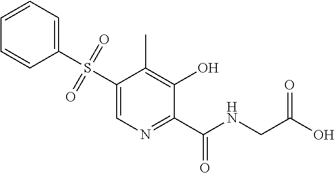 [SMILES: Cc1c(S(=O)(=O)c2ccccc2)cnc(C(=O)NCC(=O)O)c1O]

LC-MS (ESI+): m/z 351 (M+H)⁺; ¹H-NMR (300 MHz, DMSO-d₆) (13.08 (s, 1H), 12.88 (s, 1H), 9.67 (s, 1H), 8.76 (s, 1H), 8.05-7.90 (m, 2H), 7.80-7.61 (m, 3H), 4.01 (d, J=6.3 Hz, 2H), 2.30 (s, 3H).

### Example 54: Preparation of Compound 55

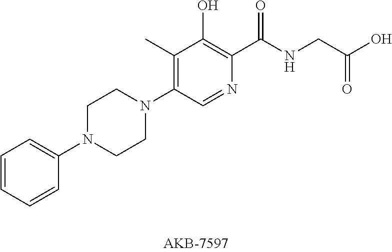 [SMILES: Cc1c(N2CCN(c3ccccc3)CC2)cnc(C(=O)NCC(=O)O)c1O]

Compound 55 (AKE-7597) was prepared according to the preparation of AKB-7560 (Compound 65). LC-MS (ESI+): m/z 371 (M+H)⁺. ¹H-NMR (300 MHz, CD3OD) δ 7.97 (s, 1H), 7.35-7.25 (m, 2H), 7.06-7.01 (m, 2H), 6.88 (t, J=7.2 Hz, 1H), 4.12 (s, 2H), 3.35-3.30 (m, 4H), 3.28-3.20 (m, 4H), 2.21 (s, 3H).

### Example 55: Preparation of Compound 56

### Preparation of Compound 56 (AKE-7596)

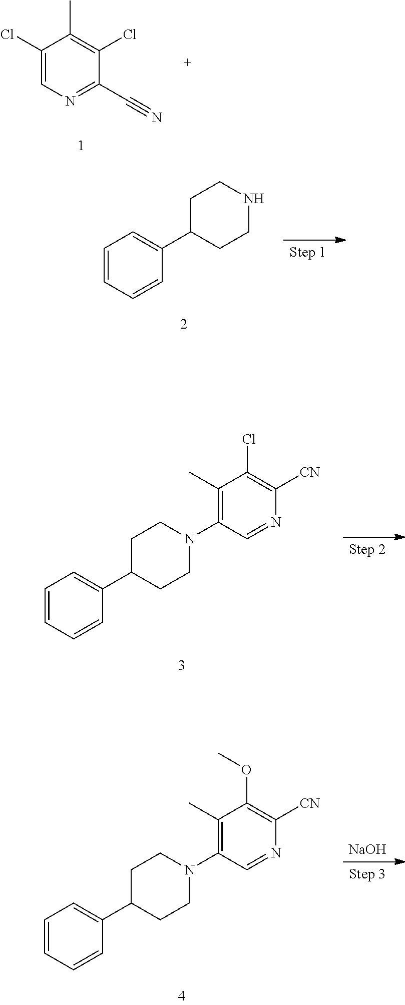

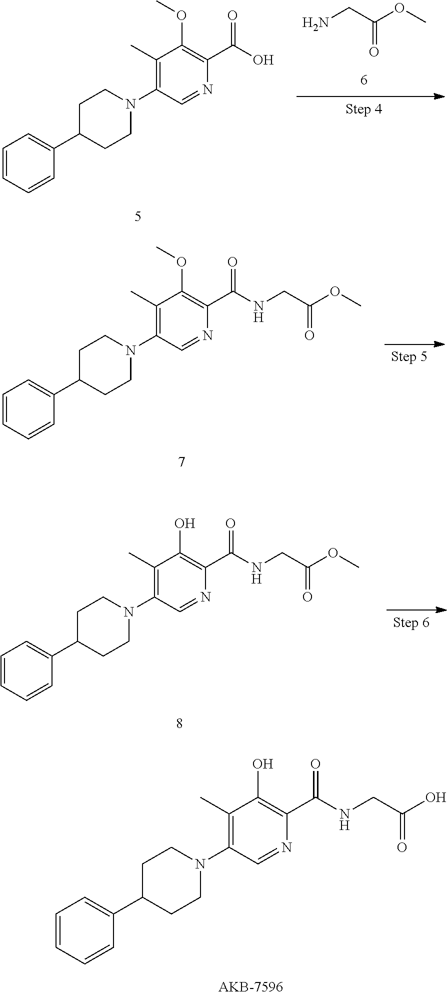

Step 1:

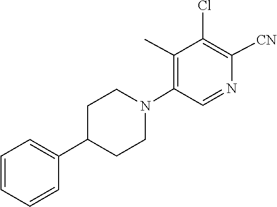 [SMILES: Cc1c(N2CCC(c3ccccc3)CC2)cnc(C#N)c1Cl]

Under nitrogen protection, to a solution of 3,5-dichloro-4-methylpicolinonitrile (2.18 g, 11.78 mmol) in t-BuOH (20 ml) was added 4-phenylpiperidine (950 mg, 5.89 mmol), RuPhos (412 mg, 0.88 mmol), Cs₂CO₃ (5.76 g, 17.67 mmol) and Pd(OAc)₂ (198 mg, 0.88 mmol). The reaction was stirred at 110° C. overnight. After the reaction was completed as indicated by TLC, the resulting mixture was cooled to rt, diluted with water (50 mL) and extracted with EtOAc (50 mL×3). The combined organic phases were dried with Na₂SO₄ (50 g), filtered and concentrated. The residue was purified by flash silica-gel chromatography (EA: Hex=1:50 to 1:2) to give 310 mg of the title compound as yellow solid. LC-MS (ESI+): m/z 312 (M+H)⁺; ¹H-NMR (300 MHz, DMSO-d₆) δ 8.38 (s, 1H), 7.40-7.31 (m, 4H), 7.28-7.18 (m, 1H), 3.48-3.36 (m, 2H), 3.07-2.95 (m, 2H), 2.80-2.68 (m, 1H), 2.38 (s, 3H), 1.95-1.78 (m, 4H).

Step 2:

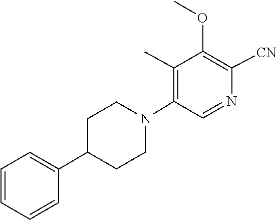 [SMILES: COc1c(C#N)ncc(N2CCC(c3ccccc3)CC2)c1C]

The compound was synthesized according to the step 3 of Preparation of AKB-7588 (Compound 57). LC-MS (ESI+): m/z 308 (M+H)⁺; ¹H-NMR (300 MHz, CDCl₃) δLC-MS (ESI+): m/z 308 (M+H)⁺; ¹H-NMR (300 MHz, CDCl₃) δ 8.15 (s, 1H), 7.40-7.25 (m, 5H), 4.04 (s, 3H), 3.49-3.31 (m, 2H), 3.05-2.85 (m, 4H), 2.78-2.64 (m, 1H), 2.27 (s, 3H), 2.05-1.80 (m, 4H).

Step 3:

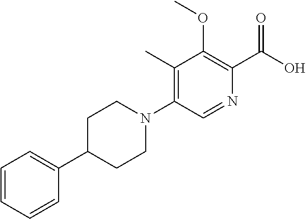 [SMILES: COc1c(C(=O)O)ncc(N2CCC(c3ccccc3)CC2)c1C]

The compound was synthesized according to the step 5 of Preparation of AKB-7588 (Compound 57). LC-MS (ESI+): m/z 327 (M+H)⁺; ¹H-NMR (300 MHz, DMSO-d₆) δ 8.11 (s, 1H), 7.40-7.15 (m, 5H), 3.80 (s, 3H), 3.35 (m, 2H), 2.98-2.90 (m, 2H), 2.79-2.62 (m, 1H), 2.27 (s, 3H), 1.99-1.78 (m, 4H).

Step 4:

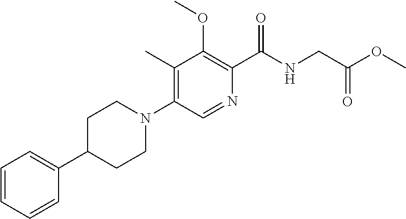 [SMILES: COC(=O)CNC(=O)c1ncc(N2CCC(c3ccccc3)CC2)c(C)c1OC]

The compound was synthesized according to the step 6 of Preparation of AKB-7588 (Compound 57). LC-MS (ESI+): m/z 398 (M+H)⁺; ¹H-NMR (300 MHz, CDCl₃) δ 8.28 (t, J=5.4 Hz, 1H), 8.10 (s, 1H), 7.41-7.20 (m, 5H), 4.32-4.24 (d, J=5.4 Hz, 2H), 3.92 (s, 3H), 3.79 (s, 3H), 3.43-3.31 (m, 2H), 3.01-2.90 (m, 2H), 2.77-2.65 (m, 1H), 2.30 (s, 3H), 2.05-1.86 (m, 4H).

Step 5:

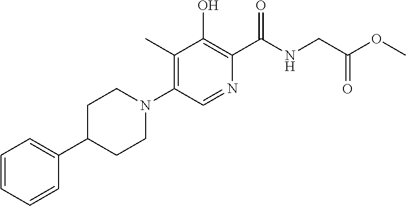 [SMILES: COC(=O)CNC(=O)c1ncc(N2CCC(c3ccccc3)CC2)c(C)c1O]

The compound was synthesized according to the step 7 of Preparation of AKB-7588 (Compound 57). LC-MS (ESI+): m/z 384 (M+H)⁺.

Step 6:

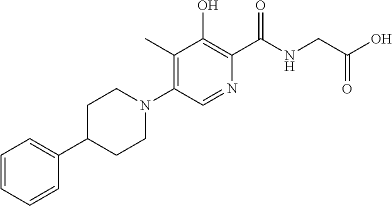 [SMILES: Cc1c(N2CCC(c3ccccc3)CC2)cnc(C(=O)NCC(=O)O)c1O]

The compound was synthesized according to the step 8 of Preparation of AKB-7588 (Compound 57). LC-MS (ESI+): m/z 370 (M+H)⁺; ¹H-NMR (300 MHz, DMSO-d₆) δ 12.81 (s, 1H), 12.55 (s, 1H), 9.13 (t, J=5.4 Hz, 1H), 7.93 (s, 1H), 7.39-7.18 (m, 5H), 3.96 (d, J=5.4 Hz, 2H), 3.01-2.90 (m, 2H), 2.80-2.68 (m, 1H), 2.16 (s, 3H), 1.98-1.75 (m, 4H).

### Example 56: Preparation of Compound 57

### Preparation of Compound 57 (AKE-7588)

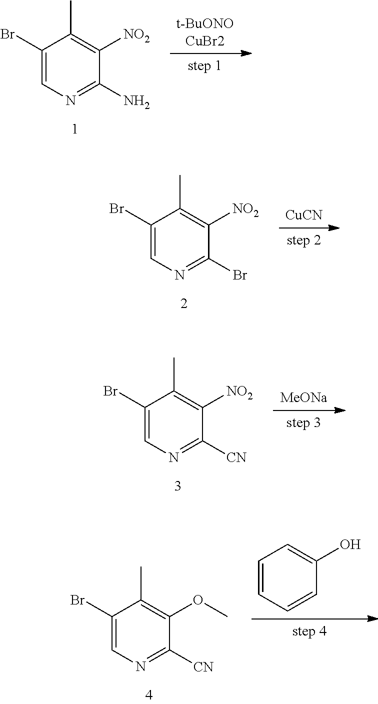

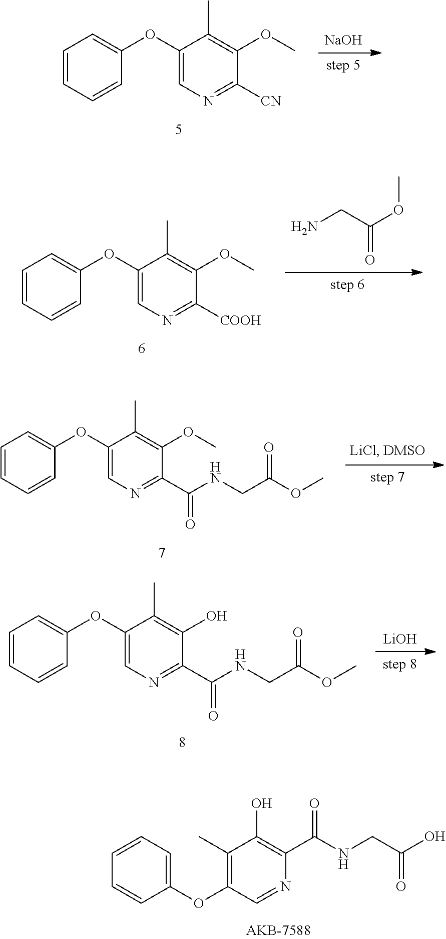

Step 1:

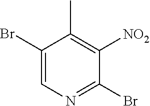

To a solution of 5-bromo-4-methyl-3-nitropyridin-2-amine (8.75 g, 0.038 mol) in ACN (200 mL) at 80° C. was added CuBr₂ (10.18 g, 0.046 mol). After the resulting mixture was stirred at 80° C. for 20 min, t-BuONO (6.22 g, 0.060 mol) was added dropwise to the reaction over 5 mins. The reaction was continued to stir at 80° C. for 1 hr. After the reaction was completed as indicated by TLC analysis, the reaction was cooled to rt, quenched with water (200 mL) and extracted with EtOAc (200 mL×2). The combined organic phases were dried with anhydrous Na₂SO₄ (50 g), filtered and concentrated in vacuo. The residue was purified by silica-gel column chromatography (EA: Hex=1: 30˜1:20) to give 4.6 g of the desired product as yellow solid. LC-MS (ESI+): m/z 295 (M+H)⁺; ¹H-NMR (300 MHz, CDCl₃) δ 8.58 (s, 1H), 2.42 (s, 3H).

Step 2:

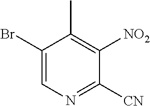

A suspension of 2,5-dibromo-4-methyl-3-nitropyridine (4.4 g, 0.015 mol) and CuCN (2.68 g, 0.030 mol) in DMF (100 mL) was stirred at 120° C. for 2 hrs. After the reaction was completed as indicated by TLC analysis, the reaction was cooled to rt, quenched with water (200 mL) and extracted with EtOAc (200 mL×2). The combined organic phase was washed with water (100 mL) and brine (100 mL), dried with anhydrous Na₂SO₄ (100 g), filtered and concentrated in vacuo. The residue was purified by silica-gel column chromatography (EA: Hex=1: 30 to 1:10) to give 1.29 of the title compound as yellow solid. ¹H-NMR (300 MHz, CDCl₃) (8.91 (s, 1H), 2.54 (s, 3H).

Step 3:

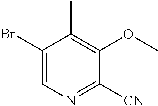

A solution of 5-bromo-4-methyl-3-nitropicolinonitrile (1.33 g, 5.52 mmol) in methanol (30 mL) at rt was added a solution of MeONa in MeOH (993 mg, 5.52 mmol, 30 wt %). The reaction was stirred at 50° C. for 7 hrs. After the reaction was completed as indicated by TLC analysis, the reaction was quenched with water (50 mL) and extracted with EtOAc (50 mL×2). The combined organic phases were dried with anhydrous Na₂SO₄ (100 g), filtered and concentrated in vacuo. The residue was purified by silica-gel column chromatography (EA: Hex=1: 30 to 1:10) to give 640 mg of the title product as yellow solid. LC-MS (ESI+): m/z 227, 229 (M+H)⁺; ¹H-NMR (300 MHz, CDCl₃) δ 8.50 (s, 1H), 4.09 (s, 3H), 2.41 (s, 3H).

Step 4:

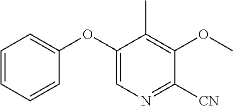 [SMILES: COc1c(C#N)ncc(Oc2ccccc2)c1C]

Under nitrogen protection, to a solution of phenol (100 mg, 1.06 mmol) in DMF (3 mL) at rt was added NaH (71 mg, 1.77 mmol) portion wise over 2 mins. After the solution was stirred at rt for 30 minutes, the reaction mixture was added dropwise to a solution of 5-bromo-3-methoxy-4-methylpicolinonitrile (200 mg, 0.86 mmol) in DMF (2 mL) over 5 mins. The reaction was stirred at 100° C. for 2 hrs. After the reaction was completed as indicated by TLC analysis, the reaction was cooled to rt, quenched with brine (5 mL) and extracted with EtOAc (5 mL×3). The combined organic phase was washed with brine, dried with anhydrous Na₂SO₄, filtered and concentrated in vacuo. The residue was purified by silica-gel column chromatography (PE: EtOAc=20:1) to give 110 mg of the crude title compound as light yellow oil. ¹H NMR (300 MHz, CDCl₃) δ 7.91 (s, 1H), 7.41 (t, J=7.9 Hz, 2H), 6.93 (t, J=7.4 Hz, 1H), 6.84 (d, J=7.6 Hz, 2H), 4.11 (s, 3H), 2.29 (s, 3H).

Step 5:

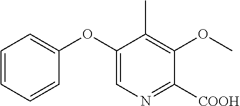 [SMILES: COc1c(C(=O)O)ncc(Oc2ccccc2)c1C]

To a solution of 3-methoxy-4-methyl-5-phenoxypicolinonitrile (100 mg, 0.42 mmol) in EtOH (3 mL) and H₂O (2 mL) at rt was added NaOH (167 mg, 4.17 mmol) in one portion. The reaction was stirred at 80° C. for 7 hrs. After the reaction was completed as indicated by LCMS analysis, the reaction was adjusted to pH 2-3 with a diluted HCl solution (2N). A large amount of the solid was precipitated. The solid was collected by filtration and slurried in ethanol for 1 hr. After filtration and drying, 74 mg of the title compound as yellow oil was obtained. ¹H NMR (300 MHz, DMSO-d₆) δ 7.94 (s, 1H), 7.42 (t, J=7.7 Hz, 2H), 7.18 (t, J=7.7 Hz, 1H), 7.04 (d, J=8.1 Hz, 2H), 3.83 (s, 3H), 2.18 (s, 3H).

Step 6:

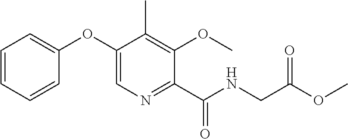 [SMILES: COC(=O)CNC(=O)c1ncc(Oc2ccccc2)c(C)c1OC]

To a solution of 3-methoxy-4-methyl-5-phenoxypicolinic acid (74 mg, 0.29 mmol) in DCM (2 mL) at room temperature was added glycine methyl ester hydrochloride (54 mg, 0.43 mmol), EDCl (110 mg, 0.57 mmol), HOBt (77 mg, 0.57 mmol) and DIEA (74 mg, 0.57 mmol). The reaction was stirred at rt for 2 hrs. After the reaction was completed as indicated by TLC analysis, the reaction was quenched with brine (2 mL) and extracted with CH₂Cl₂ (2 mL×3). The combined organic phase was washed with brine, dried with anhydrous Na₂SO₄, filtered and concentrated in vacuo. The residue was purified by silica-gel column chromatography (PE: EtOAc=3:1) to give 31 mg of the title compound. LC-MS (ESI+): m/z 331 (M+H)⁺.

Step 7:

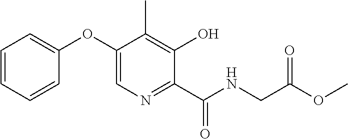 [SMILES: COC(=O)CNC(=O)c1ncc(Oc2ccccc2)c(C)c1O]

A suspension of methyl 2-(3-methoxy-4-methyl-5-phenoxypicolinamido)acetate (29 mg, 0.09 mmol) and LiCl (74 mg, 1.76 mmol) in DMA (1 mL) was stirred at 85° C. for 6 hrs. After the reaction was completed as indicated by TLC analysis, the reaction was cooled to rt, quenched with brine (2 mL) and extracted with DCM (2 mL×3). The combined organic phase was washed with brine, dried with anhydrous Na₂SO₄, filtered and concentrated in vacuo to give 29 mg of the title compound as yellow oil. LC-MS (ESI+): m/z 317 (M+H)⁺.

Step 8:

### 2-(3-hydroxy-4-methyl-5-phenoxypicolinamido)acetic acid

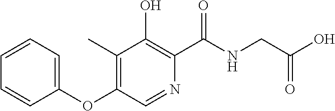 [SMILES: Cc1c(Oc2ccccc2)cnc(C(=O)NCC(=O)O)c1O]

The compound was synthesized according to the step 12 of Preparation of AKB-7560 (Compound 65). ¹H NMR (300 MHz, DMSO-d₆) δ 12.86 (s, 1H), 12.79 (brs, 1H), 9.23 (t, J=6.2 Hz, 1H), 7.76 (s, 1H), 7.45-7.36 (m, 2H), 7.17 (t, J=7.4 Hz, 1H), 7.05 (d, J=7.9 Hz, 2H), 3.97 (d, J=6.2 Hz, 2H), 2.11 (s, 3H).

### Example 57: Preparation of Compound 58

### Preparation of Compound 58 (AKE-7591)

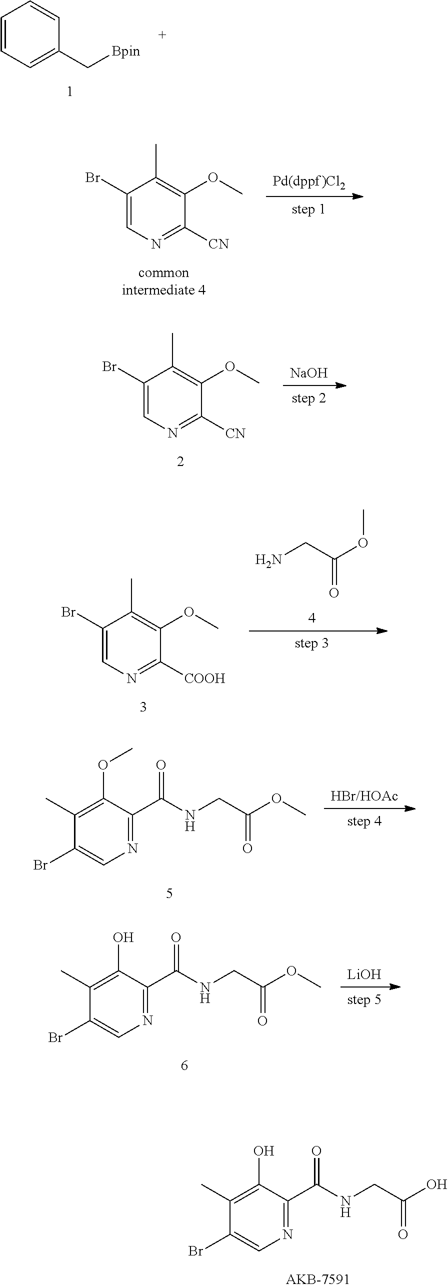

Step 1:

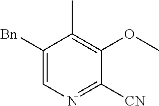 [SMILES: COc1c(C#N)ncc(Cc2ccccc2)c1C]

Under nitrogen protection, a reaction mixture of 5-bromo-3-methoxy-4-methylpicolinonitrile (157 mg, 0.691), benzyl Bpin (226 mg, 1.04 mmol), Pd(dppf)Cl₂(26 mg, 0.035 mmol) and K₃PO₄·3H₂O (553 mg, 2.074 mmol) in dioxane (10 mL) and H₂O (1 mL) was stirred at 75° C. for about 7 hrs. After the reaction was completed as indicated by TLC, the reaction was concentrated to dryness and the residue was purified by column chromatography to give 170 mg of the title compound as oil. LC-MS (ESI+): m/z 239(M+H)⁺.

Step 2:

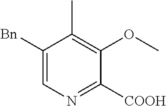 [SMILES: COc1c(C(=O)O)ncc(Cc2ccccc2)c1C]

The compound was synthesized according to the step 5 of Preparation of AKB-7588 (Compound 57). LC-MS (ESI+): m/z 258(M+H)⁺;

Step 3:

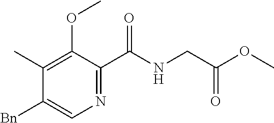

The compound was synthesized according to the step 6 of Preparation of AKB-7588 (Compound 57). LC-MS (ESI+): m/z 329 (M+H)+

Step 4:

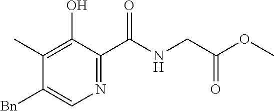

The compound was synthesized according to the step 7 of Preparation of AKB-7588 (Compound 57). LC-MS (ESI+): m/z 315(M+H)⁺;

Step 5:

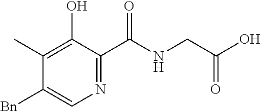 [SMILES: Cc1c(Cc2ccccc2)cnc(C(=O)NCC(=O)O)c1O]

The compound was synthesized according to the step 8 of Preparation of AKB-7588 (Compound 57). LC-MS (ESI+): m/z 301(M+H)⁺; ¹H-NMR (300 MHz, DMSO-d₆) δ 12.86 (brs, 1H), 12.64 (s, 1H), 9.13 (t, J=5.7 Hz, 1H), 8.05 (s, 1H), 7.39-7.18 (m, 5H), 4.05 (s, 2H), 3.99 (d, J=5.7 Hz, 2H), 2.10 (s, 3H).

### Example 58: Preparation of Compound 59

### Preparation of Compound 59 (AKE-7589)

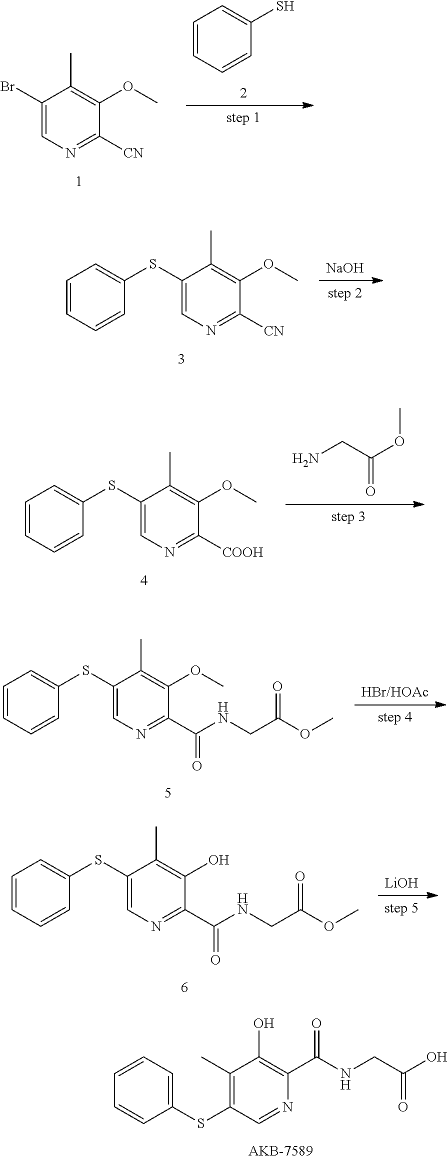

Step 1:

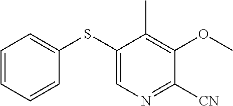 [SMILES: COc1c(C#N)ncc(Sc2ccccc2)c1C]

To a solution of benzenethiol (88 mg, 0.80 mmol) in THF (3 mL) at rt was added sodium hydride (53 mg, 1.32 mmol) in one portion. The reaction was stirred at rt for about 0.5 hr. A solution of 5-bromo-3-methoxy-4-methylpicolinonitrile (150 mg, 0.66 mmol) in THF (2 mL) was added to the solution above dropwise over 5 mins. The resulting mixture was continued to stir at rt for 1 hr. After the reaction was completed as indicated by TLC analysis, the reaction was quenched with water (5 mL) and extracted with EtOAc (3×5 ml). The combined organic phases were dried with anhydrous Na₂SO₄, filtered and concentrated in vacuo. The residue was purified by silica-gel column chromatography (EA: Hex=1: 50 to 1:30) to give 140 mg of the title compound as white oil. LC-MS (ESI+): m/z 257 (M+H)⁺; ¹H-NMR (300 MHz, CDCl₃) δ 7.82 (s, 1H), 7.55-7.42 (m, 5H), 4.09 (s, 3H), 2.35 (s, 3H).

Step 2:

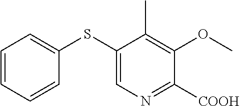 [SMILES: COc1c(C(=O)O)ncc(Sc2ccccc2)c1C]

The compound was synthesized according to the step 5 of Preparation of AKB-7588 (Compound 57). ¹H-NMR (300 MHz, DMSO-d₆) (13.35 (s, 1H), 8.04 (s, 1H), 7.50-7.35 (m, 5H), 3.80 (s, 3H), 2.31 (s, 3H).

Step 3:

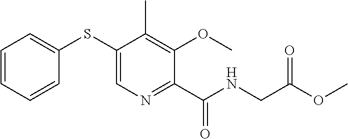 [SMILES: COC(=O)CNC(=O)c1ncc(Sc2ccccc2)c(C)c1OC]

To a solution of 3-methoxy-4-methyl-5-(phenylthio)picolinic acid (110 mg, 0.40 mmol) in DCM (5 ml) at rt was added methyl glycinate (75 mg, 0.60 mmol), EDCl (154 mg, 0.80 mmol), HOBt (108 mg, 0.80 mmol) and DIEA (103 mg, 0.80 mmol). The reaction was stirred at 45° C. overnight. After the reaction was completed as indicated by TLC, the resulting mixture was diluted with water (10 mL) and extracted with DCM (10 mL×3). The combined organic phased were dried with Na₂SO₄(10 g), filtered and concentrated. The residue was purified by flash silica-gel chromatography (EA: Hex=1: 3 to 1:2) to give 105 mg of the title compound as yellow solid. LC-MS (ESI+): m/z 347 (M+H)⁺; ¹H-NMR (300 MHz, CDCl₃) δ 8.15 (s, 1H), 7.94 (s, 1H), 7.39 (s, 5H), 4.22 (d, J=6.3 Hz, 2H), 3.92 (s, 3H), 3.78 (s, 3H), 2.38 (s, 3H).

Step 4:

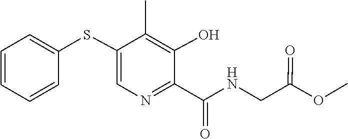 [SMILES: COC(=O)CNC(=O)c1ncc(Sc2ccccc2)c(C)c1O]

To a solution of methyl (3-methoxy-4-methyl-5-(phenylthio)picolinoyl)glycinate (100 mg, 0.29 mmol) in AcOH (8 ml) was added HBr/AcOH solution (1 mL, 30%). The reaction was stirred at 60° C. overnight. After the reaction was completed as indicated by TLC analysis, the resulting mixture was concentrated to dryness to give 250 mg of crude title compound. LC-MS (ESI+): m/z 333 (M+H)⁺.

Step 5:

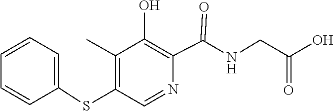 [SMILES: Cc1c(Sc2ccccc2)cnc(C(=O)NCC(=O)O)c1O]

The compound was synthesized according to the step 12 of Preparation of AKB-7560 (Compound 65). LC-MS (ESI+): m/z 319 (M+H)⁺; ¹H-NMR (300 MHz, DMSO-d₆) δ 13.00-12.61 (m, 2H), 9.30 (s, 1H), 7.81 (s, 1H), 7.51-7.35 (m, 5H), 4.01-3.90 (m, 2H), 2.31 (s, 3H).

### Example 59: Preparation of Compound 60

### Preparation of Compound 60 (AKE-7566)

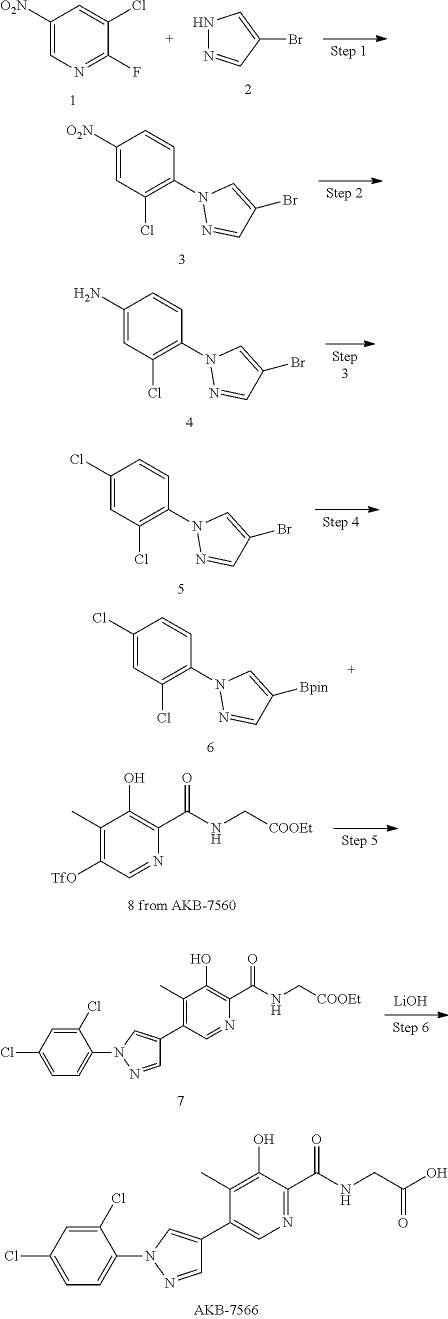

Step 1:

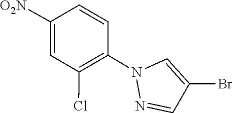 [SMILES: O=[N+]([O-])c1ccc(-n2cc(Br)cn2)c(Cl)c1]

To a solution of 4-brompyrazole (1.00 g, 6.80 mmol) in DMF (20 mL) at rt was added 2-chloro-1-fluoro-4-nitrobenzene (1.44 g, 8.16 mmol) and Cs₂CO₃ (3.33 g, 10.20 mmol). The reaction was stirred at 100° C. for 3 hrs. After the reaction was completed as indicated by TLC analysis, the reaction was cooled to rt, quenched with brine (20 mL) and extracted with EtOAc (20 mL×3). The combined organic phase was washed with brine, dried with anhydrous Na₂SO₄, filtered and concentrated in vacuo. The residue was slurried in Et₂O (20 mL) for 1 hr. After filtration, 1.61 g of the title compound as white solid was obtained. ¹H NMR (300 MHz, CDCl₃) δ 8.44 (d, J=2.5 Hz, 1H), 8.26 (dd, J=8.9, 2.5 Hz, 1H), 8.16 (s, 1H), 7.88 (d, J=8.9 Hz, 1H), 7.77 (s, 1H).

Step 2:

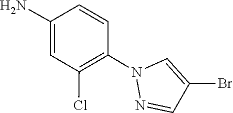 [SMILES: Nc1ccc(-n2cc(Br)cn2)c(Cl)c1]

To a saturated NH₄Cl solution (8 mL) at 60° C. was added iron powder (1.49 g, 26.67 mmol). The suspension was stirred at 80° C. for 30 mins. A solution of 4-bromo-1-(2-chloro-4-nitrophenyl)-1H-pyrazole (1.61 g, 5.33 mmol) in ethanol (30 mL) and THF (15 mL) was added dropwise to the reaction above over 5 mins. The reaction was continued to stir under reflux for 2 hrs. After the reaction was completed as indicated by TLC analysis, the reaction mixture was filtered through a pad of Celite. The filtrate was concentrated to dryness in vacuo. The residue was diluted with water (30 mL) and extracted with EtOAc (20 mL×3). The combined organic phase was washed with brine, dried with anhydrous Na₂SO₄, filtered and concentrated in vacuo to give 1.67 g of the crude title compound as yellow solid. ¹H NMR (300 MHz, CDCl₃) δ 7.71 (s, 1H), 7.65 (s, 1H), 7.23 (d, J=8.5 Hz, 2H), 6.76 (d, J=2.5 Hz, 1H), 6.60 (dd, J=8.5, 2.5 Hz, 1H), 3.58 (brs, 2H).

Step 3:

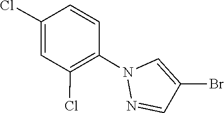 [SMILES: Clc1ccc(-n2cc(Br)cn2)c(Cl)c1]

Under nitrogen protection, to a suspension of CuCl₂ (1.43 g, 8.38 mmol) and t-BuONO (921 mg, 8.94 mmol) in anhydrous DMF (20 mL) at 50° C. was added a solution of 4-(4-bromo-1H-pyrazol-1-yl)-3-chloroaniline (1.52 g, 5.59 mmol) in anhydrous DMF (10 mL) drowpsie over 20 mins. After addition, the reaction was continued to stir at 50° C. for 1.5 hrs. After the reaction was completed as indicated by TLC analysis, the reaction was quenched with brine (30 mL) and extracted with EtOAc (30 mL×3). The combined organic phase was washed with brine, dried with anhydrous Na₂SO₄, filtered and concentrated in vacuo. The residue was purified by silica-gel column chromatography (PE: EtOAc=150: 1) to give 970 mg of the title compound as white solid. ¹H NMR (300 MHz, CDCl₃) δ 7.89 (s, 1H), 7.70 (s, 1H), 7.57-7.48 (m, 2H), 7.37 (dd, J=8.6, 2.3 Hz, 1H).

Step 4:

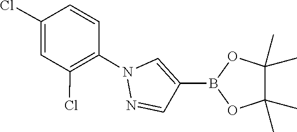 [SMILES: CC1(C)OB(c2cnn(-c3ccc(Cl)cc3Cl)c2)OC1(C)C]

Under nitrogen protection, to a solution of 4-bromo-1-(2,4-dichlorophenyl)-1H-pyrazole (430 mg, 1.47 mmol) in dioxane (15 mL) at rt was added bis(pinacolato)diboron (748 mg, 2.95 mmol), Pd(dppf)Cl₂ (108 mg, 0.15 mmol) and KOAc (433 mg, 4.42 mmol). The reaction was stirred at 100° C. overnight. After the reaction was completed as indicated by TLC analysis, the reaction was cooled to rt, quenched with brine (20 mL) and extracted with EtOAc (20 mL×3). The combined organic phase was washed with brine, dried with anhydrous Na₂SO₄, filtered and concentrated in vacuo. The residue was purified by silica-gel column chromatography (PE: EtOAc=20:1 to 5:1) to give 345 mg of the crude title compound as off-white solid. ¹H NMR (300 MHz, CDCl₃) δ 8.14 (s, 1H), 7.99 (s, 1H), 7.56-7.48 (m, 2H), 7.35 (dd, J=8.6, 2.3 Hz, 1H), 1.34 (s, 12H).

Step 5:

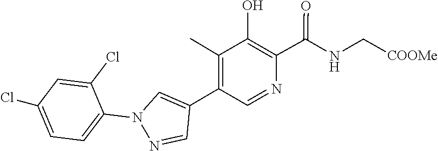 [SMILES: COC(=O)CNC(=O)c1ncc(-c2cnn(-c3ccc(Cl)cc3Cl)c2)c(C)c1O]

Under nitrogen protection, to a solution of methyl 2-(3-hydroxy-4-methyl-5-(((trifluoromethyl) sulfonyl)oxy)picolinamido)acetate (137 mg, 0.37 mmol) in dioxane (4 mL) and water (0.2 mL) at rt was added 1-(2,4-dichlorophenyl)-4-(4,4,5,5-tetramethyl-1,3,2-dioxaborolan-2-yl)-1H-pyrazole (400 mg, 1.18 mmol), Pd(dppf)Cl₂ (32 mg, 0.04 mmol) and K₃PO₄ (234 mg, 1.11 mmol). The reaction was stirred at 90° C. for 24 hrs. After the reaction was completed as indicated by TLC analysis, the reaction was cooled to rt, quenched with brine (5 mL) and extracted with EtOAc (5 mL×3). The combined organic phase was washed with brine, dried with anhydrous Na₂SO₄, filtered and concentrated in vacuo. The residue was purified by silica-gel column chromatography (PE: EtOAc=5:1 to 3:1) to give 64 mg of the crude title compound. The crude title compound was slurried in MeOH (0.5 mL) for 1 hr. After filtration and drying, 24 mg of the title compound as white solid was obtained. ¹H NMR (300 MHz, CDCl₃) δ 12.16 (s, 1H), 8.45 (s, 1H), 8.13 (s, 1H), 8.06 (s, 1H), 7.92 (s, 1H), 7.67-7.56 (m, 2H), 7.42 (dd, J=8.6, 2.3 Hz, 1H), 4.26 (d, J=5.7 Hz, 2H), 3.82 (s, 3H), 2.39 (d, J=1.2 Hz, 3H), 1.56 (s, 3H).

Step 6:

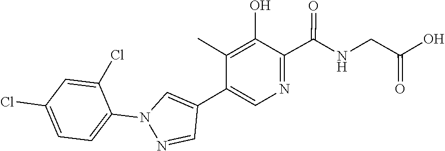 [SMILES: Cc1c(-c2cnn(-c3ccc(Cl)cc3Cl)c2)cnc(C(=O)NCC(=O)O)c1O]

The compound was synthesized according to the step 12 of Preparation of AKB-7560 (Compound 65). ¹H NMR (300 MHz, DMSO-d₆) δ 12.81 (s, 2H), 9.33 (t, J=6.2 Hz, 1H), 8.64 (s, 1H), 8.32 (s, 1H), 8.23 (s, 1H), 7.95 (d, J=2.2 Hz, 1H), 7.75 (d, J=8.6 Hz, 1H), 7.65 (dd, J=8.6, 2.2 Hz, 1H), 4.00 (d, J=6.1 Hz, 2H), 2.36 (s, 3H).

### Example 60: Preparation of Compound 61

### Preparation of Compound 61 (AKE-7593)

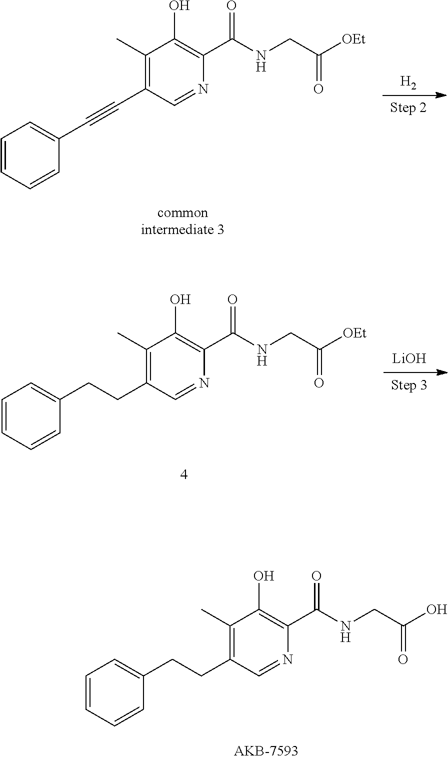

Step 1:

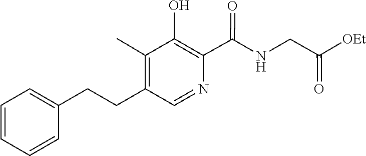 [SMILES: CCOC(=O)CNC(=O)c1ncc(CCc2ccccc2)c(C)c1O]

A suspension of ethyl (3-hydroxy-4-methyl-5-(phenylethynyl)picolinoyl)glycinate (130 mg, 0.40 mmol) and Pd/C (20 mg, 10 wt %) in EtOAc (8 mL) and MeOH (2 mL) was stirred at rt under 1 Mpa hydrogen pressure overnight. After the reaction was completed as indicated by TLC, the suspension was filtered through a pad of Celite. The filtrate was concentrated to dryness to give 150 mg of the title compound as a brown oil. LC-MS (ESI+): m/z 329(M+H)⁺.

Step 2:

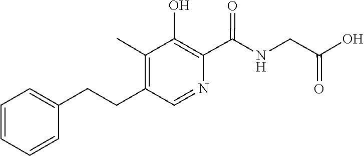 [SMILES: Cc1c(CCc2ccccc2)cnc(C(=O)NCC(=O)O)c1O]

The compound was synthesized according to the step 8 of Preparation of AKB-7588 (Compound 57). LC-MS (ESI+): m/z 315(M+H)⁺; ¹H-NMR (300 MHz, DMSO-d₆) δ 12.86 (brs, 1H), 12. 70 (s, 1H), 9.26 (t, J=6.0 Hz, 1H), 7.90 (s, 1H), 7.32-7.15 (m, 5H), 3.97 (d, J=6.0 Hz, 2H), 2.95 (t, J=6.3 Hz, 2H), 2.85 (t, J=6.3 Hz, 2H), 2.40 (s, 3H).

### Example 61: Preparation of Compound 62

### Preparation of Compound 62 (AKE-7592)

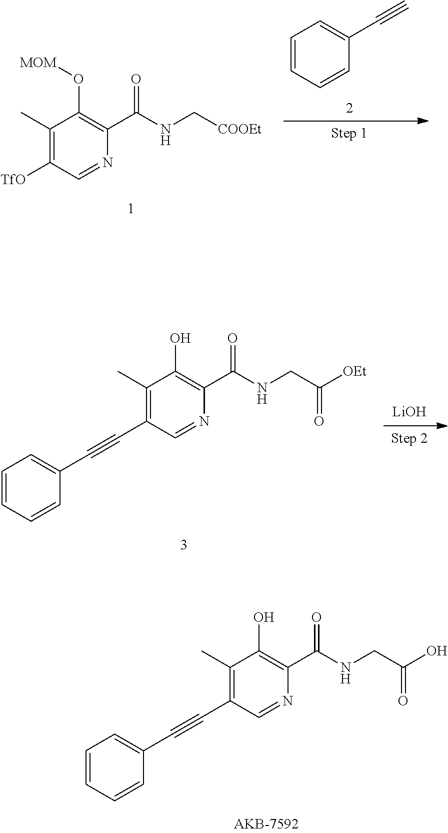

Step 1:

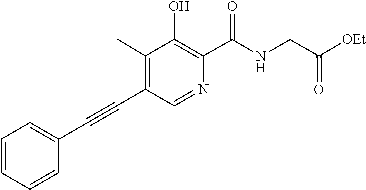 [SMILES: CCOC(=O)CNC(=O)c1ncc(C#Cc2ccccc2)c(C)c1O]

Under nitrogen protection, a reaction mixture of ethyl (3-(methoxymethoxy)-4-methyl-5-(((trifluoromethyl) sulfonyl)oxy)picolinoyl)glycinate (248 mg, 0.6 mmol), ethynylbenzene (122 mg, 1.20 mmol), Pd(pph₃)₂Cl₂ (21 mg, 0.030 mmol), CuI (6 mg, 0.030 mmol) and TEA (302 mg, 2.98 mmol) in DMF (8 mL) was stirred at 85° C. for about 16 hrs. After the reaction was completed as indicated by TLC, the reaction was cooled to rt, quenched with water (30 mL) and extracted with EtOAc (30 mL×3). The combined organic phase was washed with water (20 mL), dried and concentrated. The residue was purified by column chromatography (EA: PE=1: 20) to give 165 mg of the title compound as a yellow solid. LC-MS (ESI+): m/z 325 (M+H)⁺.

Step 2:

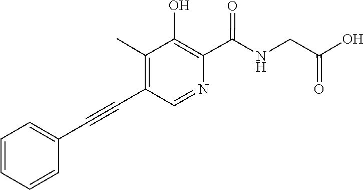 [SMILES: Cc1c(C#Cc2ccccc2)cnc(C(=O)NCC(=O)O)c1O]

The compound was synthesized according to the step 8 of Preparation of AKB-7588 (Compound 57). LC-MS (ESI+): m/z 339(M+H)⁺; ¹H-NMR (300 MHz, DMSO-d₆) δ 12.86 (s, 2H), 9.43 (t, J=6.0 Hz, 1H), 8.30 (s, 1H), 7.72-7.64 (m, 2H), 7.59-7.48 (m, 3H), 3.99 (d, J=6.0 Hz, 2H), 2.40 (s, 3H).

### Example 62: Preparation of Compound 63

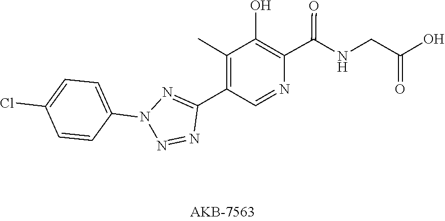 [SMILES: Cc1c(-c2nnn(-c3ccc(Cl)cc3)n2)cnc(C(=O)NCC(=O)O)c1O]

Compound 63 (AKE-7563) was prepared according to the preparation of AKB-7560 (Compound 65). LC-MS (ESI+): m/z 389 (M+H)⁺. ¹H-NMR (300 MHz, DMSO-d₆) δ 13.10-12.82 (m, 2H), 9.55 (d, J=6.6 Hz, 1H), 8.76 (s, 1H), 8.25 (d, J=8.7 Hz, 2H), 7.80 (d, J=8.7 Hz, 2H), 4.02 (d, J=6 Hz, 2H), 2.80 (s, 3H).

### Example 63: Preparation of Compound 64

### Preparation of Compound 64 (AKE-7561)

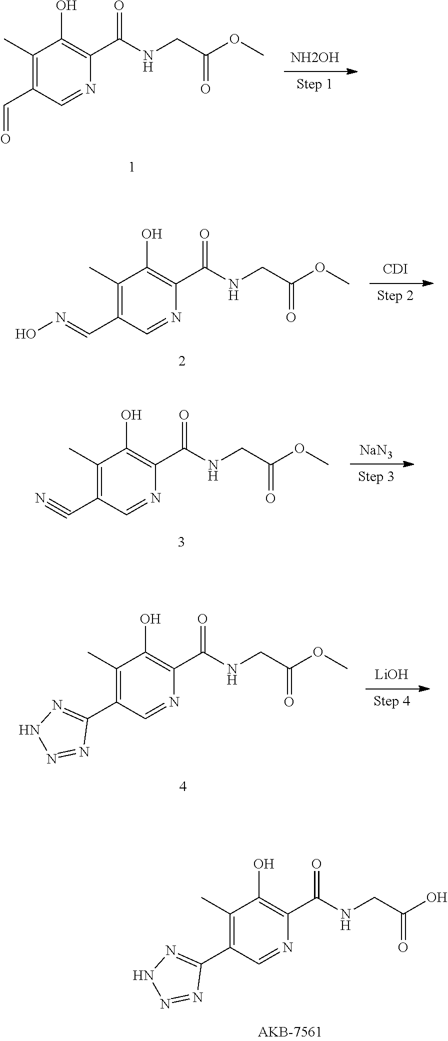

Step 1:

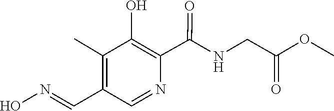 [SMILES: COC(=O)CNC(=O)c1ncc(/C=N/O)c(C)c1O]

A suspension of methyl (5-formyl-3-hydroxy-4-methylpicolinoyl)glycinate (200 mg, 0.79 mmol), NH₂OH. HCl (82 mg, 1.2 mmol) and NaHCO₃(166 mg, 2 mmol) in methanol (2 mL) was stirred at rt for about 3 hrs. After the reaction was completed as indicated by TLC analysis, the reaction was diluted with water 5 (mL), adjusted pH to 4 and extracted with DCM (10 mL×2). The combined organic phase was dried, filtered and concentrated to give 180 mg of the title compound as white solid. ¹H-NMR (300 MHz, DMSO-d₆) δ 12.70 (s, 1H), 11.85 (s, 1H), 9.52 (brs, 1H), 8.41 (s, 1H), 8.39 (s 1H), 4.08 (d, J=6.3 Hz, 2H), 3.67 (s, 3H).

Step 2:

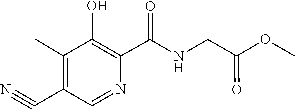 [SMILES: COC(=O)CNC(=O)c1ncc(C#N)c(C)c1O]

A solution of methyl (E)-(3-hydroxy-5-((hydroxyimino)methyl)-4-methylpicolinoyl)glycinate (180 mg, 0.67 mmol) and CDI (164 mg, 1.01 mmol) in DCM (8 mL) was stirred under reflux for about 2 hrs. After the reaction was completed as indicated by TLC, the reaction was quenched with water (5 mL) and extracted with DCM (10 mL×3). The combined organic phase was dried and concentrated to give 207 mg of crude product. The crude product was further slurried in EtOAc (2 mL) and filtered to give 105 mg of desired product. ¹H-NMR (300 MHz, CDCl₃) δ 8.55 (brs, 1H), 8.25 (s, 1H), 7.73 (s, 1H), 4.48 (d, J=6 Hz, 2H), 3.82 (s, 3H), 2.55 (s, 3H).

Step 3:

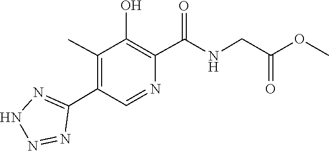 [SMILES: COC(=O)CNC(=O)c1ncc(-c2nn[nH]n2)c(C)c1O]

A suspension of methyl (5-cyano-3-hydroxy-4-methylpicolinoyl)glycinate (85 mg, 0.34 mmol), NH₄Cl (55 mg, 1.02 mmol) and NaN₃ (67 mg, 1.02 mmol) in DMF (1 mL) was stirred at 100° C. overnight. After the reaction was completed as indicated by TLC, the reaction was quenched with water (5 mL) and adjusted to pH 3 with a diluted HCl solution. A large amount of yellow solid was precipitated. The solid was collected by filtration to give 55 mg of the title compound. LC-MS (ESI+): m/z 293 (M+H)⁺.

Step 4:

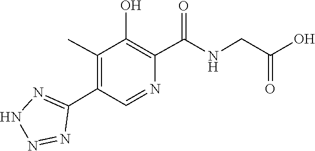 [SMILES: Cc1c(-c2nn[nH]n2)cnc(C(=O)NCC(=O)O)c1O]

The compound was synthesized according to the step 12 of Preparation of AKB-7560 (Compound 65). LC-MS (ESI+): m/z 279 (M+H)⁺; ¹H-NMR (300 MHz, DMSO-d₆) δ 12.95 (s, 1H), 12.88 (brs, 1H), 9.55 (t, J=6.3 Hz, 1H), 8.51 (s, 1H), 4.03 (d, J=6.3 Hz, 2 H), 2.45 (s, 3H).

### Example 64: Preparation of Compound 65

### Preparation of Compound 65 (AKE-7560)

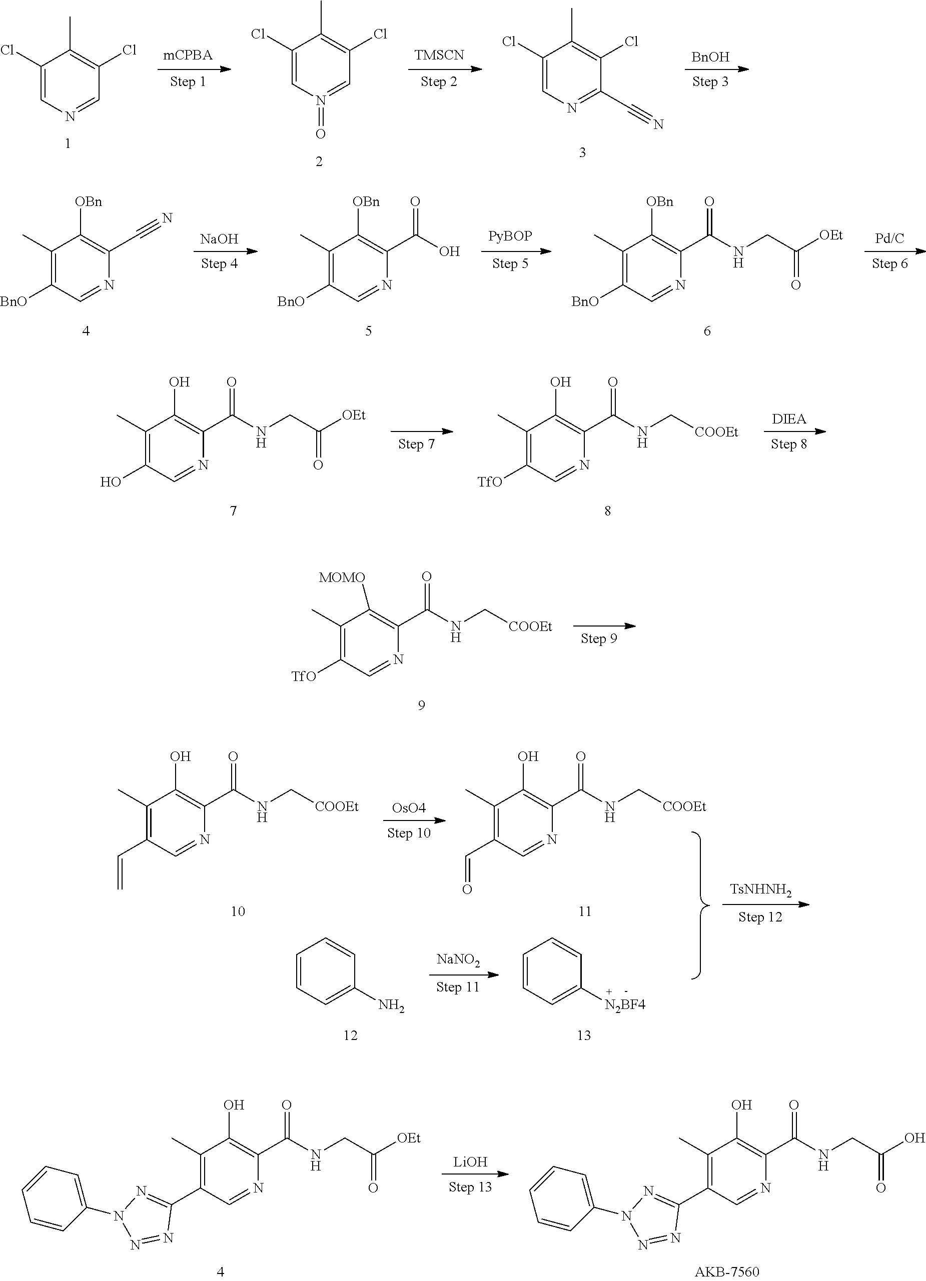

Step 1:

### 3,5-dichloro-4-methylpyridine 1-oxide

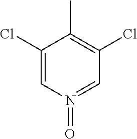

To a solution of 3,5-dichloro-4-methylpyridine (9.4 g, 57.7 mmol) in DCM (150 mL) at rt was added m-CPBA (12.9 g, 74.7 mmol) portion wise over 2 mins. The reaction was stirred at rt overnight. After the reaction was completed as indicated by TLC analysis, to the reaction was added K₂CO₃ (12 g, 87 mmol) in one portion and stirred at rt for about 2 hrs. After the resulting suspension was filtered, the filtrate was concentrated to dryness. The residue was slurried in a mixed solvent (PE:EtOAc=50:1, 50 mL) and filtered to afford 7.4 g of the title compound. LC-MS (ESI+): m/z 178 (M+H)⁺.

Step 2:

### 3,5-dichloro-4-methylpicolinonitrile

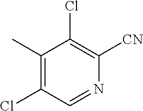

To a solution of 3,5-dichloro-4-methylpyridine 1-oxide (8 g, 44.94 mmol) in MeCN (150 mL) at room temperature was added TMS-CN (9 g, 89.88 mmol) and TEA (9.4 mL). The reaction was refluxed overnight. After the reaction was completed as indicated by TLC analysis, the reaction was cooled to rt, quenched with brine (150 mL) and extracted with EA (150 mL×3). The combined organic phase was washed with brine, dried with anhydrous Na₂SO₄ (60 g), filtered and concentrated in vacuo. The residue was purified by silica-gel column chromatography (PE:EtOAc=10:1) to give 6.8 g of the title compound. ¹H-NMR (300 MHz, CD₃Cl) δ 8.52 (s, 1H), 2.57 (s, 3H)).

Step 3:

### 3,5-bis(benzyloxy)-4-methylpicolinonitrile

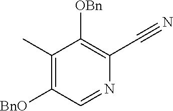 [SMILES: Cc1c(OCc2ccccc2)cnc(C#N)c1OCc1ccccc1]

Under nitrogen protection, to a suspension of NaH (60 wt %, 13.6 g, 0.34 mol) in DMF (200 mL) at 0° C. was added BnOH (36.8 g, 0.34 mol) dropwise over 20 mins. After addition, the resulting mixture was continued to stir at 0° C. for 15 mins. A solution of 3,5-dichloro-4-methyl-pyridine-2-carbonitrile (21.2 g, 113.37 mmol) in DMF (40 mL) was added dropwise to the suspension above over 20 mins. The reaction was stirred at rt overnight. After the reaction was completed as indicated by LCMS analysis, the reaction was cooled to 0° C. again and quenched with water (3 L) slowly. A large amount of solid was precipitated. The solid was collected by filtration and purified by column chromatography (PE: EtOAc=1:0 to 5:1) to give 15 g of the title compound. LC-MS (ESI+): m/z 331(M+H)⁺.

Step 4:

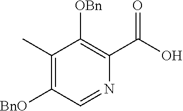 [SMILES: Cc1c(OCc2ccccc2)cnc(C(=O)O)c1OCc1ccccc1]

A solution of 3,5-dibenzyloxy-4-methyl-pyridine-2-carbonitrile (15 g, 45.4 mmol) and NaOH (18.16 g, 454.0 mmol) in ethanol (400 mL) and H₂O (150 mL) was stirred at 70° C. overnight. After the reaction was completed by LCMS analysis, the reaction was concentrated first to remove the excess amount of ethanol. The residue was acidified with conc. HCl to pH˜3 and a large amount of solid was precipitated. The white solid was collected by filtration, washed with EtOAc (100 mL), dried under vacuum to give 15.3 g of the title compound. LC-MS (ESI+): m/z 350 (M+H)⁺.

Step 5:

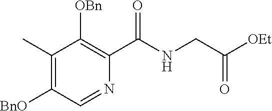 [SMILES: CCOC(=O)CNC(=O)c1ncc(OCc2ccccc2)c(C)c1OCc1ccccc1]

To a suspension of 3,5-dibenzyloxy-4-methyl-pyridine-2-carboxylic acid (15.3 g, 43.76 mmol) in DCM (500 mL) at rt was added PyBOP (27.35 g, 52.55 mmol) and TEA (22.16 g, 218.95 mmol) in one portion. After the suspension was stirred for 5 min, a clear solution was observed. To the solution was then added methyl-2-aminoacetate HCl salt (8.25 g, 65.69 mmol) portion wise over 2 mins. The resulting mixture was stirred at rt overnight. After the reaction was completed as indicated by LCMS analysis, the reaction mixture was concentrated directly and the residue was purified by column chromatography (PE: EtOAc=1:0 to 1:1) to give 3.6 g of the title compound. LC-MS (ESI+): m/z 421 (M+H)⁺.

Step 6:

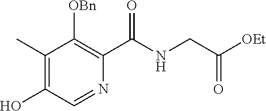 [SMILES: CCOC(=O)CNC(=O)c1ncc(O)c(C)c1OCc1ccccc1]

A suspension of methyl 2-[(3.5-dihenzyloxy-4-methy-pyidine-2-carbony)amino]acetate (16.9 g, 40.24 mmol) and Pd/C (1.1 g, 10 wt %) in THF (300 ml) and methanol (200 mL) was purged with hydrogen for 3 three times and stirred under atmospheric hydrogen pressure overnight. After the reaction was completed as indicated by LCMS analysis, the suspension was filtered through a pad of Celite. The filtrate was concentrated directly to afford 9.6 g of the title compound as a white solid. LC-MS (ESI+): m/z 241(M+H)⁺.

Step 7:

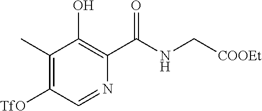 [SMILES: CCOC(=O)CNC(=O)c1ncc(OS(=O)(=O)C(F)(F)F)c(C)c1O]

To a suspension of methyl 2-(3,5-dihydroxy-4-methyl-pyridine-2-carbonyl)amino]acetate (9.6 g, 40.0 mmol) in methanol (500 mL) was added TEA (6.08 g, 60.0 mmol) and 1,1,1-trifluoro-N-phenyl-N— (trifluoromethylsulfonyl)methanesulfonamide (17.15 g, 48.0 mmol) in one portion. After the reaction was stirred at rt overnight, a clear brown homogeneous solution was observed. The completion of the reaction was depended on TLC analysis. The reaction was concentrated directly and purified by column chromatography (PE: EtOAc=1:0 to 1:1) to give 14.2 g of the title compound. LC-MS (ESI+): m/z 373 (M+H)⁺.

Step 8:

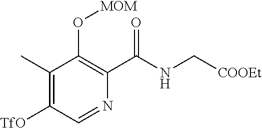

To a stirred solution of ethyl (3-hydroxy-4-methyl-5-(((trifluoromethyl)sulfonyl)oxy)picolinoyl) glycinate (14.2 g, 38.14 mmol) in DCM (350 mL) at 0° C. was added DIPEA (12.3 g, 95.36 mmol) and MOMCl (4.61 g, 57.22 mmol) sequentially. The reaction was continued to stir at 0° C. for 15 mins and stirred at rt for about 1 hr. After the reaction was completed as indicated by TLC analysis, the reaction was quenched with cold water (180 mL). After separation, the organic phase was dried over Na₂SO₄, and concentrated. The residue was purified by column chromatography (PE: EtOAc=1:0 to 1:1) to give 6.61 g of the title compound. LC-MS (ESI+): m/z 417(M+H)⁺.

Step 9:

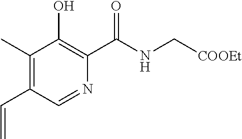 [SMILES: C=Cc1cnc(C(=O)NCC(=O)OCC)c(O)c1C]

A reaction of ethyl (3-(methoxymethoxy)-4-methyl-5-(((trifluoromethyl)sulfonyl)oxy)picolinoyl) glycinate (5.15 g, 13.83 mmol), tributyl(vinyl)stannane (5.26 g, 16.60 mmol), LiCl (1.76 g, 41.50 mmol) and Pd(PPh₃)₂Cl₂ (243 mg, 0.35 mmol) in DMF (250 mL) was stirred at 70° C. for 6 hrs. After the reaction was completed as indicated by TLC analysis, the reaction was concentrated to dryness. The residue was purified by column chromatography (PE: EtOAc=1:0 to 1:1) afford 1.84 g of the title compound as a white solid. LC-MS (ESI+): m/z 251(M+H)⁺.

Step 10:

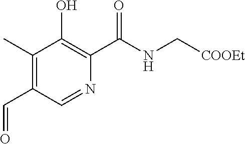 [SMILES: CCOC(=O)CNC(=O)c1ncc(C=O)c(C)c1O]

A solution of ethyl (3-hydroxy-4-methyl-5-vinylpicolinoyl)glycinate (1.74 g, 6.95 mmol), OsO₄ (50 mg), pyridine (550 mg, 6.95 mmol) and NaIO₄ (7.44 g, 34.77 mmol) in acetone (20 mL), THF (40 mL) and water (20 ml) was stirred at rt for 16 hrs. After ethyl (3-hydroxy-4-methyl-5-vinylpicolinoyl)glycinate was consumed by TLC, the reaction was extracted with EtOAc (100 mL×2). The combined organic phase was dried and concentrated. The residue was purified by column chromatography (PE: EtOAc=1:0 to 1:1) to give 1.5 g of the title compound as a white solid. LC-MS (ESI+): m/z 285(M+Na)⁺; ¹H-NMR (300 MHz, CDCl₃) δ 12.27 (s, 1H), 10.42 (s, 1H), 8.55 (brs, 1H), 8.42 (s, 1H), 4.25 (d, J=6.3 Hz, 2H), 3.82 (s, 3H), 2.61 (s, 3H).

Step 11:

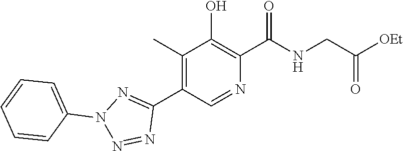 [SMILES: CCOC(=O)CNC(=O)c1ncc(-c2nnn(-c3ccccc3)n2)c(C)c1O]

A solution of ethyl (5-formyl-3-hydroxy-4-methylpicolinoyl)glycinate (100 mg, 0.396 mmol) and 4-methylbenzenesulfonohydrazide (89 mg, 0.476 mmol) in DCM (8 mL) and THF (4 mL) was stirred at rt overnight. The reaction was then concentrated to dryness. To the residue in pyridine (5 mL) at −5° C. was added a solution of (E)-1-phenyl-2-(tetrafluoro-λ⁵-boranyl)diazene (153 mg, 0.793 mmol) in ethanol (4 mL) and water (3 mL) was added dropwise over 5 mins. The resulting reaction mixture was stirred at rt for 3 h. After the reaction was completed as indicated by TLC analysis, the reaction was diluted with water (40 mL), extracted with ethyl acetate (20 mL×3). The organic phase was dried and concentrated. The residue was purified by column chromatography (0-100% ethyl acetate in DCM) to give 158 mg of the title compound. LC-MS (ESI+): m/z 369 (M+H)+.

Step 12:

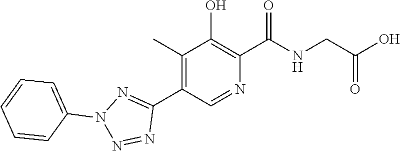 [SMILES: Cc1c(-c2nnn(-c3ccccc3)n2)cnc(C(=O)NCC(=O)O)c1O]

A solution of ethyl (3-hydroxy-4-methyl-5-(2-phenyl-2H-tetrazol-5-yl)picolinoyl)glycinate (158 mg, 0.429 mmol) in THF (6 mL) and MeOH (6 mL) was added a solution of NaOH (86 mg) in 4 mL of water. The reaction was stirred at rt for 3.5 h. After the reaction was completed by TLC analysis, the reaction was acidified with a diluted HCl solution (1N) to pH 3. A large amount of solid was precipitated. The solid was collected by filtration. After drying, 94 mg of the title compound was obtained. LC-MS (ESI+): m/z 355 (M+H)⁺; ¹H-NMR (300 MHz, DMSO-d₆) δ 13.10-12.82 (m, 2H), 9.55 (t, J=6.6 Hz, 1H), 8.77 (s, 1H), 8.25 (d, J=8.7 Hz, 2H), 7.76-7.64 (m, 3H), 4.01 (d, J=6.6 Hz, 2H), 2.60 (s, 3H).

### Example 65: Preparation of Compound 66

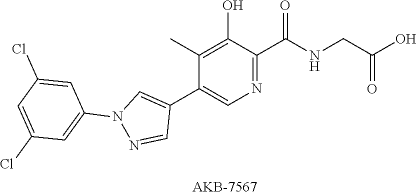 [SMILES: Cc1c(-c2cnn(-c3cc(Cl)cc(Cl)c3)c2)cnc(C(=O)NCC(=O)O)c1O]

Compound 66 (AKE-7567) was prepared according to the preparation of Compound 65 (AKB-7560). LC-MS (ESI+): m/z 421(M+H)+. ¹H-NMR (300 MHz, DMSO-d6) δ 12.82 (s, 2H), 9.34 (t, J=6.0 Hz, 1H), 9.07 (s, 1H), 8.32 (s, 1H), 8.28 (s, 1H), 8.07 (d, J=1.9 Hz, 2H), 7.62 (s, 1H), 4.00 (d, J=6.0 Hz, 2H), 2.37 (s, 3H).

### Example 66: Preparation of Compound 67

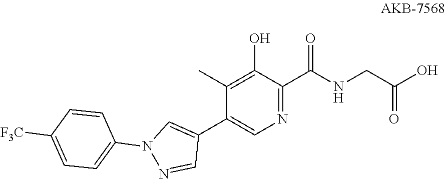 [SMILES: Cc1c(-c2cnn(-c3ccc(C(F)(F)F)cc3)c2)cnc(C(=O)NCC(=O)O)c1O]

Compound 67 (AKE-7568) was prepared according to the preparation of Compound 65 (AKB-7560). LC-MS (ESI+): m/z 421 (M+H)+. ¹H-NMR (300 MHz, DMSO-d6) δ 12.85 (s, 1H), 9.38-9.30 (m, 1H), 9.09 (s, 1H), 8.34 (s, 1H), 8.28 (s, 1H), 8.18 (d, J=8.4 Hz, 2H), 7.95 (d, J=8.4 Hz, 2H), 4.01 (d, J=6.0 Hz, 2H), 2.38 (s, 3H).

### Example 67: Preparation of Compound 68

### Preparation of Compound 68 (AKE-7565)

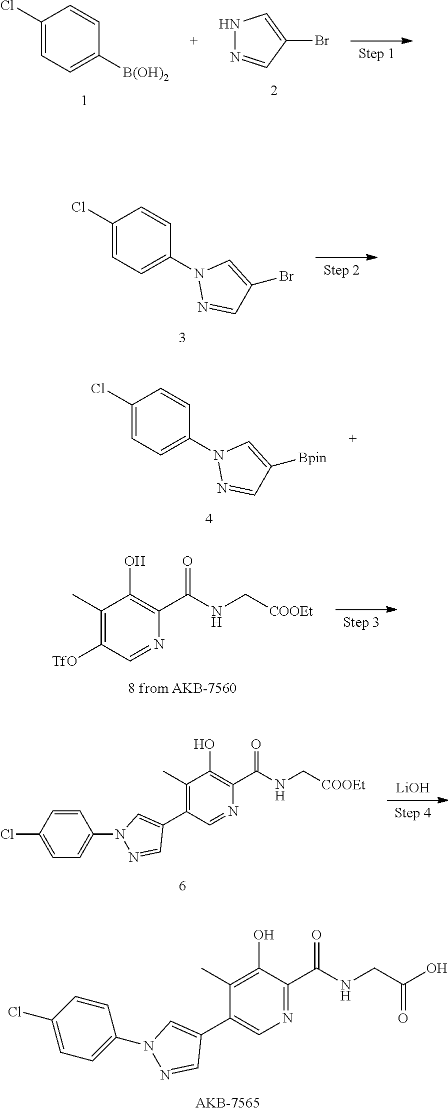

Step 1:

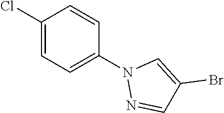 [SMILES: Clc1ccc(-n2cc(Br)cn2)cc1]

To a solution of 4-brompyrazole (500 mg, 3.40 mmol) in DCM (10 mL) at rt was added 4-chlorophenylboronic acid (1.06 g, 6.80 mmol), Cu(OAc)₂ (1.24 g, 6.80 mmol) and pyridine (806 mg, 10.20 mmol). The reaction was stirred at rt overnight. After the reaction was completed as indicated by TLC analysis, the suspension was filtered through a pad of Celite. The filtrate was quenched with brine (10 mL) and extracted with DCM (10 mL×3). The combined organic phase was washed with brine, dried with anhydrous Na₂SO₄, filtered and concentrated in vacuo. The residue was purified by silica-gel column chromatography (PE: EtOAc=100:1 to 50:1) to give 800 mg of the title compound as white solid. ¹H NMR (300 MHz, CDCl₃) δ 7.91 (s, 1H), 7.67 (s, 1H), 7.59 (d, J=8.9 Hz, 2H), 7.43 (d, J=8.9 Hz, 2H).

Step 2:

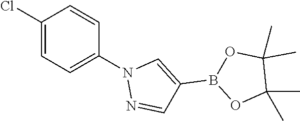 [SMILES: CC1(C)OB(c2cnn(-c3ccc(Cl)cc3)c2)OC1(C)C]

Under nitrogen protection, to a solution of 4-bromo-1-(4-chlorophenyl)-1H-pyrazole (1.00 g, 3.90 mmol) in dioxane (20 mL) at rt was added Bis(pinacolato)diboron (2.48 g, 9.76 mmol), Pd(dppf)Cl₂ (286 mg, 0.39 mmol) and KOAc (1.15 g, 11.72 mmol). The reaction was stirred at 100° C. for 2 hrs. After the reaction was completed as indicated by TLC analysis, the reaction was cooled to rt, quenched with brine (20 mL) and extracted with EtOAc (20 mL×3). The combined organic phase was washed with brine, dried with anhydrous Na₂SO₄, filtered and concentrated in vacuo. The residue was purified by silica-gel column chromatography (PE: EtOAc=20: 1) to give 1.4 g of the crude title compound as yellow solid. LC-MS (ESI+): m/z 305 (M+H)⁺.

Step 3:

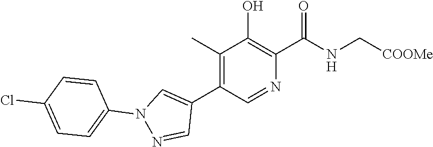 [SMILES: COC(=O)CNC(=O)c1ncc(-c2cnn(-c3ccc(Cl)cc3)c2)c(C)c1O]

Under nitrogen protection, to a solution of methyl 2-(3-hydroxy-4-methyl-5-(((trifluoromethyl) sulfonyl)oxy)picolinamido)acetate (100 mg, 0.27 mmol) in dioxane (5 mL) and water (0.25 mL) at room temperature was added crude 1-(4-chlorophenyl)-4-(4,4,5,5-tetramethyl-1,3,2-dioxaborolan-2-yl)-1H-pyrazole (98 mg, 0.32 mmol), Pd(dppf)Cl₂ (20 mg, 0.03 mmol) and K₃PO₄ (114 mg, 0.54 mmol). The reaction was stirred at 100° C. for 24 hrs. After the reaction was completed as indicated by TLC analysis, the reaction was quenched with brine (5 mL) and extracted with EtOAc (5 mL×3). The combined organic phase was washed with brine, dried with anhydrous Na₂SO₄, filtered and concentrated in vacuo. The residue was purified by silica-gel column chromatography (PE: EtOAc=5: 1) to give 28 mg of the title compound as white solid. ¹H NMR (300 MHz, CDCl₃) δ 12.24 (s, 1H), 8.53 (t, J=5.8 Hz, 1H), 8.19 (s, 1H), 8.13 (s, 1H), 7.98 (s, 1H), 7.78 (d, J=8.9 Hz, 2H), 7.55 (d, J=8.9 Hz, 2H), 4.34 (d, J=5.8 Hz, 2H), 3.90 (s, 3H), 2.47 (s, 3H).

Step 4:

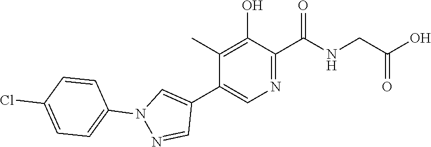 [SMILES: Cc1c(-c2cnn(-c3ccc(Cl)cc3)c2)cnc(C(=O)NCC(=O)O)c1O]

To a solution of methyl 2-(5-(1-(4-chlorophenyl)-1H-pyrazol-4-yl)-3-hydroxy-4-methylpicolinamido) acetate (28 mg, 0.07 mmol) in THF (0.25 mL) and water (0.25 mL) at room temperature was added LiOH·H₂O (6 mg, 0.14 mmol). The reaction was stirred at 40° C. for 1 hr. After the reaction was completed as indicated by LCMS analysis, the reaction was quenched with water (5 mL) and extracted with EtOAc (5 mL×2). The aqueous phase was adjusted to pH 3˜4 with a diluted HCl solution (2N). A large amount of the solid was precipitated. After filtration and drying, 7.8 mg of the title compound as light brown solid was obtained. ¹H NMR (300 MHz, DMSO-d₆) δ 12.81 (s, 2H), 9.33 (t, J=6.2 Hz, 1H), 8.97 (s, 1H), 8.33 (s, 1H), 8.22 (s, 1H), 7.98 (d, J=8.9 Hz, 2H), 7.63 (d, J=8.9 Hz, 2H), 4.01 (d, J=6.1 Hz, 3H), 2.38 (s, 3H).

### Example 68: Preparation of Compound 69

### Preparation of Compound 69 (CP-0285)

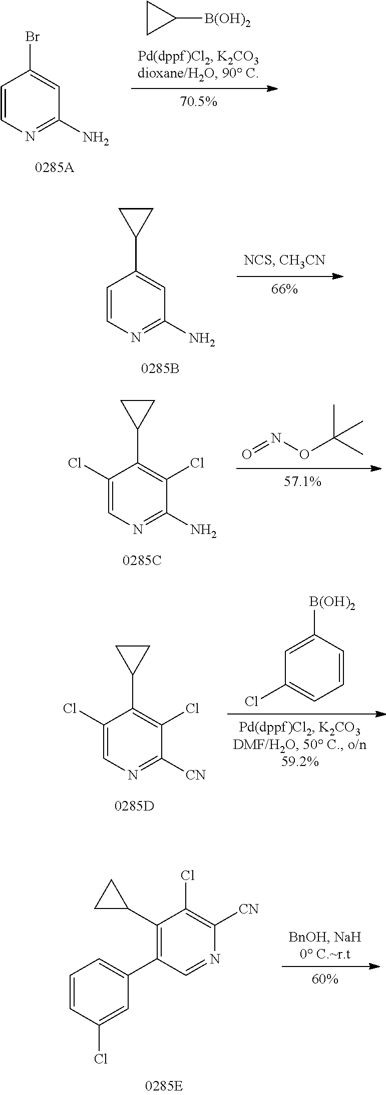

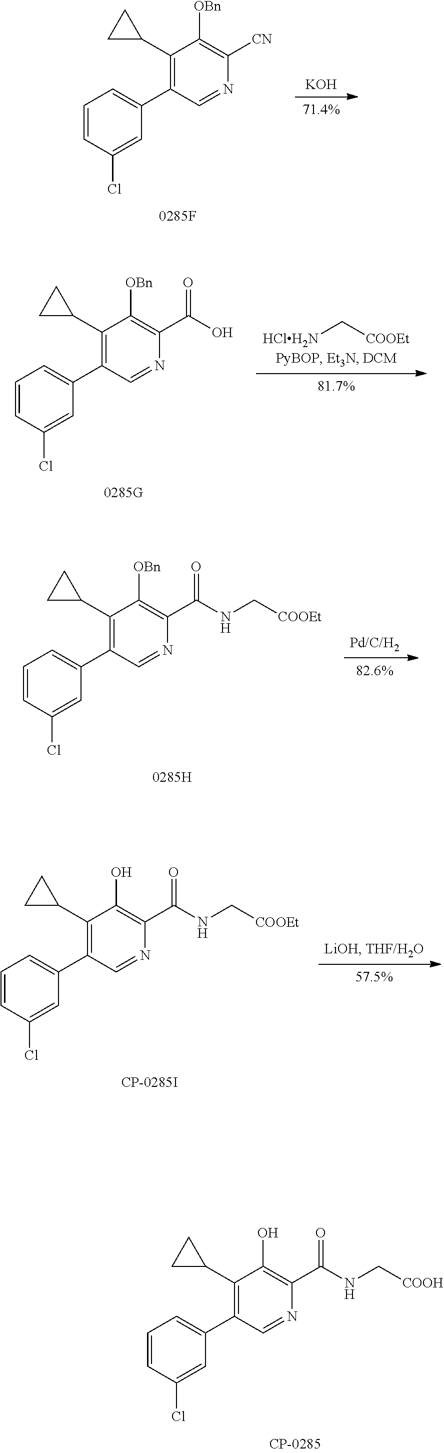

### Step 1: 4-Cyclopropylpyridin-2-amine

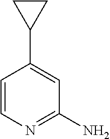

To a solution of 4-bromopyridin-2-amine (10.0 g, 58.0 mmol), cyclopropylboronic acid (7.5 g, 87.0 mmol) and potassium carbonate (16.0 g, 116 mmol) was added [1,1′-bis(diphenylphosphino)ferrocene]dichloropalladium(II) (4.3 g, 5.8 mmol) in 1,4-dioxane/water (50.0 mL/10.0 mL). The mixture was stirred at 90° C. for 12.0 h under nitrogen and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=3/1) to afford 4-cyclopropylpyridin-2-amine (5.5 g, 70.5% yield) as yellow solid. LC-MS: m/z=135.0 [M+H]⁺, retention time 1.47 min (Method A).

### Step 2: 3,5-Dichloro-4-cyclopropylpyridin-2-amine

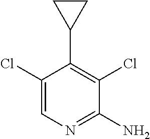 [SMILES: Nc1ncc(Cl)c(C2CC2)c1Cl]

To a solution of 4-cyclopropylpyridin-2-amine (5.0 g, 37.3 mmol) in N,N-dimethylformamide (100.0 mL) was added N-chlorosuccinimide (9.9 g, 74.0 mmol). The mixture was stirred for 2.0 h at 50° C. The reaction was diluted with water and extracted twice with ethyl acetate. The organic layer was separated, washed with brine, dried over sodium sulfate and concentrated. The residue was purified by flash chromatography (petroleum ether/ethyl acetate=5/1) to give 3,5-dichloro-4-cyclopropylpyridin-2-amine (4.9 g, 66% yield) as yellow solid. LC-MS: m/z=203.0 (M+H)⁺, retention time 1.94 min (Method A).

### Step 3: 3,5-Dichloro-4-cyclopropylpicolinonitrile

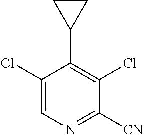 [SMILES: N#Cc1ncc(Cl)c(C2CC2)c1Cl]

To a solution of 3,5-dichloro-4-cyclopropylpyridin-2-amine (2.0 g, 10.0 mmol) and copper(I) cyanide in dimethyl sulfoxide (30.0 mL) was added tert-butyl nitrite (1.55 g, 15.0 mmol). The mixture was stirred for 12.0 h at 40° C. The reaction was diluted with water and extracted twice with ethyl acetate. The organic layer was separated, washed with brine, dried over sodium sulfate and concentrated. The 3,5-dichloro-4-cyclopropylpicolinonitrile (1.25 g, 57.1% yield) was obtained as yellow solid. LC-MS: m/z=213.0 (M+H)⁺, retention time 2.05 min (Method A).

### Step 4: 3-Chloro-5-(3-chlorophenyl)-4-cyclopropylpicolinonitrile

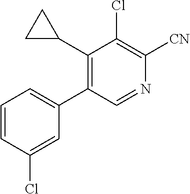 [SMILES: N#Cc1ncc(-c2cccc(Cl)c2)c(C2CC2)c1Cl]

To a solution of 3,5-dichloro-4-cyclopropylpicolinonitrile (1.0 g, 4.7 mmol), (3-chlorophenyl)boronic acid (0.68 g, 4.7 mmol) and potassium carbonate (1.2 g, 8.7 mmol) was added [1,1′-bis(diphenylphosphino)ferrocene]dichloropalladium(II) (0.32 g, 0.44 mmol) in N,N-dimethylformamide/water (20.0 mL/5.0 mL). The mixture was stirred at 50° C. for 12.0 h under nitrogen and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=3/1) to afford 3-chloro-5-(3-chlorophenyl)-4-cyclopropylpicolinonitrile (800 mg, 59.2% yield) as yellow solid. LC-MS: m/z=289.0 (M+H)⁺, retention time 2.25 min (Method A).

### Step 5: 3-(Benzyloxy)-5-(3-chlorophenyl)-4-cyclopropylpicolinonitrile

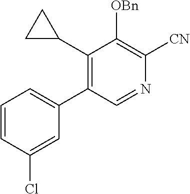 [SMILES: N#Cc1ncc(-c2cccc(Cl)c2)c(C2CC2)c1OCc1ccccc1]

To a solution of 3-chloro-5-(3-chlorophenyl)-4-cyclopropylpicolinonitrile (0.8 g, 2.8 mmol) in N,N-dimethylformamide (10.0 mL) was added sodium hydride (0.14 g 3.4 mmol, 60 percent in oil) at 0° C. The mixture was stirred at 0° C. for 20 min and benzyl alcohol (0.3 g, 2.8 mmol) was added. The mixture was allowed to warm up to room temperature and left stirring for another 0.5 h. The reaction was quenched with ice-water and extracted twice with ethyl acetate. The organic layer was separated, washed with brine, dried over sodium sulfate and concentrated under reduced pressure. The crude product was purified by flash chromatography (petroleum ether/ethyl acetate=5/1) to obtain 3-(benzyloxy)-5-(3-chlorophenyl)-4-cyclopropylpicolinonitrile (600 mg, 60.5% yield) as yellow oil. LC-MS: m/z=361.0 (M+H)⁺, retention time 2.29 min (Method A).

### Step 6: 3-(Benzyloxy)-5-(3-chlorophenyl)-4-cyclopropylpicolinic Acid

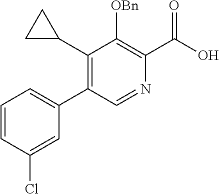 [SMILES: O=C(O)c1ncc(-c2cccc(Cl)c2)c(C2CC2)c1OCc1ccccc1]

To a solution of 3-(benzyloxy)-5-(3-chlorophenyl)-4-cyclopropylpicolinonitrile (400 mg, 1.1 mmol) in tetrahydrofuran/water (20.0 mL/10.0 mL) was added potassium hydroxide (200 mg, 3.6 mmol). The mixture was stirred at room temperature overnight and concentrated to remove tetrahydrofuran. The resulting aqueous solution was acidified with 3N/L hydrochloric acid to pH=3˜4. The precipitate was filtered, washed with water and dried to obtain 3-(benzyloxy)-5-(3-chlorophenyl)-4-cyclopropylpicolinic acid (296 mg, 71.4% yield) as white solid. LC-MS: m/z=380.0 (M+H)⁺, retention time 1.68 min (Method A).

### Step 7: Ethyl (3-(benzyloxy)-5-(3-chlorophenyl)-4-cyclopropylpicolinoyl)glycinate

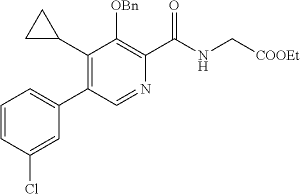 [SMILES: CCOC(=O)CNC(=O)c1ncc(-c2cccc(Cl)c2)c(C2CC2)c1OCc1ccccc1]

A mixture of 3-(benzyloxy)-5-(3-chlorophenyl)-4-cyclopropylpicolinic acid (296 mg, 0.8 mmol), ethyl glycinate hydrochloride (140 mg, 0.96 mmol), benzotriazole-1-yl-oxytripyrrolidinophosphonium hexafluorophosphate (500 mg, 0.96 mmol) and triethylamine (404 mg, 4.0 mmol) in dichloromethane (10.0 mL) was stirred at room temperature overnight. The reaction was quenched with water and extracted with dichloromethane. The organic layer was separated, washed with brine, dried over sodium sulfate and concentrated under reduced pressure. The residue was purified by flash chromatography (petroleum ether/ethyl acetate=2/1) to obtain ethyl (3-(benzyloxy)-5-(3-chlorophenyl)-4-cyclopropylpicolinoyl)glycinate (306 mg, 81.7% yield) as yellow solid. LC-MS: m/z=465.0 (M+H)⁺, retention time 2.26 min (Method A).

### Step 8: Ethyl (5-(3-chlorophenyl)-4-cyclopropyl-3-hydroxypicolinoyl)glycinate

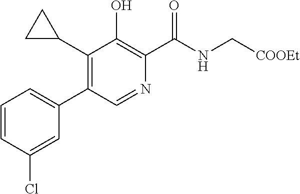 [SMILES: CCOC(=O)CNC(=O)c1ncc(-c2cccc(Cl)c2)c(C2CC2)c1O]

A mixture of ethyl (3-(benzyloxy)-5-(3-chlorophenyl)-4-cyclopropylpicolinoyl)glycinate (300 mg, 0.64 mmol) and 10% palladium-carbon (20.0 mg) in tetrahydrofuran (20.0 mL) was stirred at room temperature under hydrogen atmosphere for 5.0 h. The mixture was filtered and the filtrate was concentrated. Ethyl (5-(3-chlorophenyl)-4-cyclopropyl-3-hydroxypicolinoyl)glycinate (210 mg, 82.6% yield) was obtained as white solid. LC-MS: m/z=376.0 (M+H)⁺, retention time 2.55 min (Method A).

### Step 9: (5-(3-Chlorophenyl)-4-cyclopropyl-3-hydroxypicolinoyl)glycine

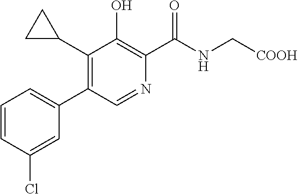 [SMILES: O=C(O)CNC(=O)c1ncc(-c2cccc(Cl)c2)c(C2CC2)c1O]

To a solution of ethyl (5-(3-chlorophenyl)-4-cyclopropyl-3-hydroxypicolinoyl)glycinate (200 mg, 0.52 mmol) in tetrahydrofuran/water (10.0 mL/5.0 mL) was added lithium hydroxide monohydrate (210 mg, 5.0 mmol). The mixture was stirred at room temperature overnight and concentrated to remove tetrahydrofuran. The resulting aqueous solution was acidified with 3N/L hydrochloric acid to pH=3˜4. The precipitate was filtered, washed with water and dried to obtain (5-(3-chlorophenyl)-4-cyclopropyl-3-hydroxypicolinoyl)glycine (106.5 mg, 57.5% yield) as white solid. LC-MS: m/z=347.0 (M+H)⁺, retention time 5.03 min (Method A). ¹H NMR (400 MHz, DMSO-d₆) δ 12.88 (s, 1H), 9.37 (t, J=6.1 Hz, 1H), 8.03 (s, 1H), 7.58 (s, 1H), 7.55-7.51 (m, 2H), 7.50-7.44 (m, 1H), 4.01 (t, J=10.3 Hz, 2H), 1.84 (tt, J=8.7, 5.7 Hz, 1H), 0.81-0.67 (m, 2H), 0.67-0.52 (m, 2H).

### Example 69: Preparation of Compound 70

### Preparation of Compound 70 (CP-0287)

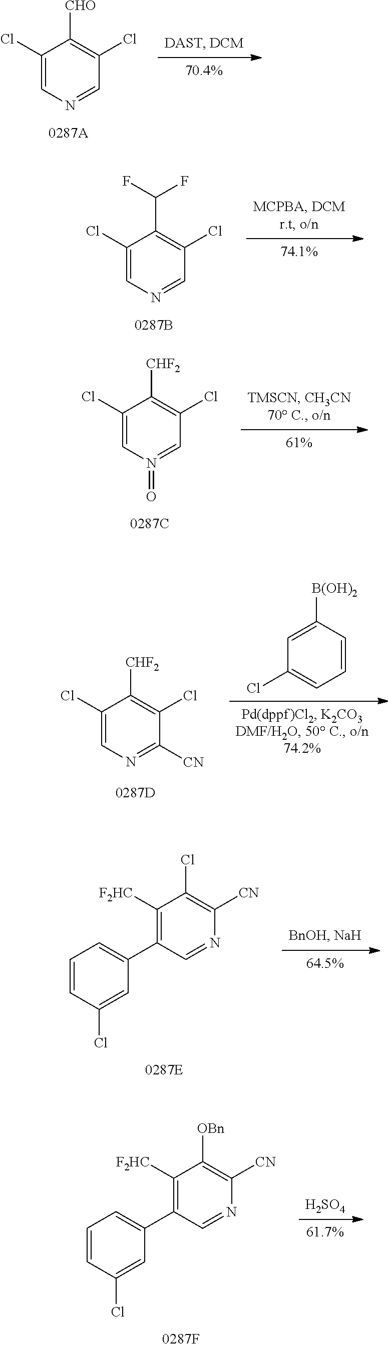

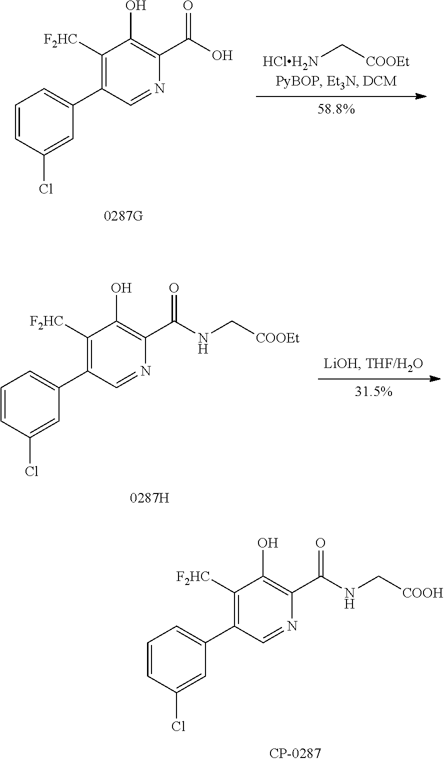

### Step 1: 3,5-Dichloro-4-(difluoromethyl)pyridine

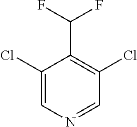 [SMILES: FC(F)c1c(Cl)cncc1Cl]

To a solution of 3,5-dichloroisonicotinaldehyde (1.75 g, 10.0 mmol) in dichloromethane (25.0 mL) was added diethylaminosulfur trifluoride (3.2 g, 20 mmol) dropwise at −20° C. The mixture was stirred for 12.0 h at room temperature. The reaction was diluted with ice-water and extracted twice with dichloromethane. The organic layer was separated, washed with brine, dried over sodium sulfate and concentrated. The desired product (1.05 g, 70.4%) was obtained as yellow solid. LC-MS: m/z=198.0 (M+H)⁺, retention time 1.95 min (Method A).

### Step 2: 3,5-Dichloro-4-(difluoromethyl)pyridine 1-oxide

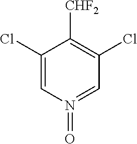 [SMILES: [O-][n+]1cc(Cl)c(C(F)F)c(Cl)c1]

To a solution of 3,5-dichloro-4-(difluoromethyl)pyridine (1.0 g, 5.0 mmol) in dichloromethane (25.0 mL) was added 3-chlorobenzoperoxoic acid (2.02 g, 10 mmol, 85 percent). The mixture was stirred overnight at room temperature. The reaction was diluted with water and extracted twice with dichloromethane. The organic layer was separated, washed with brine, dried over sodium sulfate and concentrated. The residue was purified by flash chromatography (dichloromethane/methanol=100/3) to give 3,5-dichloro-4-(difluoromethyl)pyridine 1-oxide (0.8 g, 74.1% yield) as yellow solid. LC-MS: m/z=214.0 (M+H)⁺, retention time 1.57 min (Method A).

### Step 3: 3,5-Dichloro-4-(difluoromethyl)picolinonitrile

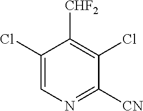 [SMILES: N#Cc1ncc(Cl)c(C(F)F)c1Cl]

To a solution of 3,5-dichloro-4-(difluoromethyl)pyridine 1-oxide (0.8 g, 3.7 mmol) and triethylamine (0.75 g, 7.4 mmol) in acetonitrile (30.0 mL) was added trimethylsilanecarbonitrile (0.74 g, 7.4 mmol) dropwise at 0° C.. The mixture was stirred overnight at 80° C. The reaction was diluted with water and extracted twice with ethyl acetate. The organic layer was separated, washed with brine, dried over sodium sulfate and concentrated. The residue was purified by flash chromatography (petroleum ether/ethyl acetate=10/1) to afford 3,5-dichloro-4-(difluoromethyl)picolinonitrile (0.35 g, 61% yield) as yellow oil. LC-MS: m/z=223.0 (M+H)⁺, retention time 1.93 min (Method A)

### Step 4: 3-Chloro-5-(3-chlorophenyl)-4-(difluoromethyl)picolinonitrile

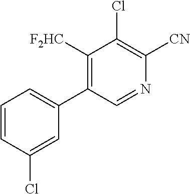 [SMILES: N#Cc1ncc(-c2cccc(Cl)c2)c(C(F)F)c1Cl]

To a solution of 3,5-dichloro-4-(difluoromethyl)picolinonitrile (0.49 g, 2.2 mmol), (3-chlorophenyl)boronic acid (0.37 g, 2.2 mmol) and potassium carbonate (0.6 g, 4.4 mmol) was added [1,1′-bis(diphenylphosphino)ferrocene]dichloropalladium(II) (0.15 g, 0.22 mmol) in N,N-dimethylformamide/water (20.0 mL/5.0 mL). The mixture was stirred at 50° C. for 12.0 h under nitrogen and cooled to room temperature. Ethyl acetate and water were added to the solution, and the layers were separated. The organic layer was washed with brine, dried over sodium sulfate and concentrated to dryness. The residue was purified by flash chromatography (petroleum ether/ethyl acetate=3/1) to afford 3-chloro-5-(3-chlorophenyl)-4-(difluoromethyl)picolinonitrile (0.48 g, 74.2% yield) as yellow solid. LC-MS: m/z=299.0 (M+H)⁺, retention time 2.10 min (Method A).

### Step 5: 3-(benzyloxy)-5-(3-chlorophenyl)-4-(difluoromethyl)picolinonitrile

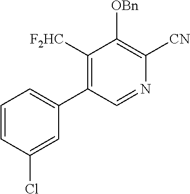 [SMILES: N#Cc1ncc(-c2cccc(Cl)c2)c(C(F)F)c1OCc1ccccc1]

To a solution of 3-chloro-5-(3-chlorophenyl)-4-(difluoromethyl)picolinonitrile (0.48 g, 1.6 mmol) in N,N-dimethylformamide (10.0 mL) was added sodium hydride (76.8 mg, 1.92 mmol, 60 percent in oil) at 0° C. The mixture was stirred at 0° C. for 20 min and benzyl alcohol (0.18 g, 1.6 mmol) was added. The mixture was allowed to warm up to room temperature and left stirring for another 0.5 h. The reaction was quenched with ice-water and extracted twice with ethyl acetate. The organic layer was separated, washed with brine, dried over sodium sulfate and concentrated under reduced pressure. The crude product was purified by flash chromatography (petroleum ether/ethyl acetate=10/1) to obtain 3-(benzyloxy)-5-(3-chlorophenyl)-4-(difluoromethyl)picolinonitrile (400 mg, 64.5% yield) as yellow oil. LC-MS: m/z=371.0 (M+H)⁺, retention time 2.20 min (Method A).

### Step 6: 5-(3-Chlorophenyl)-4-(difluoromethyl)-3-hydroxypicolinic Acid

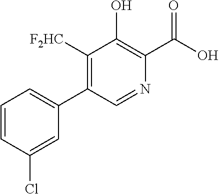 [SMILES: O=C(O)c1ncc(-c2cccc(Cl)c2)c(C(F)F)c1O]

A mixture of 3-(benzyloxy)-5-(3-chlorophenyl)-4-(difluoromethyl)picolinonitrile (370 mg, 1.0 mmol) in 50% sulfuric acid (10.0 mL) was stirred at 100° C. for 3.0 h. The solution was diluted with ice-water. The resulting solid was filtered, washed with water and dried to obtain 5-(3-chlorophenyl)-4-(difluoromethyl)-3-hydroxypicolinic acid (200 mg, 61.7% yield) as grey solid. LC-MS: m/z=300.0 (M+H)⁺, retention time 2.09 min (Method A).

### Step 7: Ethyl (5-(3-chlorophenyl)-4-(difluoromethyl)-3-hydroxypicolinoyl)glycinate

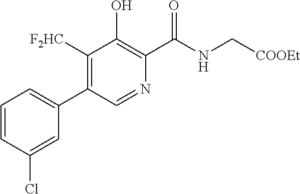 [SMILES: CCOC(=O)CNC(=O)c1ncc(-c2cccc(Cl)c2)c(C(F)F)c1O]

A mixture of 5-(3-chlorophenyl)-4-(difluoromethyl)-3-hydroxypicolinic acid (200 mg, 0.66 mmol), ethyl glycinate hydrochloride (100 mg, 0.75 mmol), benzotriazole-1-yl-oxytripyrrolidinophosphonium hexafluorophosphate (390 mg, 0.75 mmol) and triethylamine (0.34 g, 3.3 mmol) in dichloromethane (10.0 mL) was stirred at room temperature overnight. The reaction was quenched with water and extracted with dichloromethane. The organic layer was separated, washed with brine, dried over sodium sulfate and concentrated under reduced pressure. The residue was purified by flash chromatography (petroleum ether/ethyl acetate=1/1) to obtain ethyl (5-(3-chlorophenyl)-4-(difluoromethyl)-3-hydroxypicolinoyl)glycinate (150 mg, 58.8% yield) as yellow solid. LC-MS: m/z=385.0 (M+H)⁺, retention time 2.14 min (Method A).

### Step 8: (5-(3-Chlorophenyl)-4-(difluoromethyl)-3-hydroxypicolinoyl)glycine

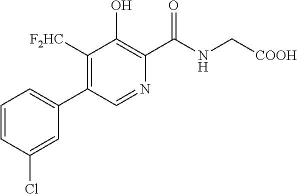 [SMILES: O=C(O)CNC(=O)c1ncc(-c2cccc(Cl)c2)c(C(F)F)c1O]

To a solution of ethyl (5-(3-chlorophenyl)-4-(difluoromethyl)-3-hydroxypicolinoyl)-Glycinate (130 mg, 0.33 mmol) in tetrahydrofuran/water (10.0 mL/5.0 mL) was added lithium hydroxide monohydrate (138 mg, 3.3 mmol). The mixture was stirred at room temperature overnight and concentrated to remove tetrahydrofuran. The resulting aqueous solution was acidified with 3N/L hydrochloric acid to pH=3˜4. The precipitate was filtered, washed with water and dried to obtain (5-(3-chlorophenyl)-4-(difluoromethyl)-3-hydroxypicolinoyl)glycine (38.1 mg, 31.5% yield) as white solid. LC-MS: m/z=357.0 (M+H)⁺, retention time 4.91 min (Method A). ¹H NMR (400 MHz, DMSO-d₆) δ 13.35 (s, 1H), 9.66-9.44 (m, 1H), 8.20 (s, 1H), 7.64-7.47 (m, 3H), 7.40 (d, J=7.3 Hz, 1H), 7.21-6.81 (m, 1H), 4.03 (d, J=6.1 Hz, 2H).

### Vitro Assays Demonstrate PHD Inhibition

Enzymatic half maximal inhibitory concentration (IC₅₀) values were determined on selected compounds of the invention.

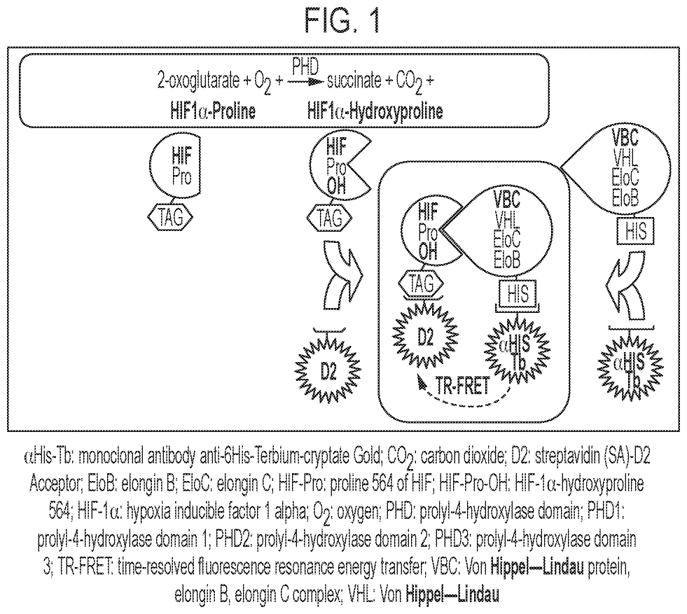

Time-resolved fluorescence resonance energy transfer (TR-FRET) assay was utilized to determine the enzymatic half maximal inhibitory concentration (IC₅₀) value of PHD inhibitors against the full-length human prolyl-4-hydroxylase domain (PHD) enzymes, PHD1, PHD2, and PHD3. The TR-FRET assay was developed based on the specific binding of hydroxylated HIF-1α peptide with the complex formed by VHL, EloB and EloC (VBC), to generate a fluorescent signal. Terbium (Tb)-Donor (monoclonal antibody anti-6His-Tb-cryptate Gold) and D2-acceptor (streptavidin [SA]-D2) of TR-FRET are linked to the VBC complex and to HIF-1α peptide, respectively. The VBC complex binds specifically to the HIF-1α peptide when it is hydroxylated, allowing energy transfer from TR-FRET donor to acceptor (FIG. 1).

Materials and Methods

All chemicals and materials unless otherwise noted were of standard laboratory grade and were purchased from Sigma-Aldrich (St. Louis, MO, USA).

### Reagents

TR-FRET Reagents

Monoclonal antibody anti-6His-Tb-cryptate Gold (catalog #61HI2TLA) and streptavidin (SA)-D2 (catalog #610SADLA) were purchased from CisBio International (Bedford, MA, USA).

N-terminus biotinylated HIF-1α C35 synthetic peptide representing amino acids 547 to 581 and including the proline 564 PHD2 hydroxylation site was purchased from California Peptide Research (Salt Lake City, UT, USA).

Recombinant Proteins

VBC Complex

His-tagged recombinant VHL protein, EloB, EloC complex (His-VBC) was supplied by Axxam (Milan, Italy). Recombinant human VHL (National Center for Biotechnology Information [NCBI] accession number NP_00542.1) contained a His tag at the C-terminus of amino acids 55 to 213 and is referred to as VHL-His. VHL-His was co-expressed in E. coli with full-length human EloB (NCBI accession number Q15370.1) and full-length human EloC (NCBI accession number Q15369.1) and purified by affinity chromatography on a nickel-nitrilotriacetic acid (Ni-NTA) column as the His-VBC complex. Purity (˜80%) was assessed by sodium dodecyl sulfate polyacrylamide gel electrophoresis (SDS-PAGE).

PHD1

Recombinant human PHD1 protein (catalog #81064, Lot #24717001) was purchased from Active Motif (Carlsbad, CA, USA). PHD1 was expressed in a baculovirus expression system as the full-length protein (NCBI accession number NP_542770.2) with an N-terminal FLAG tag (molecular weight 44.9 kDa). Purity (>90%) was assessed by SDS-PAGE.

PHD2

The full-length human PHD2 enzyme was produced with a baculovirus infected insect cell (BIIC) expression system by Beryllium (Bedford, MA, USA). The PHD2 construct contained amino acids 1 to 426 of PHD2 (UniProt Knowledgebase[UniProtKB]/Swiss-Prot accession number Q9GZT9.1), and a His tag and a Tobacco Etch Virus (TEV) protease cleavage site at the N-terminus. The construct was expressed in Sf9 insect cells, purified by Ni-NTA column and digested with TEV protease to remove the His tag. The purity of final cleaved protein was assessed by SDS-PAGE and was found to be >94% pure.

PHD3

Recombinant human PHD3 protein (molecular weight 31.1 kDa) was purchased from Active Motif (Carlsbad, CA, USA). It was expressed in E. coli as the full-length protein (NCBI accession number NP_071356.1) with an N-terminal 6-His tag (catalog #81033, Lot #24417001). Purity was assessed by SDS-PAGE and was found to be >75% pure.

PHD Inhibitors.

Small molecule PHD inhibitors were synthesized and their identities were confirmed as described herein.

### TR-FRET Assay Procedure

PHD inhibitor compound was preincubated with PHD enzyme in a 10 μL reaction volume in white 384-well Optiplate microplates (catalog #6007290, Perkin Elmer, Waltham, MA, USA). For this, 5 μL PHD inhibitor compound was serially diluted with dilution buffer (50 mM HEPES [4-(2-hydroxyethyl)-1-piperazineethanesulfonic acid] pH 7.5, 50 mM sodium chloride [NaCl], 0.01% Tween-20, 0.01% purified bovine serum albumin [BSA]) and mixed with 5 μL PHD enzyme mix prepared as a 4X concentrate in the dilution buffer containing PHD enzyme (60 nM PHD1,20 nM PHD2, 140 nM PHD3), 40 μM ferrous ammonium sulfate (FAS), 4 mM sodium (Na) ascorbate. The plates were incubated for 30 minutes at room temperature without rotation.

Five microliters of the VBC/anti-6His-Tb-cryptate Gold mix prepared as a 4X concentrate in dilution buffer containing 20 nM His-VBC, 1.32 nM monoclonal antibody anti-6His-Tb-cryptate Gold was then added. This step was followed immediately by the addition of 5 μL of the HIF-1α C35 substrate mix prepared as a 4X concentrate in the dilution buffer containing 120 nM biotin-labeled HIF-1α C35, 132 nM SA-D2, 4 μM 2-oxoglutarate (2-OG) to reach a final reaction volume of 20 μL.

The final assay reaction contained 50 mM HEPES, pH 7.5, 50 mM NaCl, 1 μM 2-OG, 10 μM FAS, 1 mM Na ascorbate, 0.01% Tween-20, 0.01% purified BSA, 30 nM biotin-labeled HIF-1α C35, 5 nM His-VBC, 0.33 nM monoclonal antibody anti-6His-Tb-cryptate Gold, 33 nM SA-D2 and PHD enzyme (15 nM PHD1, 5 nM PHD2, or 35 nM PHD3) with the diluted compound.

For the measurement of the IC₅₀ of PHD inhibitor compound, reactions were incubated for 10 minutes at room temperature and then read on a Perkin Elmer EnVision (Waltham, MA, USA) at an excitation wavelength of 340 nm and at emission wavelengths of 615 nm and 665 nm. The data represent the quotient of the signal intensity at 665 nm and 615 nm, automatically calculated by Envision Manager software (Perkin Elmer, Waltham, MA, USA). The IC₅₀ values (mean, standard deviation, standard error of the mean, geometric mean and 95% confidence interval) were determined using a four-parameter curve-fit using GraphPad Prism 7.0 (GraphPad, La Jolla, CA, USA) and represent the compound concentration plotted against the calculated ratio of 665 nm and 615 nm. TR-FRET assays were performed in triplicate at each concentration of compound and the assays were repeated independently three times.

Kis were calculated from IC₅₀s based on the Cheng Prussoff equation:

Ki=IC50/(1+[2−OGH]/Km)

The final concentration of 2-OG in both the PHD1 and PHD2 assays is 1 uM. The Km of 2-OG for PHD 1 was determined to be 12.7 nM, while the Km of 2-OG for PHD2 was determined to be 22.6 nM.

IC₅₀ data for exemplary compounds are described in Table 1.

TABLE 1
IC₅₀ Data for Exemplary Compounds

| | | PHD1 | PHD2 | PHD3 |
|---|---|---|---|---|
| Cmpd. No. | Structure | IC₅₀ (nM) | IC₅₀ (nM) | IC₅₀ (nM) |
| 1 | 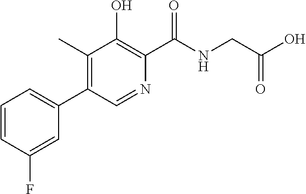 [SMILES: Cc1c(-c2cccc(F)c2)cnc(C(=O)NCC(=O)O)c1O] | A | A | — |
| 2. | 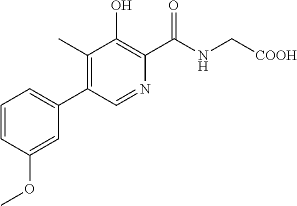 [SMILES: COc1cccc(-c2cnc(C(=O)NCC(=O)O)c(O)c2C)c1] | A | A | A |
| 3. | 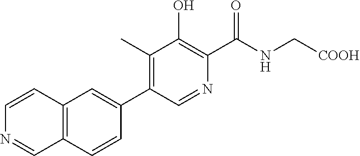 [SMILES: Cc1c(-c2ccc3cnccc3c2)cnc(C(=O)NCC(=O)O)c1O] | A | A | A |
| 4. | 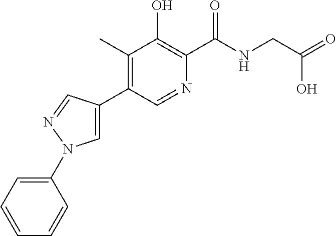 [SMILES: Cc1c(-c2cnn(-c3ccccc3)c2)cnc(C(=O)NCC(=O)O)c1O] | A | A | — |
| 5. | 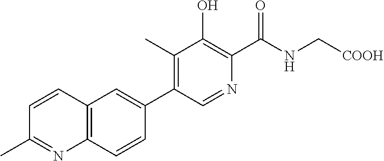 [SMILES: Cc1ccc2cc(-c3cnc(C(=O)NCC(=O)O)c(O)c3C)ccc2n1] | A | A | A |
| 6 | 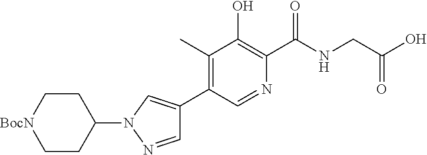 [SMILES: Cc1c(-c2cnn(C3CCN(C(=O)OC(C)(C)C)CC3)c2)cnc(C(=O)NCC(=O)O)c1O] | A | A | — |
| 7. | 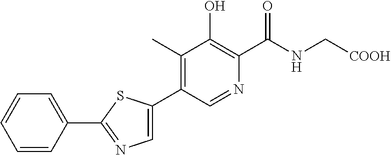 [SMILES: Cc1c(-c2cnc(-c3ccccc3)s2)cnc(C(=O)NCC(=O)O)c1O] | A | A | A |
| 8. | 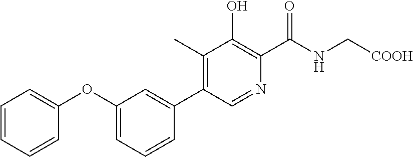 [SMILES: Cc1c(-c2cccc(Oc3ccccc3)c2)cnc(C(=O)NCC(=O)O)c1O] | A | A | — |
| 9. | 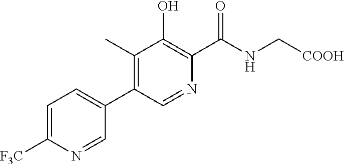 [SMILES: Cc1c(-c2ccc(C(F)(F)F)nc2)cnc(C(=O)NCC(=O)O)c1O] | A | A | A |
| 10. | 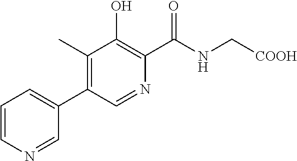 [SMILES: Cc1c(-c2cccnc2)cnc(C(=O)NCC(=O)O)c1O] | A | A | A |
| 11. | 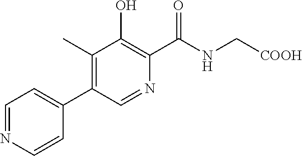 [SMILES: Cc1c(-c2ccncc2)cnc(C(=O)NCC(=O)O)c1O] | A | A | — |
| 12. | 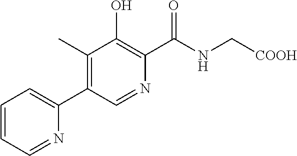 [SMILES: Cc1c(-c2ccccn2)cnc(C(=O)NCC(=O)O)c1O] | A | A | — |
| 13. | 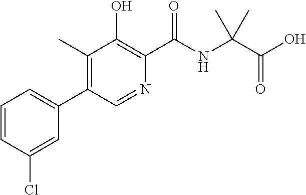 [SMILES: Cc1c(-c2cccc(Cl)c2)cnc(C(=O)NC(C)(C)C(=O)O)c1O] | D | D | — |
| 14. | 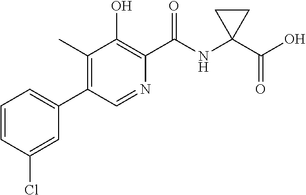 [SMILES: Cc1c(-c2cccc(Cl)c2)cnc(C(=O)NC2(C(=O)O)CC2)c1O] | C | C | — |
| 15. | 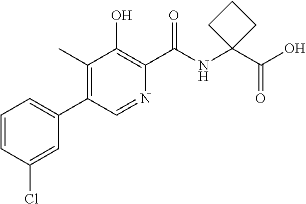 [SMILES: Cc1c(-c2cccc(Cl)c2)cnc(C(=O)NC2(C(=O)O)CCC2)c1O] | D | D | — |
| 16. | 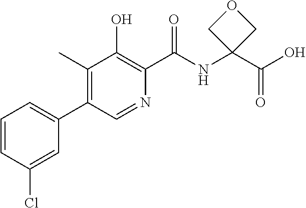 [SMILES: Cc1c(-c2cccc(Cl)c2)cnc(C(=O)NC2(C(=O)O)COC2)c1O] | C | D | — |
| 17. | 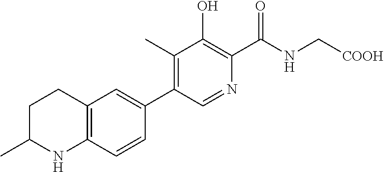 [SMILES: Cc1c(-c2ccc3c(c2)CCC(C)N3)cnc(C(=O)NCC(=O)O)c1O] | A | A | — |
| 18. | 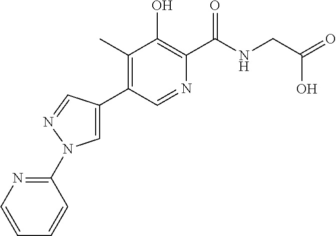 [SMILES: Cc1c(-c2cnn(-c3ccccn3)c2)cnc(C(=O)NCC(=O)O)c1O] | A | A | A |
| 19. | 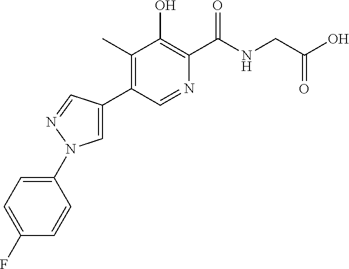 [SMILES: Cc1c(-c2cnn(-c3ccc(F)cc3)c2)cnc(C(=O)NCC(=O)O)c1O] | A | A | A |
| 20. | 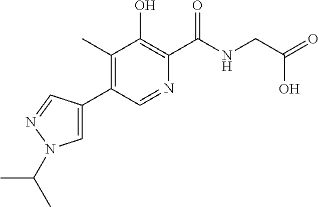 [SMILES: Cc1c(-c2cnn(C(C)C)c2)cnc(C(=O)NCC(=O)O)c1O] | A | A | A |
| 21. | 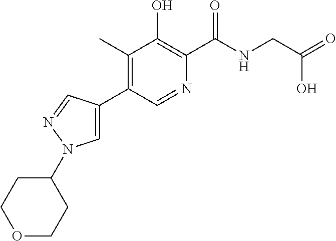 [SMILES: Cc1c(-c2cnn(C3CCOCC3)c2)cnc(C(=O)NCC(=O)O)c1O] | A | A | A |
| 22. | 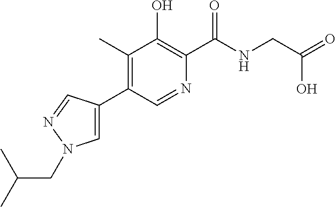 [SMILES: Cc1c(-c2cnn(CC(C)C)c2)cnc(C(=O)NCC(=O)O)c1O] | A | A | A |
| 23. | 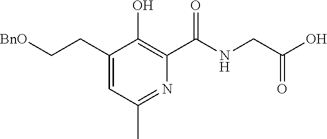 [SMILES: Cc1cc(CCOCc2ccccc2)c(O)c(C(=O)NCC(=O)O)n1] | B | B | — |
| 24. | 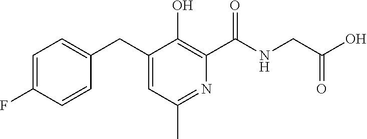 [SMILES: Cc1cc(Cc2ccc(F)cc2)c(O)c(C(=O)NCC(=O)O)n1] | B | B | — |
| 25. | 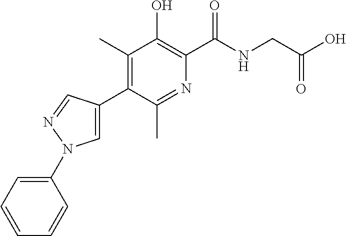 [SMILES: Cc1nc(C(=O)NCC(=O)O)c(O)c(C)c1-c1cnn(-c2ccccc2)c1] | A | A | A |
| 26. | 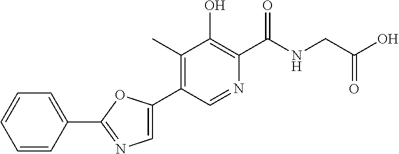 [SMILES: Cc1c(-c2cnc(-c3ccccc3)o2)cnc(C(=O)NCC(=O)O)c1O] | A | A | A |
| 27. | 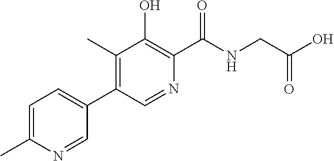 [SMILES: Cc1ccc(-c2cnc(C(=O)NCC(=O)O)c(O)c2C)cn1] | A | A | B |
| 28. | 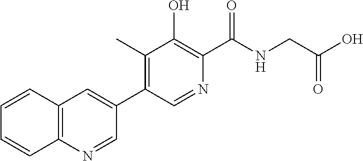 [SMILES: Cc1c(-c2cnc3ccccc3c2)cnc(C(=O)NCC(=O)O)c1O] | A | A | A |
| 29. | 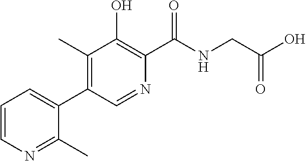 [SMILES: Cc1ncccc1-c1cnc(C(=O)NCC(=O)O)c(O)c1C] | B | B | — |
| 30. | 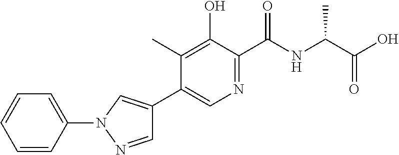 [SMILES: Cc1c(-c2cnn(-c3ccccc3)c2)cnc(C(=O)N[C@H](C)C(=O)O)c1O] | C | C | — |
| 31. | 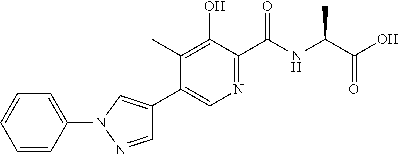 [SMILES: Cc1c(-c2cnn(-c3ccccc3)c2)cnc(C(=O)N[C@@H](C)C(=O)O)c1O] | A | A | A |
| 32. | 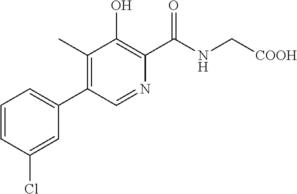 [SMILES: Cc1c(-c2cccc(Cl)c2)cnc(C(=O)NCC(=O)O)c1O] | A | A | A |
| 33. | 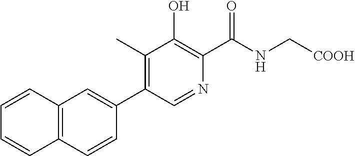 [SMILES: Cc1c(-c2ccc3ccccc3c2)cnc(C(=O)NCC(=O)O)c1O] | A | A | A |
| 34. | 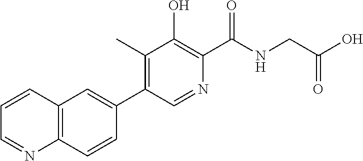 [SMILES: Cc1c(-c2ccc3ncccc3c2)cnc(C(=O)NCC(=O)O)c1O] | A | A | — |
| 35. | 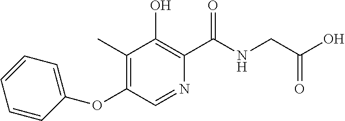 [SMILES: Cc1c(Oc2ccccc2)cnc(C(=O)NCC(=O)O)c1O] | B | B | — |
| 36. | 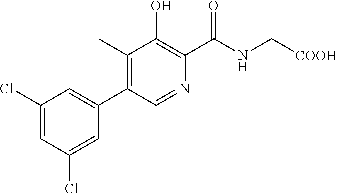 [SMILES: Cc1c(-c2cc(Cl)cc(Cl)c2)cnc(C(=O)NCC(=O)O)c1O] | A | A | A |
| 37. | 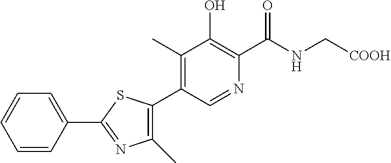 [SMILES: Cc1nc(-c2ccccc2)sc1-c1cnc(C(=O)NCC(=O)O)c(O)c1C] | A | A | A |
| 38. | 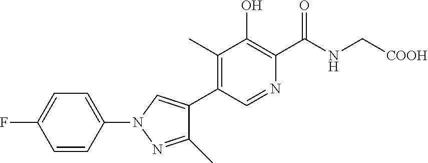 [SMILES: Cc1nn(-c2ccc(F)cc2)cc1-c1cnc(C(=O)NCC(=O)O)c(O)c1C] | A | A | A |
| 39. | 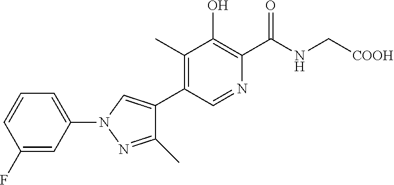 [SMILES: Cc1nn(-c2cccc(F)c2)cc1-c1cnc(C(=O)NCC(=O)O)c(O)c1C] | A | A | A |
| 40. | 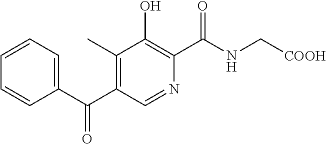 [SMILES: Cc1c(C(=O)c2ccccc2)cnc(C(=O)NCC(=O)O)c1O] | B | B | — |
| 41. | 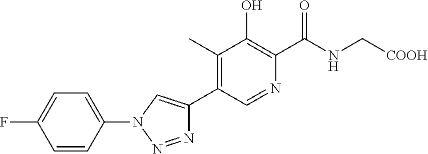 [SMILES: Cc1c(-c2cn(-c3ccc(F)cc3)nn2)cnc(C(=O)NCC(=O)O)c1O] | A | A | A |
| 42. | 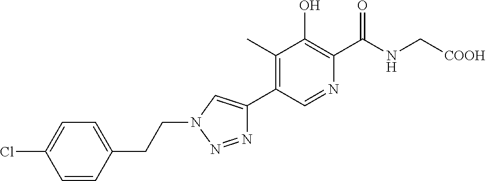 [SMILES: Cc1c(-c2cn(CCc3ccc(Cl)cc3)nn2)cnc(C(=O)NCC(=O)O)c1O] | A | A | A |
| 43. | 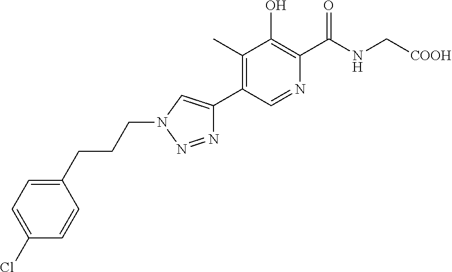 [SMILES: Cc1c(-c2cn(CCCc3ccc(Cl)cc3)nn2)cnc(C(=O)NCC(=O)O)c1O] | A | A | A |
| 44. | 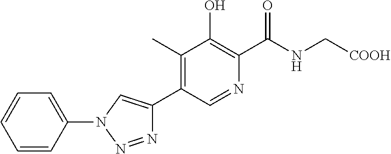 [SMILES: Cc1c(-c2cn(-c3ccccc3)nn2)cnc(C(=O)NCC(=O)O)c1O] | A | A | A |
| 45. | 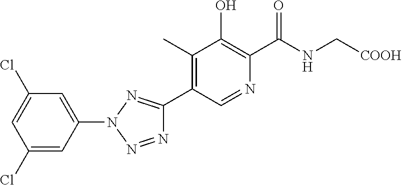 [SMILES: Cc1c(-c2nnn(-c3cc(Cl)cc(Cl)c3)n2)cnc(C(=O)NCC(=O)O)c1O] | A | A | — |
| 46. | 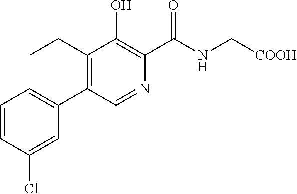 [SMILES: CCc1c(-c2cccc(Cl)c2)cnc(C(=O)NCC(=O)O)c1O] | A | A | — |
| 47. | 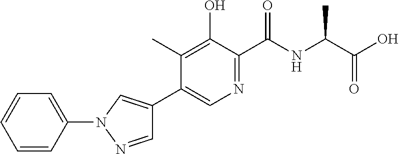 [SMILES: Cc1c(-c2cnn(-c3ccccc3)c2)cnc(C(=O)N[C@@H](C)C(=O)O)c1O] | A | A | A |
| 48. | 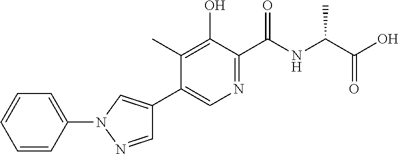 [SMILES: Cc1c(-c2cnn(-c3ccccc3)c2)cnc(C(=O)N[C@H](C)C(=O)O)c1O] | C | C | — |
| 49. | 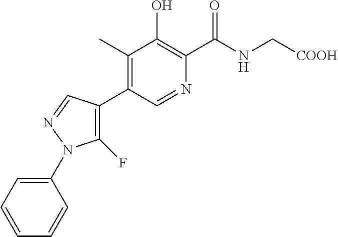 [SMILES: Cc1c(-c2cnn(-c3ccccc3)c2F)cnc(C(=O)NCC(=O)O)c1O] | A | A | — |
| 50. | 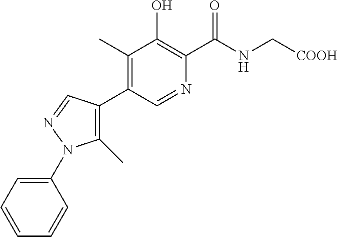 [SMILES: Cc1c(-c2cnn(-c3ccccc3)c2C)cnc(C(=O)NCC(=O)O)c1O] | B | B | — |
| 51. | 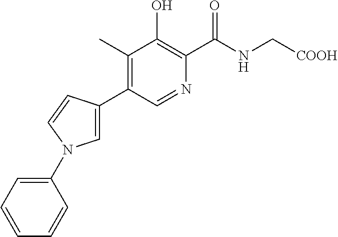 [SMILES: Cc1c(-c2ccn(-c3ccccc3)c2)cnc(C(=O)NCC(=O)O)c1O] | A | B | — |
| 52. | 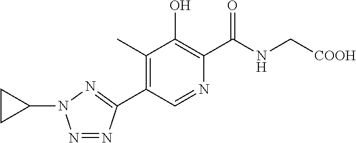 [SMILES: Cc1c(-c2nnn(C3CC3)n2)cnc(C(=O)NCC(=O)O)c1O] | A | A | — |
| 53. | 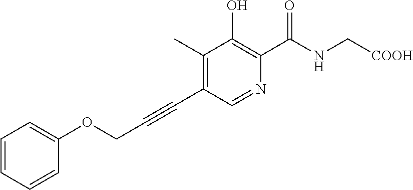 [SMILES: Cc1c(C#CCOc2ccccc2)cnc(C(=O)NCC(=O)O)c1O] | A | A | — |
| 54. | 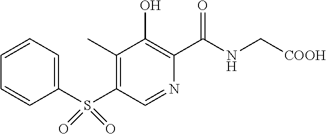 [SMILES: Cc1c(S(=O)(=O)c2ccccc2)cnc(C(=O)NCC(=O)O)c1O] | B | B | — |
| 55. | 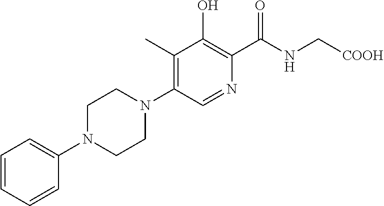 [SMILES: Cc1c(N2CCN(c3ccccc3)CC2)cnc(C(=O)NCC(=O)O)c1O] | A | B | — |
| 56. | 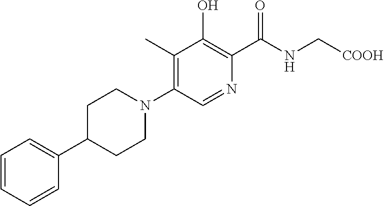 [SMILES: Cc1c(N2CCC(c3ccccc3)CC2)cnc(C(=O)NCC(=O)O)c1O] | A | A | — |
| 57. | 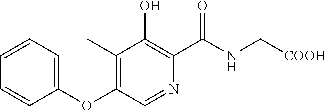 [SMILES: Cc1c(Oc2ccccc2)cnc(C(=O)NCC(=O)O)c1O] | C | B | — |
| 58. | 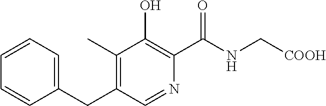 [SMILES: Cc1c(Cc2ccccc2)cnc(C(=O)NCC(=O)O)c1O] | C | B | — |
| 59. | 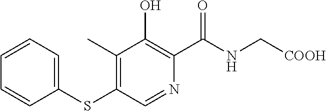 [SMILES: Cc1c(Sc2ccccc2)cnc(C(=O)NCC(=O)O)c1O] | C | B | — |
| 60. | 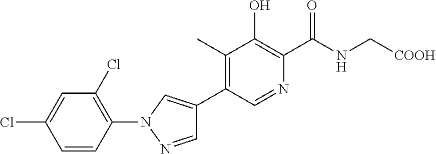 [SMILES: Cc1c(-c2cnn(-c3ccc(Cl)cc3Cl)c2)cnc(C(=O)NCC(=O)O)c1O] | A | A | — |
| 61. | 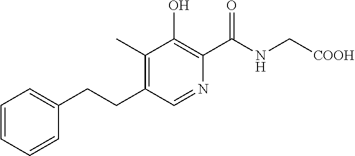 [SMILES: Cc1c(CCc2ccccc2)cnc(C(=O)NCC(=O)O)c1O] | B | B | — |
| 62. | 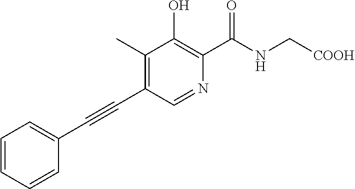 [SMILES: Cc1c(C#Cc2ccccc2)cnc(C(=O)NCC(=O)O)c1O] | A | A | — |
| 63. | 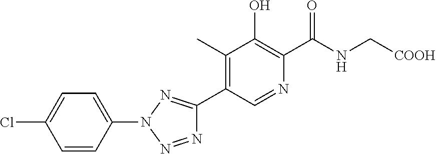 [SMILES: Cc1c(-c2nnn(-c3ccc(Cl)cc3)n2)cnc(C(=O)NCC(=O)O)c1O] | A | A | — |
| 64. | 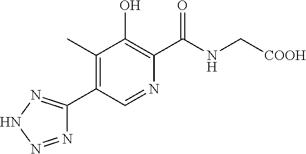 [SMILES: Cc1c(-c2nn[nH]n2)cnc(C(=O)NCC(=O)O)c1O] | B | B | — |
| 65. | 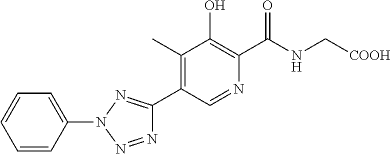 [SMILES: Cc1c(-c2nnn(-c3ccccc3)n2)cnc(C(=O)NCC(=O)O)c1O] | A | A | — |
| 66. | 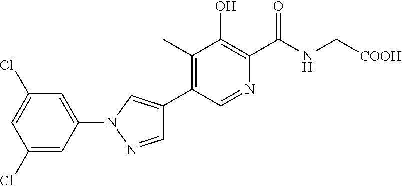 [SMILES: Cc1c(-c2cnn(-c3cc(Cl)cc(Cl)c3)c2)cnc(C(=O)NCC(=O)O)c1O] | A | A | — |
| 67. | 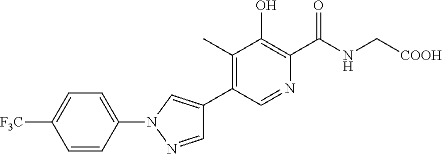 [SMILES: Cc1c(-c2cnn(-c3ccc(C(F)(F)F)cc3)c2)cnc(C(=O)NCC(=O)O)c1O] | A | A | — |
| 68. | 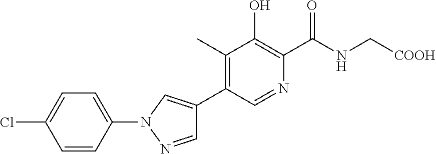 [SMILES: Cc1c(-c2cnn(-c3ccc(Cl)cc3)c2)cnc(C(=O)NCC(=O)O)c1O] | A | A | — |
| 69. | 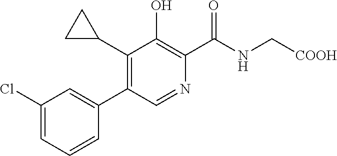 [SMILES: O=C(O)CNC(=O)c1ncc(-c2cccc(Cl)c2)c(C2CC2)c1O] | B | B | — |
| 70. | 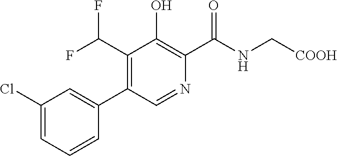 [SMILES: O=C(O)CNC(=O)c1ncc(-c2cccc(Cl)c2)c(C(F)F)c1O] | B | B | — |

Legend:
A = IC₅₀ < 100 nM
B =100 nM ≤ IC₅₀ < 1000 nM
C = 1000 nM ≤ IC₅₀ < 10000 nM
D = IC₅₀ ≥ 10000 nM

From the ongoing description, one skilled in the art can easily ascertain the essential characteristics of this invention, and without departing from the spirit and scope thereof, can make various changes and modifications of the invention to adapt it to various usages and conditions.

All references, patents or applications, U.S. or foreign, cited in the application are hereby incorporated by reference as if written herein in their entireties. Where any inconsistencies arise, material literally disclosed herein controls.

What is claimed is:

1. A compound of Formula (I) 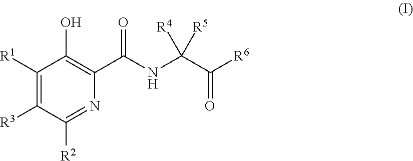
or a pharmaceutically acceptable salt thereof, wherein:
R¹ is optionally substituted C₁₋₃ alkyl, optionally substituted C₃₋₆ cycloalkyl, or optionally substituted 3- to 6-membered heterocycloalkyl;
R² is hydrogen, optionally substituted C₁₋₃ alkyl, halogen, CN, or optionally substituted cycloalkyl;
R³ is hydrogen, optionally substituted aryl, optionally substituted heteroaryl, optionally substituted cycloalkyl, optionally substituted heterocycloalkyl, carbonyl, ether, thioether, optionally substituted arylsulfonyl, optionally substituted heteroarylsulfonyl, optionally substituted arylalkyl, optionally substituted alkynyl, or optionally substituted heteroalkynyl;
R⁴ and R⁵ are independently hydrogen, optionally substituted C₁₋₃ alkyl, or R⁴ and R⁵ together with the carbon to which they are attached form an optionally substituted cycloalkyl or heterocycloalkyl; and
R⁶ is OH or ester.

2. The compound of claim 1, having a structure according to Formula (I) 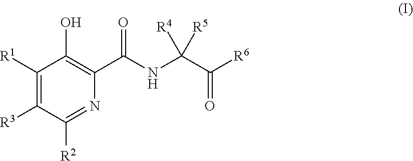
or a pharmaceutically acceptable salt thereof, wherein:
R¹ is C₁₋₃ alkyl optionally substituted with OR⁷, halogen, or aryl, which is optionally substituted with halogen, and wherein R⁷ is C₁₋₃ alkyl optionally substituted with aryl, or R¹ is C₃₋₆ cycloalkyl or 3- to 6-membered heterocycloalkyl;
R² is hydrogen, halogen, CN, or C₁₋₃ alkyl optionally substituted with one or more halogens;
R³ is selected from the group consisting of:
hydrogen; 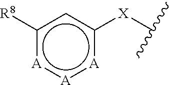
wherein X is a covalent bond, O, S, SO₂, C₁₋₄ alkylene, C₂₋₄ alkynylene, or C₂₋₄ heteroalkynylene; each A is independently N or CR⁹, R⁸ and R⁹ are independently hydrogen, halogen, OR¹⁰, or C₁₋₃ alkyl optionally substituted with one or more halogens, and R¹⁰ is C₁₋₃ alkyl or aryl; 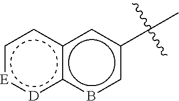
wherein B is N or CR¹¹, D is N, NH, or CR¹¹, E is N, CR¹¹, or CHR¹², and R¹¹ and R¹² are independently hydrogen or C₁₋₃ alkyl, and wherein the dashed circle represents the presence or absence of a conjugated system; 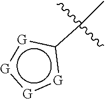
wherein each G is independently N, NH, NR¹³, or CR¹⁴; R¹³ is C₃₋₆ cycloalkyl, 3- to 6-membered heterocycloalkyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens, and R¹⁴ is hydrogen, halogen, C₃₋₆ cycloalkyl, 3- to 6-membered heterocycloalkyl, or C₁₋₃ alkyl; 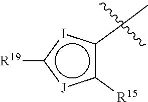
wherein I is O, S, or CH, J is N or CH, R¹⁵ is hydrogen, C₃₋₆ cycloalkyl, 3- to 6-membered heterocycloalkyl, or C₁₋₃ alkyl, and R¹⁹ is hydrogen, C₃₋₆ cycloalkyl, 3- to 6-membered heterocycloalkyl, or aryl;
OR¹⁶ wherein R¹⁶ is aryl; 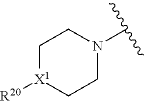
wherein X¹ is N or CH, and R²⁰ is optionally substituted aryl; and
COR¹⁷ wherein R¹⁷ is aryl;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl; and
R⁶ is OH or OR¹⁸, wherein R¹⁸ is C₁₋₆ alkyl.

3. The compound of claim 1 or 2, having a structure according to Formula (II) 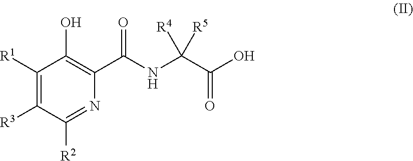
or a pharmaceutically acceptable salt thereof, wherein:
R¹ is C₁₋₃ alkyl optionally substituted with OR⁷, halogen, or aryl, which is optionally substituted with halogen, and wherein R⁷ is C₁₋₃ alkyl optionally substituted with aryl, or R¹ is C₃₋₆ cycloalkyl or 3- to 6-membered heterocycloalkyl;
R² is hydrogen, halogen, CN, or C₁₋₃ alkyl optionally substituted with one or more halogens;
R³ is selected from the group consisting of:
hydrogen; 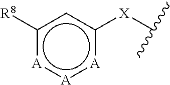
wherein X is a covalent bond, O, S, SO₂, C₁₋₄ alkylene, C₂₋₄ alkynylene, or C₂₋₄ heteroalkynylene; each A is independently N or CR⁹, R⁸ and R⁹ are independently hydrogen, halogen, OR¹⁰, or C₁₋₃ alkyl optionally substituted with one or more halogens, and R¹⁰ is C₁₋₃ alkyl or aryl; 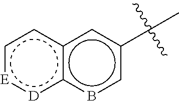
wherein B is N or CR¹¹, D is N, NH, or CR¹¹, E is N, CR¹¹, or CHR¹², and R¹¹ and R¹² are independently hydrogen or C₁₋₃ alkyl, and wherein the dashed circle represents the presence or absence of a conjugated system; 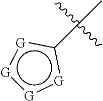
wherein each G is independently N, NR¹³, CR¹⁴, R¹³ is C₃₋₆ cycloalkyl or 3- to 6-membered heterocycloalkyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens, and R¹⁴ is hydrogen, halogen, C₃₋₆ cycloalkyl, 3- to 6-membered heterocycloalkyl, or C₁₋₃ alkyl; 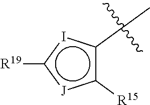
R¹ wherein I is O, S, or CH, J is N or CH, R¹⁵ is hydrogen, C₃₋₆ cycloalkyl, 3- to 6-membered heterocycloalkyl, or C₁₋₃ alkyl, and R¹⁹ is hydrogen C₃₋₆ cycloalkyl, 3- to 6-membered heterocycloalkyl, or aryl;
OR¹⁶ wherein R¹⁶ is aryl; 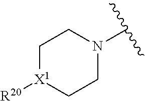
wherein X¹ is N or CH, and R²⁰ is optionally substituted aryl; and
COR¹⁷ wherein R¹⁷ is aryl;
and
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl.

4. The compound of any one of claims 1-3, wherein
R¹ is optionally substituted C₁₋₃ alkyl; and/or
R³ is hydrogen, optionally substituted aryl, optionally substituted heteroaryl, optionally substituted cycloalkyl, optionally substituted heterocycloalkyl, carbonyl, or ether.

5. The compound of any one of claims 1-3, wherein
R¹ is C₁₋₃ alkyl optionally substituted with OR⁷ or aryl, which is optionally substituted with halogen, and wherein R⁷ is C₁₋₃ alkyl optionally substituted with aryl; and/or
R³ is selected from the group consisting of: hydrogen,

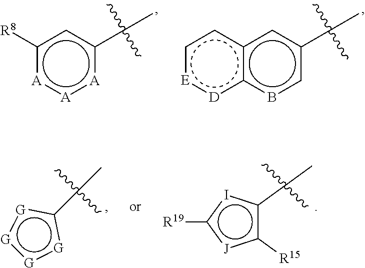

6. The compound of any one of claims 1-3, wherein R³ is

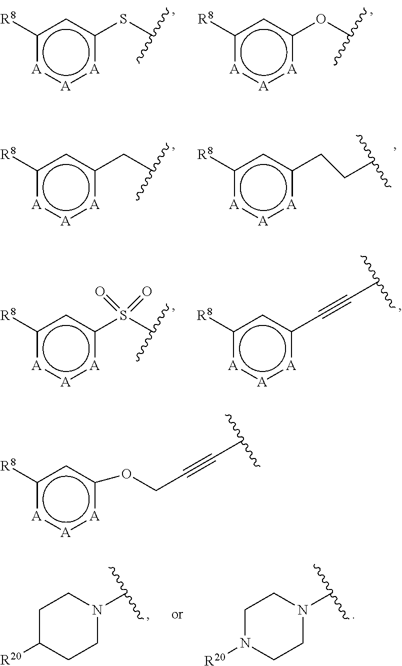

7. The compound of any one of claims 1-6, wherein R¹ is unsubstituted C₁₋₃ alkyl, R² is hydrogen, R⁴ and R⁵ are each hydrogen, and R⁶ is OH.

8. The compound of any one of claims 1-6, wherein each R¹ and R² is unsubstituted C₃ alkyl, R⁴ and R⁵ are each hydrogen, and R⁶ is OH.

9. The compound of any one of claims 1-6, wherein R² is unsubstituted C₁₋₃ alkyl, R³ is hydrogen, R⁴ and R⁵ are each hydrogen, and R⁶ is OH.

10. The compound of any one of claims 1-3, having a structure according to Formula (III) 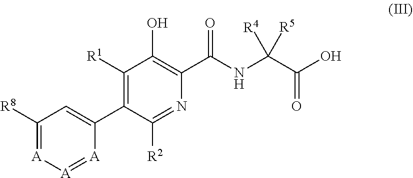
or a pharmaceutically acceptable salt thereof, wherein
each A is independently N or CR⁹;
R¹ is C₁₋₃ alkyl optionally substituted with OR⁷, halogen, or aryl, which is optionally substituted with halogen, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R⁷ is C₁₋₃ alkyl optionally substituted with aryl;
R⁸ and R⁹ are independently hydrogen, halogen, OR¹⁰, or C₁₋₃ alkyl optionally substituted with one or more halogens; and
R¹⁰ is C₁₋₃ alkyl or aryl.

11. The compound of any one of claims 1-3 and 10, having a structure according to Formula (IV) 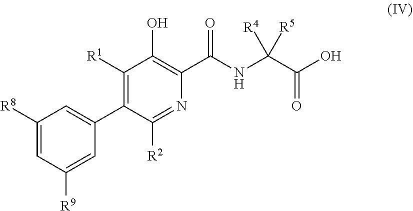
or a pharmaceutically acceptable salt thereof, wherein
R¹ is C₁₋₃ alkyl optionally substituted with OR⁷, halogen, or aryl, which is optionally substituted with halogen, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R⁷ is C₁₋₃ alkyl optionally substituted with aryl;
R⁸ and R⁹ are independently hydrogen, halogen, OR¹⁰, or C₁₋₃ alkyl optionally substituted with one or more halogens; and
R¹⁰ is C₁₋₃ alkyl or aryl.

12. The compound of any one of claims 1-3 and 10, having a structure according to Formula (V) 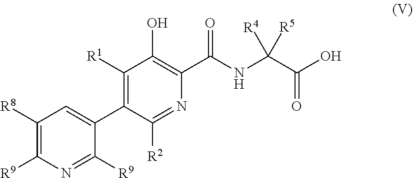
or a pharmaceutically acceptable salt thereof, wherein:
R¹ is C₁₋₃ alkyl optionally substituted with OR⁷, halogen, or aryl, which is optionally substituted with halogen, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R⁷ is C₁₋₃ alkyl optionally substituted with aryl;
R⁸ and each R⁹ are independently hydrogen, halogen, OR¹⁰, or C₁₋₃ alkyl optionally substituted with one or more halogens; and
R¹⁰ is C₁₋₃ alkyl or aryl.

13. The compound of any one of claims 1-3, having a structure according to Formula (VI) 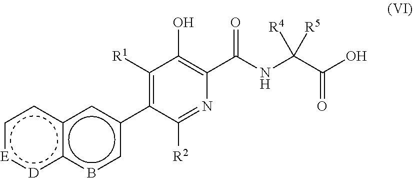
or a pharmaceutically acceptable salt thereof, wherein:
B is N or CR¹¹;
D is N, NH, or CR¹¹;
E is N, CR¹¹, or CHR¹²;
R¹ is C₁₋₃ alkyl optionally substituted with OR⁷, halogen or aryl, which is optionally substituted with halogen, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R⁷ is C₁₋₃ alkyl optionally substituted with aryl;
R¹¹ and R¹² are independently hydrogen or C₁₋₃ alkyl; and
wherein the dashed circle represents the presence or absence of a conjugated system.

14. The compound of claim 13, having a structure according to Formula (VII) 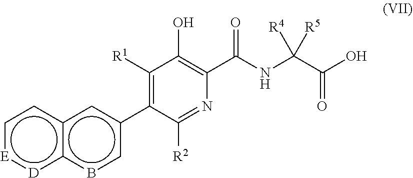
or a pharmaceutically acceptable salt thereof.

15. The compound of claim 13, having a structure according to Formula (VIII) 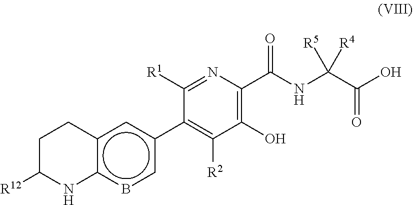
or a pharmaceutically acceptable salt thereof, wherein:
B is N or CR¹¹;
R¹ is C₁₋₃ alkyl optionally substituted with OR⁷, halogen, or aryl, which is optionally substituted with halogen, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R⁷ is C₁₋₃ alkyl optionally substituted with aryl; and
R¹² is hydrogen or C₁₋₃ alkyl.

16. The compound of any one of claims 1-3, having a structure according to Formula (IX) 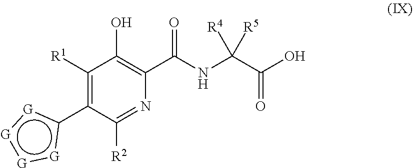
or a pharmaceutically acceptable salt thereof, wherein:
each G is independently N, NH, NR¹³, or CR¹⁴;
R¹ is C₁₋₃ alkyl optionally substituted with OR⁷, halogen, or aryl, which is optionally substituted with halogen, and wherein R⁷ is C₁₋₃ alkyl optionally substituted with aryl, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens; and
R¹⁴ is hydrogen, halogen, cyclopropyl, or C₁₋₃ alkyl.

17. The compound of claim 16, having a structure according to Formula (X) 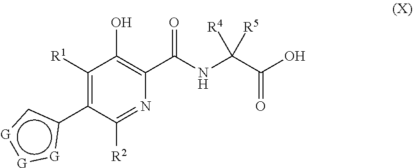
or a pharmaceutically acceptable salt thereof, wherein:
each G is independently N, NR¹³, or CR¹⁴;
R¹ is C₁₋₃ alkyl optionally substituted with OR⁷, halogen, or aryl which is optionally substituted with halogen, and wherein R⁷ is C₁₋₃ alkyl optionally substituted with aryl, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens; and
R¹⁴ is hydrogen, halogen, cyclopropyl, or C₁₋₃ alkyl.

18. The compound of claim 16 or 17, having a structure according to Formula (XI) 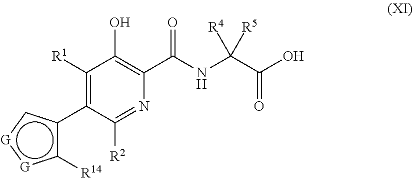
or a pharmaceutically acceptable salt thereof, wherein
each G is independently N or NR³;
R¹ is C₁₋₃ alkyl optionally substituted with OR⁷, halogen, or aryl, which is optionally substituted with halogen, and wherein R⁷ is C₁₋₃ alkyl optionally substituted with aryl, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R¹³ is cyclopropyl, heteroaryl, aryl optionally substituted with one or more halogens, aryl optionally substituted with one or more optionally substituted C₁₋₃ alkyls, heterocycloalkyl optionally substituted with t-butyloxycarbonyl, C₁₋₄ alkyl optionally substituted with aryl, which is optionally substituted with one or more halogens; and
R¹⁴ is hydrogen, halogen, cyclopropyl, or C₁₋₃ alkyl.

19. The compound of any one of claims 16-18, having a structure according to Formula (XIIa) or Formula (XIIb) 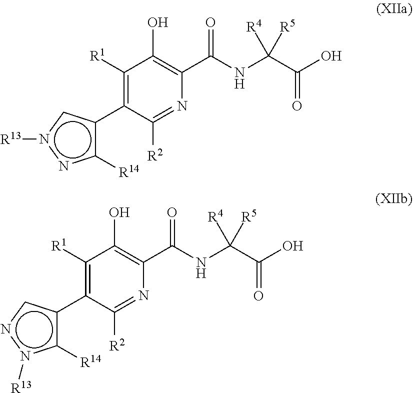
or a pharmaceutically acceptable salt thereof, wherein:
R¹ is C₁₋₃ alkyl optionally substituted with OR⁷, halogen, or aryl, which is optionally substituted with halogen, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R⁷ is C₁₋₃ alkyl optionally substituted with aryl;
R¹³ is cyclopropyl, heteroaryl, aryl optionally substituted with one or more halogens, aryl optionally substituted with one or more optionally substituted C₁₋₃ alkyls, heterocycloalkyl optionally substituted with t-butyloxycarbonyl, C₁₋₄ alkyl optionally substituted with aryl, which is optionally substituted with one or more halogens; and
R¹⁴ is hydrogen, halogen, cyclopropyl, or C₁₋₃ alkyl.

20. The compound of any one of claims 16-19, having a structure according to Formula (XIII) 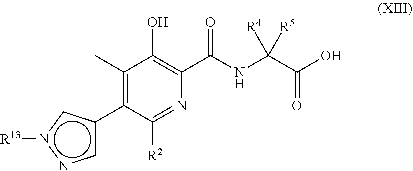
or a pharmaceutically acceptable salt thereof, wherein:
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl; and
R¹³ is aryl or heteroaryl.

21. The compound of any one of claims 1-3, having a structure according to Formula (XIV) 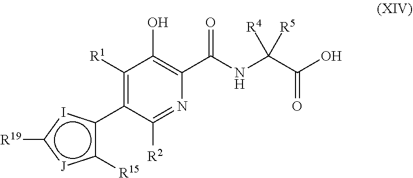
or a pharmaceutically acceptable salt thereof, wherein:
I is O, S, or CH;
J is N or CH;
R¹ is C₁₋₃ alkyl optionally substituted with OR⁷, halogen, or aryl, which is optionally substituted with halogen, and wherein R⁷ is C₁₋₃ alkyl optionally substituted with aryl, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R¹⁵ is hydrogen or C₁₋₃ alkyl; and
R¹⁹ is hydrogen or aryl.

22. The compound of claim 21, having a structure according to Formula (XV) 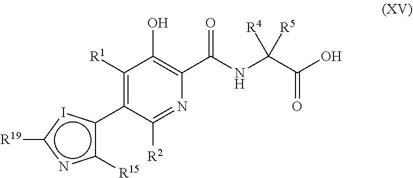
or a pharmaceutically acceptable salt thereof, wherein:
I is O, S, or CH;
R¹ is C₁₋₃ alkyl optionally substituted with OR⁷, halogen, or aryl, which is optionally substituted with halogen, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R⁷ is C₁₋₃ alkyl optionally substituted with aryl; and
R¹⁵ is hydrogen or C₁₋₃ alkyl.
R¹⁹ is hydrogen or aryl.

23. The compound of any one of claims 1-3 and 10, having a structure according to Formula (XVI) 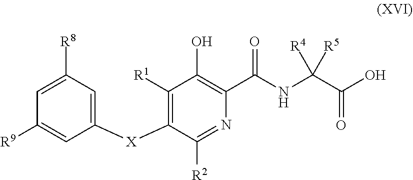
or a pharmaceutically acceptable salt thereof, wherein
X is O, S, or SO₂;
R¹ is C₁₋₃ alkyl optionally substituted with OR⁷, halogen, or aryl which is optionally substituted with halogen, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R⁷ is C₁₋₃ alkyl optionally substituted with aryl;
R⁸ and R⁹ are independently hydrogen, halogen, OR¹⁰, or C₁₋₃ alkyl optionally substituted with one or more halogens; and
R¹⁰ is C₁₋₃ alkyl or aryl.

24. The compound of any one of claims 1-3 and 10, having a structure according to Formula (XVII) 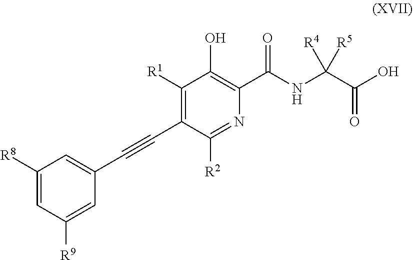
or a pharmaceutically acceptable salt thereof, wherein
R¹ is C₁₋₃ alkyl optionally substituted with OR⁷, halogen, or aryl, which is optionally substituted with halogen, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R⁷ is C₁₋₃ alkyl optionally substituted with aryl;
R⁸ and R⁹ are independently hydrogen, halogen, OR¹⁰, or C₁₋₃ alkyl optionally substituted with one or more halogens; and
R¹⁰ is C₁₋₃ alkyl or aryl.

25. The compound of any one of claims 1-3 and 10, having a structure according to Formula (XVIII) 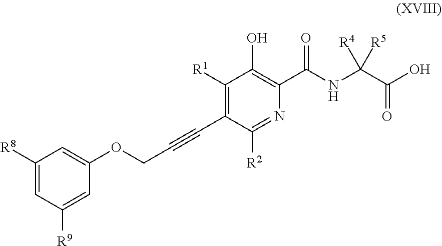
or a pharmaceutically acceptable salt thereof, wherein
R¹ is C₁₋₃ alkyl optionally substituted with OR⁷, halogen, or aryl, which is optionally substituted with halogen, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R⁷ is C₁₋₃ alkyl optionally substituted with aryl;
R⁸ and R⁹ are independently hydrogen, halogen, OR¹⁰, or C₁₋₃ alkyl optionally substituted with one or more halogens; and
R¹⁰ is C₁₋₃ alkyl or aryl.

26. The compound of claim 16, having a structure according to Formula (XIX) 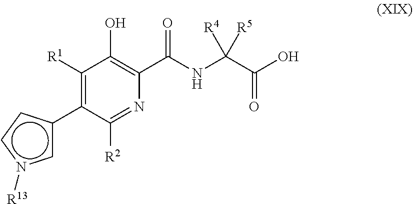
or a pharmaceutically acceptable salt thereof, wherein:
R¹ is C₁₋₃ alkyl optionally substituted with OR⁷, halogen, or aryl, which is optionally substituted with halogen, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R⁷ is C₁₋₃ alkyl optionally substituted with aryl; and
R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens.

27. The compound of claim 16, having a structure according to Formula (XX) 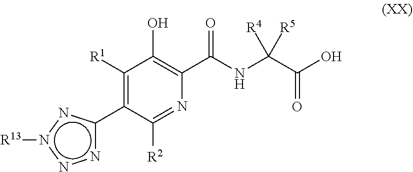
or a pharmaceutically acceptable salt thereof, wherein:
R¹ is C₁₋₃ alkyl optionally substituted with OR⁷, halogen, or aryl, which is optionally substituted with halogen, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R⁷ is C₁₋₃ alkyl optionally substituted with aryl; and
R¹³ is cyclopropyl, aryl optionally substituted with one or more halogens, aryl optionally substituted with one or more optionally substituted C₁₋₃ alkyls, heteroaryl, heterocycloalkyl optionally substituted with t-butyloxycarbonyl, C₁₋₄ alkyl optionally substituted with aryl, which is optionally substituted with one or more halogens.

28. The compound of claim 16, having a structure according to Formula (XXI) 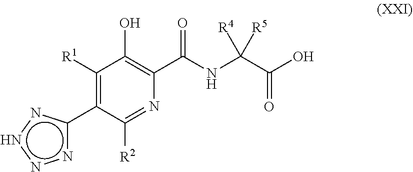
or a pharmaceutically acceptable salt thereof, wherein:
R¹ is C₁₋₃ alkyl optionally substituted with OR⁷, halogen, or aryl, which is optionally substituted with halogen, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl; and
R⁷ is C₁₋₃ alkyl optionally substituted with aryl.

29. The compound of any one of claims 1-3, having a structure according to Formula (XXII) 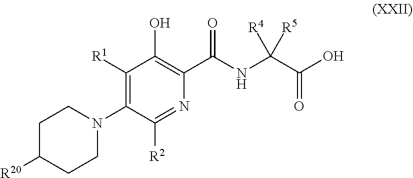
or a pharmaceutically acceptable salt thereof, wherein:
R¹ is C₁₋₃ alkyl optionally substituted with OR⁷, halogen, or aryl, which is optionally substituted with halogen, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R⁷ is C₁₋₃ alkyl optionally substituted with aryl; and
R²⁰ is optionally substituted aryl.

30. The compound of any one of claims 1-3, having a structure according to Formula (XXIII) 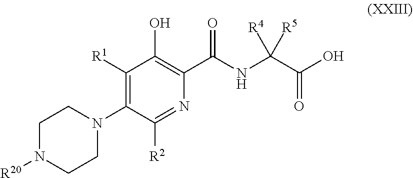
or a pharmaceutically acceptable salt thereof, wherein:
R¹ is C₁₋₃ alkyl optionally substituted with OR⁷, halogen, or aryl, which is optionally substituted with halogen, or R¹ is cyclopropyl;
R² is hydrogen, halogen, CN, or C₁₋₃ alkyl optionally substituted with one or more halogens;
R⁴ and R⁵ are independently hydrogen, C₁₋₃ alkyl optionally substituted with one or more halogens, or R⁴ and R⁵ together with the carbon to which they are attached form an optionally substituted cycloalkyl or heterocycloalkyl;
R⁷ is C₁₋₃ alkyl optionally substituted with aryl; and
R²⁰ is optionally substituted aryl.

31. The compound of any one of claims 1-6, wherein R³ is not hydrogen.

32. The compound of any one of claims 1-6, wherein R³ is unsubstituted phenyl, fluorophenyl, chlorophenyl, difluorophenyl, dichlorophenyl, or trifluoromethylphenyl.

33. The compound of any one of claims 1-6, wherein R³ is OR¹⁶, SR¹⁶, SO₂R¹⁶, CH₂R¹⁶, CH₂CH₂R¹⁶, C≡CR¹⁶, or C≡CCH₂OR¹⁶, and wherein R¹⁶ is aryl.

34. The compound of claim 33, wherein R¹⁶ is phenyl.

35. The compound of any one of claims 1-6, wherein R³ is pyrrolyl, tetrazolyl, triazolyl, or pyrazolyl, optionally substituted by aryl or cycloalkyl.

36. The compound of any one of claims 1-6, wherein R³ is piperidinyl or piperazinyl, optionally substituted by aryl.

37. The compound of claim 35 or 36, wherein R³ is unsubstituted or substituted by cyclopropyl, unsubstituted phenyl, fluorophenyl, chlorophenyl, difluorophenyl, dichlorophenyl, or trifluoromethylphenyl.

38. The compound of any one of claims 1-6, wherein R³ is COR¹⁷, and wherein R¹⁷ is aryl.

39. The compound of claim 38, wherein R¹⁷ is phenyl.

40. The compound of any one of claims 1-39, wherein R¹ is cyclopropyl or substituted C₁₋₃ alkyl.

41. The compound of claim 40, wherein R¹ is cyclopropyl or difluoromethyl.

42. The compound of any one of claims 1-39, wherein R¹ is unsubstituted C₁₋₃ alkyl.

43. The compound claim 42, wherein R¹ is CH₂CH₃.

44. The compound of claim 42, wherein R¹ is CH₃.

45. The compound of any one of claims 1-6 and 9-39, wherein R¹ is C₁₋₃ alkyl substituted with aryl, which is substituted with halogen.

46. The compound of claim 45, wherein R¹ is

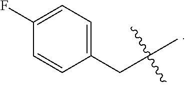 [SMILES: *Cc1ccc(F)cc1]

47. The compound of any one of claims 1-6 and 9-39, wherein R¹ is C₁₋₃ alkyl substituted with OBn.

48. The compound of claim 47, wherein R¹ is CH₂CH₂OBn.

49. The compound of any one of claims 1-7 and 10-48, wherein R² is hydrogen.

50. The compound of any one of claims 1-6 and 8-48, wherein R² is unsubstituted C₁₋₃ alkyl.

51. The compound of claim 50, wherein R² is CH₃.

52. The compound of any one of claims 1-51, wherein R⁴ is hydrogen and R⁵ is hydrogen.

53. The compound of any one of claims 1-6 and 10-51, wherein R⁴ is hydrogen and R⁵ is C₁₋₃ alkyl.

54. The compound of claim 53, wherein R⁵ is CH₃.

55. The compound of any one of claims 1-3 and 10-51, wherein R⁴ is C₁₋₃ alkyl and R⁵ is C₁₋₃ alkyl.

56. The compound of claim 55, wherein R⁴ is CH₃ and R⁵ is CH₃.

57. The compound of any one of claims 1-6 and 10-51, wherein R⁴ and R⁵ together with the carbon to which they are attached form a cycloalkyl or a heterocycloalkyl.

58. The compound of claim 57, wherein the cycloalkyl is cyclopropyl.

59. The compound of claim 57, wherein the cycloalkyl is cyclobutyl.

60. The compound of claim 57, wherein the heterocycloalkyl is

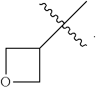 [SMILES: *C1COC1]

61. The compound of any one of claims 1-6, having a structure according to any one of compounds 1-50:

| Cmpd No. | Structure |
|---|---|
| 1 | 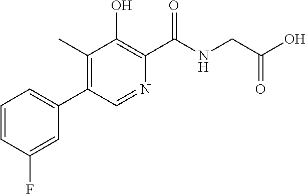 [SMILES: Cc1c(-c2cccc(F)c2)cnc(C(=O)NCC(=O)O)c1O] |
| 2 | 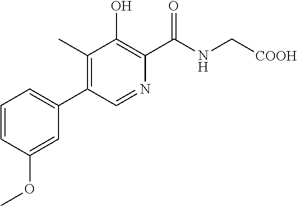 [SMILES: COc1cccc(-c2cnc(C(=O)NCC(=O)O)c(O)c2C)c1] |
| 3 | 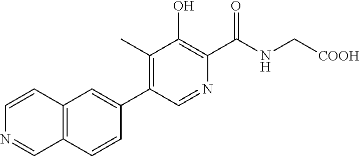 [SMILES: Cc1c(-c2ccc3cnccc3c2)cnc(C(=O)NCC(=O)O)c1O] |
| 4 | 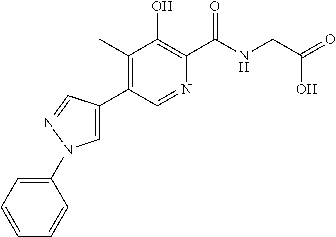 [SMILES: Cc1c(-c2cnn(-c3ccccc3)c2)cnc(C(=O)NCC(=O)O)c1O] |
| 5 | 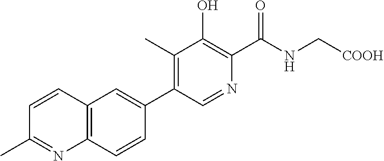 [SMILES: Cc1ccc2cc(-c3cnc(C(=O)NCC(=O)O)c(O)c3C)ccc2n1] |
| 6 | 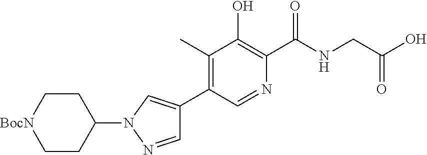 [SMILES: Cc1c(-c2cnn(C3CCN(C(=O)OC(C)(C)C)CC3)c2)cnc(C(=O)NCC(=O)O)c1O] |
| 7 | 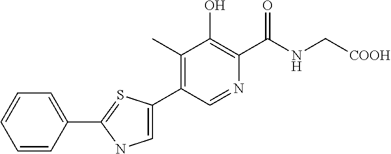 |
| 8 | 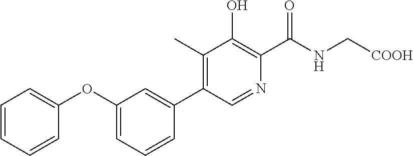 [SMILES: Cc1c(-c2cccc(Oc3ccccc3)c2)cnc(C(=O)NCC(=O)O)c1O] |
| 9 | 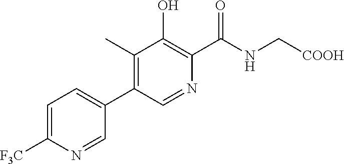 [SMILES: Cc1c(-c2ccc(C(F)(F)F)nc2)cnc(C(=O)NCC(=O)O)c1O] |
| 10 | 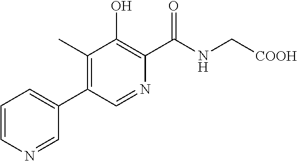 [SMILES: Cc1c(-c2cccnc2)cnc(C(=O)NCC(=O)O)c1O] |
| 11 | 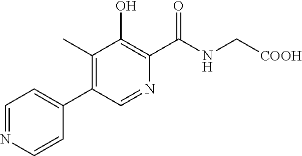 [SMILES: Cc1c(-c2ccncc2)cnc(C(=O)NCC(=O)O)c1O] |
| 12 | 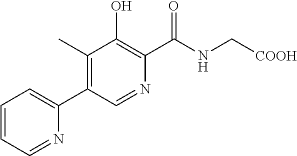 [SMILES: Cc1c(-c2ccccn2)cnc(C(=O)NCC(=O)O)c1O] |
| 13 | 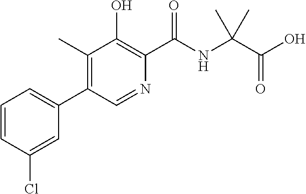 [SMILES: Cc1c(-c2cccc(Cl)c2)cnc(C(=O)NC(C)(C)C(=O)O)c1O] |
| 14 | 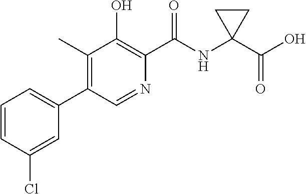 [SMILES: Cc1c(-c2cccc(Cl)c2)cnc(C(=O)NC2(C(=O)O)CC2)c1O] |
| 15 | 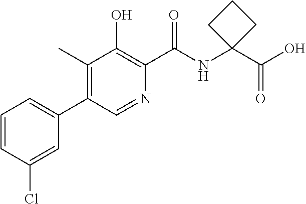 [SMILES: Cc1c(-c2cccc(Cl)c2)cnc(C(=O)NC2(C(=O)O)CCC2)c1O] |
| 16 | 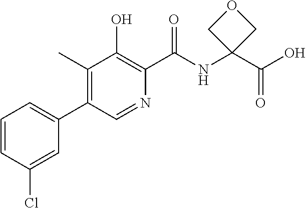 [SMILES: Cc1c(-c2cccc(Cl)c2)cnc(C(=O)NC2(C(=O)O)COC2)c1O] |
| 17 | 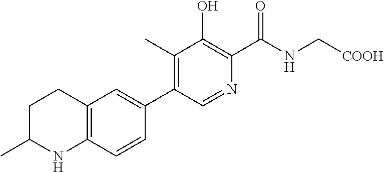 [SMILES: Cc1c(-c2ccc3c(c2)CCC(C)N3)cnc(C(=O)NCC(=O)O)c1O] |
| 18 | 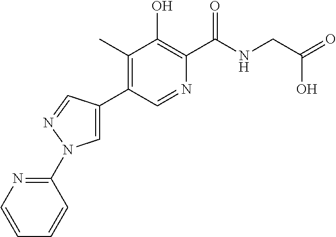 [SMILES: Cc1c(-c2cnn(-c3ccccn3)c2)cnc(C(=O)NCC(=O)O)c1O] |
| 19 | 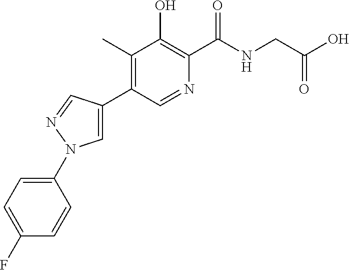 [SMILES: Cc1c(-c2cnn(-c3ccc(F)cc3)c2)cnc(C(=O)NCC(=O)O)c1O] |
| 20 | 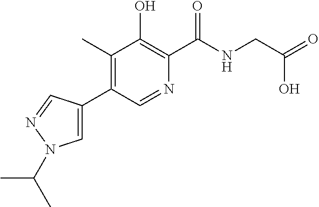 [SMILES: Cc1c(-c2cnn(C(C)C)c2)cnc(C(=O)NCC(=O)O)c1O] |
| 21 | 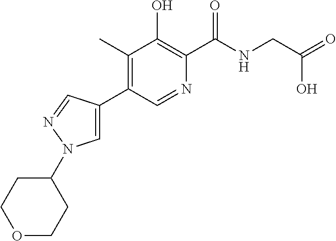 [SMILES: Cc1c(-c2cnn(C3CCOCC3)c2)cnc(C(=O)NCC(=O)O)c1O] |
| 22 | 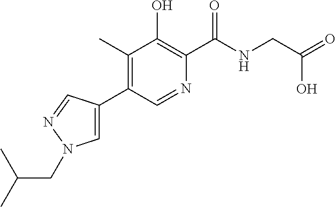 [SMILES: Cc1c(-c2cnn(CC(C)C)c2)cnc(C(=O)NCC(=O)O)c1O] |
| 23 | 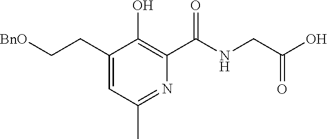 [SMILES: Cc1cc(CCOCc2ccccc2)c(O)c(C(=O)NCC(=O)O)n1] |
| 24 | 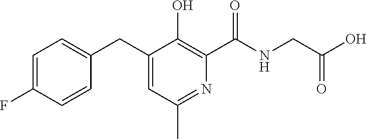 [SMILES: Cc1cc(Cc2ccc(F)cc2)c(O)c(C(=O)NCC(=O)O)n1] |
| 25 | 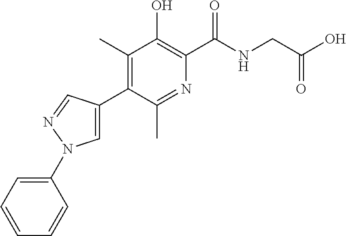 [SMILES: Cc1nc(C(=O)NCC(=O)O)c(O)c(C)c1-c1cnn(-c2ccccc2)c1] |
| 26 | 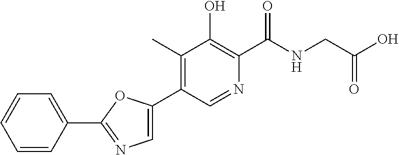 [SMILES: Cc1c(-c2cnc(-c3ccccc3)o2)cnc(C(=O)NCC(=O)O)c1O] |
| 27 | 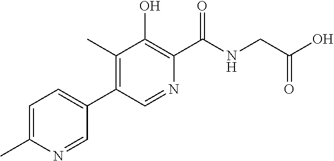 [SMILES: Cc1ccc(-c2cnc(C(=O)NCC(=O)O)c(O)c2C)cn1] |
| 28 | 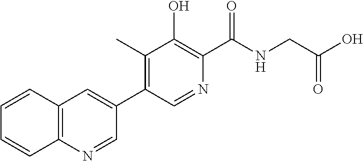 [SMILES: Cc1c(-c2cnc3ccccc3c2)cnc(C(=O)NCC(=O)O)c1O] |
| 29 | 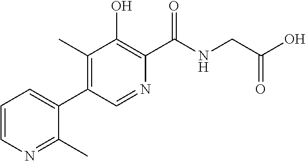 [SMILES: Cc1ncccc1-c1cnc(C(=O)NCC(=O)O)c(O)c1C] |
| 30 | 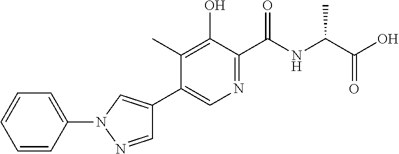 [SMILES: Cc1c(-c2cnn(-c3ccccc3)c2)cnc(C(=O)N[C@H](C)C(=O)O)c1O] |
| 31 | 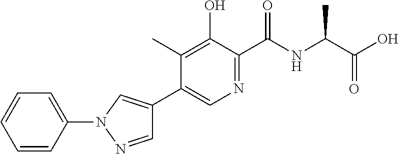 [SMILES: Cc1c(-c2cnn(-c3ccccc3)c2)cnc(C(=O)N[C@@H](C)C(=O)O)c1O] |
| 32 | 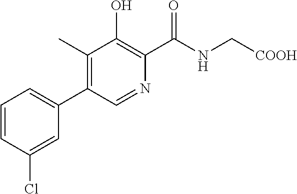 [SMILES: Cc1c(-c2cccc(Cl)c2)cnc(C(=O)NCC(=O)O)c1O] |
| 33 | 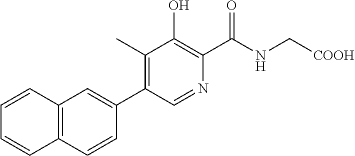 [SMILES: Cc1c(-c2ccc3ccccc3c2)cnc(C(=O)NCC(=O)O)c1O] |
| 34 | 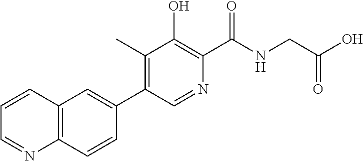 [SMILES: Cc1c(-c2ccc3ncccc3c2)cnc(C(=O)NCC(=O)O)c1O] |
| 35 | 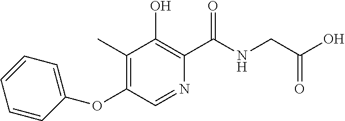 [SMILES: Cc1c(Oc2ccccc2)cnc(C(=O)NCC(=O)O)c1O] |
| 36 | 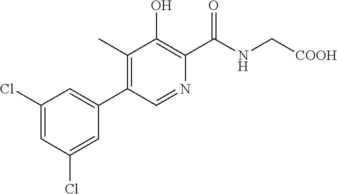 [SMILES: Cc1c(-c2cc(Cl)cc(Cl)c2)cnc(C(=O)NCC(=O)O)c1O] |
| 37 | 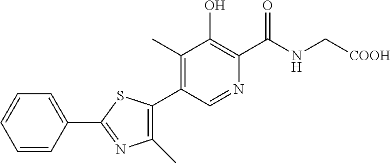 [SMILES: Cc1nc(-c2ccccc2)sc1-c1cnc(C(=O)NCC(=O)O)c(O)c1C] |
| 38 | 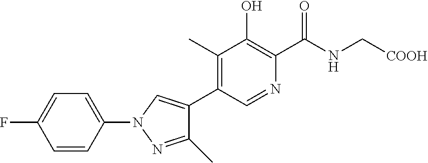 [SMILES: Cc1nn(-c2ccc(F)cc2)cc1-c1cnc(C(=O)NCC(=O)O)c(O)c1C] |
| 39 | 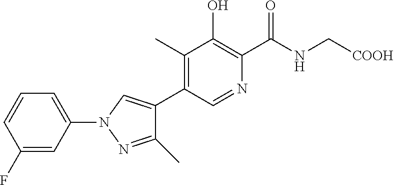 [SMILES: Cc1nn(-c2cccc(F)c2)cc1-c1cnc(C(=O)NCC(=O)O)c(O)c1C] |
| 40 | 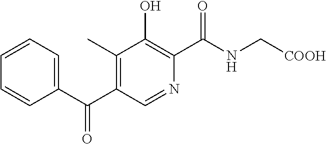 [SMILES: Cc1c(C(=O)c2ccccc2)cnc(C(=O)NCC(=O)O)c1O] |
| 41 | 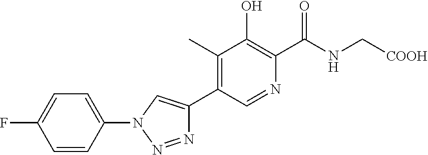 [SMILES: Cc1c(-c2cn(-c3ccc(F)cc3)nn2)cnc(C(=O)NCC(=O)O)c1O] |
| 42 | 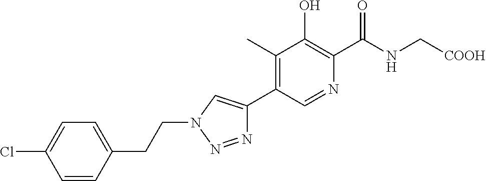 [SMILES: Cc1c(-c2cn(CCc3ccc(Cl)cc3)nn2)cnc(C(=O)NCC(=O)O)c1O] |
| 43 | 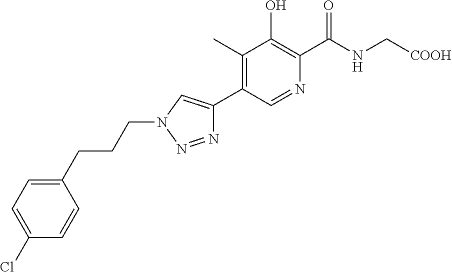 [SMILES: Cc1c(-c2cn(CCCc3ccc(Cl)cc3)nn2)cnc(C(=O)NCC(=O)O)c1O] |
| 44 | 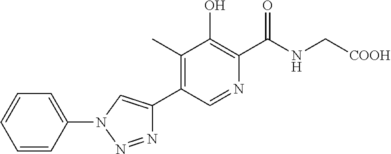 [SMILES: Cc1c(-c2cn(-c3ccccc3)nn2)cnc(C(=O)NCC(=O)O)c1O] |
| 45 | 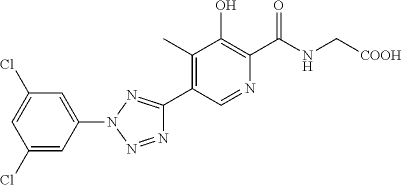 [SMILES: Cc1c(-c2nnn(-c3cc(Cl)cc(Cl)c3)n2)cnc(C(=O)NCC(=O)O)c1O] |
| 46 | 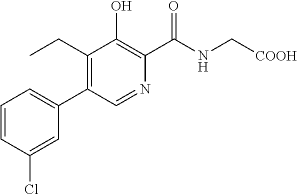 [SMILES: CCc1c(-c2cccc(Cl)c2)cnc(C(=O)NCC(=O)O)c1O] |
| 47 | 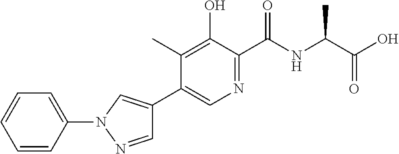 [SMILES: Cc1c(-c2cnn(-c3ccccc3)c2)cnc(C(=O)N[C@@H](C)C(=O)O)c1O] |
| 48 | 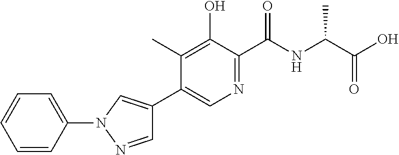 [SMILES: Cc1c(-c2cnn(-c3ccccc3)c2)cnc(C(=O)N[C@H](C)C(=O)O)c1O] |
| 49 | 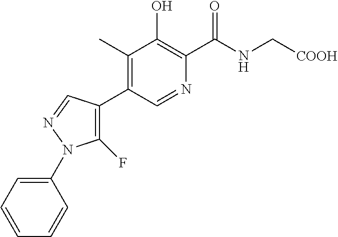 [SMILES: Cc1c(-c2cnn(-c3ccccc3)c2F)cnc(C(=O)NCC(=O)O)c1O] |
| 50 | 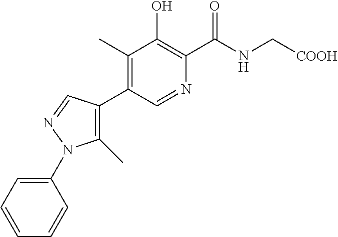 [SMILES: Cc1c(-c2cnn(-c3ccccc3)c2C)cnc(C(=O)NCC(=O)O)c1O] |

or a pharmaceutically acceptable salt thereof.

62. The compound of any one of claims 1-6, having a structure according to any one of compounds 51-70:

| Cmpd No. | Structure |
|---|---|
| 51 | 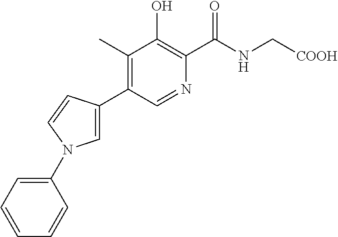 [SMILES: Cc1c(-c2ccn(-c3ccccc3)c2)cnc(C(=O)NCC(=O)O)c1O] |
| 52 | 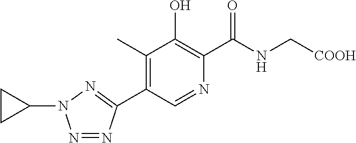 [SMILES: Cc1c(-c2nnn(C3CC3)n2)cnc(C(=O)NCC(=O)O)c1O] |
| 53 | 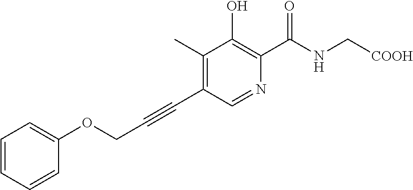 [SMILES: Cc1c(C#CCOc2ccccc2)cnc(C(=O)NCC(=O)O)c1O] |
| 54 | 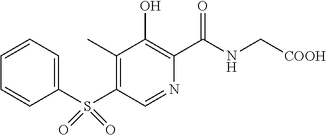 [SMILES: Cc1c(S(=O)(=O)c2ccccc2)cnc(C(=O)NCC(=O)O)c1O] |
| 55 | 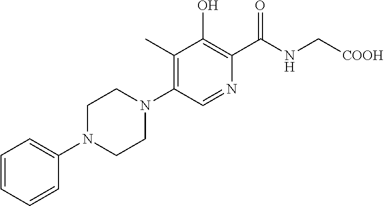 [SMILES: Cc1c(N2CCN(c3ccccc3)CC2)cnc(C(=O)NCC(=O)O)c1O] |
| 56 | 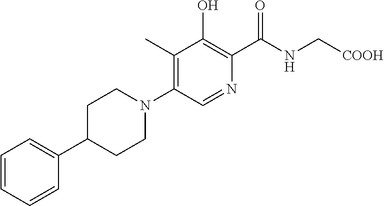 [SMILES: Cc1c(N2CCC(c3ccccc3)CC2)cnc(C(=O)NCC(=O)O)c1O] |
| 57 | 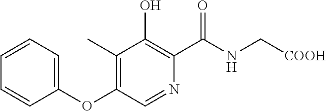 [SMILES: Cc1c(Oc2ccccc2)cnc(C(=O)NCC(=O)O)c1O] |
| 58 | 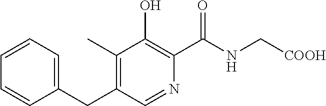 [SMILES: Cc1c(Cc2ccccc2)cnc(C(=O)NCC(=O)O)c1O] |
| 59 | 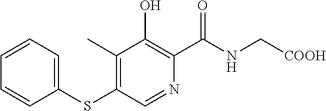 [SMILES: Cc1c(Sc2ccccc2)cnc(C(=O)NCC(=O)O)c1O] |
| 60 | 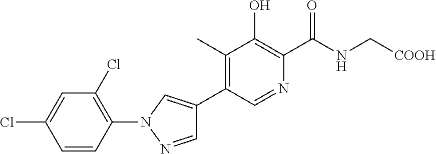 [SMILES: Cc1c(-c2cnn(-c3ccc(Cl)cc3Cl)c2)cnc(C(=O)NCC(=O)O)c1O] |
| 61 | 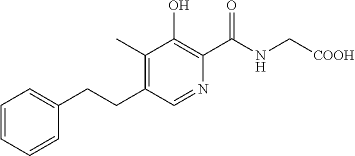 [SMILES: Cc1c(CCc2ccccc2)cnc(C(=O)NCC(=O)O)c1O] |
| 62 | 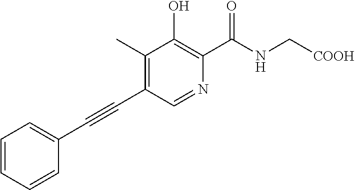 [SMILES: Cc1c(C#Cc2ccccc2)cnc(C(=O)NCC(=O)O)c1O] |
| 63 | 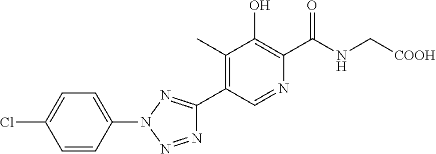 [SMILES: Cc1c(-c2nnn(-c3ccc(Cl)cc3)n2)cnc(C(=O)NCC(=O)O)c1O] |
| 64 | 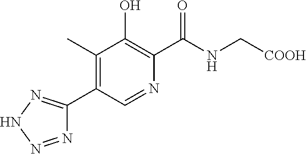 [SMILES: Cc1c(-c2nn[nH]n2)cnc(C(=O)NCC(=O)O)c1O] |
| 65 | 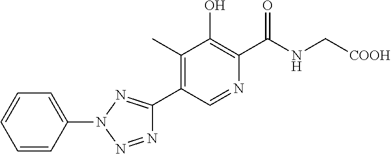 [SMILES: Cc1c(-c2nnn(-c3ccccc3)n2)cnc(C(=O)NCC(=O)O)c1O] |
| 66 | 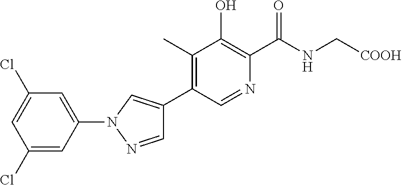 [SMILES: Cc1c(-c2cnn(-c3cc(Cl)cc(Cl)c3)c2)cnc(C(=O)NCC(=O)O)c1O] |
| 67 | 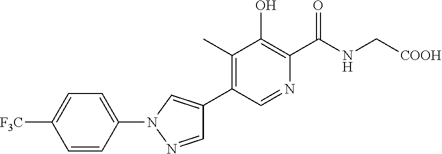 [SMILES: Cc1c(-c2cnn(-c3ccc(C(F)(F)F)cc3)c2)cnc(C(=O)NCC(=O)O)c1O] |
| 68 | 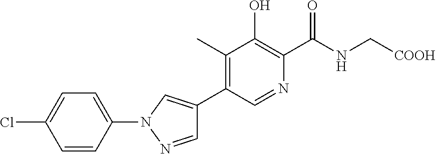 [SMILES: Cc1c(-c2cnn(-c3ccc(Cl)cc3)c2)cnc(C(=O)NCC(=O)O)c1O] |
| 69 | 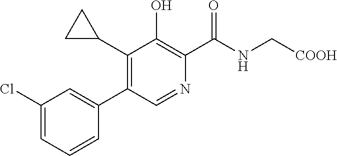 [SMILES: O=C(O)CNC(=O)c1ncc(-c2cccc(Cl)c2)c(C2CC2)c1O] |
| 70 | 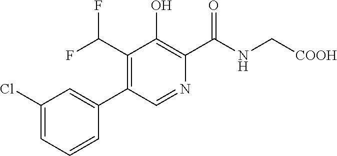 [SMILES: O=C(O)CNC(=O)c1ncc(-c2cccc(Cl)c2)c(C(F)F)c1O] |

or a pharmaceutically acceptable salt thereof.

63. The compound of any one of claims 1-62, or a pharmaceutically acceptable salt thereof, wherein at least one hydrogen atom is replaced with a deuterium atom.

64. A pharmaceutical composition comprising the compound of any one of claims 1-62, or a pharmaceutically acceptable salt thereof, and a pharmaceutically acceptable excipient.

65. A method for treating a disease mediated by PHD activity comprising administering to a subject the compound of any one of claims 1-62, or a pharmaceutically acceptable salt thereof.

66. The method of claim 65, wherein the disease mediated by PHD activity is an ischemic reperfusion injury.

67. The method of claim 66, wherein the ischemic reperfusion injury is selected from stroke, myocardial infarction, and acute kidney injury.

68. The method of claim 65, wherein the disease mediated by PHD activity is inflammatory bowel disease.

69. The method of claim 68, wherein the inflammatory bowel disease is ulcerative colitis.

70. The method of claim 68, wherein the inflammatory bowel disease is Crohn's disease.

71. The method of claim 65, wherein the disease mediated by PHD activity is cancer.

72. The method of claim 71, wherein the cancer is colorectal cancer.

73. The method of claim 65, wherein the disease mediated by PHD activity is liver disease.

74. The method of claim 65, wherein the disease mediated by PHD activity is atherosclerosis.

75. The method of claim 65, wherein the disease mediated by PHD activity is cardiovascular disease.

76. The method of claim 65, wherein the disease mediated by PHD activity is a disease or condition of the eye.

77. The method of claim 76, wherein the disease or condition of the eye is selected from radiation retinopathy, retinopathy of prematurity, diabetic retinopathy, age-related macular degeneration, and ocular ischemia.

78. The method of claim 65, wherein the disease is anemia.

79. The method of claim 78, wherein the anemia is anemia associated with chronic kidney disease.

80. The method of claim 65, wherein the disease is chronic kidney disease.

81. The method of claim 65, wherein the disease is associated with hyperoxia.

82. The method of claim 81, wherein the disease is retinopathy of prematurity.

83. The method of claim 81, wherein the disease is bronchopulmonary dysplasia (BPD).

84. The method of claim 65, wherein the disease is selected from ischemic heart disease, valvular heart disease, congestive heart failure, acute lung injury, pulmonary fibrosis, pulmonary hypertension, chronic obstructive pulmonary disease (COPD), acute liver failure, liver fibrosis, and cirrhosis.

85. The method of claim 65, wherein the disease is a respiratory disease, a lung disease, a respiratory viral infection, or a pulmonary viral infection.

86. The method of claim 85, wherein the respiratory disease is selected from respiratory infection, acute respiratory distress syndrome, lung inflammation, pneumonia, and acute lung injury.

87. The method of claim 85, wherein the lung disease is acute lung injury (ALI), bronchitis, pneumonia, pulmonary fibrosis, asthma, or acute respiratory distress syndrome (ARDS).

88. The method of claim 65, wherein the disease is injury to and/or failure of one or more organs.

89. The method of claim 88, wherein the disease is acute organ injury.

90. The method of claim 88, wherein the disease is organ failure.
